SEQUENCE LISTING (63 sequences)

SEQ ID NO: 1 - DNA, 812 bp, synthetic construct
  misc_feature 1..812: note = CBH promoter sequence
  source 1..812: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 2 - DNA, 6846 bp, Homo sapiens
  source 1..6846: mol_type = other DNA
aagctgttcctgcagccgatatcaggatgtgccgaaatgaaacggacaaggcaactgttaacattatagaccccagagttttaacacaggtcctctgatgacaaggttgtgatttttctctgtcttttgtcagcagcatctagctcaccgctgccaacacgacttccactgtactcttgatcaatttaccttgatgcactaccggtgaagaacggggactcgaattcccttacaaacgcctccagcttgtagaggcggtcgtggaggacccagaggaggagacgaaggggaaggaggcggtggtggaggaggcaaaggccttggacgaccattgttggcgaggggcaccactccgggagaggcggcgctgggcgtcttgggggtgcgcgccgggagcctgcagcgggaccagcgtgggaacgcggctggcaggctgtggacctcgtcctcaccaccatggtcgggctccttttgttttttttcccagcgatctttttggaggtgtcccttctccccagaagccccggcaggaaagtgttgctggcaggagcgtcgtctcagcgctcggtggccagaatggacggagatgtcatcattggagccctcttctcagtccatcaccagcctccggccgagaaagtgcccgagaggaagtgtggggagatcagggagcagtatggcatccagagggtggaggccatgttccacacgttggataagatcaacgcggacccggtcctcctgcccaacatcaccctgggcagtgagatccgggactcctgctggcactcttccgtggctctggaacagagcattgagttcattagggactctctgatttccattcgagatgagaaggatgggatcaaccggtgtctgcctgacggccagtccctccccccaggcaggactaagaagcccattgcgggagtgatcggtcccggctccagctctgtagccattcaagtgcagaacctgctccagctcttcgacatcccccagatcgcttattcagccacaagcatcgacctgagtgacaaaactttgtacaaatacttcctgagggttgtcccttctgacactttgcaggcaagggccatgcttgacatagtcaaacgttacaattggacctatgtctctgcagtccacacggaagggaattatggggagagcggaatggacgctttcaaagagctggctgcccaggaaggcctctgtatcgcccattctgacaaaatctacagcaacgctggggagaagagctttgaccgactcttgcgcaaactccgagagaggcttcccaaggctagagtggtggtctgcttctgtgaaggcatgacagtgcgaggactcctgagcgccatgcggcgccttggcgtcgtgggcgagttctcactcattggaagtgatggatgggcagacagagatgaagtcattgaaggttatgaggtggaagccaacgggggaatcacgataaagctgcagtctccagaggtcaggtcatttgatgattatttcctgaaactgaggctggacactaacacgaggaatccctggttccctgagttctggcaacatcggttccagtgccgccttccaggacaccttctggaaaatcccaactttaaacgaatctgcacaggcaatgaaagcttagaagaaaactatgtccaggacagtaagatggggtttgtcatcaatgccatctatgccatggcacatgggctgcagaacatgcaccatgccctctgccctggccacgtgggcctctgcgatgccatgaagcccatcgacggcagcaagctgctggacttcctcatcaagtcctcattcattggagtatctggagaggaggtgtggtttgatgagaaaggagacgctcctggaaggtatgatatcatgaatctgcagtacactgaagctaatcgctatgactatgtgcacgttggaacctggcatgaaggagtgctgaacattgatgattacaaaatccagatgaacaagagtggagtggtgcggtctgtgtgcagtgagccttgcttaaagggccagattaaggttatacggaaaggagaagtgagctgctgctggatttgcacggcctgcaaagagaatgaatatgtgcaagatgagttcacctgcaaagcttgtgacttgggatggtggcccaatgcagatctaacaggctgtgagcccattcctgtgcgctatcttgagtggagcaacatcgaatccattatagccatcgccttttcatgcctgggaatccttgttaccttgtttgtcaccctaatctttgtactgtaccgggacacaccagtggtcaaatcctccagtcgggagctctgctacatcatcctagctggcatcttccttggttatgtgtgcccattcactctcattgccaaacctactaccacctcctgctacctccagcgcctcttggttggcctctcctctgcgatgtgctactctgctttagtgactaaaaccaatcgtattgcacgcatcctggctggcagcaagaagaagatctgcacccggaagcccaggttcatgagtgcctgggctcaggtgatcattgcctcaattctgattagtgtgcaactaaccctggtggtaaccctgatcatcatggaaccccctatgcccattctgtcctacccaagtatcaaggaagtctaccttatctgcaataccagcaacctgggtgtggtggcccctttgggctacaatggactcctcatcatgagctgtacctactatgccttcaagacccgcaacgtgcccgccaacttcaacgaggccaaatatatcgcgttcaccatgtacaccacctgtatcatctggctagcttttgtgcccatttactttgggagcaactacaagatcatcacaacttgctttgcagtgagtctcagtgtaacagtggctctggggtgcatgttcactcccaagatgtacatcattattgccaagcctgagaggaatgtccgcagtgccttcaccacctctgatgttgtccgcatgcatgttggcgatggcaagctgccctgccgctccaacactttcctcaacatcttccgaagaaagaaggcaggggcagggaatgccaattctaatggcaagtctgtgtcatggtctgaaccaggtggaggacaggtgcccaagggacagcatatgtggcaccgcctctctgtgcacgtgaagaccaatgagacggcctgcaaccaaacagccgtcatcaagcccctcactaaaagttaccaaggctctggcaagagcctgaccttttcagataccagcaccaagaccctttacaacgtagaggaggaggaggatgcccagccgattcgctttagcccgcctggtagcccttccatggtggtgcacaggcgcgtgccaagcgcggcgaccactccgcctctgccgtcccacctgaccgcagaggagacccccctcttcctggccgaaccagccctccccaagggcttgccccctcctctccagcagcagcagcaaccccctccacagcagaaatcgctgatggaccagctccagggagtggtcagcaacttcagtaccgcgatcccggattttcacgcggtgctggcaggccccggtggtcccgggaacgggctgcggtccctgtacccgcccccgccacctccgcagcacctgcagatgctgccgctgcagctgagcacctttggggaggagctggtctccccgcccgcggacgacgacgacgacagcgagaggtttaagctcctccaggagtacgtgtatgagcacgagcgggaagggaacacggaagaagacgaactggaagaggaggaggaggacctgcaggcggccagcaaactgaccccggatgattcgcctgcgctgacgcctccgtcgcctttccgcgactcggtggcctcgggcagctcggtgcccagctcccccgtgtccgagtcggtgctctgcacccctcccaacgtatcctacgcctctgtcattctgcgggactacaagcaaagctcttccaccctgtaagggggaagggtccacatagaaaagcaagacaagccagagatctcccacacctccagagatgtgcaaacagctgggaggaaaagcctgggagtggggggcctcgtcgggaggacaggagaccgctgctgctgctgccgctactgctgctgctgccttaagtaggaagagagggaaggacaccaagcaaaaaatgttccaggccaggattcggattcttgaattactcgaagccttctctgggaagaaagggaattctgacaaagcacaattccatatggtatgtaacttttatcacaaatcaaatagtgacatcacaaacataatgtcctcttttgcacaattgtgcatagatatatatatgcccacacacactgggccatgcttgccaaggaacagcccacgtggacatgccagtcggatcatgagttcacctgatggcattcggagtgagctggtggagccagacagagcaggtgcggggaagggaagggcccaggccagacccatcccaaacggatgatgggatgatgggacagcagctccttgctcagaagcccttctccccgctgggctgacagactcctcatcttcaggagactcaggaatggagcggcacaggggtctctcttcatccactgcaacccatccagtgccagctttgagattgcacttgaagaaaggtgcatggaccccctgctgctctgcagattccctttatttaggaaaacaggaataagagcaaaattatcaccaaaaagtgcttcatcaggcgtgctacaggaggaaggagctagaaatagaacaatccatcagcatgagactttgaaaaaaaaacacatgatcagcttctcatgttccatattcacttattggcgatttggggaaaaggccggaacaagagattgttacgagagtggcagaaacccttttgtagattgacttgtgtttgtgccaagcgggctttccattgaccttcagttaaagaacaaaccatgtgacaaaattgttaccttccacttactgtagcaaataatacctacaagttgaacttctaagatgcgtatatgtacaatttggtgccattatttctcctacgtattagagaaacaaatccatctttgaatctaatggtgtactcatagcaactattactggtttaaatgacaaataattctatcctattgtcactgaagtccttgtaactagcgagtgaatgtgttcctgtgtccttgtatatgtgcgatcgtaaaatttgtgcaatgtaatgtcaaattgactggtcaatgtcaacctagtagtcaatctaactgcaattagaaattgtcttttgaatatactatatatattttttatgttccaataatgttttgtacatcattgtcatcaatatctacagaagctctttgacggtttgaatactatggctcaaggttttcatatgcagctcggatggacatttttcttctaagatggaacttatttttcagatattttctgatgtggagatatgttattaatgaagtggtttgaaaatttgttatattaaaagtgcacaaaaactgagagtgaaaataaaaggtacattttataagcttgcacacattattaacacataagattgaacaaagcatttagattattccaggttatatcatttttttaaagattttccacagctacttgagtgtctaacatacagtaacatctaactcagctaataatttgtaaaatctttatcaatcacattttgccttcttttaatttttatgttcatggacttttattcctgtgtcttggctgtcataactttttatttctgctatttgctgttgtgtaatatccatggacatgtaatccacttactccatctttacaatccctttttaccaccaataaaaggatttttcttgctgttttgatttcttctattatttgtggaatgaattataccccccttaaatatctttgtttatgccttatgttcagtcatattttaatatgcttccttcatattgaagctgctgatttctcagccaaaaatcatcttagaatctttaaatatccattgcatcatttgttcagaatttaacatccattccaatgttggaggcttgtattacttatatttcatcatattctattgccaagtttagtcagttccacaccaagaatgaactgcatttcctttaaaaattattttaaaacacctttattgaaaagatctcatgactgagatgtggactttggttccatgttttcattgtaagaaagcagagagcggaaaatcaatggctccagtgattaatagatgggtttttagtaattgacaaattcatgagggaaagcatatgatctctttattagtgaatcatgcttattttttactcttaatgccactaatatacatccctaatatcacagggcttgtgcattcagatttttaaaaaattaggatagataaggaaacaacttatattcaagtgtaagatgatatcaggttggtctaagacttttggtgaacacgttcattcaactgtgatcactttattactctgaatgcctactattatcctgattatggggtctcctgaataaatagagtattagtccttatgtcatcattgttcaaaattggagatgtacacatacataccctataccaagagggccgaaactcttcaccttgatgtatgttctgatacaagttgttcagcttcttgtaaatgtgttttccttcggcttgttactgccttttgtcaaataatcttgacaatgctgtataataaatattttctatttattaaa

SEQ ID NO: 3 - protein, 1194 aa, Homo sapiens
MVGLLLFFFPAIFLEVSLLPRSPGRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGINRCLPDGQSLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKRICTGNESLEENYVQDSKMGFVINAIYAMAHGLQNMHHALCPGHVGLCDAMKPIDGSKLLDFLIKSSFIGVSGEEVWFDEKGDAPGRYDIMNLQYTEANRYDYVHVGTWHEGVLNIDDYKIQMNKSGVVRSVCSEPCLKGQIKVIRKGEVSCCWICTACKENEYVQDEFTCKACDLGWWPNADLTGCEPIPVRYLEWSNIESIIAIAFSCLGILVTLFVTLIFVLYRDTPVVKSSSRELCYIILAGIFLGYVCPFTLIAKPTTTSCYLQRLLVGLSSAMCYSALVTKTNRIARILAGSKKKICTRKPRFMSAWAQVIIASILISVQLTLVVTLIIMEPPMPILSYPSIKEVYLICNTSNLGVVAPLGYNGLLIMSCTYYAFKTRNVPANFNEAKYIAFTMYTTCIIWLAFVPIYFGSNYKIITTCFAVSLSVTVALGCMFTPKMYIIIAKPERNVRSAFTTSDVVRMHVGDGKLPCRSNTFLNIFRRKKAGAGNANSNGKSVSWSEPGGGQVPKGQHMWHRLSVHVKTNETACNQTAVIKPLTKSYQGSGKSLTFSDTSTKTLYNVEEEEDAQPIRFSPPGSPSMVVHRRVPSAATTPPLPSHLTAEETPLFLAEPALPKGLPPPLQQQQQPPPQQKSLMDQLQGVVSNFSTAIPDFHAVLAGPGGPGNGLRSLYPPPPPPQHLQMLPLQLSTFGEELVSPPADDDDDSERFKLLQEYVYEHEREGNTEEDELEEEEEDLQAASKLTPDDSPALTPPSPFRDSVASGSSVPSSPVSESVLCTPPNVSYASVILRDYKQSSSTL

SEQ ID NO: 4 - DNA, 6917 bp, Homo sapiens
  source 1..6917: mol_type = other DNA
agagctgaggcgtctgcaagccgagcgtggccacggtcctctggccccgggaccatagcgctgtctaccccgactcaggtactcaggtatgtctcaagtccatgtcctccaaacagactcagcatctagctcaccgctgccaacacgacttccactgtactcttgatcaatttaccttgatgcactaccggtgaagaacggggactcgaattcccttacaaacgcctccagcttgtagaggcggtcgtggaggacccagaggaggagacgaaggggaaggaggcggtggtggaggaggcaaaggccttggacgaccattgttggcgaggggcaccactccgggagaggcggcgctgggcgtcttgggggtgcgcgccgggagcctgcagcgggaccagcgtgggaacgcggctggcaggctgtggacctcgtcctcaccaccatggtcgggctccttttgttttttttcccagcgatctttttggaggtgtcccttctccccagaagccccggcaggaaagtgttgctggcaggagcgtcgtctcagcgctcggtggccagaatggacggagatgtcatcattggagccctcttctcagtccatcaccagcctccggccgagaaagtgcccgagaggaagtgtggggagatcagggagcagtatggcatccagagggtggaggccatgttccacacgttggataagatcaacgcggacccggtcctcctgcccaacatcaccctgggcagtgagatccgggactcctgctggcactcttccgtggctctggaacagagcattgagttcattagggactctctgatttccattcgagatgagaaggatgggatcaaccggtgtctgcctgacggccagtccctccccccaggcaggactaagaagcccattgcgggagtgatcggtcccggctccagctctgtagccattcaagtgcagaacctgctccagctcttcgacatcccccagatcgcttattcagccacaagcatcgacctgagtgacaaaactttgtacaaatacttcctgagggttgtcccttctgacactttgcaggcaagggccatgcttgacatagtcaaacgttacaattggacctatgtctctgcagtccacacggaagggaattatggggagagcggaatggacgctttcaaagagctggctgcccaggaaggcctctgtatcgcccattctgacaaaatctacagcaacgctggggagaagagctttgaccgactcttgcgcaaactccgagagaggcttcccaaggctagagtggtggtctgcttctgtgaaggcatgacagtgcgaggactcctgagcgccatgcggcgccttggcgtcgtgggcgagttctcactcattggaagtgatggatgggcagacagagatgaagtcattgaaggttatgaggtggaagccaacgggggaatcacgataaagctgcagtctccagaggtcaggtcatttgatgattatttcctgaaactgaggctggacactaacacgaggaatccctggttccctgagttctggcaacatcggttccagtgccgccttccaggacaccttctggaaaatcccaactttaaacgaatctgcacaggcaatgaaagcttagaagaaaactatgtccaggacagtaagatggggtttgtcatcaatgccatctatgccatggcacatgggctgcagaacatgcaccatgccctctgccctggccacgtgggcctctgcgatgccatgaagcccatcgacggcagcaagctgctggacttcctcatcaagtcctcattcattggagtatctggagaggaggtgtggtttgatgagaaaggagacgctcctggaaggtatgatatcatgaatctgcagtacactgaagctaatcgctatgactatgtgcacgttggaacctggcatgaaggagtgctgaacattgatgattacaaaatccagatgaacaagagtggagtggtgcggtctgtgtgcagtgagccttgcttaaagggccagattaaggttatacggaaaggagaagtgagctgctgctggatttgcacggcctgcaaagagaatgaatatgtgcaagatgagttcacctgcaaagcttgtgacttgggatggtggcccaatgcagatctaacaggctgtgagcccattcctgtgcgctatcttgagtggagcaacatcgaatccattatagccatcgccttttcatgcctgggaatccttgttaccttgtttgtcaccctaatctttgtactgtaccgggacacaccagtggtcaaatcctccagtcgggagctctgctacatcatcctagctggcatcttccttggttatgtgtgcccattcactctcattgccaaacctactaccacctcctgctacctccagcgcctcttggttggcctctcctctgcgatgtgctactctgctttagtgactaaaaccaatcgtattgcacgcatcctggctggcagcaagaagaagatctgcacccggaagcccaggttcatgagtgcctgggctcaggtgatcattgcctcaattctgattagtgtgcaactaaccctggtggtaaccctgatcatcatggaaccccctatgcccattctgtcctacccaagtatcaaggaagtctaccttatctgcaataccagcaacctgggtgtggtggcccctttgggctacaatggactcctcatcatgagctgtacctactatgccttcaagacccgcaacgtgcccgccaacttcaacgaggccaaatatatcgcgttcaccatgtacaccacctgtatcatctggctagcttttgtgcccatttactttgggagcaactacaagatcatcacaacttgctttgcagtgagtctcagtgtaacagtggctctggggtgcatgttcactcccaagatgtacatcattattgccaagcctgagaggaatgtccgcagtgccttcaccacctctgatgttgtccgcatgcatgttggcgatggcaagctgccctgccgctccaacactttcctcaacatcttccgaagaaagaaggcaggggcagggaatgccaagaagaggcagccagaattctcgcccaccagccaatgtccgtcggcacatgtgcagctttgaaaacccccacactgcagtgaatgtttctaatggcaagtctgtgtcatggtctgaaccaggtggaggacaggtgcccaagggacagcatatgtggcaccgcctctctgtgcacgtgaagaccaatgagacggcctgcaaccaaacagccgtcatcaagcccctcactaaaagttaccaaggctctggcaagagcctgaccttttcagataccagcaccaagaccctttacaacgtagaggaggaggaggatgcccagccgattcgctttagcccgcctggtagcccttccatggtggtgcacaggcgcgtgccaagcgcggcgaccactccgcctctgccgtcccacctgaccgcagaggagacccccctcttcctggccgaaccagccctccccaagggcttgccccctcctctccagcagcagcagcaaccccctccacagcagaaatcgctgatggaccagctccagggagtggtcagcaacttcagtaccgcgatcccggattttcacgcggtgctggcaggccccggtggtcccgggaacgggctgcggtccctgtacccgcccccgccacctccgcagcacctgcagatgctgccgctgcagctgagcacctttggggaggagctggtctccccgcccgcggacgacgacgacgacagcgagaggtttaagctcctccaggagtacgtgtatgagcacgagcgggaagggaacacggaagaagacgaactggaagaggaggaggaggacctgcaggcggccagcaaactgaccccggatgattcgcctgcgctgacgcctccgtcgcctttccgcgactcggtggcctcgggcagctcggtgcccagctcccccgtgtccgagtcggtgctctgcacccctcccaacgtatcctacgcctctgtcattctgcgggactacaagcaaagctcttccaccctgtaagggggaagggtccacatagaaaagcaagacaagccagagatctcccacacctccagagatgtgcaaacagctgggaggaaaagcctgggagtggggggcctcgtcgggaggacaggagaccgctgctgctgctgccgctactgctgctgctgccttaagtaggaagagagggaaggacaccaagcaaaaaatgttccaggccaggattcggattcttgaattactcgaagccttctctgggaagaaagggaattctgacaaagcacaattccatatggtatgtaacttttatcacaaatcaaatagtgacatcacaaacataatgtcctcttttgcacaattgtgcatagatatatatatgcccacacacactgggccatgcttgccaaggaacagcccacgtggacatgccagtcggatcatgagttcacctgatggcattcggagtgagctggtggagccagacagagcaggtgcggggaagggaagggcccaggccagacccatcccaaacggatgatgggatgatgggacagcagctccttgctcagaagcccttctccccgctgggctgacagactcctcatcttcaggagactcaggaatggagcggcacaggggtctctcttcatccactgcaacccatccagtgccagctttgagattgcacttgaagaaaggtgcatggaccccctgctgctctgcagattccctttatttaggaaaacaggaataagagcaaaattatcaccaaaaagtgcttcatcaggcgtgctacaggaggaaggagctagaaatagaacaatccatcagcatgagactttgaaaaaaaaacacatgatcagcttctcatgttccatattcacttattggcgatttggggaaaaggccggaacaagagattgttacgagagtggcagaaacccttttgtagattgacttgtgtttgtgccaagcgggctttccattgaccttcagttaaagaacaaaccatgtgacaaaattgttaccttccacttactgtagcaaataatacctacaagttgaacttctaagatgcgtatatgtacaatttggtgccattatttctcctacgtattagagaaacaaatccatctttgaatctaatggtgtactcatagcaactattactggtttaaatgacaaataattctatcctattgtcactgaagtccttgtaactagcgagtgaatgtgttcctgtgtccttgtatatgtgcgatcgtaaaatttgtgcaatgtaatgtcaaattgactggtcaatgtcaacctagtagtcaatctaactgcaattagaaattgtcttttgaatatactatatatattttttatgttccaataatgttttgtacatcattgtcatcaatatctacagaagctctttgacggtttgaatactatggctcaaggttttcatatgcagctcggatggacatttttcttctaagatggaacttatttttcagatattttctgatgtggagatatgttattaatgaagtggtttgaaaatttgttatattaaaagtgcacaaaaactgagagtgaaaataaaaggtacattttataagcttgcacacattattaacacataagattgaacaaagcatttagattattccaggttatatcatttttttaaagattttccacagctacttgagtgtctaacatacagtaacatctaactcagctaataatttgtaaaatctttatcaatcacattttgccttcttttaatttttatgttcatggacttttattcctgtgtcttggctgtcataactttttatttctgctatttgctgttgtgtaatatccatggacatgtaatccacttactccatctttacaatccctttttaccaccaataaaaggatttttcttgctgttttgatttcttctattatttgtggaatgaattataccccccttaaatatctttgtttatgccttatgttcagtcatattttaatatgcttccttcatattgaagctgctgatttctcagccaaaaatcatcttagaatctttaaatatccattgcatcatttgttcagaatttaacatccattccaatgttggaggcttgtattacttatatttcatcatattctattgccaagtttagtcagttccacaccaagaatgaactgcatttcctttaaaaattattttaaaacacctttattgaaaagatctcatgactgagatgtggactttggttccatgttttcattgtaagaaagcagagagcggaaaatcaatggctccagtgattaatagatgggtttttagtaattgacaaattcatgagggaaagcatatgatctctttattagtgaatcatgcttattttttactcttaatgccactaatatacatccctaatatcacagggcttgtgcattcagatttttaaaaaattaggatagataaggaaacaacttatattcaagtgtaagatgatatcaggttggtctaagacttttggtgaacacgttcattcaactgtgatcactttattactctgaatgcctactattatcctgattatggggtctcctgaataaatagagtattagtccttatgtcatcattgttcaaaattggagatgtacacatacataccctataccaagagggccgaaactcttcaccttgatgtatgttctgatacaagttgttcagcttcttgtaaatgtgttttccttcggcttgttactgccttttgtcaaataatcttgacaatgctgtataataaatattttctatttattaaa

SEQ ID NO: 5 - protein, 906 aa, Homo sapiens
MVGLLLFFFPAIFLEVSLLPRSPGRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGINRCLPDGQSLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKRICTGNESLEENYVQDSKMGFVINAIYAMAHGLQNMHHALCPGHVGLCDAMKPIDGSKLLDFLIKSSFIGVSGEEVWFDEKGDAPGRYDIMNLQYTEANRYDYVHVGTWHEGVLNIDDYKIQMNKSGVVRSVCSEPCLKGQIKVIRKGEVSCCWICTACKENEYVQDEFTCKACDLGWWPNADLTGCEPIPVRYLEWSNIESIIAIAFSCLGILVTLFVTLIFVLYRDTPVVKSSSRELCYIILAGIFLGYVCPFTLIAKPTTTSCYLQRLLVGLSSAMCYSALVTKTNRIARILAGSKKKICTRKPRFMSAWAQVIIASILISVQLTLVVTLIIMEPPMPILSYPSIKEVYLICNTSNLGVVAPLGYNGLLIMSCTYYAFKTRNVPANFNEAKYIAFTMYTTCIIWLAFVPIYFGSNYKIITTCFAVSLSVTVALGCMFTPKMYIIIAKPERNVRSAFTTSDVVRMHVGDGKLPCRSNTFLNIFRRKKAGAGNAKKRQPEFSPTSQCPSAHVQL

SEQ ID NO: 6 - DNA, 6672 bp, Homo sapiens
  source 1..6672: mol_type = other DNA
catctagctcaccgctgccaacacgacttccactgtactcttgatcaatttaccttgatgcactaccggtgaagaacggggactcgaattcccttacaaacgcctccagcttgtagaggcggtcgtggaggacccagaggaggagacgaaggggaaggaggcggtggtggaggaggcaaaggccttggacgaccattgttggcgaggggcaccactccgggagaggcggcgctgggcgtcttgggggtgcgcgccgggagcctgcagcgggaccagcgtgggaacgcggctggcaggctgtggacctcgtcctcaccaccatggtcgggctccttttgttttttttcccagcgatctttttggaggtgtcccttctccccagaagccccggcaggaaagtgttgctggcaggagcgtcgtctcagcgctcggtggccagaatggacggagatgtcatcattggagccctcttctcagtccatcaccagcctccggccgagaaagtgcccgagaggaagtgtggggagatcagggagcagtatggcatccagagggtggaggccatgttccacacgttggataagatcaacgcggacccggtcctcctgcccaacatcaccctgggcagtgagatccgggactcctgctggcactcttccgtggctctggaacagagcattgagttcattagggactctctgatttccattcgagatgagaaggatgggatcaaccggtgtctgcctgacggccagtccctccccccaggcaggactaagaagcccattgcgggagtgatcggtcccggctccagctctgtagccattcaagtgcagaacctgctccagctcttcgacatcccccagatcgcttattcagccacaagcatcgacctgagtgacaaaactttgtacaaatacttcctgagggttgtcccttctgacactttgcaggcaagggccatgcttgacatagtcaaacgttacaattggacctatgtctctgcagtccacacggaagggaattatggggagagcggaatggacgctttcaaagagctggctgcccaggaaggcctctgtatcgcccattctgacaaaatctacagcaacgctggggagaagagctttgaccgactcttgcgcaaactccgagagaggcttcccaaggctagagtggtggtctgcttctgtgaaggcatgacagtgcgaggactcctgagcgccatgcggcgccttggcgtcgtgggcgagttctcactcattggaagtgatggatgggcagacagagatgaagtcattgaaggttatgaggtggaagccaacgggggaatcacgataaagctgcagtctccagaggtcaggtcatttgatgattatttcctgaaactgaggctggacactaacacgaggaatccctggttccctgagttctggcaacatcggttccagtgccgccttccaggacaccttctggaaaatcccaactttaaacgaatctgcacaggcaatgaaagcttagaagaaaactatgtccaggacagtaagatggggtttgtcatcaatgccatctatgccatggcacatgggctgcagaacatgcaccatgccctctgccctggccacgtgggcctctgcgatgccatgaagcccatcgacggcagcaagctgctggacttcctcatcaagtcctcattcattggagtatctggagaggaggtgtggtttgatgagaaaggagacgctcctggaaggtatgatatcatgaatctgcagtacactgaagctaatcgctatgactatgtgcacgttggaacctggcatgaaggagtgctgaacattgatgattacaaaatccagatgaacaagagtggagtggtgcggtctgtgtgcagtgagccttgcttaaagggccagattaaggttatacggaaaggagaagtgagctgctgctggatttgcacggcctgcaaagagaatgaatatgtgcaagatgagttcacctgcaaagcttgtgacttgggatggtggcccaatgcagatctaacaggctgtgagcccattcctgtgcgctatcttgagtggagcaacatcgaatccattatagccatcgccttttcatgcctgggaatccttgttaccttgtttgtcaccctaatctttgtactgtaccgggacacaccagtggtcaaatcctccagtcgggagctctgctacatcatcctagctggcatcttccttggttatgtgtgcccattcactctcattgccaaacctactaccacctcctgctacctccagcgcctcttggttggcctctcctctgcgatgtgctactctgctttagtgactaaaaccaatcgtattgcacgcatcctggctggcagcaagaagaagatctgcacccggaagcccaggttcatgagtgcctgggctcaggtgatcattgcctcaattctgattagtgtgcaactaaccctggtggtaaccctgatcatcatggaaccccctatgcccattctgtcctacccaagtatcaaggaagtctaccttatctgcaataccagcaacctgggtgtggtggcccctttgggctacaatggactcctcatcatgagctgtacctactatgccttcaagacccgcaacgtgcccgccaacttcaacgaggccaaatatatcgcgttcaccatgtacaccacctgtatcatctggctagcttttgtgcccatttactttgggagcaactacaagatcatcacaacttgctttgcagtgagtctcagtgtaacagtggctctggggtgcatgttcactcccaagatgtacatcattattgccaagcctgagaggaatgtccgcagtgccttcaccacctctgatgttgtccgcatgcatgttggcgatggcaagctgccctgccgctccaacactttcctcaacatcttccgaagaaagaaggcaggggcagggaatgccaagtggaggacaggtgcccaagggacagcatatgtggcaccgcctctctgtgcacgtgaagaccaatgagacggcctgcaaccaaacagccgtcatcaagcccctcactaaaagttaccaaggctctggcaagagcctgaccttttcagataccagcaccaagaccctttacaacgtagaggaggaggaggatgcccagccgattcgctttagcccgcctggtagcccttccatggtggtgcacaggcgcgtgccaagcgcggcgaccactccgcctctgccgtcccacctgaccgcagaggagacccccctcttcctggccgaaccagccctccccaagggcttgccccctcctctccagcagcagcagcaaccccctccacagcagaaatcgctgatggaccagctccagggagtggtcagcaacttcagtaccgcgatcccggattttcacgcggtgctggcaggccccggtggtcccgggaacgggctgcggtccctgtacccgcccccgccacctccgcagcacctgcagatgctgccgctgcagctgagcacctttggggaggagctggtctccccgcccgcggacgacgacgacgacagcgagaggtttaagctcctccaggagtacgtgtatgagcacgagcgggaagggaacacggaagaagacgaactggaagaggaggaggaggacctgcaggcggccagcaaactgaccccggatgattcgcctgcgctgacgcctccgtcgcctttccgcgactcggtggcctcgggcagctcggtgcccagctcccccgtgtccgagtcggtgctctgcacccctcccaacgtatcctacgcctctgtcattctgcgggactacaagcaaagctcttccaccctgtaagggggaagggtccacatagaaaagcaagacaagccagagatctcccacacctccagagatgtgcaaacagctgggaggaaaagcctgggagtggggggcctcgtcgggaggacaggagaccgctgctgctgctgccgctactgctgctgctgccttaagtaggaagagagggaaggacaccaagcaaaaaatgttccaggccaggattcggattcttgaattactcgaagccttctctgggaagaaagggaattctgacaaagcacaattccatatggtatgtaacttttatcacaaatcaaatagtgacatcacaaacataatgtcctcttttgcacaattgtgcatagatatatatatgcccacacacactgggccatgcttgccaaggaacagcccacgtggacatgccagtcggatcatgagttcacctgatggcattcggagtgagctggtggagccagacagagcaggtgcggggaagggaagggcccaggccagacccatcccaaacggatgatgggatgatgggacagcagctccttgctcagaagcccttctccccgctgggctgacagactcctcatcttcaggagactcaggaatggagcggcacaggggtctctcttcatccactgcaacccatccagtgccagctttgagattgcacttgaagaaaggtgcatggaccccctgctgctctgcagattccctttatttaggaaaacaggaataagagcaaaattatcaccaaaaagtgcttcatcaggcgtgctacaggaggaaggagctagaaatagaacaatccatcagcatgagactttgaaaaaaaaacacatgatcagcttctcatgttccatattcacttattggcgatttggggaaaaggccggaacaagagattgttacgagagtggcagaaacccttttgtagattgacttgtgtttgtgccaagcgggctttccattgaccttcagttaaagaacaaaccatgtgacaaaattgttaccttccacttactgtagcaaataatacctacaagttgaacttctaagatgcgtatatgtacaatttggtgccattatttctcctacgtattagagaaacaaatccatctttgaatctaatggtgtactcatagcaactattactggtttaaatgacaaataattctatcctattgtcactgaagtccttgtaactagcgagtgaatgtgttcctgtgtccttgtatatgtgcgatcgtaaaatttgtgcaatgtaatgtcaaattgactggtcaatgtcaacctagtagtcaatctaactgcaattagaaattgtcttttgaatatactatatatattttttatgttccaataatgttttgtacatcattgtcatcaatatctacagaagctctttgacggtttgaatactatggctcaaggttttcatatgcagctcggatggacatttttcttctaagatggaacttatttttcagatattttctgatgtggagatatgttattaatgaagtggtttgaaaatttgttatattaaaagtgcacaaaaactgagagtgaaaataaaaggtacattttataagcttgcacacattattaacacataagattgaacaaagcatttagattattccaggttatatcatttttttaaagattttccacagctacttgagtgtctaacatacagtaacatctaactcagctaataatttgtaaaatctttatcaatcacattttgccttcttttaatttttatgttcatggacttttattcctgtgtcttggctgtcataactttttatttctgctatttgctgttgtgtaatatccatggacatgtaatccacttactccatctttacaatccctttttaccaccaataaaaggatttttcttgctgttttgatttcttctattatttgtggaatgaattataccccccttaaatatctttgtttatgccttatgttcagtcatattttaatatgcttccttcatattgaagctgctgatttctcagccaaaaatcatcttagaatctttaaatatccattgcatcatttgttcagaatttaacatccattccaatgttggaggcttgtattacttatatttcatcatattctattgccaagtttagtcagttccacaccaagaatgaactgcatttcctttaaaaattattttaaaacacctttattgaaaagatctcatgactgagatgtggactttggttccatgttttcattgtaagaaagcagagagcggaaaatcaatggctccagtgattaatagatgggtttttagtaattgacaaattcatgagggaaagcatatgatctctttattagtgaatcatgcttattttttactcttaatgccactaatatacatccctaatatcacagggcttgtgcattcagatttttaaaaaattaggatagataaggaaacaacttatattcaagtgtaagatgatatcaggttggtctaagacttttggtgaacacgttcattcaactgtgatcactttattactctgaatgcctactattatcctgattatggggtctcctgaataaatagagtattagtccttatgtcatcattgttcaaaattggagatgtacacatacataccctataccaagagggccgaaactcttcaccttgatgtatgttctgatacaagttgttcagcttcttgtaaatgtgttttccttcggcttgttactgccttttgtcaaataatcttgacaatgctgtataataaatattttctatttatt

SEQ ID NO: 7 - protein, 908 aa, Homo sapiens
MVGLLLFFFPAIFLEVSLLPRSPGRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGINRCLPDGQSLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKRICTGNESLEENYVQDSKMGFVINAIYAMAHGLQNMHHALCPGHVGLCDAMKPIDGSKLLDFLIKSSFIGVSGEEVWFDEKGDAPGRYDIMNLQYTEANRYDYVHVGTWHEGVLNIDDYKIQMNKSGVVRSVCSEPCLKGQIKVIRKGEVSCCWICTACKENEYVQDEFTCKACDLGWWPNADLTGCEPIPVRYLEWSNIESIIAIAFSCLGILVTLFVTLIFVLYRDTPVVKSSSRELCYIILAGIFLGYVCPFTLIAKPTTTSCYLQRLLVGLSSAMCYSALVTKTNRIARILAGSKKKICTRKPRFMSAWAQVIIASILISVQLTLVVTLIIMEPPMPILSYPSIKEVYLICNTSNLGVVAPLGYNGLLIMSCTYYAFKTRNVPANFNEAKYIAFTMYTTCIIWLAFVPIYFGSNYKIITTCFAVSLSVTVALGCMFTPKMYIIIAKPERNVRSAFTTSDVVRMHVGDGKLPCRSNTFLNIFRRKKAGAGNAKWRTGAQGTAYVAPPLCAREDQ

SEQ ID NO: 8 - DNA, 3356 bp, Homo sapiens
  source 1..3356: mol_type = other DNA
gagcgcagagccagcgagccagcgagcgagcgagcgggagccggagcctcgcgccccccgctccactccgattctctccgcgccagagccagcgcgccaggagctgggtcccttcgcatctctcttcttgtctgtcctttcctggtccctgtttcctcctctctttgccttcgctgcttctaatctcatcccctggagacccaggtctgcgggacccatccatcccctttggggccatgggatcgctgcttgcgctcctggcactgctgctgctgtggggtgctgtggctgagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatctcgggccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacaatgtgctcctcatcgcgctctgcacgctttatgccttcaagactcgcaagtgccccgaaaacttcaacgaggccaagttcattggcttcaccatgtacaccacctgcatcatctggctggcattcctgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctttgaagaccccatactcccgccctgacacagctgctcctgggaacctagtgcagacccacgtccagggccaggaggaagttggctggagcactgcaataatttattacccaccctatgtctgcccccaaagtcacttacccacctccttaccccagctcttcagactcagaagtcaggagccttggccaggagcctctgcagtggccactaactgcccttgtagctgtgtttcctcctggccaggcccagggctcagagaggagcaagccagggttcactctgccctggacccgggtggctgaggacggcaggccccagtcctaaccagcaaaggtgcttccagcccagcccctccccccaactagggccttttttattttttatataagttactctgggatggggagggtggttattgtgggggctgcccctccccctgcacagtagtttgtcctgtggtttattttgtattacctgtaaataaagtggctttattttaaaaaa

SEQ ID NO: 9 - protein, 872 aa, Homo sapiens [sequence omitted]

SEQ ID NO: 10 - DNA, 4260 bp, Homo sapiens
  source 1..4260: mol_type = other DNA
gtgcagttgagtcgcgagtacggctgagctgcgtaccggcctccctggctctcacactccctctctgctcccgctctcctaatctcctctggcatgcggtcagccccctgcccagggaccacaggagagttcttgtaaggactgttagtccctgcttacctgaaagccaagcgctctagcagagctttaaagttggagccgccaccctccctaccgccccatgccccttcaccccactccgaaattcaccgacctttgcatgcactgcctaaggatttcagagtgaggcaaagcagtcggcaaatctaccctggcttttcgtataaaaatcctctcgtctaggtaccctggctcactgaagactctgcagatatacccttataagagggagggtgggggagggaaaagaacgagagagggaggaaagaatgaaaaggagaggatgccaggaggtccgtgcttctgccaagagtcccaattagatgcgacggcttcagcctggtcaaggtgaaggaaagttgcttccgcgcctaggaagtgggtttgcctgataagagaaggaggaggggactcggctgggaagagctcccctcccctccgcggaagaccactgggtcccctctttccccaacctcctccctctcttctactccacccctccgttttcccactccccactgactcggatgcctggatgttctgccaccgggcagtggtccagcgtgcagccgggagggggcaggggcagggggcactgtgacaggaagctgcgcgcacaagttggccatttcgagggcaaaataagttctcccttggatttggaaaggacaaagccagtaagctacctcttttgtgtcggatgaggaggaccaaccatgagccagagcccgggtgcaggctcaccgccgccgctgccaccgcggtcagctccagttcctgccaggagttgtcggtgcgaggaattttgtgacaggctctgttagtctgttcctcccttatttgaaggacaggccaaagatccagtttggaaatgagagaggactagcatgacacattggctccaccattgatatctcccagaggtacagaaacaggattcatgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggctggggcgcgcaggagagcatcatcaagggcagcgagcatgtggcctacggcgccatcaccctggagctggcctcccagcctgtccgccagttcgaccgctacttccagagcctcaacccctacaacaaccaccgcaacccctggttccgggacttctgggagcaaaagtttcagtgcagcctccagaacaaacgcaaccacaggcgcgtctgcgacaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtggaaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatcctggtgcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacgatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtgattgtgaattgcagttcagttcttgtgtttttagactgttagacaaaagtgctcacgtgcagctccagaatatggaaacagagcaaaagaacaaccctagtacctttttttagaaacagtacgataaattatttttgaggactgtatatagtgatgtgctagaactttctaggctgagtctagtgcccctattattaacaattcccccagaacatggaaataaccattgtttacagagctgagcattggtgacagggtctgacatggtcagtctactaaaaaacaaaaaaaaaaaacaaaaaaaaaaaaacaaaagaaaaaaataaaaatacggtggcaatattatgtaaccttttttcctatgaagttttttgtaggtccttgttgtaactaatttaggatgagtttctatgttgtatattaaagttacattatgtgtaacagattgattttctcagcacaaaataaaaagcatctgtattaatgtaaagatactgagaataaaaccttcaaggttttcca

SEQ ID NO: 11 - protein, 879 aa, Homo sapiens
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGAITLELASQPVRQFDRYFQSLNPYNNHRNPWFRDFWEQKFQCSLQNKRNHRRVCDKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLILVQIVMVSVWLILEAPGTRRYTLAEKRETVILKCNVKDSSMLISLTYDVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSL

SEQ ID NO: 12 - DNA, 3201 bp, Homo sapiens
  source 1..3201: mol_type = other DNA
gcagtgtgcagttgagtcgcgagtacggctgagctgcgtaccggcctccctggctctcacactccctctctgctcccgctctcctaatctcctctggcatgcggtcagccccctgcccagggaccacaggagagttcttgtaaggactgttagtccctgcttacctgaaagccaagcgctctagcagagctttaaagttggagccgccaccctccctaccgccccatgccccttcaccccactccgaaattcaccgacctttgcatgcactgcctaaggatttcagagtgaggcaaagcagtcggcaaatctaccctggcttttcgtataaaaatcctctcgtctaggtaccctggctcactgaagactctgcagatatacccttataagagggagggtgggggagggaaaagaacgagagagggaggaaagaatgaaaaggagaggatgccaggaggtccgtgcttctgccaagagtcccaattagatgcgacggcttcagcctggtcaaggtgaaggaaagttgcttccgcgcctaggaagtgggtttgcctgataagagaaggaggaggggactcggctgggaagagctcccctcccctccgcggaagaccactgggtcccctctttccccaacctcctccctctcttctactccacccctccgttttcccactccccactgactcggatgcctggatgttctgccaccgggcagtggtccagcgtgcagccgggagggggcaggggcagggggcactgtgacaggaagctgcgcgcacaagttggccatttcgagggcaaaataagttctcccttggatttggaaaggacaaagccagtaagctacctcttttgtgtcggatgaggaggaccaaccatgagccagagcccgggtgcaggctcaccgccgccgctgccaccgcggtcagctccagttcctgccaggagttgtcggtgcgaggaattttgtgacaggctctgttagtctgttcctcccttatttgaaggacaggccaaagatccagtttggaaatgagagaggactagcatgacacattggctccaccattgatatctcccagaggtacagaaacaggattcatgaagatgttgacaagactgcaagttcttaccttagctttgttttcaaagggatttttactctctttaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggctggggcgcgcaggagagcatcatcaagggcagcgagcatgtggcctacggcgccatcaccctggagctggcctcccagcctgtccgccagttcgaccgctacttccagagcctcaacccctacaacaaccaccgcaacccctggttccgggacttctgggagcaaaagtttcagtgcagcctccagaacaaacgcaaccacaggcgcgtctgcgacaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacgggtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctgtgattgtgaattgcagttcagttcttgtgtttttagactgttagacaaaagtgctcacgtgcagctccagaatatggaaacagagcaaaagaacaaccctagtacctttttttagaaacagtacgataaattatttttgaggactgtatatagtgatgtgctagaactttctaggctgagtctagtgcccctattattaacaattcccccagaacatggaaataaccattgtttacagagctgagcattggtgacagggtctgacatggtcagtctactaaaaaacaaaaaaaaaaaacaaaaaaaaaaaaacaaaagaaaaaaataaaaatacggtggcaatattatgtaaccttttttcctatgaagttttttgtaggtccttgttgtaactaatttaggatgagtttctatgttgtatattaaagttacattatgtgtaacagattgattttctcagcacaaaataaaaagcatctgtattaatgtaaagatactgagaataaaaccttcaaggttttccagca

SEQ ID NO: 13 - protein, 537 aa, Homo sapiens
MKMLTRLQVLTLALFSKGFLLSLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGAITLELASQPVRQFDRYFQSLNPYNNHRNPWFRDFWEQKFQCSLQNKRNHRRVCDKHLAIDSSNYEQESKIMFVVNAVYAMAHALHKMQRTLCPNTTKLCDAMKILDGKKLYKDYLLKINFTGADDNHVHLCQPEWLCGLGLFVCTQGSHHPVSTPEECCHTQTAPQQVQCQWNWDHILSVLCKHVCANGVQWAGSPRLHHLISVIVNCSSVLVFLDC

SEQ ID NO: 14 - DNA, 7368 bp, Homo sapiens
  source 1..7368: mol_type = other DNA
gctgtacttttctgggtgtgtgttagggaggctatgttcctgaccctccccctctggggtgagaaggggtccccgccatgtcctcggggttggtaggaggagaggattggagctgttttctccttgatgccaagatacgccaagctaggagcattctgccctttccacagtcatccaccgagaacaggcctgcaggacgggacaaggatcagagccttcctgcaaccccggccactgcctgctgtctgtgggcctggactgtgcgggcaactgtgcttggcccgagtgacaaggaggtgggagagggtagcagcatgggctacgcggttggctgccctcagtccccctgctgctgaagctgccctgcccatgcccacccaggccgtggggccaggggcctgccagggctaggagtgggcctgccgttcatgggtctctagggatttccgagatgcctgggaagagaggcttgggctggtggtgggcccggctgcccctttgcctgctcctcagcctttacggcccctggatgccttcctccctgggaaagcccaaaggccaccctcacatgaattccatccgcatagatggggacatcacactgggaggcctgttcccggtgcatggccggggctcagagggcaagccctgtggagaacttaagaaggaaaagggcatccaccggctggaggccatgctgttcgccctggatcgcatcaacaacgacccggacctgctgcctaacatcacgctgggcgcccgcattctggacacctgctccagggacacccatgccctcgagcagtcgctgacctttgtgcaggcgctcatcgagaaggatggcacagaggtccgctgtggcagtggcggcccacccatcatcaccaagcctgaacgtgtggtgggtgtcatcggtgcttcagggagctcggtctccatcatggtggccaacatccttcgcctcttcaagataccccagatcagctacgcctccacagcgccagacctgagtgacaacagccgctacgacttcttctcccgcgtggtgccctcggacacgtaccaggcccaggccatggtggacatcgtccgtgccctcaagtggaactatgtgtccacagtggcctcggagggcagctatggtgagagcggtgtggaggccttcatccagaagtcccgtgaggacgggggcgtgtgcatcgcccagtcggtgaagataccacgggagcccaaggcaggcgagttcgacaagatcatccgccgcctcctggagacttcgaacgccagggcagtcatcatctttgccaacgaggatgacatcaggcgtgtgctggaggcagcacgaagggccaaccagacaggccatttcttctggatgggctctgacagctggggctccaagattgcacctgtgctgcacctggaggaggtggctgagggtgctgtcacgatcctccccaagaggatgtccgtacgaggcttcgaccgctacttctccagccgcacgctggacaacaaccggcgcaacatctggtttgccgagttctgggaggacaacttccactgcaagctgagccgccacgccctcaagaagggcagccacgtcaagaagtgcaccaaccgtgagcgaattgggcaggattcagcttatgagcaggaggggaaggtgcagtttgtgatcgatgccgtgtacgccatgggccacgcgctgcacgccatgcaccgtgacctgtgtcccggccgcgtggggctctgcccgcgcatggaccctgtagatggcacccagctgcttaagtacatccgaaacgtcaacttctcaggcatcgcagggaaccctgtgaccttcaatgagaatggagatgcgcctgggcgctatgacatctaccaataccagctgcgcaacgattctgccgagtacaaggtcattggctcctggactgaccacctgcaccttagaatagagcggatgcactggccggggagcgggcagcagctgccccgctccatctgcagcctgccctgccaaccgggtgagcggaagaagacagtgaagggcatgccttgctgctggcactgcgagccttgcacagggtaccagtaccaggtggaccgctacacctgtaagacgtgtccctatgacatgcggcccacagagaaccgcacgggctgccggcccatccccatcatcaagcttgagtggggctcgccctgggccgtgctgcccctcttcctggccgtggtgggcatcgctgccacgttgttcgtggtgatcacctttgtgcgctacaacgacacgcccatcgtcaaggcctcgggccgtgaactgagctacgtgctgctggcaggcatcttcctgtgctatgccaccaccttcctcatgatcgctgagcccgaccttggcacctgctcgctgcgccgaatcttcctgggactagggatgagcatcagctatgcagccctgctcaccaagaccaaccgcatctaccgcatcttcgagcagggcaagcgctcggtcagtgccccacgcttcatcagccccgcctcacagctggccatcaccttcagcctcatctcgctgcagctgctgggcatctgtgtgtggtttgtggtggacccctcccactcggtggtggacttccaggaccagcggacactcgacccccgcttcgccaggggtgtgctcaagtgtgacatctcggacctgtcgctcatctgcctgctgggctacagcatgctgctcatggtcacgtgcaccgtgtatgccatcaagacacgcggcgtgcccgagaccttcaatgaggccaagcccattggcttcaccatgtacaccacttgcatcgtctggctggccttcatccccatcttctttggcacctcgcagtcggccgacaagctgtacatccagacgacgacgctgacggtctcggtgagtctgagcgcctcggtgtccctgggaatgctctacatgcccaaagtctacatcatcctcttccacccggagcagaacgtgcccaagcgcaagcgcagcctcaaagccgtcgttacggcggccaccatgtccaacaagttcacgcagaagggcaacttccggcccaacggagaggccaagtctgagctctgcgagaaccttgaggccccagcgctggccaccaaacagacttacgtcacttacaccaaccatgcaatctagcgagtccatggagctgagcagcaggaggaggagccgtgaccctgtggaaggtgcgtcgggccagggccacacccaagggcccagctgtcttgcctgcccgtgggcacccacggacgtggcttggtgctgaggatagcagagcccccagccatcactgctggcagcctgggcaaaccgggtgagcaacaggaggacgaggggccggggcggtgccaggctaccacaagaacctgcgtcttggaccattgcccctcccggccccaaaccacaggggctcaggtcgtgtgggccccagtgctagatctctccctcccttcgtctctgtctgtgctgttggcgacccctctgtctgtctccagccctgtctttctgttctcttatctctttgtttcaccttttccctctctggcgtccccggctgcttgtactcttggccttttctgtgtctcctttctggctcttgcctccgcctctctctctcatcctctttgtcctcagctcctcctgctttcttgggtcccaccagtgtcacttttctgccgttttctttcctgttctcctctgcttcattctcgtccagccattgctcccctctccctgccacccttccccagttcaccaaaccttacatgttgcaaaagagaaaaaaggaaaaaaaatcaaaacacaaaaaagccaaaacgaaaacaaatctcgagtgtgttgccaagtgctgcgtcctcctggtggcctctgtgtgtgtccctgtggcccgcagcctgcccgcctgccccgcccatctgccgtgtgtcttgcccgcctgccccgcccgtctgccgtctgtcttgcccgcctgcccgcctgcccctcctgccgaccacacggagttcagtgcctgggtgtttggtgatggttattgacgacaatgtgtagcgcatgattgtttttataccaagaacatttctaataaaaataaacacatggttttgcacccgggctccacatccactgagggtcctgccatgggaccacaggctcagcctgcagctggagggcttagacctagagggaagcgggaactgggctctggagacccagggcttgggggctgtggagactgctccctaggctgggatctagtgtggtgtggtgaggccttgggcatggaggggccagattcccaggtaaggggcagggacattgcaggaaattccaggaatcagcacctagtagtcccctaattagggggtatgctctgtcccctgccctgcagccctgggagggtaacatttctgccttgcctgtcctctgtctcacacccctcacacctgggactgcccttccacccctgcccccataacctgtgcctctctccttccagccaggaagtcctcttcttgagaagttagcttcccgggctgccagcactcatagccgtcccctcctgcttgtgttggctccaggctcgggtgctaagaagatgtgtgtctgtcctggagatcagtgtgttgttatgtgtccacgtgggcccacaagtgcacggcacaggcatggccgtgtggctgtgttggctgtgttggctgtgtgtctgtgtgcacgtccagcgcctccatgcgcatgcgtgcctgtcttgtttgcgtgtctgatcatctgtttgggccccggtggctcatgcagatgcctgtctcaggcccatggcgagtgttcacctcagctggcttccctggcaggttgggaggtgggaaacaggagcgcttaggggctgggctctggctggggtaaattatagagccagaaacacaatgaggccataggcagcagctggagcctgggctgcctgtgccgtcccctcctgccctgcccctgggtcctgcaccccctcccacctccaggctagctgacagcgctatggagcacagtggaagggactggaggaaccctaggcagggggccacgcagggacagagtatgagagtgtgtgtataactgaggctgggacattgaatcatgccaggtatgtcttctccatcagcccactcttactcctggcctgggcatctcacacatctgtgcataggaaatctcttcttccctggggtctgtgtgcagcacctagtagatgctcaataaatgtttgtgtgagggaaggagacaggaaaggaagtgtctcgctgatcatcttgcggaatggttcctaagacctctgcccaggaaagattccacccagtgctccagcccggtcaggcagaactaggttgccagatcaagggtatctcccaaaagcttccagggcagttgggggtgggggggtggggggtagggatggggaatgcagaagcgggtgcagccagctctcccccagggtgactctggcagcacccccatcctgggcaccctgcctgctctgtggctcacgcccctcctgaagtgactgatgctctgaggcccaaggctaggtccagggcagggcctgcaggggtttcatgctcagtccaggacttgcctaggtccccctacatctgtggggcccccatctaggttctaacaggagaatcacctctccaagggggatgctgcccctcggctccccttggctctcaggaggggccctcagggactaccagtcccctgccagtgggaagaaatagccctgccctcagggagcttccagtgtgatgggggagatacagcagactgtgtcccaaagtaaaatgactgttagaatgaggtgggtggaggagggaagccttgggtgggtgtgactttgggcatctgagcctggggtgcagaggtgggctctgtgggcctgaggtggacaggagggaaccaggccctagcaagacttttgccagctagacctgctgcagcagttgggagggtgggtgctgctggagtcctgggtccatcacctagaaggctcaggccagtgcagccagggctggggcccacagctggcctgggtgggacctgccctgatgcccatggcaggagggacgcctggcccttcacaattggcttggctgctcacctttgctctcatcctcaattattaatgactggagaaagctgctaagtatcttcagaatgttagatttcaacaagatggggggttcagggtccctggcaccctggatagggagccagcggcccctagagacctttgctgtgtgcagggggtatgtgctcacccccgtggcctcagcctcctcaatgtctgaatgaaggattgggctagcagacatcccaccccacagcacactttctaaccagcaggggaacttctagacaatagagacgctgggctccctccagaacactggacctgaacttctggggggagggctgggcacgggcatattttaaaagctccccagcagatgggccgtgcagtcaagtgggccaagagtggcaccagactttggggcttgtgaagtcaggagggagcaacagtgcccactcgagcttgcctggggctcaagcccaaggctgggctgctgccagcctgagcagacacccaggagcttccaggccagctggatgcacagggcacctttgtggaactcctaggaccctggggagacccacctcaggagcagagtctcaggtcccttccggctctgaggggctgttctgagctctaatgtcttatggtctgcccctcccatccttacttctcaggccctggaggcagaggcatagagccaggcaggacagaggtctcagtgggccacatgccagctgcccccacactgcctcagcctccaggcctccaaggggtcctggggagcccctgagaagatgctgagcctgcataaggctgggcgcccctctttctgacaccctcactggctccacggctcccccttcccatcccaggtttccatctgcccactgaacagggaggggaaactgaggcactcccctggcactgagggctccttctgtcatcctgcctgccctggatggtcctggctgcccctcagggcttggccctggcactgtgagcctcacagggctcagacccccacccccaacccagcactaaatggcactcggcaccagaatctcacttcagttggcaaaagcagcaattagcatgtaatgaggcttcttgctttatttttaggtaacctccaaggccctgcctgtgtaattcagcccgccattgctcggtggataattaaagcatgtcaccataa

SEQ ID NO: 15 - protein, 912 aa, Homo sapiens
MPGKRGLGWWWARLPLCLLLSLYGPWMPSSLGKPKGHPHMNSIRIDGDITLGGLFPVHGRGSEGKPCGELKKEKGIHRLEAMLFALDRINNDPDLLPNITLGARILDTCSRDTHALEQSLTFVQALIEKDGTEVRCGSGGPPIITKPERVVGVIGASGSSVSIMVANILRLFKIPQISYASTAPDLSDNSRYDFFSRVVPSDTYQAQAMVDIVRALKWNYVSTVASEGSYGESGVEAFIQKSREDGGVCIAQSVKIPREPKAGEFDKIIRRLLETSNARAVIIFANEDDIRRVLEAARRANQTGHFFWMGSDSWGSKIAPVLHLEEVAEGAVTILPKRMSVRGFDRYFSSRTLDNNRRNIWFAEFWEDNFHCKLSRHALKKGSHVKKCTNRERIGQDSAYEQEGKVQFVIDAVYAMGHALHAMHRDLCPGRVGLCPRMDPVDGTQLLKYIRNVNFSGIAGNPVTFNENGDAPGRYDIYQYQLRNDSAEYKVIGSWTDHLHLRIERMHWPGSGQQLPRSICSLPCQPGERKKTVKGMPCCWHCEPCTGYQYQVDRYTCKTCPYDMRPTENRTGCRPIPIIKLEWGSPWAVLPLFLAVVGIAATLFVVITFVRYNDTPIVKASGRELSYVLLAGIFLCYATTFLMIAEPDLGTCSLRRIFLGLGMSISYAALLTKTNRIYRIFEQGKRSVSAPRFISPASQLAITFSLISLQLLGICVWFVVDPSHSVVDFQDQRTLDPRFARGVLKCDISDLSLICLLGYSMLLMVTCTVYAIKTRGVPETFNEAKPIGFTMYTTCIVWLAFIPIFFGTSQSADKLYIQTTTLTVSVSLSASVSLGMLYMPKVYIILFHPEQNVPKRKRSLKAVVTAATMSNKFTQKGNFRPNGEAKSELCENLEAPALATKQTYVTYTNHAI

SEQ ID NO: 16 - skipped

SEQ ID NO: 17 - protein, 772 aa, Homo sapiens
MVQTLPKLFPHDGAKRKKRTLRTSGPCFGGGGQIPQISYASTAPDLSDNSRYDFFSRVVPSDTYQAQAMVDIVRALKWNYVSTVASEGSYGESGVEAFIQKSREDGGVCIAQSVKIPREPKAGEFDKIIRRLLETSNARAVIIFANEDDIRRVLEAARRANQTGHFFWMGSDSWGSKIAPVLHLEEVAEGAVTILPKRMSVRGFDRYFSSRTLDNNRRNIWFAEFWEDNFHCKLSRHALKKGSHVKKCTNRERIGQDSAYEQEGKVQFVIDAVYAMGHALHAMHRDLCPGRVGLCPRMDPVDGTQLLKYIRNVNFSGIAGNPVTFNENGDAPGRYDIYQYQLRNDSAEYKVIGSWTDHLHLRIERMHWPGSGQQLPRSICSLPCQPGERKKTVKGMPCCWHCEPCTGYQYQVDRYTCKTCPYDMRPTENRTGCRPIPIIKLEWGSPWAVLPLFLAVVGIAATLFVVITFVRYNDTPIVKASGRELSYVLLAGIFLCYATTFLMIAEPDLGTCSLRRIFLGLGMSISYAALLTKTNRIYRIFEQGKRSVSAPRFISPASQLAITFSLISLQLLGICVWFVVDPSHSVVDFQDQRTLDPRFARGVLKCDISDLSLICLLGYSMLLMVTCTVYAIKTRGVPETFNEAKPIGFTMYTTCIVWLAFIPIFFGTSQSADKLYIQTTTLTVSVSLSASVSLGMLYMPKVYIILFHPEQNVPKRKRSLKAVVTAATMSNKFTQKGNFRPNGEAKSELCENLEAPALATKQTYVTYTNHAI

SEQ ID NO: 18 - DNA, 6518 bp, Homo sapiens
  source 1..6518: mol_type = other DNA
gatgccctggggactcacgggctgcaggtgctaccagcccagcaccccggcacagcggggaggctgaggcagacacacgctcctggagagacatgtcatgtaagataccccagatcagctacgcctccacagcgccagacctgagtgacaacagccgctacgacttcttctcccgcgtggtgccctcggacacgtaccaggcccaggccatggtggacatcgtccgtgccctcaagtggaactatgtgtccacagtggcctcggagggcagctatggtgagagcggtgtggaggccttcatccagaagtcccgtgaggacgggggcgtgtgcatcgcccagtcggtgaagataccacgggagcccaaggcaggcgagttcgacaagatcatccgccgcctcctggagacttcgaacgccagggcagtcatcatctttgccaacgaggatgacatcaggcgtgtgctggaggcagcacgaagggccaaccagacaggccatttcttctggatgggctctgacagctggggctccaagattgcacctgtgctgcacctggaggaggtggctgagggtgctgtcacgatcctccccaagaggatgtccgtacgaggcttcgaccgctacttctccagccgcacgctggacaacaaccggcgcaacatctggtttgccgagttctgggaggacaacttccactgcaagctgagccgccacgccctcaagaagggcagccacgtcaagaagtgcaccaaccgtgagcgaattgggcaggattcagcttatgagcaggaggggaaggtgcagtttgtgatcgatgccgtgtacgccatgggccacgcgctgcacgccatgcaccgtgacctgtgtcccggccgcgtggggctctgcccgcgcatggaccctgtagatggcacccagctgcttaagtacatccgaaacgtcaacttctcaggcatcgcagggaaccctgtgaccttcaatgagaatggagatgcgcctgggcgctatgacatctaccaataccagctgcgcaacgattctgccgagtacaaggtcattggctcctggactgaccacctgcaccttagaatagagcggatgcactggccggggagcgggcagcagctgccccgctccatctgcagcctgccctgccaaccgggtgagcggaagaagacagtgaagggcatgccttgctgctggcactgcgagccttgcacagggtaccagtaccaggtggaccgctacacctgtaagacgtgtccctatgacatgcggcccacagagaaccgcacgggctgccggcccatccccatcatcaagcttgagtggggctcgccctgggccgtgctgcccctcttcctggccgtggtgggcatcgctgccacgttgttcgtggtgatcacctttgtgcgctacaacgacacgcccatcgtcaaggcctcgggccgtgaactgagctacgtgctgctggcaggcatcttcctgtgctatgccaccaccttcctcatgatcgctgagcccgaccttggcacctgctcgctgcgccgaatcttcctgggactagggatgagcatcagctatgcagccctgctcaccaagaccaaccgcatctaccgcatcttcgagcagggcaagcgctcggtcagtgccccacgcttcatcagccccgcctcacagctggccatcaccttcagcctcatctcgctgcagctgctgggcatctgtgtgtggtttgtggtggacccctcccactcggtggtggacttccaggaccagcggacactcgacccccgcttcgccaggggtgtgctcaagtgtgacatctcggacctgtcgctcatctgcctgctgggctacagcatgctgctcatggtcacgtgcaccgtgtatgccatcaagacacgcggcgtgcccgagaccttcaatgaggccaagcccattggcttcaccatgtacaccacttgcatcgtctggctggccttcatccccatcttctttggcacctcgcagtcggccgacaagctgtacatccagacgacgacgctgacggtctcggtgagtctgagcgcctcggtgtccctgggaatgctctacatgcccaaagtctacatcatcctcttccacccggagcagaacgtgcccaagcgcaagcgcagcctcaaagccgtcgttacggcggccaccatgtccaacaagttcacgcagaagggcaacttccggcccaacggagaggccaagtctgagctctgcgagaaccttgaggccccagcgctggccaccaaacagacttacgtcacttacaccaaccatgcaatctagcgagtccatggagctgagcagcaggaggaggagccgtgaccctgtggaaggtgcgtcgggccagggccacacccaagggcccagctgtcttgcctgcccgtgggcacccacggacgtggcttggtgctgaggatagcagagcccccagccatcactgctggcagcctgggcaaaccgggtgagcaacaggaggacgaggggccggggcggtgccaggctaccacaagaacctgcgtcttggaccattgcccctcccggccccaaaccacaggggctcaggtcgtgtgggccccagtgctagatctctccctcccttcgtctctgtctgtgctgttggcgacccctctgtctgtctccagccctgtctttctgttctcttatctctttgtttcaccttttccctctctggcgtccccggctgcttgtactcttggccttttctgtgtctcctttctggctcttgcctccgcctctctctctcatcctctttgtcctcagctcctcctgctttcttgggtcccaccagtgtcacttttctgccgttttctttcctgttctcctctgcttcattctcgtccagccattgctcccctctccctgccacccttccccagttcaccaaaccttacatgttgcaaaagagaaaaaaggaaaaaaaatcaaaacacaaaaaagccaaaacgaaaacaaatctcgagtgtgttgccaagtgctgcgtcctcctggtggcctctgtgtgtgtccctgtggcccgcagcctgcccgcctgccccgcccatctgccgtgtgtcttgcccgcctgccccgcccgtctgccgtctgtcttgcccgcctgcccgcctgcccctcctgccgaccacacggagttcagtgcctgggtgtttggtgatggttattgacgacaatgtgtagcgcatgattgtttttataccaagaacatttctaataaaaataaacacatggttttgcacccgggctccacatccactgagggtcctgccatgggaccacaggctcagcctgcagctggagggcttagacctagagggaagcgggaactgggctctggagacccagggcttgggggctgtggagactgctccctaggctgggatctagtgtggtgtggtgaggccttgggcatggaggggccagattcccaggtaaggggcagggacattgcaggaaattccaggaatcagcacctagtagtcccctaattagggggtatgctctgtcccctgccctgcagccctgggagggtaacatttctgccttgcctgtcctctgtctcacacccctcacacctgggactgcccttccacccctgcccccataacctgtgcctctctccttccagccaggaagtcctcttcttgagaagttagcttcccgggctgccagcactcatagccgtcccctcctgcttgtgttggctccaggctcgggtgctaagaagatgtgtgtctgtcctggagatcagtgtgttgttatgtgtccacgtgggcccacaagtgcacggcacaggcatggccgtgtggctgtgttggctgtgttggctgtgtgtctgtgtgcacgtccagcgcctccatgcgcatgcgtgcctgtcttgtttgcgtgtctgatcatctgtttgggccccggtggctcatgcagatgcctgtctcaggcccatggcgagtgttcacctcagctggcttccctggcaggttgggaggtgggaaacaggagcgcttaggggctgggctctggctggggtaaattatagagccagaaacacaatgaggccataggcagcagctggagcctgggctgcctgtgccgtcccctcctgccctgcccctgggtcctgcaccccctcccacctccaggctagctgacagcgctatggagcacagtggaagggactggaggaaccctaggcagggggccacgcagggacagagtatgagagtgtgtgtataactgaggctgggacattgaatcatgccaggtatgtcttctccatcagcccactcttactcctggcctgggcatctcacacatctgtgcataggaaatctcttcttccctggggtctgtgtgcagcacctagtagatgctcaataaatgtttgtgtgagggaaggagacaggaaaggaagtgtctcgctgatcatcttgcggaatggttcctaagacctctgcccaggaaagattccacccagtgctccagcccggtcaggcagaactaggttgccagatcaagggtatctcccaaaagcttccagggcagttgggggtgggggggtggggggtagggatggggaatgcagaagcgggtgcagccagctctcccccagggtgactctggcagcacccccatcctgggcaccctgcctgctctgtggctcacgcccctcctgaagtgactgatgctctgaggcccaaggctaggtccagggcagggcctgcaggggtttcatgctcagtccaggacttgcctaggtccccctacatctgtggggcccccatctaggttctaacaggagaatcacctctccaagggggatgctgcccctcggctccccttggctctcaggaggggccctcagggactaccagtcccctgccagtgggaagaaatagccctgccctcagggagcttccagtgtgatgggggagatacagcagactgtgtcccaaagtaaaatgactgttagaatgaggtgggtggaggagggaagccttgggtgggtgtgactttgggcatctgagcctggggtgcagaggtgggctctgtgggcctgaggtggacaggagggaaccaggccctagcaagacttttgccagctagacctgctgcagcagttgggagggtgggtgctgctggagtcctgggtccatcacctagaaggctcaggccagtgcagccagggctggggcccacagctggcctgggtgggacctgccctgatgcccatggcaggagggacgcctggcccttcacaattggcttggctgctcacctttgctctcatcctcaattattaatgactggagaaagctgctaagtatcttcagaatgttagatttcaacaagatggggggttcagggtccctggcaccctggatagggagccagcggcccctagagacctttgctgtgtgcagggggtatgtgctcacccccgtggcctcagcctcctcaatgtctgaatgaaggattgggctagcagacatcccaccccacagcacactttctaaccagcaggggaacttctagacaatagagacgctgggctccctccagaacactggacctgaacttctggggggagggctgggcacgggcatattttaaaagctccccagcagatgggccgtgcagtcaagtgggccaagagtggcaccagactttggggcttgtgaagtcaggagggagcaacagtgcccactcgagcttgcctggggctcaagcccaaggctgggctgctgccagcctgagcagacacccaggagcttccaggccagctggatgcacagggcacctttgtggaactcctaggaccctggggagacccacctcaggagcagagtctcaggtcccttccggctctgaggggctgttctgagctctaatgtcttatggtctgcccctcccatccttacttctcaggccctggaggcagaggcatagagccaggcaggacagaggtctcagtgggccacatgccagctgcccccacactgcctcagcctccaggcctccaaggggtcctggggagcccctgagaagatgctgagcctgcataaggctgggcgcccctctttctgacaccctcactggctccacggctcccccttcccatcccaggtttccatctgcccactgaacagggaggggaaactgaggcactcccctggcactgagggctccttctgtcatcctgcctgccctggatggtcctggctgcccctcagggcttggccctggcactgtgagcctcacagggctcagacccccacccccaacccagcactaaatggcactcggcaccagaatctcacttcagttggcaaaagcagcaattagcatgtaatgaggcttcttgctttatttttaggtaacctccaaggccctgcctgtgtaattcagcccgccattgctcggtggataattaaagcatgtcaccataaaaaaaaaaaaaaaaaa

SEQ ID NO: 19 - protein, 743 aa, Homo sapiens
MSCKIPQISYASTAPDLSDNSRYDFFSRVVPSDTYQAQAMVDIVRALKWNYVSTVASEGSYGESGVEAFIQKSREDGGVCIAQSVKIPREPKAGEFDKIIRRLLETSNARAVIIFANEDDIRRVLEAARRANQTGHFFWMGSDSWGSKIAPVLHLEEVAEGAVTILPKRMSVRGFDRYFSSRTLDNNRRNIWFAEFWEDNFHCKLSRHALKKGSHVKKCTNRERIGQDSAYEQEGKVQFVIDAVYAMGHALHAMHRDLCPGRVGLCPRMDPVDGTQLLKYIRNVNFSGIAGNPVTFNENGDAPGRYDIYQYQLRNDSAEYKVIGSWTDHLHLRIERMHWPGSGQQLPRSICSLPCQPGERKKTVKGMPCCWHCEPCTGYQYQVDRYTCKTCPYDMRPTENRTGCRPIPIIKLEWGSPWAVLPLFLAVVGIAATLFVVITFVRYNDTPIVKASGRELSYVLLAGIFLCYATTFLMIAEPDLGTCSLRRIFLGLGMSISYAALLTKTNRIYRIFEQGKRSVSAPRFISPASQLAITFSLISLQLLGICVWFVVDPSHSVVDFQDQRTLDPRFARGVLKCDISDLSLICLLGYSMLLMVTCTVYAIKTRGVPETFNEAKPIGFTMYTTCIVWLAFIPIFFGTSQSADKLYIQTTTLTVSVSLSASVSLGMLYMPKVYIILFHPEQNVPKRKRSLKAVVTAATMSNKFTQKGNFRPNGEAKSELCENLEAPALATKQTYVTYTNHAI

SEQ ID NO: 20 - DNA, 6711 bp, Homo sapiens
  source 1..6711: mol_type = other DNA
agagatgctggagctgtggacctgggcggtcctggtggaggagtgcctgacacatcccagtctttcaccgtggtgcctgtggacagatgaggagactgaggctcacaaaggaggtgacttgcccaaggtcacacagcgaggcagcttctcacctatgggagttggactttgagccctccttggaaaagggggcatggctgtgccccttggggctccttgctgggcctctgccctgcctgcctgggctcctcccggcctcccccacagatcactgttgacaaggttactgagtcagcatgtaaaacctgcaaaaataccccagatcagctacgcctccacagcgccagacctgagtgacaacagccgctacgacttcttctcccgcgtggtgccctcggacacgtaccaggcccaggccatggtggacatcgtccgtgccctcaagtggaactatgtgtccacagtggcctcggagggcagctatggtgagagcggtgtggaggccttcatccagaagtcccgtgaggacgggggcgtgtgcatcgcccagtcggtgaagataccacgggagcccaaggcaggcgagttcgacaagatcatccgccgcctcctggagacttcgaacgccagggcagtcatcatctttgccaacgaggatgacatcaggcgtgtgctggaggcagcacgaagggccaaccagacaggccatttcttctggatgggctctgacagctggggctccaagattgcacctgtgctgcacctggaggaggtggctgagggtgctgtcacgatcctccccaagaggatgtccgtacgaggcttcgaccgctacttctccagccgcacgctggacaacaaccggcgcaacatctggtttgccgagttctgggaggacaacttccactgcaagctgagccgccacgccctcaagaagggcagccacgtcaagaagtgcaccaaccgtgagcgaattgggcaggattcagcttatgagcaggaggggaaggtgcagtttgtgatcgatgccgtgtacgccatgggccacgcgctgcacgccatgcaccgtgacctgtgtcccggccgcgtggggctctgcccgcgcatggaccctgtagatggcacccagctgcttaagtacatccgaaacgtcaacttctcaggcatcgcagggaaccctgtgaccttcaatgagaatggagatgcgcctgggcgctatgacatctaccaataccagctgcgcaacgattctgccgagtacaaggtcattggctcctggactgaccacctgcaccttagaatagagcggatgcactggccggggagcgggcagcagctgccccgctccatctgcagcctgccctgccaaccgggtgagcggaagaagacagtgaagggcatgccttgctgctggcactgcgagccttgcacagggtaccagtaccaggtggaccgctacacctgtaagacgtgtccctatgacatgcggcccacagagaaccgcacgggctgccggcccatccccatcatcaagcttgagtggggctcgccctgggccgtgctgcccctcttcctggccgtggtgggcatcgctgccacgttgttcgtggtgatcacctttgtgcgctacaacgacacgcccatcgtcaaggcctcgggccgtgaactgagctacgtgctgctggcaggcatcttcctgtgctatgccaccaccttcctcatgatcgctgagcccgaccttggcacctgctcgctgcgccgaatcttcctgggactagggatgagcatcagctatgcagccctgctcaccaagaccaaccgcatctaccgcatcttcgagcagggcaagcgctcggtcagtgccccacgcttcatcagccccgcctcacagctggccatcaccttcagcctcatctcgctgcagctgctgggcatctgtgtgtggtttgtggtggacccctcccactcggtggtggacttccaggaccagcggacactcgacccccgcttcgccaggggtgtgctcaagtgtgacatctcggacctgtcgctcatctgcctgctgggctacagcatgctgctcatggtcacgtgcaccgtgtatgccatcaagacacgcggcgtgcccgagaccttcaatgaggccaagcccattggcttcaccatgtacaccacttgcatcgtctggctggccttcatccccatcttctttggcacctcgcagtcggccgacaagctgtacatccagacgacgacgctgacggtctcggtgagtctgagcgcctcggtgtccctgggaatgctctacatgcccaaagtctacatcatcctcttccacccggagcagaacgtgcccaagcgcaagcgcagcctcaaagccgtcgttacggcggccaccatgtccaacaagttcacgcagaagggcaacttccggcccaacggagaggccaagtctgagctctgcgagaaccttgaggccccagcgctggccaccaaacagacttacgtcacttacaccaaccatgcaatctagcgagtccatggagctgagcagcaggaggaggagccgtgaccctgtggaaggtgcgtcgggccagggccacacccaagggcccagctgtcttgcctgcccgtgggcacccacggacgtggcttggtgctgaggatagcagagcccccagccatcactgctggcagcctgggcaaaccgggtgagcaacaggaggacgaggggccggggcggtgccaggctaccacaagaacctgcgtcttggaccattgcccctcccggccccaaaccacaggggctcaggtcgtgtgggccccagtgctagatctctccctcccttcgtctctgtctgtgctgttggcgacccctctgtctgtctccagccctgtctttctgttctcttatctctttgtttcaccttttccctctctggcgtccccggctgcttgtactcttggccttttctgtgtctcctttctggctcttgcctccgcctctctctctcatcctctttgtcctcagctcctcctgctttcttgggtcccaccagtgtcacttttctgccgttttctttcctgttctcctctgcttcattctcgtccagccattgctcccctctccctgccacccttccccagttcaccaaaccttacatgttgcaaaagagaaaaaaggaaaaaaaatcaaaacacaaaaaagccaaaacgaaaacaaatctcgagtgtgttgccaagtgctgcgtcctcctggtggcctctgtgtgtgtccctgtggcccgcagcctgcccgcctgccccgcccatctgccgtgtgtcttgcccgcctgccccgcccgtctgccgtctgtcttgcccgcctgcccgcctgcccctcctgccgaccacacggagttcagtgcctgggtgtttggtgatggttattgacgacaatgtgtagcgcatgattgtttttataccaagaacatttctaataaaaataaacacatggttttgcacccgggctccacatccactgagggtcctgccatgggaccacaggctcagcctgcagctggagggcttagacctagagggaagcgggaactgggctctggagacccagggcttgggggctgtggagactgctccctaggctgggatctagtgtggtgtggtgaggccttgggcatggaggggccagattcccaggtaaggggcagggacattgcaggaaattccaggaatcagcacctagtagtcccctaattagggggtatgctctgtcccctgccctgcagccctgggagggtaacatttctgccttgcctgtcctctgtctcacacccctcacacctgggactgcccttccacccctgcccccataacctgtgcctctctccttccagccaggaagtcctcttcttgagaagttagcttcccgggctgccagcactcatagccgtcccctcctgcttgtgttggctccaggctcgggtgctaagaagatgtgtgtctgtcctggagatcagtgtgttgttatgtgtccacgtgggcccacaagtgcacggcacaggcatggccgtgtggctgtgttggctgtgttggctgtgtgtctgtgtgcacgtccagcgcctccatgcgcatgcgtgcctgtcttgtttgcgtgtctgatcatctgtttgggccccggtggctcatgcagatgcctgtctcaggcccatggcgagtgttcacctcagctggcttccctggcaggttgggaggtgggaaacaggagcgcttaggggctgggctctggctggggtaaattatagagccagaaacacaatgaggccataggcagcagctggagcctgggctgcctgtgccgtcccctcctgccctgcccctgggtcctgcaccccctcccacctccaggctagctgacagcgctatggagcacagtggaagggactggaggaaccctaggcagggggccacgcagggacagagtatgagagtgtgtgtataactgaggctgggacattgaatcatgccaggtatgtcttctccatcagcccactcttactcctggcctgggcatctcacacatctgtgcataggaaatctcttcttccctggggtctgtgtgcagcacctagtagatgctcaataaatgtttgtgtgagggaaggagacaggaaaggaagtgtctcgctgatcatcttgcggaatggttcctaagacctctgcccaggaaagattccacccagtgctccagcccggtcaggcagaactaggttgccagatcaagggtatctcccaaaagcttccagggcagttgggggtgggggggtggggggtagggatggggaatgcagaagcgggtgcagccagctctcccccagggtgactctggcagcacccccatcctgggcaccctgcctgctctgtggctcacgcccctcctgaagtgactgatgctctgaggcccaaggctaggtccagggcagggcctgcaggggtttcatgctcagtccaggacttgcctaggtccccctacatctgtggggcccccatctaggttctaacaggagaatcacctctccaagggggatgctgcccctcggctccccttggctctcaggaggggccctcagggactaccagtcccctgccagtgggaagaaatagccctgccctcagggagcttccagtgtgatgggggagatacagcagactgtgtcccaaagtaaaatgactgttagaatgaggtgggtggaggagggaagccttgggtgggtgtgactttgggcatctgagcctggggtgcagaggtgggctctgtgggcctgaggtggacaggagggaaccaggccctagcaagacttttgccagctagacctgctgcagcagttgggagggtgggtgctgctggagtcctgggtccatcacctagaaggctcaggccagtgcagccagggctggggcccacagctggcctgggtgggacctgccctgatgcccatggcaggagggacgcctggcccttcacaattggcttggctgctcacctttgctctcatcctcaattattaatgactggagaaagctgctaagtatcttcagaatgttagatttcaacaagatggggggttcagggtccctggcaccctggatagggagccagcggcccctagagacctttgctgtgtgcagggggtatgtgctcacccccgtggcctcagcctcctcaatgtctgaatgaaggattgggctagcagacatcccaccccacagcacactttctaaccagcaggggaacttctagacaatagagacgctgggctccctccagaacactggacctgaacttctggggggagggctgggcacgggcatattttaaaagctccccagcagatgggccgtgcagtcaagtgggccaagagtggcaccagactttggggcttgtgaagtcaggagggagcaacagtgcccactcgagcttgcctggggctcaagcccaaggctgggctgctgccagcctgagcagacacccaggagcttccaggccagctggatgcacagggcacctttgtggaactcctaggaccctggggagacccacctcaggagcagagtctcaggtcccttccggctctgaggggctgttctgagctctaatgtcttatggtctgcccctcccatccttacttctcaggccctggaggcagaggcatagagccaggcaggacagaggtctcagtgggccacatgccagctgcccccacactgcctcagcctccaggcctccaaggggtcctggggagcccctgagaagatgctgagcctgcataaggctgggcgcccctctttctgacaccctcactggctccacggctcccccttcccatcccaggtttccatctgcccactgaacagggaggggaaactgaggcactcccctggcactgagggctccttctgtcatcctgcctgccctggatggtcctggctgcccctcagggcttggccctggcactgtgagcctcacagggctcagacccccacccccaacccagcactaaatggcactcggcaccagaatctcacttcagttggcaaaagcagcaattagcatgtaatgaggcttcttgctttatttttaggtaacctccaaggccctgcctgtgtaattcagcccgccattgctcggtggataattaaagcatgtcaccataa

SEQ ID NO: 21 - protein, 779 aa, Homo sapiens
MAVPLGAPCWASALPAWAPPGLPHRSLLTRLLSQHVKPAKIPQISYASTAPDLSDNSRYDFFSRVVPSDTYQAQAMVDIVRALKWNYVSTVASEGSYGESGVEAFIQKSREDGGVCIAQSVKIPREPKAGEFDKIIRRLLETSNARAVIIFANEDDIRRVLEAARRANQTGHFFWMGSDSWGSKIAPVLHLEEVAEGAVTILPKRMSVRGFDRYFSSRTLDNNRRNIWFAEFWEDNFHCKLSRHALKKGSHVKKCTNRERIGQDSAYEQEGKVQFVIDAVYAMGHALHAMHRDLCPGRVGLCPRMDPVDGTQLLKYIRNVNFSGIAGNPVTFNENGDAPGRYDIYQYQLRNDSAEYKVIGSWTDHLHLRIERMHWPGSGQQLPRSICSLPCQPGERKKTVKGMPCCWHCEPCTGYQYQVDRYTCKTCPYDMRPTENRTGCRPIPIIKLEWGSPWAVLPLFLAVVGIAATLFVVITFVRYNDTPIVKASGRELSYVLLAGIFLCYATTFLMIAEPDLGTCSLRRIFLGLGMSISYAALLTKTNRIYRIFEQGKRSVSAPRFISPASQLAITFSLISLQLLGICVWFVVDPSHSVVDFQDQRTLDPRFARGVLKCDISDLSLICLLGYSMLLMVTCTVYAIKTRGVPETFNEAKPIGFTMYTTCIVWLAFIPIFFGTSQSADKLYIQTTTLTVSVSLSASVSLGMLYMPKVYIILFHPEQNVPKRKRSLKAVVTAATMSNKFTQKGNFRPNGEAKSELCENLEAPALATKQTYVTYTNHAI

SEQ ID NO: 22 - DNA, 6801 bp, Homo sapiens
  source 1..6801: mol_type = other DNA
aatcccagcgctttgctggaggatcgcttgagcccaggaattcaagaccagcctgggcaacatggcgagaccctgtctgtcccaaaacaaaaaaacagattttaattggtttgggatgcggcctggacacagggaaattttaaagctctctaggtaactccaatgtgcgtcgaggttgcgaaccgccggcctgtcacacagagggagctcaggagatgctgagagtgggaggatgccggcttgggagcctggagttgcagcttcctgcggctggcgcgcccctccttgctcccctctacgcctctgcatcgcaccccacccctgtaccccaccttcctcccaccaaggaaacctcacctgccgcctcccgcccaggtcctttggattttgccggtgtgtgtgggtgcgcacaagaggcccctccctgccaggagagcaggtgagcctggccgcgcacgaatccgagggggcggccgcccagctgggaagcagccctgaaatagacccccgccgcccccgctgcctccttccggaatctgctcagataccccagatcagctacgcctccacagcgccagacctgagtgacaacagccgctacgacttcttctcccgcgtggtgccctcggacacgtaccaggcccaggccatggtggacatcgtccgtgccctcaagtggaactatgtgtccacagtggcctcggagggcagctatggtgagagcggtgtggaggccttcatccagaagtcccgtgaggacgggggcgtgtgcatcgcccagtcggtgaagataccacgggagcccaaggcaggcgagttcgacaagatcatccgccgcctcctggagacttcgaacgccagggcagtcatcatctttgccaacgaggatgacatcaggcgtgtgctggaggcagcacgaagggccaaccagacaggccatttcttctggatgggctctgacagctggggctccaagattgcacctgtgctgcacctggaggaggtggctgagggtgctgtcacgatcctccccaagaggatgtccgtacgagaccgtgagcgaattgggcaggattcagcttatgagcaggaggggaaggtgcagtttgtgatcgatgccgtgtacgccatgggccacgcgctgcacgccatgcaccgtgacctgtgtcccggccgcgtggggctctgcccgcgcatggaccctgtagatggcacccagctgcttaagtacatccgaaacgtcaacttctcaggcatcgcagggaaccctgtgaccttcaatgagaatggagatgcgcctgggcgctatgacatctaccaataccagctgcgcaacgattctgccgagtacaaggtcattggctcctggactgaccacctgcaccttagaatagagcggatgcactggccggggagcgggcagcagctgccccgctccatctgcagcctgccctgccaaccgggtgagcggaagaagacagtgaagggcatgccttgctgctggcactgcgagccttgcacagggtaccagtaccaggtggaccgctacacctgtaagacgtgtccctatgacatgcggcccacagagaaccgcacgggctgccggcccatccccatcatcaagcttgagtggggctcgccctgggccgtgctgcccctcttcctggccgtggtgggcatcgctgccacgttgttcgtggtgatcacctttgtgcgctacaacgacacgcccatcgtcaaggcctcgggccgtgaactgagctacgtgctgctggcaggcatcttcctgtgctatgccaccaccttcctcatgatcgctgagcccgaccttggcacctgctcgctgcgccgaatcttcctgggactagggatgagcatcagctatgcagccctgctcaccaagaccaaccgcatctaccgcatcttcgagcagggcaagcgctcggtcagtgccccacgcttcatcagccccgcctcacagctggccatcaccttcagcctcatctcgctgcagctgctgggcatctgtgtgtggtttgtggtggacccctcccactcggtggtggacttccaggaccagcggacactcgacccccgcttcgccaggggtgtgctcaagtgtgacatctcggacctgtcgctcatctgcctgctgggctacagcatgctgctcatggtcacgtgcaccgtgtatgccatcaagacacgcggcgtgcccgagaccttcaatgaggccaagcccattggcttcaccatgtacaccacttgcatcgtctggctggccttcatccccatcttctttggcacctcgcagtcggccgacaagctgtacatccagacgacgacgctgacggtctcggtgagtctgagcgcctcggtgtccctgggaatgctctacatgcccaaagtctacatcatcctcttccacccggagcagaacgtgcccaagcgcaagcgcagcctcaaagccgtcgttacggcggccaccatgtccaacaagttcacgcagaagggcaacttccggcccaacggagaggccaagtctgagctctgcgagaaccttgaggccccagcgctggccaccaaacagacttacgtcacttacaccaaccatgcaatctagcgagtccatggagctgagcagcaggaggaggagccgtgaccctgtggaaggtgcgtcgggccagggccacacccaagggcccagctgtcttgcctgcccgtgggcacccacggacgtggcttggtgctgaggatagcagagcccccagccatcactgctggcagcctgggcaaaccgggtgagcaacaggaggacgaggggccggggcggtgccaggctaccacaagaacctgcgtcttggaccattgcccctcccggccccaaaccacaggggctcaggtcgtgtgggccccagtgctagatctctccctcccttcgtctctgtctgtgctgttggcgacccctctgtctgtctccagccctgtctttctgttctcttatctctttgtttcaccttttccctctctggcgtccccggctgcttgtactcttggccttttctgtgtctcctttctggctcttgcctccgcctctctctctcatcctctttgtcctcagctcctcctgctttcttgggtcccaccagtgtcacttttctgccgttttctttcctgttctcctctgcttcattctcgtccagccattgctcccctctccctgccacccttccccagttcaccaaaccttacatgttgcaaaagagaaaaaaggaaaaaaaatcaaaacacaaaaaagccaaaacgaaaacaaatctcgagtgtgttgccaagtgctgcgtcctcctggtggcctctgtgtgtgtccctgtggcccgcagcctgcccgcctgccccgcccatctgccgtgtgtcttgcccgcctgccccgcccgtctgccgtctgtcttgcccgcctgcccgcctgcccctcctgccgaccacacggagttcagtgcctgggtgtttggtgatggttattgacgacaatgtgtagcgcatgattgtttttataccaagaacatttctaataaaaataaacacatggttttgcacccgggctccacatccactgagggtcctgccatgggaccacaggctcagcctgcagctggagggcttagacctagagggaagcgggaactgggctctggagacccagggcttgggggctgtggagactgctccctaggctgggatctagtgtggtgtggtgaggccttgggcatggaggggccagattcccaggtaaggggcagggacattgcaggaaattccaggaatcagcacctagtagtcccctaattagggggtatgctctgtcccctgccctgcagccctgggagggtaacatttctgccttgcctgtcctctgtctcacacccctcacacctgggactgcccttccacccctgcccccataacctgtgcctctctccttccagccaggaagtcctcttcttgagaagttagcttcccgggctgccagcactcatagccgtcccctcctgcttgtgttggctccaggctcgggtgctaagaagatgtgtgtctgtcctggagatcagtgtgttgttatgtgtccacgtgggcccacaagtgcacggcacaggcatggccgtgtggctgtgttggctgtgttggctgtgtgtctgtgtgcacgtccagcgcctccatgcgcatgcgtgcctgtcttgtttgcgtgtctgatcatctgtttgggccccggtggctcatgcagatgcctgtctcaggcccatggcgagtgttcacctcagctggcttccctggcaggttgggaggtgggaaacaggagcgcttaggggctgggctctggctggggtaaattatagagccagaaacacaatgaggccataggcagcagctggagcctgggctgcctgtgccgtcccctcctgccctgcccctgggtcctgcaccccctcccacctccaggctagctgacagcgctatggagcacagtggaagggactggaggaaccctaggcagggggccacgcagggacagagtatgagagtgtgtgtataactgaggctgggacattgaatcatgccaggtatgtcttctccatcagcccactcttactcctggcctgggcatctcacacatctgtgcataggaaatctcttcttccctggggtctgtgtgcagcacctagtagatgctcaataaatgtttgtgtgagggaaggagacaggaaaggaagtgtctcgctgatcatcttgcggaatggttcctaagacctctgcccaggaaagattccacccagtgctccagcccggtcaggcagaactaggttgccagatcaagggtatctcccaaaagcttccagggcagttgggggtgggggggtggggggtagggatggggaatgcagaagcgggtgcagccagctctcccccagggtgactctggcagcacccccatcctgggcaccctgcctgctctgtggctcacgcccctcctgaagtgactgatgctctgaggcccaaggctaggtccagggcagggcctgcaggggtttcatgctcagtccaggacttgcctaggtccccctacatctgtggggcccccatctaggttctaacaggagaatcacctctccaagggggatgctgcccctcggctccccttggctctcaggaggggccctcagggactaccagtcccctgccagtgggaagaaatagccctgccctcagggagcttccagtgtgatgggggagatacagcagactgtgtcccaaagtaaaatgactgttagaatgaggtgggtggaggagggaagccttgggtgggtgtgactttgggcatctgagcctggggtgcagaggtgggctctgtgggcctgaggtggacaggagggaaccaggccctagcaagacttttgccagctagacctgctgcagcagttgggagggtgggtgctgctggagtcctgggtccatcacctagaaggctcaggccagtgcagccagggctggggcccacagctggcctgggtgggacctgccctgatgcccatggcaggagggacgcctggcccttcacaattggcttggctgctcacctttgctctcatcctcaattattaatgactggagaaagctgctaagtatcttcagaatgttagatttcaacaagatggggggttcagggtccctggcaccctggatagggagccagcggcccctagagacctttgctgtgtgcagggggtatgtgctcacccccgtggcctcagcctcctcaatgtctgaatgaaggattgggctagcagacatcccaccccacagcacactttctaaccagcaggggaacttctagacaatagagacgctgggctccctccagaacactggacctgaacttctggggggagggctgggcacgggcatattttaaaagctccccagcagatgggccgtgcagtcaagtgggccaagagtggcaccagactttggggcttgtgaagtcaggagggagcaacagtgcccactcgagcttgcctggggctcaagcccaaggctgggctgctgccagcctgagcagacacccaggagcttccaggccagctggatgcacagggcacctttgtggaactcctaggaccctggggagacccacctcaggagcagagtctcaggtcccttccggctctgaggggctgttctgagctctaatgtcttatggtctgcccctcccatccttacttctcaggccctggaggcagaggcatagagccaggcaggacagaggtctcagtgggccacatgccagctgcccccacactgcctcagcctccaggcctccaaggggtcctggggagcccctgagaagatgctgagcctgcataaggctgggcgcccctctttctgacaccctcactggctccacggctcccccttcccatcccaggtttccatctgcccactgaacagggaggggaaactgaggcactcccctggcactgagggctccttctgtcatcctgcctgccctggatggtcctggctgcccctcagggcttggccctggcactgtgagcctcacagggctcagacccccacccccaacccagcactaaatggcactcggcaccagaatctcacttcagttggcaaaagcagcaattagcatgtaatgaggcttcttgctttatttttaggtaacctccaaggccctgcctgtgtaattcagcccgccattgctcggtggataattaaagcatgtcaccataa

SEQ ID NO: 23 - protein, 796 aa, Homo sapiens
MPAWEPGVAASCGWRAPPCSPLRLCIAPHPCTPPSSHQGNLTCRLPPRSFGFCRCVWVRTRGPSLPGEQVSLAAHESEGAAAQLGSSPEIDPRRPRCLLPESAQIPQISYASTAPDLSDNSRYDFFSRVVPSDTYQAQAMVDIVRALKWNYVSTVASEGSYGESGVEAFIQKSREDGGVCIAQSVKIPREPKAGEFDKIIRRLLETSNARAVIIFANEDDIRRVLEAARRANQTGHFFWMGSDSWGSKIAPVLHLEEVAEGAVTILPKRMSVRDRERIGQDSAYEQEGKVQFVIDAVYAMGHALHAMHRDLCPGRVGLCPRMDPVDGTQLLKYIRNVNFSGIAGNPVTFNENGDAPGRYDIYQYQLRNDSAEYKVIGSWTDHLHLRIERMHWPGSGQQLPRSICSLPCQPGERKKTVKGMPCCWHCEPCTGYQYQVDRYTCKTCPYDMRPTENRTGCRPIPIIKLEWGSPWAVLPLFLAVVGIAATLFVVITFVRYNDTPIVKASGRELSYVLLAGIFLCYATTFLMIAEPDLGTCSLRRIFLGLGMSISYAALLTKTNRIYRIFEQGKRSVSAPRFISPASQLAITFSLISLQLLGICVWFVVDPSHSVVDFQDQRTLDPRFARGVLKCDISDLSLICLLGYSMLLMVTCTVYAIKTRGVPETFNEAKPIGFTMYTTCIVWLAFIPIFFGTSQSADKLYIQTTTLTVSVSLSASVSLGMLYMPKVYIILFHPEQNVPKRKRSLKAVVTAATMSNKFTQKGNFRPNGEAKSELCENLEAPALATKQTYVTYTNHAI

SEQ ID NO: 24 - DNA, 7996 bp, Homo sapiens
  source 1..7996: mol_type = other DNA
gcgtctgcccgagcgcggcacgtgctcccggcgctggcgcgagagagcgagcgccaccgccgcgggccccgcagccgttctgcctgctgtcaccgctgcctccatcgccgacactagcgctccagctgcagccaaggccgctacgagagcgcaggaagccctcgaggagcggctgcctgggggcgcaaggctcagggcgcgcacctggtttagaagatcatgaccacatggatcatctaactaaatggtacatggggacaaaatggtcctttagaaaatacatctgaattgctggctaatttcttgatttgcgactcaacgtaggacatcgcttgttcgtagctatcagaaccctcctgaattttccccaccatgctatctttattggcttgaactcctttcctaaaatggtccttctgttgatcctgtcagtcttacttttgaaagaagatgtccgtgggagtgcacagtccagtgagaggagggtggtggctcacatgccgggtgacatcattattggagctctcttttctgttcatcaccagcctactgtggacaaagttcatgagaggaagtgtggggcggtccgtgaacagtatggcattcagagagtggaggccatgctgcataccctggaaaggatcaattcagaccccacactcttgcccaacatcacactgggctgtgagataagggactcctgctggcattcggctgtggccctagagcagagcattgagttcataagagattccctcatttcttcagaagaggaagaaggcttggtacgctgtgtggatggctcctcctcttccttccgctccaagaagcccatagtaggggtcattgggcctggctccagttctgtagccattcaggtccagaatttgctccagcttttcaacatacctcagattgcttactcagcaaccagcatggatctgagtgacaagactctgttcaaatatttcatgagggttgtgccttcagatgctcagcaggcaagggccatggtggacatagtgaagaggtacaactggacctatgtatcagccgtgcacacagaaggcaactatggagaaagtgggatggaagccttcaaagatatgtcagcgaaggaagggatttgcatcgcccactcttacaaaatctacagtaatgcaggggagcagagctttgataagctgctgaagaagctcacaagtcacttgcccaaggcccgggtggtggcctgcttctgtgagggcatgacggtgagaggtctgctgatggccatgaggcgcctgggtctagcgggagaatttctgcttctgggcagtgatggctgggctgacaggtatgatgtgacagatggatatcagcgagaagctgttggtggcatcacaatcaagctccaatctcccgatgtcaagtggtttgatgattattatctgaagctccggccagaaacaaaccaccgaaacccttggtttcaagaattttggcagcatcgttttcagtgccgactggaagggtttccacaggagaacagcaaatacaacaagacttgcaatagttctctgactctgaaaacacatcatgttcaggattccaaaatgggatttgtgatcaacgccatctattcgatggcctatgggctccacaacatgcagatgtccctctgcccaggctatgcaggactctgtgatgccatgaagccaattgatggacggaaacttttggagtccctgatgaaaaccaattttactggggtttctggagatacgatcctattcgatgagaatggagactctccaggaaggtatgaaataatgaatttcaaggaaatgggaaaagattactttgattatatcaacgttggaagttgggacaatggagaattaaaaatggatgatgatgaagtatggtccaagaaaagcaacatcatcagatctgtgtgcagtgaaccatgtgagaaaggccagatcaaggtgatccgaaagggagaagtcagctgttgttggacctgtacaccttgtaaggagaatgagtatgtctttgatgagtacacatgcaaggcatgccaactggggtcttggcccactgatgatctcacaggttgtgacttgatcccagtacagtatcttcgatggggtgaccctgaacccattgcagctgtggtgtttgcctgccttggcctcctggccaccctgtttgttactgtagtcttcatcatttaccgtgatacaccagtagtcaagtcctcaagcagggaactctgctacattatccttgctggcatctgcctgggctacttatgtaccttctgcctcattgcgaagcccaaacagatttactgctaccttcagagaattggcattggtctctccccagccatgagctactcagcccttgtaacaaagaccaaccgtattgcaaggatcctggctggcagcaagaagaagatctgtaccaaaaagcccagattcatgagtgcctgtgcccagctagtgattgctttcattctcatatgcatccagttgggcatcatcgttgccctctttataatggagcctcctgacataatgcatgactacccaagcattcgagaagtctacctgatctgtaacaccaccaacctaggagttgtcactccacttggatacaatggattgttgattttgagctgcaccttctatgcgttcaagaccagaaatgttccagctaacttcaacgaggccaagtatatcgccttcacaatgtacacgacctgcattatatggctagcttttgtgccaatctactttggcagcaactacaaaatcatcaccatgtgtttctcggtcagcctcagtgccacagtggccctaggctgcatgtttgtgccgaaggtgtacatcatcctggccaaaccagagagaaacgtgcgcagcgccttcaccacatctaccgtggtgcgcatgcatgtaggggatggcaagtcatcctccgcagccagcagatccagcagcctagtcaacctgtggaagagaaggggctcctctggggaaaccttaaggtacaaagacaggagactggcccagcacaagtcggaaatagagtgtttcacccccaaagggagtatggggaatggtgggagagcaacaatgagcagttccaatggaaaatccgtcacgtgggcccagaatgagaagagcagccgggggcagcacctgtggcagcgcctgtccatccacatcaacaagaaagaaaaccccaaccaaacggccgtcatcaagcccttccccaagagcacggagagccgtggcctgggcgctggcgctggcgcaggcgggagcgctgggggcgtgggggccacgggcggtgcgggctgcgcaggcgccggcccaggcgggcccgagtccccagacgccggccccaaggcgctgtatgatgtggccgaggctgaggagcacttcccggcgcccgcgcggccgcgctcaccgtcgcccatcagcacgctgagccaccgcgcgggctcggccagccgcacggacgacgatgtgccgtcgctgcactcggagcctgtggcgcgcagcagctcctcgcagggctccctcatggagcagatcagcagtgtggtcacccgcttcacggccaacatcagcgagctcaactccatgatgctgtccaccgcggcccccagccccggcgtcggcgccccgctctgctcgtcctacctgatccccaaagagatccagttgcccacgaccatgacgacctttgccgaaatccagcctctgccggccatcgaagtcacgggaggcgcgcagcccgcggcaggggcgcaggcggctggggacgcggcccgggagagccccgcggccggtcccgaggctgcggccgccaagccagacctggaggagctggtggctctcaccccgccgtcccccttcagagactcggtggactcggggagcacaacccccaactcgccagtgtccgagtcggccctctgtatcccgtcgtctcccaaatatgacactcttatcataagagattacactcagagctcctcgtcgttgtgaatgtccctggaaagcacgccggcctgcgcgtgcggagcggagccccccgtgttcacacacacacaatggcaagcatagtcgcctggttacggcccagggggaagatgccaagggcaccccttaatggaaacacgagatcagtagtgctatctcatgacaaccgacgaagaaaccgacgacaaatcttttggcagattttcttctagtggccttagaaaacatgggcttttaagaaacacggctgatatctttgagggctgacaaggcgtctcttcaaacagttccataccaagtgctttgctctagggaagcagtgcgtgtgaaacagcgtaacggagggtgaagagcatagttaataagcaactgtaaaaagttttatttgtttactttaattcttttcccagaagagtctttgattcaccaaacatgaatgtacattttctaacaaactcaaaatctgggaccaaaacatcaacttttttctttcttttttctttctttttgttttttctttcctgtaaagaccttgaaaagcagtaacttgggtccagtatttacggaggcgttgtgaatgtgtcccatgcataacacactactggatagtgagtgctgcgctaatgtactacgtagggcttctaccagagattttcctctccaattgggttgtgaaatactcttccaaaagcctgcatcggggattccacctacttatttcagattcacctccattaaccaagaaaaccagtggaagatttcttgactatttcaccatgttgccaatcaatactggagtagcaaaaaaaatattttctggaatactgttttgtaattccctcactggggtgcattgtagctggaaattctctttataaaaatcattcttgagctccagcctggctatctctttcaagaaacatggccactctttaggaatgctgttgcgtttgcattgccaactaaaatattaaaatatgcattggggcttcttcattcctttattttgagaacctgatgcacaaagagctcctttgttcttttcgagtcccaccactggaagagtggtccatagaccccatgaagacattgtcatgatttgagagactgttgttgaaaggattaacacaatcttaatacactgaaaattttaactgtgtcaagtcagcttagtggagatttagctatgccagtgagcagtgattttaactattcttggctgcttaaacagggcagctatgaactatgacaaatgtagatttttcaaagcaatacaaaatactaaaaaagaggaaccttaatgaatattaaccacacagtctttcttagccattccaaaaagaggcaaagcaattcttattttcttttttaaaataatgattaatatgattttgtgcacttcatactgtcactttttaaaactacagaaaagagatttagagtataacagaaacaagtgtgctttgatagtctcaaataggtagaattcatagttcaagacctgaatccactgtcatctctttcttcctcccattgcagctatcctcaggtaccaaatgttttgatttttaaataaggatagtaataaatggaggaggtgtcctataaatttaaagttcagttgacccagccttatacttaagatagccttatgaaaaatatgtgctgtgaggcagaagtatattttggcagagagaataataaataaaactttttcttttagctcaatatccttactttggtaagtatttttttttatttcacatctacttaacagaaaataaactgagaaatagaagtcagtccattggcataatttatcattcttcactttaaaaaattctaataaatattctgcttgagttttcttttctgctatttgttcttacttgcaactttaagtcaaacctcccaatacaaaacattaaaagctaacattaatgtactaaagtattaatttaaaagaaatcgaacctcccatgctagatttgaaaataacatcatcacagcaccctgatcccaaatattacaccgaggcttttaaaatgtaagtgaaatctagctaagtttcatggtttcattaaaagcaaatgtctgcctctatctgaaaaacaaatggaaatcttttgaggtgttaataccctttggatcctcatcaaaaggatggcattcacctgaggattcctatcttgacttcttaggtattaaaaacctttcttgatatgctctacattttaaaatttgttttataaaatccttatgttgattttcattttattctcaagtacaatacgtttcactctagaccagttgaagaacatgtttaaactttgttcatggtcaaattcattttctatttttttagtaacatatctcttaaaaagcacactaccttataaaaaacttcatcagaaattaaatttaatgcaagtaaattgccatctgatacttccacatgctatcataatcaactgtaataataaaaatgatttatccaattagaaaaggacaagatatatttttctctgtatttctataacttttgccactccattgaatacattgtatgttggacataagattattagtaatgcattcttgagatcttttattttggaatgatgctaactctgtctctttgccaattctaataccaggttccaagtaataactctacagtacaaagagaactgaatattcattctagggctataggatatgaacttcacaattcatttgggtacattctcattgaatttccttcaaaacaatctgttcctggtgcccagtgataattcagtcgggaccagcatgactaaaaggaaggggatatgctaaggctcagcaaagtgaccctaaatgagagatatgtcccaggatggaaagaagaagacgtggtttaaccaagttatactgactaatctaagcagtccactcatccttccattttgggaaaggagtgggggcagcctaagaagaacatatctggattgggaagaaccgtctttctgggctagggatggggaacagaaagggagtatggaaagaaaaattataagagatttgactgaagcaaggaaaaaaagcaaatccccaaacgtgctaatccttgaaagtaactatctttcccaaactactgctgttaccagcaagtgatcaggaagactaggagctatttctgactgtaaatgaattgtataatagctctgctgcagttctgtgacttccaagccaggaattaaatgctctttttaagaataacaaaaaacaaaagcatttcctatgctagtctcccagtaaaatgtacatgttttggagacttcaaaggtattatgtgagttcacatttagcaacagcttattaataaccctcaagctgtcagaatctctatagttaccatttacaattttatactgtgaaaaaatacagatcagtgaaagcataaagacaagtcagaattcactttgaagagggtctgaggcctgggagagtctctactgtctattgaagaatgaggcatgtataaaatagttggttgaatttcactgatcttcccaatgtgaacaaatatactatgtatattgtgtgtatttctagaaatcaatggcagctgctgatggtgttgtaattagaaatctatatagattatagatgttttagaaagatggtgccaatcctaaaagatttgtgtgggctaaaagtgcttgtacttacttttttctgcacttataactgatttggttttaaaattgtgtgcgtgtatctgttctttctctgttgtggcagcttgtactattaaaataatagagaatgttaaattattttgatgtgaactgcaaatgattttttttcataaagtttaacatttttatcagcattgttttgctttgtacttgtataaatatgttttattttagcacttcaaaatatacttgcctgtttctcagttgtctaaa

SEQ ID NO: 25 - protein, 1212 aa, Homo sapiens
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCNSSLTLKTHHVQDSKMGFVINAIYSMAYGLHNMQMSLCPGYAGLCDAMKPIDGRKLLESLMKTNFTGVSGDTILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPKGSMGNGGRATMSSSNGKSVTWAQNEKSSRGQHLWQRLSIHINKKENPNQTAVIKPFPKSTESRGLGAGAGAGGSAGGVGATGGAGCAGAGPGGPESPDAGPKALYDVAEAEEHFPAPARPRSPSPISTLSHRAGSASRTDDDVPSLHSEPVARSSSSQGSLMEQISSVVTRFTANISELNSMMLSTAAPSPGVGAPLCSSYLIPKEIQLPTTMTTFAEIQPLPAIEVTGGAQPAAGAQAAGDAARESPAAGPEAAAAKPDLEELVALTPPSPFRDSVDSGSTTPNSPVSESALCIPSSPKYDTLIIRDYTQSSSSL

SEQ ID NO: 26 - DNA, 7900 bp, Homo sapiens
  source 1..7900: mol_type = other DNA
gcgtctgcccgagcgcggcacgtgctcccggcgctggcgcgagagagcgagcgccaccgccgcgggccccgcagccgttctgcctgctgtcaccgctgcctccatcgccgacactagcgctccagctgcagccaaggccgctacgagagcgcaggaagccctcgaggagcggctgcctgggggcgcaaggctcagggcgcgcacctggtttagaagatcatgaccacatggatcatctaactaaatggtacatggggacaaaatggtcctttagaaaatacatctgaattgctggctaatttcttgatttgcgactcaacgtaggacatcgcttgttcgtagctatcagaaccctcctgaattttccccaccatgctatctttattggcttgaactcctttcctaaaatggtccttctgttgatcctgtcagtcttacttttgaaagaagatgtccgtgggagtgcacagtccagtgagaggagggtggtggctcacatgccgggtgacatcattattggagctctcttttctgttcatcaccagcctactgtggacaaagttcatgagaggaagtgtggggcggtccgtgaacagtatggcattcagagagtggaggccatgctgcataccctggaaaggatcaattcagaccccacactcttgcccaacatcacactgggctgtgagataagggactcctgctggcattcggctgtggccctagagcagagcattgagttcataagagattccctcatttcttcagaagaggaagaaggcttggtacgctgtgtggatggctcctcctcttccttccgctccaagaagcccatagtaggggtcattgggcctggctccagttctgtagccattcaggtccagaatttgctccagcttttcaacatacctcagattgcttactcagcaaccagcatggatctgagtgacaagactctgttcaaatatttcatgagggttgtgccttcagatgctcagcaggcaagggccatggtggacatagtgaagaggtacaactggacctatgtatcagccgtgcacacagaaggcaactatggagaaagtgggatggaagccttcaaagatatgtcagcgaaggaagggatttgcatcgcccactcttacaaaatctacagtaatgcaggggagcagagctttgataagctgctgaagaagctcacaagtcacttgcccaaggcccgggtggtggcctgcttctgtgagggcatgacggtgagaggtctgctgatggccatgaggcgcctgggtctagcgggagaatttctgcttctgggcagtgatggctgggctgacaggtatgatgtgacagatggatatcagcgagaagctgttggtggcatcacaatcaagctccaatctcccgatgtcaagtggtttgatgattattatctgaagctccggccagaaacaaaccaccgaaacccttggtttcaagaattttggcagcatcgttttcagtgccgactggaagggtttccacaggagaacagcaaatacaacaagacttgcaatagttctctgactctgaaaacacatcatgttcaggattccaaaatgggatttgtgatcaacgccatctattcgatggcctatgggctccacaacatgcagatgtccctctgcccaggctatgcaggactctgtgatgccatgaagccaattgatggacggaaacttttggagtccctgatgaaaaccaattttactggggtttctggagatacgatcctattcgatgagaatggagactctccaggaaggtatgaaataatgaatttcaaggaaatgggaaaagattactttgattatatcaacgttggaagttgggacaatggagaattaaaaatggatgatgatgaagtatggtccaagaaaagcaacatcatcagatctgtgtgcagtgaaccatgtgagaaaggccagatcaaggtgatccgaaagggagaagtcagctgttgttggacctgtacaccttgtaaggagaatgagtatgtctttgatgagtacacatgcaaggcatgccaactggggtcttggcccactgatgatctcacaggttgtgacttgatcccagtacagtatcttcgatggggtgaccctgaacccattgcagctgtggtgtttgcctgccttggcctcctggccaccctgtttgttactgtagtcttcatcatttaccgtgatacaccagtagtcaagtcctcaagcagggaactctgctacattatccttgctggcatctgcctgggctacttatgtaccttctgcctcattgcgaagcccaaacagatttactgctaccttcagagaattggcattggtctctccccagccatgagctactcagcccttgtaacaaagaccaaccgtattgcaaggatcctggctggcagcaagaagaagatctgtaccaaaaagcccagattcatgagtgcctgtgcccagctagtgattgctttcattctcatatgcatccagttgggcatcatcgttgccctctttataatggagcctcctgacataatgcatgactacccaagcattcgagaagtctacctgatctgtaacaccaccaacctaggagttgtcactccacttggatacaatggattgttgattttgagctgcaccttctatgcgttcaagaccagaaatgttccagctaacttcaacgaggccaagtatatcgccttcacaatgtacacgacctgcattatatggctagcttttgtgccaatctactttggcagcaactacaaaatcatcaccatgtgtttctcggtcagcctcagtgccacagtggccctaggctgcatgtttgtgccgaaggtgtacatcatcctggccaaaccagagagaaacgtgcgcagcgccttcaccacatctaccgtggtgcgcatgcatgtaggggatggcaagtcatcctccgcagccagcagatccagcagcctagtcaacctgtggaagagaaggggctcctctggggaaaccttaagttccaatggaaaatccgtcacgtgggcccagaatgagaagagcagccgggggcagcacctgtggcagcgcctgtccatccacatcaacaagaaagaaaaccccaaccaaacggccgtcatcaagcccttccccaagagcacggagagccgtggcctgggcgctggcgctggcgcaggcgggagcgctgggggcgtgggggccacgggcggtgcgggctgcgcaggcgccggcccaggcgggcccgagtccccagacgccggccccaaggcgctgtatgatgtggccgaggctgaggagcacttcccggcgcccgcgcggccgcgctcaccgtcgcccatcagcacgctgagccaccgcgcgggctcggccagccgcacggacgacgatgtgccgtcgctgcactcggagcctgtggcgcgcagcagctcctcgcagggctccctcatggagcagatcagcagtgtggtcacccgcttcacggccaacatcagcgagctcaactccatgatgctgtccaccgcggcccccagccccggcgtcggcgccccgctctgctcgtcctacctgatccccaaagagatccagttgcccacgaccatgacgacctttgccgaaatccagcctctgccggccatcgaagtcacgggaggcgcgcagcccgcggcaggggcgcaggcggctggggacgcggcccgggagagccccgcggccggtcccgaggctgcggccgccaagccagacctggaggagctggtggctctcaccccgccgtcccccttcagagactcggtggactcggggagcacaacccccaactcgccagtgtccgagtcggccctctgtatcccgtcgtctcccaaatatgacactcttatcataagagattacactcagagctcctcgtcgttgtgaatgtccctggaaagcacgccggcctgcgcgtgcggagcggagccccccgtgttcacacacacacaatggcaagcatagtcgcctggttacggcccagggggaagatgccaagggcaccccttaatggaaacacgagatcagtagtgctatctcatgacaaccgacgaagaaaccgacgacaaatcttttggcagattttcttctagtggccttagaaaacatgggcttttaagaaacacggctgatatctttgagggctgacaaggcgtctcttcaaacagttccataccaagtgctttgctctagggaagcagtgcgtgtgaaacagcgtaacggagggtgaagagcatagttaataagcaactgtaaaaagttttatttgtttactttaattcttttcccagaagagtctttgattcaccaaacatgaatgtacattttctaacaaactcaaaatctgggaccaaaacatcaacttttttctttcttttttctttctttttgttttttctttcctgtaaagaccttgaaaagcagtaacttgggtccagtatttacggaggcgttgtgaatgtgtcccatgcataacacactactggatagtgagtgctgcgctaatgtactacgtagggcttctaccagagattttcctctccaattgggttgtgaaatactcttccaaaagcctgcatcggggattccacctacttatttcagattcacctccattaaccaagaaaaccagtggaagatttcttgactatttcaccatgttgccaatcaatactggagtagcaaaaaaaatattttctggaatactgttttgtaattccctcactggggtgcattgtagctggaaattctctttataaaaatcattcttgagctccagcctggctatctctttcaagaaacatggccactctttaggaatgctgttgcgtttgcattgccaactaaaatattaaaatatgcattggggcttcttcattcctttattttgagaacctgatgcacaaagagctcctttgttcttttcgagtcccaccactggaagagtggtccatagaccccatgaagacattgtcatgatttgagagactgttgttgaaaggattaacacaatcttaatacactgaaaattttaactgtgtcaagtcagcttagtggagatttagctatgccagtgagcagtgattttaactattcttggctgcttaaacagggcagctatgaactatgacaaatgtagatttttcaaagcaatacaaaatactaaaaaagaggaaccttaatgaatattaaccacacagtctttcttagccattccaaaaagaggcaaagcaattcttattttcttttttaaaataatgattaatatgattttgtgcacttcatactgtcactttttaaaactacagaaaagagatttagagtataacagaaacaagtgtgctttgatagtctcaaataggtagaattcatagttcaagacctgaatccactgtcatctctttcttcctcccattgcagctatcctcaggtaccaaatgttttgatttttaaataaggatagtaataaatggaggaggtgtcctataaatttaaagttcagttgacccagccttatacttaagatagccttatgaaaaatatgtgctgtgaggcagaagtatattttggcagagagaataataaataaaactttttcttttagctcaatatccttactttggtaagtatttttttttatttcacatctacttaacagaaaataaactgagaaatagaagtcagtccattggcataatttatcattcttcactttaaaaaattctaataaatattctgcttgagttttcttttctgctatttgttcttacttgcaactttaagtcaaacctcccaatacaaaacattaaaagctaacattaatgtactaaagtattaatttaaaagaaatcgaacctcccatgctagatttgaaaataacatcatcacagcaccctgatcccaaatattacaccgaggcttttaaaatgtaagtgaaatctagctaagtttcatggtttcattaaaagcaaatgtctgcctctatctgaaaaacaaatggaaatcttttgaggtgttaataccctttggatcctcatcaaaaggatggcattcacctgaggattcctatcttgacttcttaggtattaaaaacctttcttgatatgctctacattttaaaatttgttttataaaatccttatgttgattttcattttattctcaagtacaatacgtttcactctagaccagttgaagaacatgtttaaactttgttcatggtcaaattcattttctatttttttagtaacatatctcttaaaaagcacactaccttataaaaaacttcatcagaaattaaatttaatgcaagtaaattgccatctgatacttccacatgctatcataatcaactgtaataataaaaatgatttatccaattagaaaaggacaagatatatttttctctgtatttctataacttttgccactccattgaatacattgtatgttggacataagattattagtaatgcattcttgagatcttttattttggaatgatgctaactctgtctctttgccaattctaataccaggttccaagtaataactctacagtacaaagagaactgaatattcattctagggctataggatatgaacttcacaattcatttgggtacattctcattgaatttccttcaaaacaatctgttcctggtgcccagtgataattcagtcgggaccagcatgactaaaaggaaggggatatgctaaggctcagcaaagtgaccctaaatgagagatatgtcccaggatggaaagaagaagacgtggtttaaccaagttatactgactaatctaagcagtccactcatccttccattttgggaaaggagtgggggcagcctaagaagaacatatctggattgggaagaaccgtctttctgggctagggatggggaacagaaagggagtatggaaagaaaaattataagagatttgactgaagcaaggaaaaaaagcaaatccccaaacgtgctaatccttgaaagtaactatctttcccaaactactgctgttaccagcaagtgatcaggaagactaggagctatttctgactgtaaatgaattgtataatagctctgctgcagttctgtgacttccaagccaggaattaaatgctctttttaagaataacaaaaaacaaaagcatttcctatgctagtctcccagtaaaatgtacatgttttggagacttcaaaggtattatgtgagttcacatttagcaacagcttattaataaccctcaagctgtcagaatctctatagttaccatttacaattttatactgtgaaaaaatacagatcagtgaaagcataaagacaagtcagaattcactttgaagagggtctgaggcctgggagagtctctactgtctattgaagaatgaggcatgtataaaatagttggttgaatttcactgatcttcccaatgtgaacaaatatactatgtatattgtgtgtatttctagaaatcaatggcagctgctgatggtgttgtaattagaaatctatatagattatagatgttttagaaagatggtgccaatcctaaaagatttgtgtgggctaaaagtgcttgtacttacttttttctgcacttataactgatttggttttaaaattgtgtgcgtgtatctgttctttctctgttgtggcagcttgtactattaaaataatagagaatgttaaattattttgatgtgaactgcaaatgattttttttcataaagtttaacatttttatcagcattgttttgctttgtacttgtataaatatgttttattttagcacttcaaaatatacttgcctgtttctcagttgtctaaa

SEQ ID NO: 27 - protein, 1180 aa, Homo sapiens
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCNSSLTLKTHHVQDSKMGFVINAIYSMAYGLHNMQMSLCPGYAGLCDAMKPIDGRKLLESLMKTNFTGVSGDTILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLSSNGKSVTWAQNEKSSRGQHLWQRLSIHINKKENPNQTAVIKPFPKSTESRGLGAGAGAGGSAGGVGATGGAGCAGAGPGGPESPDAGPKALYDVAEAEEHFPAPARPRSPSPISTLSHRAGSASRTDDDVPSLHSEPVARSSSSQGSLMEQISSVVTRFTANISELNSMMLSTAAPSPGVGAPLCSSYLIPKEIQLPTTMTTFAEIQPLPAIEVTGGAQPAAGAQAAGDAARESPAAGPEAAAAKPDLEELVALTPPSPFRDSVDSGSTTPNSPVSESALCIPSSPKYDTLIIRDYTQSSSSL

SEQ ID NO: 28 - skipped

SEQ ID NO: 29 - protein, 942 aa, Homo sapiens
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCNSSLTLKTHHVQDSKMGFVINAIYSMAYGLHNMQMSLCPGYAGLCDAMKPIDGRKLLESLMKTNFTGVSGDTILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPPSPFRDSVDSGSTTPNSPVSESALCIPSSPKYDTLIIRDYTQSSSSL

SEQ ID NO: 30 - DNA, 6143 bp, Homo sapiens
  source 1..6143: mol_type = other DNA
cggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccgatggcgcggccccggagagcccgggagccgctgctcgtggcgctgctgccgctggcgtggctggcgcaggcgggcctggcgcgcgcggcgggctctgtgcgcctggcgggcggcctgacgctgggcggcctgttcccggtgcacgcgcggggcgcggcgggccgggcgtgcgggcagctgaagaaggagcagggcgtgcaccggctggaggccatgctgtacgcgctggaccgcgtcaacgccgaccccgagctgctgcccggcgtgcgcctgggcgcgcggctgctggacacctgctcgcgggacacctacgcgctggagcaggcgctgagcttcgtgcaggcgctgatccgcggccgcggcgacggcgacgaggtgggcgtgcgctgcccgggaggcgtccctccgctgcgccccgcgccccccgagcgcgtcgtggccgtcgtgggcgcctcggccagctccgtctccatcatggtcgccaacgtgctgcgcctgtttgcgataccccagatcagctatgcctccacagccccggagctcagcgactccacacgctatgacttcttctcccgggtggtgccacccgactcctaccaggcgcaggccatggtggacatcgtgagggcactgggatggaactatgtgtccacgctggcctccgagggcaactatggcgaaagtggggttgaggccttcgttcagatctcccgagaggctgggggggtctgtattgcccagtctatcaagattcccagggaaccaaagccaggagagttcagcaaggtgatcaggagactcatggagacgcccaacgcccggggcatcatcatctttgccaatgaggatgacatcaggcgggtcctggaggcagctcgccaggccaacctgaccggccacttcctgtgggtcggctcagacagctggggagccaagacctcacccatcttgagcctggaggacgtggccgttggggccatcaccatcctgcccaaaagggcctccatcgacggatttgaccagtacttcatgactcgatccctggagaacaaccgcaggaacatctggttcgccgagttctgggaagagaattttaactgcaaactgaccagctcaggtacccagtcagacgattccacccgcaaatgcacaggcgaggaacgcatcggccgggactccacctacgagcaggagggcaaggtgcagtttgtgattgatgcggtgtacgccattgcccacgccctccacagcatgcaccaggcgctctgccctgggcacacaggcctgtgcccggcgatggaacccactgatgggcggatgcttctgcagtacattcgagctgtccgcttcaatggcagcgcaggaacccctgtgatgttcaacgagaacggagatgcgcccgggcggtacgacatcttccagtaccaggcgaccaatggcagtgccagcagtggcgggtaccaggcagtgggccagtgggcagagaccctcagactggatgtggaggccctgcagtggtctggcgacccccacgaggtgccctcgtctctgtgcagcctgccctgcgggccgggggagcggaagaagatggtgaagggcgtcccctgctgttggcactgcgaggcctgtgacgggtaccgcttccaggtggacgagttcacatgcgaggcctgtcctggggacatgaggcccacgcccaaccacacgggctgccgccccacacctgtggtgcgcctgagctggtcctccccctgggcagccccgccgctcctcctggccgtgctgggcatcgtggccactaccacggtggtggccaccttcgtgcggtacaacaacacgcccatcgtccgggcctcgggccgagagctcagctacgtcctcctcaccggcatcttcctcatctacgccatcaccttcctcatggtggctgagcctggggccgcggtctgtgccgcccgcaggctcttcctgggcctgggcacgaccctcagctactctgccctgctcaccaagaccaaccgtatctaccgcatctttgagcagggcaagcgctcggtcacaccccctcccttcatcagccccacctcacagctggtcatcaccttcagcctcacctccctgcaggtggtggggatgatagcatggctgggggcccggcccccacacagcgtgattgactatgaggaacagcggacggtggaccccgagcaggccagaggggtgctcaagtgcgacatgtcggatctgtctctcatcggctgcctgggctacagcctcctgctcatggtcacgtgcacagtgtacgccatcaaggcccgtggcgtgcccgagaccttcaacgaggccaagcccatcggcttcaccatgtacaccacctgcatcatctggctggcattcgtgcccatcttctttggcactgcccagtcagctgaaaagatctacatccagacaaccacgctaaccgtgtccttgagcctgagtgcctcggtgtccctcggcatgctctacgtacccaaaacctacgtcatcctcttccatccagagcagaatgtgcagaagcgaaagcggagcctcaaggccacctccacggtggcagccccacccaagggcgaggatgcagaggcccacaagtagcagggcaggtgggaacgggactgcttgctgcctctcctttcttcctcttgcctcgaggtggaagctgtatagagcccgggtccacggtgaacagtcagtggcagggagtttgccaagaccatgctccgcgtcggtggggctggccttgagaaggaactggacccagctctaccccgattccagcatgtgagcttcatgcttcctcaccacagaccagactcgcttcccatggtgggaaacagccaccgagaaggttctagctctagaaagggactaaacttattctctcatccgaagtccaaagaggatgatgaagccctgggctttgcctggtttgcgggagatttcctcccctcagtcaacccccataacctggggattgggcagtgtggaagaacgtgtagaccccagaatgaaacatggggttggagtggaggaggagctgtctcagcaagaggagacctggggctgtgcatctggatggaggcactcaggcctgggtaggattcctctggcacggagggagagaccctgggtgagacccctgtgagcatgggaagggcctgcagtgggcgcgggagtgagctgaggaactggggtgcgcccccatgagattcccaatgccatgggctttcccccatccccccgggattgggcaaggtcagacttagagtacagctgttttcctcccctctgtgtactcccttaaatcaccccaaccttggccaggcatggtggctcacacctgtaatcccagcactttgggaggccgaggcaggtggatcacctgaggtccggagttcgagaccagcctggccaatgtggtgaaaccctgtctctactaaaaatacaaaaattagccaggtgtgatggtgggtgcctgtaatcccagttacttgggaggctgaggcaggagaatcgcttgaacctgggaggtggaggttgcagtgagctgtgattgtgccactgtactccagcctgggtgacagagcgagactctgtctcaaaaaaacaaaacaaaaaaacaccaaaaaaacccccaaacctgaagaaattcagatacacgtgtgtaatgttagtgatgtgagaacaaggagcaggggtgcatttgtgttgtgttcgggttggggatgggtttaggagctccaggttgggagcagtgacagagagtcatggccgtggtgagggtgaatcccaagtggatggctcaggacgggtatggaaacccttcattcctcataggtactgggaagtccatttgcaagctgagcgccaggcctggggaggaagaggcttgggctgcagatgcacgcacatttgtttttcactgatagtttttacaaaaagcttggtttaagttatggagttttatgtccctgggagtagaatttacatttgttaaattgaccactgtttaagatcagtatacattctctagtctgtgatgtctggagctagttttgagggtgaaccacactttatccaacatacaaactttcccatgcagcttctctggtgcgcagttggttttgaccgtgggactaggtgcttctgcaggttttaagtaattaacttaaaagcttctcctctgagaaacatttctgttgcgctactgactctccttctccacatttgttgtgttcctagggcttctctatagtgcacattaggacgtttcatttgttgctgaatgctttccagaattatttattccatagggtttctctcctgtgcagctctctcatgggtaatggggcgtgttttcttgccaaaggcggttccaccctcgtgattgtatagggctcttctcctgtatgaactctgagatcagtgagctctgatctccaagggaaagttttcctgcatttgctgttttctcatgtctctcccagtgtgaattctttggtgtgcagtgcgctctggcttctagctgaaaacttttccacagttttacattcatgtggttttctccactgtgaactctgtgattcagaatcagaagcagttcttagtagaggcatttctacactgattgcactgaggatttctccccagtgtgaagtttctggcatagagtcctggcttcccgcagacgactttcacactctgccatgttcatgcctgtgggcctctctggcaggaactctgatgcaccgcgaggcccatgtactcctgtggctttctcacattcggtctacttgcagggtatctccacagcatgcaccattctgggtacagggggacatcctctgttactgaagatgttgtcatatttagtaccttcacaaggtttctctccttccagaattttctgatgtacacaaataactgacttccacaagagggcttttccacactcggtgtgtgcatacagtttctgcctgtgatcatttctttatgttattattttattttttcgagatagggtcttgctcaatttcttaggctggagtgcagtggcacgatcatagctcactgaagtttcgacctgggctcaagcaatcctcccgcttcagcctcctgagtagctggtgcgcacgaccatacccagctaatgttttattttttgtagagacgaggtctcactatgttgcccaggctggtctcgaacttctgagctcgagcgatcctcctgcctccacctcccaaagtgttcggattacaaacgtgagccatcgcacctagcctctttgatcatttctgtggtgttcagtggaggttgacagctccctaaagattttcctgtttttttgcatgcatgggtttgaattctttgaggtccaatttatttggacccctgaataaagttttgtgggttttcttctatgtgtggaatttataaggcattcttccagtgtggtttctcttatgtcgagtgagagctgacctgcaccgaaggttttgtcccatttgttgcccttgaattatttgtatgaattatatgttccagtgaaaatggagttctgggttggaggcttattccatgtttacacaattaaaattgcagtgttcctctctgggatgagagctctaaagcagagtaagattacgttctgatgtaagctttaaccacctatttataaggtctcacctgtggtccactgtgttgagacttctacagaagagcttctgtatagtaaccattttcttaggctgtctcacttgtgtgaatcttctgacacatttattatagctttgtcccatttcttatcctttttgctctttagaaatttccctttaatttattacattcattgcttactgtaaagagtccaggtaactgactttattcagttacttcctgttcaataaatttaacttttcccaaaa

SEQ ID NO: 31 - protein, 877 aa, Homo sapiens
MARPRRAREPLLVALLPLAWLAQAGLARAAGSVRLAGGLTLGGLFPVHARGAAGRACGQLKKEQGVHRLEAMLYALDRVNADPELLPGVRLGARLLDTCSRDTYALEQALSFVQALIRGRGDGDEVGVRCPGGVPPLRPAPPERVVAVVGASASSVSIMVANVLRLFAIPQISYASTAPELSDSTRYDFFSRVVPPDSYQAQAMVDIVRALGWNYVSTLASEGNYGESGVEAFVQISREAGGVCIAQSIKIPREPKPGEFSKVIRRLMETPNARGIIIFANEDDIRRVLEAARQANLTGHFLWVGSDSWGAKTSPILSLEDVAVGAITILPKRASIDGFDQYFMTRSLENNRRNIWFAEFWEENFNCKLTSSGTQSDDSTRKCTGEERIGRDSTYEQEGKVQFVIDAVYAIAHALHSMHQALCPGHTGLCPAMEPTDGRMLLQYIRAVRFNGSAGTPVMFNENGDAPGRYDIFQYQATNGSASSGGYQAVGQWAETLRLDVEALQWSGDPHEVPSSLCSLPCGPGERKKMVKGVPCCWHCEACDGYRFQVDEFTCEACPGDMRPTPNHTGCRPTPVVRLSWSSPWAAPPLLLAVLGIVATTTVVATFVRYNNTPIVRASGRELSYVLLTGIFLIYAITFLMVAEPGAAVCAARRLFLGLGTTLSYSALLTKTNRIYRIFEQGKRSVTPPPFISPTSQLVITFSLTSLQVVGMIAWLGARPPHSVIDYEEQRTVDPEQARGVLKCDMSDLSLIGCLGYSLLLMVTCTVYAIKARGVPETFNEAKPIGFTMYTTCIIWLAFVPIFFGTAQSAEKIYIQTTTLTVSLSLSASVSLGMLYVPKTYVILFHPEQNVQKRKRSLKATSTVAAPPKGEDAEAHK

SEQ ID NO: 32 - DNA, 4149 bp, Homo sapiens
  source 1..4149: mol_type = other DNA
agtgctgggctgttggagagagcgagcagcaagccggtgagcgcgagcgcggcgcgccggccggctaacccgagagcgcgaggcgccccaggctggcaggcgccgcgggacccctcaccctctctggtcgcccctccccggattcccccaccctccgtgcctgcaggagcccctgggctttcccggaggagctcgccctgaagggcccggacctcggcgagcccaccaccgttccctccagcgccgccgccgccaccgcagcagccggagcagcatggtccagctgaggaagctgctccgcgtcctgactttgatgaagttcccctgctgcgtgctggaggtgctcctgtgcgcgctggcggcggcggcgcgcggccaggagatgtacgccccgcactcaatccggatcgagggggacgtcaccctcggggggctgttccccgtgcacgccaagggtcccagcggagtgccctgcggcgacatcaagagggaaaacgggatccacaggctggaagcgatgctctacgccctggaccagatcaacagtgatcccaacctactgcccaacgtgacgctgggcgcgcggatcctggacacttgttccagggacacttacgcgctcgaacagtcgcttactttcgtccaggcgctcatccagaaggacacctccgacgtgcgctgcaccaacggcgaaccgccggttttcgtcaagccggagaaagtagttggagtgattggggcttcggggagttcggtctccatcatggtagccaacatcctgaggctcttccagatcccccagattagttatgcatcaacggcacccgagctaagtgatgaccggcgctatgacttcttctctcgcgtggtgccacccgattccttccaagcccaggccatggtagacattgtaaaggccctaggctggaattatgtgtctaccctcgcatcggaaggaagttatggagagaaaggtgtggagtccttcacgcagatttccaaagaggcaggtggactctgcattgcccagtccgtgagaatcccccaggaacgcaaagacaggaccattgactttgatagaattatcaaacagctcctggacacccccaactccagggccgtcgtgatttttgccaacgatgaggatataaagcagatccttgcagcagccaaaagagctgaccaagttggccattttctttgggtgggatcagacagctggggatccaaaataaacccactgcaccagcatgaagatatcgcagaaggggccatcaccattcagcccaagcgagccacggtggaagggtttgatgcctactttacgtcccgtacacttgaaaacaacagaagaaatgtatggtttgccgaatactgggaggaaaacttcaactgcaagttgacgattagtgggtcaaaaaaagaagacacagatcgcaaatgcacaggacaggagagaattggaaaagattccaactatgagcaggagggtaaagtccagttcgtgattgacgcagtctatgctatggctcacgcccttcaccacatgaacaaggatctctgtgctgactaccggggtgtctgcccagagatggagcaagctggaggcaagaagttgctgaagtatatacgcaatgttaatttcaatggtagtgctggcactccagtgatgtttaacaagaacggggatgcacctgggcgttatgacatctttcagtaccagaccacaaacaccagcaacccgggttaccgtctgatcgggcagtggacagacgaacttcagctcaatatagaagacatgcagtggggtaaaggagtccgagagatacccgcctcagtgtgcacactaccatgtaagccaggacagagaaagaagacacagaaaggaactccttgctgttggacctgtgagccttgcgatggttaccagtaccagtttgatgagatgacatgccagcattgcccctatgaccagaggcccaatgaaaatcgaaccggatgccaggatattcccatcatcaaactggagtggcactccccctgggctgtgattcctgtcttcctggcaatgttggggatcattgccaccatctttgtcatggccactttcatccgctacaatgacacgcccattgtccgggcatctgggcgggaactcagctatgttcttttgacgggcatctttctttgctacatcatcactttcctgatgattgccaaaccagatgtggcagtgtgttctttccggcgagttttcttgggcttgggtatgtgcatcagttatgcagccctcttgacgaaaacaaatcggatttatcgcatatttgagcagggcaagaaatcagtaacagctcccagactcataagcccaacatcacaactggcaatcacttccagtttaatatcagttcagcttctaggggtgttcatttggtttggtgttgatccacccaacatcatcatagactatgatgaacacaagacaatgaaccctgagcaagccagaggggttctcaagtgtgacattacagatctccaaatcatttgctccttgggatatagcattcttctcatggtcacatgtactgtgtatgccatcaagactcggggtgtacccgagaattttaacgaagccaagcccattggattcactatgtacacgacatgtatagtatggcttgccttcattccaattttttttggcaccgctcaatcagcggaaaagctctacatacaaactaccacgcttacaatctccatgaacctaagtgcatcagtggcgctggggatgctatacatgccgaaagtgtacatcatcattttccaccctgaactcaatgtccagaaacggaagcgaagcttcaaggcggtagtcacagcagccaccatgtcatcgaggctgtcacacaaacccagtgacagacccaacggtgaggcaaagaccgagctctgtgaaaacgtagacccaaacagccctgctgcaaaaaagaagtatgtcagttataataacctggttatctaacctgttccattccatggaaccatggaggaggaagaccctcagttattttgtcacccaacctggcataggactctttggtcctacccgcttcccatcaccggaggagcttccccggccgggagaccagtgttagaggatccaagcgacctaaacagctgctttatgaaatatccttactttatctgggcttaataagtcactgacatcagcactgccaactcggctgcaattgtggaccttccctaccaaagggagtgttgaaactcaagtcccgccctggctctttagaatggaccactgagagccacaggaccgttttggggctgacctgtcttattacgtatgtacttctaggttgcaaggttttgaaattttctgtacagtttgtgaggacctttgcactttgccatctgatgtcgtacctcggttcactgtttgttttcgaatgccttgttttcatagagccctattctctcagacggtggaatatttggaaaaattttaaaacaattaaaattttaaagcaatcttggcagactaaaacaagtacatctgtacatgactgtataattacgattatagtaccactgcacatcatgtttttttttttaagacaaaaaagatgtttaaagaccaaaaactgtgctgagaaagtatgccccacctatctttggtatatgataggttacataaaaggaaggtattggctgaactgaatagaggtcttgatctttggaatgcatgccagtaatgtattttacagtacatgtttattatgttcaatatttgtatttgtgttctcttttgttatttttaattagggtatatgaatattttgcaataattttaataattattaagctgtttgaaggaaagaatatggatttttcatgtcttgaggttttgttcatgccccctttgactgatcagtgtgataaggactttaggaaaaaaagcatgtatgttttttactgtttgtaataagtactttcgttaatcttgctgcttatgtgccaatttagtggaaaaaaacaacccttgctgaaaaattccctctttccattctctttcaattctgtgatattgtccaagaatgtatcaataaaatactttggttaactttttta

SEQ ID NO: 33 - protein, 915 aa, Homo sapiens
MVQLRKLLRVLTLMKFPCCVLEVLLCALAAAARGQEMYAPHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNSPAAKKKYVSYNNLVI

SEQ ID NO: 34 - DNA, 4241 bp, Homo sapiens
  source 1..4241: mol_type = other DNA
agtgctgggctgttggagagagcgagcagcaagccggtgagcgcgagcgcggcgcgccggccggctaacccgagagcgcgaggcgccccaggctggcaggcgccgcgggacccctcaccctctctggtcgcccctccccggattcccccaccctccgtgcctgcaggagcccctgggctttcccggaggagctcgccctgaagggcccggacctcggcgagcccaccaccgttccctccagcgccgccgccgccaccgcagcagccggagcagcatggtccagctgaggaagctgctccgcgtcctgactttgatgaagttcccctgctgcgtgctggaggtgctcctgtgcgcgctggcggcggcggcgcgcggccaggagatgtacgccccgcactcaatccggatcgagggggacgtcaccctcggggggctgttccccgtgcacgccaagggtcccagcggagtgccctgcggcgacatcaagagggaaaacgggatccacaggctggaagcgatgctctacgccctggaccagatcaacagtgatcccaacctactgcccaacgtgacgctgggcgcgcggatcctggacacttgttccagggacacttacgcgctcgaacagtcgcttactttcgtccaggcgctcatccagaaggacacctccgacgtgcgctgcaccaacggcgaaccgccggttttcgtcaagccggagaaagtagttggagtgattggggcttcggggagttcggtctccatcatggtagccaacatcctgaggctcttccagatcccccagattagttatgcatcaacggcacccgagctaagtgatgaccggcgctatgacttcttctctcgcgtggtgccacccgattccttccaagcccaggccatggtagacattgtaaaggccctaggctggaattatgtgtctaccctcgcatcggaaggaagttatggagagaaaggtgtggagtccttcacgcagatttccaaagaggcaggtggactctgcattgcccagtccgtgagaatcccccaggaacgcaaagacaggaccattgactttgatagaattatcaaacagctcctggacacccccaactccagggccgtcgtgatttttgccaacgatgaggatataaagcagatccttgcagcagccaaaagagctgaccaagttggccattttctttgggtgggatcagacagctggggatccaaaataaacccactgcaccagcatgaagatatcgcagaaggggccatcaccattcagcccaagcgagccacggtggaagggtttgatgcctactttacgtcccgtacacttgaaaacaacagaagaaatgtatggtttgccgaatactgggaggaaaacttcaactgcaagttgacgattagtgggtcaaaaaaagaagacacagatcgcaaatgcacaggacaggagagaattggaaaagattccaactatgagcaggagggtaaagtccagttcgtgattgacgcagtctatgctatggctcacgcccttcaccacatgaacaaggatctctgtgctgactaccggggtgtctgcccagagatggagcaagctggaggcaagaagttgctgaagtatatacgcaatgttaatttcaatggtagtgctggcactccagtgatgtttaacaagaacggggatgcacctgggcgttatgacatctttcagtaccagaccacaaacaccagcaacccgggttaccgtctgatcgggcagtggacagacgaacttcagctcaatatagaagacatgcagtggggtaaaggagtccgagagatacccgcctcagtgtgcacactaccatgtaagccaggacagagaaagaagacacagaaaggaactccttgctgttggacctgtgagccttgcgatggttaccagtaccagtttgatgagatgacatgccagcattgcccctatgaccagaggcccaatgaaaatcgaaccggatgccaggatattcccatcatcaaactggagtggcactccccctgggctgtgattcctgtcttcctggcaatgttggggatcattgccaccatctttgtcatggccactttcatccgctacaatgacacgcccattgtccgggcatctgggcgggaactcagctatgttcttttgacgggcatctttctttgctacatcatcactttcctgatgattgccaaaccagatgtggcagtgtgttctttccggcgagttttcttgggcttgggtatgtgcatcagttatgcagccctcttgacgaaaacaaatcggatttatcgcatatttgagcagggcaagaaatcagtaacagctcccagactcataagcccaacatcacaactggcaatcacttccagtttaatatcagttcagcttctaggggtgttcatttggtttggtgttgatccacccaacatcatcatagactatgatgaacacaagacaatgaaccctgagcaagccagaggggttctcaagtgtgacattacagatctccaaatcatttgctccttgggatatagcattcttctcatggtcacatgtactgtgtatgccatcaagactcggggtgtacccgagaattttaacgaagccaagcccattggattcactatgtacacgacatgtatagtatggcttgccttcattccaattttttttggcaccgctcaatcagcggaaaagctctacatacaaactaccacgcttacaatctccatgaacctaagtgcatcagtggcgctggggatgctatacatgccgaaagtgtacatcatcattttccaccctgaactcaatgtccagaaacggaagcgaagcttcaaggcggtagtcacagcagccaccatgtcatcgaggctgtcacacaaacccagtgacagacccaacggtgaggcaaagaccgagctctgtgaaaacgtagacccaaacaactgtataccaccagtaagaaagagtgtacaaaagtctgttacttggtacactatcccaccaacagtatagcttttgactgctttcccaaaggccctgctgcaaaaaagaagtatgtcagttataataacctggttatctaacctgttccattccatggaaccatggaggaggaagaccctcagttattttgtcacccaacctggcataggactctttggtcctacccgcttcccatcaccggaggagcttccccggccgggagaccagtgttagaggatccaagcgacctaaacagctgctttatgaaatatccttactttatctgggcttaataagtcactgacatcagcactgccaactcggctgcaattgtggaccttccctaccaaagggagtgttgaaactcaagtcccgccctggctctttagaatggaccactgagagccacaggaccgttttggggctgacctgtcttattacgtatgtacttctaggttgcaaggttttgaaattttctgtacagtttgtgaggacctttgcactttgccatctgatgtcgtacctcggttcactgtttgttttcgaatgccttgttttcatagagccctattctctcagacggtggaatatttggaaaaattttaaaacaattaaaattttaaagcaatcttggcagactaaaacaagtacatctgtacatgactgtataattacgattatagtaccactgcacatcatgtttttttttttaagacaaaaaagatgtttaaagaccaaaaactgtgctgagaaagtatgccccacctatctttggtatatgataggttacataaaaggaaggtattggctgaactgaatagaggtcttgatctttggaatgcatgccagtaatgtattttacagtacatgtttattatgttcaatatttgtatttgtgttctcttttgttatttttaattagggtatatgaatattttgcaataattttaataattattaagctgtttgaaggaaagaatatggatttttcatgtcttgaggttttgttcatgccccctttgactgatcagtgtgataaggactttaggaaaaaaagcatgtatgttttttactgtttgtaataagtactttcgttaatcttgctgcttatgtgccaatttagtggaaaaaaacaacccttgctgaaaaattccctctttccattctctttcaattctgtgatattgtccaagaatgtatcaataaaatactttggttaactttttta

SEQ ID NO: 35 - protein, 922 aa, Homo sapiens
MVQLRKLLRVLTLMKFPCCVLEVLLCALAAAARGQEMYAPHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNNCIPPVRKSVQKSVTWYTIPPTV

SEQ ID NO: 36 - skipped

SEQ ID NO: 37 - protein, 910 aa, Homo sapiens
MVQLRKLLRVLTLMKFPCCVLEVLLCALAAAARGQEMYAPHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNNFFFWLYSGTW

SEQ ID NO: 38 - skipped

SEQ ID NO: 39 - protein, 911 aa, Homo sapiens
MVQLRKLLRVLTLMKFPCCVLEVLLCALAAAARGQEMYAPHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNITSEDLSLHKED

SEQ ID NO: 40 - skipped

SEQ ID NO: 41 - protein, 906 aa, Homo sapiens
MVQLRKLLRVLTLMKFPCCVLEVLLCALAAAARGQEMYAPHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNSEKCNCY

SEQ ID NO: 42 - DNA, 3686 bp, Homo sapiens
  source 1..3686: mol_type = other DNA
gcgctcggcgctctgactgggcgtgcggcggcaggattttaaagcgctctgcctcggatcgtctgccctgggtgacctcccggacctgccctggtggaatccggacttgccccgccgagatgacgaggaataattctgctacaaggctgatttcaaggacatgaattgttgacctcatcccaacatcagaacctcagatgttctaatttttgcaccattccaggcaagttgatcttataaggaaataaaattgaaccttaggggtctgatggaaattcactgtgacattcaaatcaagaaaacttgctaatgcccacagagccttttccccatgggccctgatggtagcctccagaaggtgcagcctcaggtggtgccctttcttctgtggcaagaataaactttgggtcttggattgcaataccacctgtggagaaaatggtatgcgagggaaagcgatcagcctcttgcccttgtttcttcctcttgaccgccaagttctactggatcctcacaatgatgcaaagaactcacagccaggagtatgcccattccatacgggtggatggggacattattttggggggtctcttccctgtccacgcaaagggagagagaggggtgccttgtggggagctgaagaaggaaaaggggattcacagactggaggccatgctttatgcaattgaccagattaacaaggaccctgatctcctttccaacatcactctgggtgtccgcatcctcgacacgtgctctagggacacctatgctttggagcagtctctaacattcgtgcaggcattaatagagaaagatgcttcggatgtgaagtgtgctaatggagatccacccattttcaccaagcccgacaagatttctggcgtcataggtgctgcagcaagctccgtgtccatcatggttgctaacattttaagactttttaagatacctcaaatcagctatgcatccacagccccagagctaagtgataacaccaggtatgactttttctctcgagtggttccgcctgactcctaccaagcccaagccatggtggacatcgtgacagcactgggatggaattatgtttcgacactggcttctgaggggaactatggtgagagcggtgtggaggccttcacccagatctcgagggagattggtggtgtttgcattgctcagtcacagaaaatcccacgtgaaccaagacctggagaatttgaaaaaattatcaaacgcctgctagaaacacctaatgctcgagcagtgattatgtttgccaatgaggatgacatcaggaggatattggaagcagcaaaaaaactaaaccaaagtgggcattttctctggattggctcagatagttggggatccaaaatagcacctgtctatcagcaagaggagattgcagaaggggctgtgacaattttgcccaaacgagcatcaattgatggatttgatcgatactttagaagccgaactcttgccaataatcgaagaaatgtgtggtttgcagaattctgggaggagaattttggctgcaagttaggatcacatgggaaaaggaacagtcatataaagaaatgcacagggctggagcgaattgctcgggattcatcttatgaacaggaaggaaaggtccaatttgtaattgatgctgtatattccatggcttacgccctgcacaatatgcacaaagatctctgccctggatacattggcctttgtccacgaatgagtaccattgatgggaaagagctacttggttatattcgggctgtaaattttaatggcagtgctggcactcctgtcacttttaatgaaaacggagatgctcctggacgttatgatatcttccagtatcaaataaccaacaaaagcacagagtacaaagtcatcggccactggaccaatcagcttcatctaaaagtggaagacatgcagtgggctcatagagaacatactcacccggcgtctgtctgcagcctgccgtgtaagccaggggagaggaagaaaacggtgaaaggggtcccttgctgctggcactgtgaacgctgtgaaggttacaactaccaggtggatgagctgtcctgtgaactttgccctctggatcagagacccaacatgaaccgcacaggctgccagcttatccccatcatcaaattggagtggcattctccctgggctgtggtgcctgtgtttgttgcaatattgggaatcatcgccaccacctttgtgatcgtgacctttgtccgctataatgacacacctatcgtgagggcttcaggacgcgaacttagttacgtgctcctaacggggatttttctctgttattcaatcacgtttttaatgattgcagcaccagatacaatcatatgctccttccgacgggtcttcctaggacttggcatgtgtttcagctatgcagcccttctgaccaaaacaaaccgtatccaccgaatatttgagcaggggaagaaatctgtcacagcgcccaagttcattagtccagcatctcagctggtgatcaccttcagcctcatctccgtccagctccttggagtgtttgtctggtttgttgtggatcccccccacatcatcattgactatggagagcagcggacactagatccagagaaggccaggggagtgctcaagtgtgacatttctgatctctcactcatttgttcacttggatacagtatcctcttgatggtcacttgtactgtttatgccattaaaacgagaggtgtcccagagactttcaatgaagccaaacctattggatttaccatgtataccacctgcatcatttggttagctttcatccccatcttttttggtacagcccagtcagcagaaaagatgtacatccagacaacaacacttactgtctccatgagtttaagtgcttcagtatctctgggcatgctctatatgcccaaggtttatattataatttttcatccagaacagaatgttcaaaaacgcaagaggagcttcaaggctgtggtgacagctgccaccatgcaaagcaaactgatccaaaaaggaaatgacagaccaaatggcgaggtgaaaagtgaactctgtgagagtcttgaaaccaacacttcctctaccaagacaacatatatcagttacagcaatcattcaatctgaaacagggaaatggcacaatctgaagagatgtggtatatgatcttaaatgatgaacatgagaccgcaaaaattcactcctggagatctccgtagactacaatcaatcaaatcaatagtcagtcttgtaaggaacaaaaattagccatgagccaaaagtatcaataaacggggagtgaagaaacccgttttatacaataaaaccaatgagtgtcaagctaaagtattgcttattcatgagcagttaaaacaaatcacaaaaggaaaactaatgttagctcgtgaaaaaaaatgctgttgaaataaataatgtctgatgttattcttgtatttttctgtgattgtgagaactcccgttcctgtcccacattgtttaacttgtataagacaatgagtctgtttcttgtaatggctgaccagattgaagccctgggttgtgctaaaaataaatgcaatgattgatgcatgcaattttttatacaaataatttatttctaataataaaggaatgttttgcaaatgtt

SEQ ID NO: 43 - protein, 908 aa, Homo sapiens
MVCEGKRSASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKDLCPGYIGLCPRMSTIDGKELLGYIRAVNFNGSAGTPVTFNENGDAPGRYDIFQYQITNKSTEYKVIGHWTNQLHLKVEDMQWAHREHTHPASVCSLPCKPGERKKTVKGVPCCWHCERCEGYNYQVDELSCELCPLDQRPNMNRTGCQLIPIIKLEWHSPWAVVPVFVAILGIIATTFVIVTFVRYNDTPIVRASGRELSYVLLTGIFLCYSITFLMIAAPDTIICSFRRVFLGLGMCFSYAALLTKTNRIHRIFEQGKKSVTAPKFISPASQLVITFSLISVQLLGVFVWFVVDPPHIIIDYGEQRTLDPEKARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNTSSTKTTYISYSNHSI

SEQ ID NO: 44 - DNA, 3759 bp, Homo sapiens
  source 1..3759: mol_type = other DNA
agctcagccccctcagcccagagatcagccacaagtgcggccgctgtgctcgcctcacgcggcggcggcggcggcggcggcggcgctgacatggagctgcgggcccccggcgggcttcctcaccgcgccctctgcggggagcagggaataattctgctacaaggctgatttcaaggacatgaattgttgacctcatcccaacatcagaacctcagatgttctaatttttgcaccattccaggcaagttgatcttataaggaaataaaattgaaccttaggggtctgatggaaattcactgtgacattcaaatcaagaaaacttgctaatgcccacagagccttttccccatgggccctgatggtagcctccagaaggtgcagcctcaggtggtgccctttcttctgtggcaagaataaactttgggtcttggattgcaataccacctgtggagaaaatggtatgcgagggaaagcgatcagcctcttgcccttgtttcttcctcttgaccgccaagttctactggatcctcacaatgatgcaaagaactcacagccaggagtatgcccattccatacgggtggatggggacattattttggggggtctcttccctgtccacgcaaagggagagagaggggtgccttgtggggagctgaagaaggaaaaggggattcacagactggaggccatgctttatgcaattgaccagattaacaaggaccctgatctcctttccaacatcactctgggtgtccgcatcctcgacacgtgctctagggacacctatgctttggagcagtctctaacattcgtgcaggcattaatagagaaagatgcttcggatgtgaagtgtgctaatggagatccacccattttcaccaagcccgacaagatttctggcgtcataggtgctgcagcaagctccgtgtccatcatggttgctaacattttaagactttttaagatacctcaaatcagctatgcatccacagccccagagctaagtgataacaccaggtatgactttttctctcgagtggttccgcctgactcctaccaagcccaagccatggtggacatcgtgacagcactgggatggaattatgtttcgacactggcttctgaggggaactatggtgagagcggtgtggaggccttcacccagatctcgagggagattggtggtgtttgcattgctcagtcacagaaaatcccacgtgaaccaagacctggagaatttgaaaaaattatcaaacgcctgctagaaacacctaatgctcgagcagtgattatgtttgccaatgaggatgacatcaggaggatattggaagcagcaaaaaaactaaaccaaagtgggcattttctctggattggctcagatagttggggatccaaaatagcacctgtctatcagcaagaggagattgcagaaggggctgtgacaattttgcccaaacgagcatcaattgatggatttgatcgatactttagaagccgaactcttgccaataatcgaagaaatgtgtggtttgcagaattctgggaggagaattttggctgcaagttaggatcacatgggaaaaggaacagtcatataaagaaatgcacagggctggagcgaattgctcgggattcatcttatgaacaggaaggaaaggtccaatttgtaattgatgctgtatattccatggcttacgccctgcacaatatgcacaaagatctctgccctggatacattggcctttgtccacgaatgagtaccattgatgggaaagagctacttggttatattcgggctgtaaattttaatggcagtgctggcactcctgtcacttttaatgaaaacggagatgctcctggacgttatgatatcttccagtatcaaataaccaacaaaagcacagagtacaaagtcatcggccactggaccaatcagcttcatctaaaagtggaagacatgcagtgggctcatagagaacatactcacccggcgtctgtctgcagcctgccgtgtaagccaggggagaggaagaaaacggtgaaaggggtcccttgctgctggcactgtgaacgctgtgaaggttacaactaccaggtggatgagctgtcctgtgaactttgccctctggatcagagacccaacatgaaccgcacaggctgccagcttatccccatcatcaaattggagtggcattctccctgggctgtggtgcctgtgtttgttgcaatattgggaatcatcgccaccacctttgtgatcgtgacctttgtccgctataatgacacacctatcgtgagggcttcaggacgcgaacttagttacgtgctcctaacggggatttttctctgttattcaatcacgtttttaatgattgcagcaccagatacaatcatatgctccttccgacgggtcttcctaggacttggcatgtgtttcagctatgcagcccttctgaccaaaacaaaccgtatccaccgaatatttgagcaggggaagaaatctgtcacagcgcccaagttcattagtccagcatctcagctggtgatcaccttcagcctcatctccgtccagctccttggagtgtttgtctggtttgttgtggatcccccccacatcatcattgactatggagagcagcggacactagatccagagaaggccaggggagtgctcaagtgtgacatttctgatctctcactcatttgttcacttggatacagtatcctcttgatggtcacttgtactgtttatgccattaaaacgagaggtgtcccagagactttcaatgaagccaaacctattggatttaccatgtataccacctgcatcatttggttagctttcatccccatcttttttggtacagcccagtcagcagaaaagatgtacatccagacaacaacacttactgtctccatgagtttaagtgcttcagtatctctgggcatgctctatatgcccaaggtttatattataatttttcatccagaacagaatgttcaaaaacgcaagaggagcttcaaggctgtggtgacagctgccaccatgcaaagcaaactgatccaaaaaggaaatgacagaccaaatggcgaggtgaaaagtgaactctgtgagagtcttgaaaccaacagtaagtcatctgtagagtttccgatggtcaagagcgggagcacttcctaatagatcttcctctaccaagacaacatatatcagttacagcaatcattcaatctgaaacagggaaatggcacaatctgaagagatgtggtatatgatcttaaatgatgaacatgagaccgcaaaaattcactcctggagatctccgtagactacaatcaatcaaatcaatagtcagtcttgtaaggaacaaaaattagccatgagccaaaagtatcaataaacggggagtgaagaaacccgttttatacaataaaaccaatgagtgtcaagctaaagtattgcttattcatgagcagttaaaacaaatcacaaaaggaaaactaatgttagctcgtgaaaaaaaatgctgttgaaataaataatgtctgatgttattcttgtatttttctgtgattgtgagaactcccgttcctgtcccacattgtttaacttgtataagacaatgagtctgtttcttgtaatggctgaccagattgaagccctgggttgtgctaaaaataaatgcaatgattgatgcatgcaattttttatacaaataatttatttctaataataaaggaatgttttgcaaatgtt

SEQ ID NO: 45 - protein, 908 aa, Homo sapiens
MVCEGKRSASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKDLCPGYIGLCPRMSTIDGKELLGYIRAVNFNGSAGTPVTFNENGDAPGRYDIFQYQITNKSTEYKVIGHWTNQLHLKVEDMQWAHREHTHPASVCSLPCKPGERKKTVKGVPCCWHCERCEGYNYQVDELSCELCPLDQRPNMNRTGCQLIPIIKLEWHSPWAVVPVFVAILGIIATTFVIVTFVRYNDTPIVRASGRELSYVLLTGIFLCYSITFLMIAAPDTIICSFRRVFLGLGMCFSYAALLTKTNRIHRIFEQGKKSVTAPKFISPASQLVITFSLISVQLLGVFVWFVVDPPHIIIDYGEQRTLDPEKARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNSKSSVEFPMVKSGSTS

SEQ ID NO: 46 - protein, 501 aa, Homo sapiens
MVCEGKRSASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKDLCPGYIGLCPRMSTIDGKELLGYIRAVNFNGCRRGIQMSLPWPTLFTPSFSSSWAVLALLSLLMKTEMLLDVMISSSIK

SEQ ID NO: 47 - protein, 182 aa, synthetic construct
  REGION 1..182: note = Affinity SNAP polypeptide tag
MDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 48 - protein, 182 aa, synthetic construct
  REGION 1..182: note = Affinity SNAP polypeptide tag
MDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLG

SEQ ID NO: 49 - protein, 1061 aa, synthetic construct
MVLLLILSVLLLKEDVRGSAQSTRTRGSDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGTRKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALISGQLLIVVAWLVVEAPGTGKETAPERREVVTLRCNHRDASMLGSLAYNVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSL

SEQ ID NO: 50 - protein, 7 aa, synthetic construct
LGETTRP

SEQ ID NO: 51 - protein, 10 aa, synthetic construct
LATTSQNKPA

SEQ ID NO: 52 - protein, 10 aa, synthetic construct
LAVDGAQRSA

SEQ ID NO: 53 - protein, 10 aa, synthetic construct
LAKSDQSKPA

SEQ ID NO: 54 - protein, 10 aa, synthetic construct
LAANQPSKPA

SEQ ID NO: 55 - protein, 10 aa, synthetic construct
LAHQDTTKNS

SEQ ID NO: 56 - protein, 10 aa, synthetic construct
LAHQDSTKNA

SEQ ID NO: 57 - protein, 10 aa, synthetic construct
LAHQDATKNA

SEQ ID NO: 58 - protein, 10 aa, synthetic construct
LALSEATRPA

SEQ ID NO: 59 - protein, 10 aa, synthetic construct
LAKDETKNSA

SEQ ID NO: 60 - protein, 16 aa, synthetic construct
LQRGNRQTTTADVNTQ

SEQ ID NO: 61 - protein, 16 aa, synthetic construct
LQRGNRQATTEDVNTQ

SEQ ID NO: 62 - protein, 19 aa, synthetic construct
SRTNTPSGTTTQPTLQFSQ

SEQ ID NO: 63 - protein, 19 aa, synthetic construct
SKTDTPSGTTTQSRLQFSQ

(19) United States
(12) Patent Application Publication
(10) Pub. No.: US 2023/0064326 A1
(43) Pub. Date: Mar. 2, 2023

(54) OPTOGENETIC COMPOSITIONS COMPRISING A CBh PROMOTER SEQUENCE AND METHODS FOR USE

(71) Applicant: Vedere Bio II, Inc., Cambridge, MA (US)

(72) Inventors: Cyrus MOZAYENI, Weston, MA (US); Karen GUERIN, Braintree, MA (US); Xiaozhi REN, Cambridge, MA (US)

(21) Appl. No.: 17/811,855

(22) Filed: Jul. 11, 2022

Related U.S. Application Data
(63) Continuation-in-part of application No. PCT/US2022/012019, filed on Jan. 11, 2021.
(60) Provisional application No. 63/136,144, filed on Jan. 11, 2021.

(51) Int. Cl. C12N 15/86; C07K 14/72; A61P 27/02

(52) U.S. Cl. CPC C12N 15/86; C07K 14/723; A61P 27/02; C12N 2710/10041

(57) ABSTRACT

Disclosed are nucleic acid vectors comprising a CBh promoter operably linked to a heterologous sequence encoding a G-protein coupled receptor (GPCR). In some embodiments, composition further comprise a sequence encoding an affinity tag, optionally comprising a SNAP polypeptide. In some embodiments, the GPCR comprises a metabotropic glutamate receptor (mGluR), which is optionally, mGluR2. The disclosure also provides compositions and genetically modified cells comprising these vectors. Methods of treatment of retinal diseases and disorders comprising administering compositions, vectors, and cells of the disclosure to a subject in need are also provided.

### CROSS-REFERENCE TO RELATED APPLICATIONS

This application is a continuation-in-part of International Application No. PCT/US2022/012019, filed Jan. 11, 2022, which claims the benefit under 35 U.S.C. § 119(e) of U.S. Provisional Application No. 63/136,144, filed Jan. 11, 2021, each of which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is VEDE_009_03US_SeqList_ST26. The text file is about 168 KB, created on Jul. 11, 2022, and is being submitted electronically via Patent Center.

### FIELD OF THE DISCLOSURE

The present invention is directed to the fields of gene therapy, retinal disease and vision restoration.

### BACKGROUND

There is an unmet need for effective treatments for vision loss that address underlying condition through targeted gene therapy in particular cell types of interest. The present disclosure meets this need and offers other related advantages.

### SUMMARY

In one aspect of the present disclosure, there are provided nucleic acid vectors comprising a CBh promoter sequence operably linked to a heterologous sequence encoding a G-protein coupled receptor (GPCR). In some embodiments, the CBh promoter comprises: (i) a cytomegalovirus (CMV) enhancer sequence and (ii) a chicken beta actin (CBA) promoter sequence. In some embodiments, the CBh promoter comprises: (i) a cytomegalovirus (CMV) enhancer sequence, (ii) a chicken beta actin (CBA) promoter sequence, and (iii) an intron sequence. In more particular embodiments, the CBh promoter comprises: (i) a cytomegalovirus (CMV) enhancer sequence, (ii) a chicken beta actin (CBA) promoter sequence, and (iii) a hybrid intron sequence comprising a CBA intron sequence and a Mirabilis mosaic virus (MMV) intron sequence.

In some embodiments of the nucleic acid vectors of the disclosure, the CBh promoter comprises the sequence of SEQ ID NO: 1 or a functional fragment or variant thereof having at least 90% identity thereto, where the functional fragment or variant is capable of directing expression of the heterologous sequence in the retina. In more particular embodiments, the CBh promoter comprises the sequence of SEQ ID NO: 1.

In some embodiments of the nucleic acid vectors of the disclosure, the heterologous sequence further comprises a sequence encoding an affinity tag in addition to the GPCR. In more particular embodiments, the affinity tag comprises a SNAP polypeptide, or a functional fragment or variant thereof. In more particular embodiments, the SNAP polypeptide comprises the sequence of SEQ ID NO: 47 or SEQ ID NO: 48 or a functional fragment or variant thereof having at least 90% identity thereto. In more particular embodiments, the SNAP polypeptide is a polypeptide that binds benzylguanine (and/or to a photoswitch conjugate comprising benzylguanine).

In some embodiments of the nucleic acid vectors of the disclosure, the GPCR is an inhibitory G-protein (Gᵢ)-coupled GPCR. In some embodiments, the GPCR is a stimulatory G-protein (G_{q})-coupled GPCR. In some embodiments, the GPCR is a stimulatory G-protein (Gₛ)-coupled GPCR. In some embodiments, the GPCR comprises a metabotropic glutamate receptor (mGluR). In more specific embodiments of the invention, the GPCR sequence comprises a functional fragment or variant of a GPCR sequence. In other more specific embodiments, the functional fragment or variant thereof retains one or more desired activities of a wild type GPCR, and has at least 70%, at least 80%, at least 90%, at least 95% or at least 99% or more identity the sequence of a wild type human GPCR.

In some embodiments of the nucleic acid vectors of the disclosure, the heterologous sequence encodes a fusion protein comprising the affinity tag and the GPCR, such as wherein the fusion protein comprises, from amino (N) to carboxy (C) ends, the SNAP sequence and the GPCR sequence.

In some embodiments, the heterologous sequence may further comprise or encode additional sequences, such as signal peptides, linkers and the like. For example, in some embodiments, the heterologous sequence encodes a fusion protein, which, in addition to comprising the affinity tag and the GPCR, also comprises a signal peptide (SP) at its N-terminus. Thus, a fusion protein of the disclosure can also comprise, from amino (N) to carboxy (C) ends, a signal peptide sequence, an affinity tag sequence (e.g., a SNAP sequence) and a GPCR sequence, optionally with linker sequences between one or more of these elements. In some embodiments, the signal peptide is cleaved and is not part of the final functional protein expressed in vivo. However, in some cases, its presence is needed to facilitate proper trafficking to the membrane and/or to serve other purposes. The signal peptide may be native to the GPCR being expressed or may correspond or be derived from the signal peptide of another GPCR protein sequence.

In certain embodiments of the nucleic acid vectors of the disclosure, the GPCR used in accordance with the present invention is an mGluR polypeptide. In other more particular embodiments, the sequence encoding the mGluR polypeptide comprises one or more of: (a) a nucleic acid sequence isolated or derived from a human mGluR sequence; (b) a nucleic acid sequence having at least 70%, at least 80%, at least 90%, at least 95% or at least 99% or more identity the sequence of (a); and (c) a codon-optimized sequence derived from the sequence of any one of (a)-(c).

In some embodiments of the nucleic acid vectors of the disclosure, the mGluR comprises one or more of: (a) an amino acid sequence isolated or derived from a human mGluR sequence; (b) an amino acid sequence having at least 70%, at least 80%, at least 90%, at least 95% or at least 99% or more identity to a human mGluR sequence of (a); (c) an amino acid sequence having one or more variations conserved between a human mGluR sequence and at least one non-human mammal; and (d) an amino acid sequence having one or more silent mutations when compared to the sequence of any one of (a)-(c).

In some embodiments of the nucleic acid vectors of the disclosure, the mGluR comprises one or more of mGluR1, mGluR2, mGluR3, mGluR4, mGluR5, mGluR6, mGluR7, and mGluR8, or a functional fragment or variant thereof. In other more specific embodiments, the functional fragment or variant thereof retains one or more desired activities of a wild type mGluR, and has at least 70%, at least 80%, at least 90%, at least 95% or at least 99% or more identity the sequence of a wild type human mGluR.

In some embodiments, the mGluR comprises mGluR2. In some embodiments, the sequence encoding an mGluR comprises a sequence encoding a human mGluR2.

In some embodiments of the nucleic acid vectors of the disclosure, the sequence encoding a human mGluR2 comprises the nucleic acid sequence of SEQ ID NO: 8, or a functional fragment or variant thereof.

In some embodiments of the nucleic acid vectors of the disclosure, the human mGluR2 comprises the amino acid sequence of SEQ ID NO: 9, or a functional fragment or variant thereof.

In some embodiments of the nucleic acid vectors of the disclosure, the vector further comprises one or more of a sequence comprising an enhancer, a sequence comprising an intron or any portion thereof, a sequence comprising an exon or any portion thereof, a sequence comprising a Kozak sequence, a sequence comprising a post-transcriptional response element (PRE), a sequence comprising an inverted terminal repeat (ITR) sequence, a sequence comprising a long terminal repeat (LTR) sequence, and a poly-A sequence.

In some embodiments of the nucleic acid vectors of the disclosure, the vector further comprises a linking element in between one or more of the elements that make up the vector. For example, in some embodiments, the vectors of the disclosure comprise a linker sequence which is located between the signal peptide and the start of the affinity tag (e.g., a SNAP tag sequence). In some embodiments, the linker sequence may be part of the final functional protein but in other cases it may not. In a specific embodiment, a linker sequence comprising the amino acid sequence TRTRGS is located between the signal peptide and the start of the affinity tag sequence.

In some embodiments of the nucleic acid vectors of the disclosure, the vector further comprises a cleaving element. In more specific embodiments, the cleaving element comprises as self-cleaving element.

In some embodiments of the nucleic acid vectors of the disclosure, the vector further comprises a multicistronic element. In more specific embodiments, the multicistronic element comprises an IRES sequence.

According to another aspect of the present disclosure, there are provided delivery vectors and systems for delivering a nucleic acid vector of the disclosure comprising a CBh promoter operably linked to a sequence encoding a GPCR, such as an mGluR, to the cell type of interest. In some embodiments, the delivery vector is a viral vector. In some embodiments, the viral vector is an adeno-associated vector (AAV). In some embodiments, the AAV is a recombinant AAV (rAAV). In some embodiments, the rAAV comprises a sequence isolated or derived from an AAV of a first serotype and a sequence isolated or derived from an AAV of a second serotype. In some embodiments, the rAAV comprises a capsid sequence isolated or derived from an AAV of a serotype and a heterologous capsid insert sequence. In some embodiments, the heterologous capsid insert sequence is neither isolated nor derived from an AAV of any known serotype. In some embodiments, the heterologous capsid insert sequence comprises a random sequence.

In some embodiments of the disclosure, the delivery vector targets a retinal cell type. In some embodiments, the retinal cell type is a neuron. In some embodiments, the retinal cell type is a retinal ganglion cell, a horizontal cell, an amacrine cell, a bipolar cell or a photoreceptor cell. In some embodiments, the retinal cell type is not a photoreceptor. In some embodiments, the retinal cell type is not a retinal ganglion cell. In some embodiments, the retinal cell type is not a horizontal cell. In some embodiments, the retinal cell type is not an amacrine cell. In some embodiments, the retinal cell type is not a bipolar cell. In some embodiments, the delivery vector targets a Muller cell or an astrocyte.

According to another aspect of the present disclosure, there are provided cells, such as human cells, which have been genetically modified to contain a nucleic acid vector of the disclosure, such as a vector comprising a CBh promoter operably linked to a sequence encoding a GPCR.

According to another aspect of the present disclosure, there are provided pharmaceutical compositions comprising a nucleic acid vector, delivery vector and/or cells of the disclosure, in combination with a pharmaceutically acceptable carrier.

According to another aspect of the present disclosure, there are provided methods of treating a disease or disorder, comprising administering to a subject in need thereof, a therapeutically effective amount of a nucleic acid vector of the disclosure, an expression vector of the disclosure, a delivery vector of the disclosure, a cell of the disclosure or a pharmaceutical composition of the disclosure.

In some embodiments, the disease or disorder to be treated comprises a retinal disease or disorder. In some embodiments, the retinal disease or disorder comprises a decrease or an inhibition of a function of one or more retinal neurons. In some embodiments, the one or more retinal neurons comprise a photoreceptor cell, a cone cell, a rod cell, a ganglion cell, a bipolar cell, an amacrine cell, and a horizontal cell. In some embodiments, the one or more retinal neurons does not comprise a rod cell or a cone cell. In some embodiments, the one or more retinal neurons does not comprise a ganglion cell. In some embodiments, the one or more retinal neurons does not comprise a bipolar cell. In some embodiments, the one or more retinal neurons does not comprise an amacrine cell. In some embodiments, the one or more retinal neurons does not comprise a horizontal cell.

In some embodiments of the treatment methods of the disclosure, the subject has experienced or is at risk of experiencing a loss of visual acuity. In some embodiments, the subject has acquired condition resulting in decreased visual acuity when compared to an individual lacking the acquired condition. In some embodiments, the acquired condition comprises one or more of trauma, injury, degeneration, infection, decreased function of one or more retinal proteins, decreased activity of one or more retinal proteins, decreased expression of one or more retinal transcripts (RNA or DNA), decreased translation of one or more retinal transcripts (RNA or DNA), increased turnover of one or more retinal proteins or retinal transcripts resulting in decreased expression of one or more retinal proteins, decreased intracellular signaling of one or more retinal cell types (optionally, in response to a signal from another cell or from the environment such as light), and/or decreased intercellular signaling between retinal cells or between retinal structures (optionally, in response to a signal from another cell or from the environment such as light). In some embodiments, the subject has a congenital condition resulting in decreased visual acuity when compared to an individual lacking the congenital condition. In some embodiments, the congenital condition comprises color blindness.

In some embodiments of the methods of the disclosure, the retinal disease or disorder comprises degeneration of one or more retinal neurons or degeneration of a function of one or more retinal neurons. In some embodiments, the retinal disease or disorder comprises loss of cell viability or cell death of one or more retinal neurons.

In some embodiments of the methods of the disclosure, the administering comprises an intraocular route. In some embodiments, the intraocular route comprises an intravitreal or a subretinal route. In some embodiments, the administering comprises an injection, infusion, engraftment or implantation.

In some embodiments of the methods of the disclosure, a therapeutically effective amount of a composition of the disclosure restores or enhances visual acuity compared to a reference level of visual acuity. In some embodiments, the reference level of visual acuity comprises a medically accepted standard for an age-matched healthy individual. In some embodiments, the reference level of visual acuity comprises a baseline level of the subject measured either prior to disease onset or prior to treatment. In some embodiments, the reference level of visual acuity comprises a level of visual acuity measured in an unaffected or untreated eye of the subject.

### BRIEF DESCRIPTION OF THE DRAWINGS

FIGS. 1A-1F provide various images obtained when using AAV Var17-CBh-ChrimsonR-GFP in non-human primates. FIGS. 1A and 1B provide cSLO images taken the Heidelberg Spectralis HRA/OCT 2 weeks (A) and 6 weeks (B) following intravitreal injection of 5.0E+11 vg of AAV Var17-CBh-ChrimsonR-GFP in non-human primates.

FIG. 1C shows the extent of GFP expression in central and peripheral retina surface, by direct fluorescence imaging. FIGS. 1D-1F provide confocal images obtained from 100 um retinal section showing robust transduction of RGCs, inner neurons, Muller cells and foveal cones (1D-1E); 100 um retinal section showing transduction of RGCs, inner neurons and photoreceptors, in the peripheral retina (1F).

FIGS. 2A-2E provide images obtained when using AAV-Var17-Syn1-ChrimsonR-GFP in non-human primates. FIGS. 2A and 2B provide cSLO images taken the Heidelberg Spectralis HRA/OCT 2 weeks (A) and 6 weeks (B) following intravitreal injection of 5.0E+11 vg of AAV-Var17-Syn1-ChrimsonR-GFP in non-human primates in non-human primates. FIGS. 2C-2D provide confocal images (10×, 40×) obtained from 100 um retinal sections showing limited and specific transduction of RGCs. FIG. 2E shows a 100 um retinal section with limited and specific transduction of RGCs in the peripheral retina.

FIGS. 3A-3D provide images obtained when using AAV-Var17-CBh-SNAP-mGluR2 in non-human primates. FIGS. 3A-3D show expression results for the vector AAV-Var17-CBh-SNAP-mGluR2 in non-human primates based on immunohistochemistry against SNAP. FIGS. 3A and B provide confocal images (20× and 40×) obtained from a 100 um retinal section in the central retina showing robust transduction of RGCs. A few SNAP-positive photoreceptors are observed. FIGS. 3C and 3D provide confocal images obtained from flat-mount of the peripheral retina showing expression in RGCs (indicated by visible processes and dendritic trees).

FIGS. 4A-4D provide images obtained when using AAV-Var17-Syn1-SNAP-mGluR2 in non-human primates. FIGS. 4A-4D show expression results for the vector AAV-Var17-Syn1-SNAP-mGluR2 in non-human primates. FIGS. 4A and B provide confocal images (20× and 40×) obtained from 100 um retinal sections in the central retina showing limited transduction of RGCs when transgene expression is driven by the neuron specific Syn1 promoter. Non-specific signal can be seen in photoreceptors outer segments. FIGS. 4C-4D provide confocal images obtained from flat-mounts of the peripheral retina showing only rare cells are found to be SNAP positive.

FIGS. 5A-5B provide various images obtained when using AAV-Var17-CBh-ChrimsonR-GFP in rd1 mice. FIGS. 5A-5B show expression results for the vector AAV-Var17-CBh-ChrimsonR-GFP in rd1 mice. FIG. 5A shows 40×image of a retinal flat-mount showing ChrimsonR-GFP expression in RGCs. FIG. 5B provide confocal images (20×) obtained from 16 um cryosections showing ChrimsonR-GFP expression in RGCs and some Muller cells.

FIGS. 6A-6B provide various images obtained when using AAV-Var17-Syn1-ChrimsonR-GFP in rd1 mice. FIGS. 6A-6B show expression results for the vector AAV-Var17-Syn1-ChrimsonR-GFP in rd1 mice. FIG. 6A is a 40×image of a retinal flat-mount showing strong ChrimsonR-GFP expression in RGCs and their axons. FIG. 6B is a 20×image obtained from a 16 um cryosection showing robust and specific expression of ChrimsonR-GFP in RGCs.

FIGS. 7A-7B provide various images obtained when using AAV-Var17-CBh-SNAP-mGluR2 in rd1 mice. FIGS. 7A-7B show expression results for the vector AAV-Var17-CBh-SNAP-mGluR2 in rd1 mice. FIGS. 7A and 7B are 20× and 40×images of retinal flat-mounts showing limited number of SNAP-positive RGCs.

FIGS. 8A-8B provide various images obtained when using AAV-Var17-Syn1-SNAP-mGluR2 in rd1 mice. FIGS. 8A-8B show expression results for the vector AAV-Var17-Syn1-SNAP-mGluR2 in rd1 mice. FIGS. 8A and 8B show 20× and 40×images of retinal flat-mounts showing a significant number of SNAP-positive RGCs (significantly higher than with CBh promoter).

### DETAILED DESCRIPTION

As described herein, the present disclosure relates generally to optogenetic compositions and methods for use. Exemplary compositions according to the disclosure include nucleic acid vectors comprising a CBh promoter sequence operably linked to a heterologous sequence encoding a G-protein coupled receptor (GPCR), as well as the use of such vectors in the therapeutic treatment of ocular diseases and disorders.

### Definitions

The following definitions and descriptions are provided to better understand the present invention and to guide those of ordinary skill in the art in the practice of the present invention. Unless otherwise noted, terms are to be understood according to conventional usage by those of ordinary skill in the relevant art.

A “promoter” is generally understood as a nucleic acid sequence that is recognized by an RNA polymerase which binds to the promoter and directs transcription of a nucleic acid sequence operably linked to the promoter. A promoter can include necessary nucleic acid sequences near the start site of transcription, such as, in the case of a polymerase II type promoter, a TATA element. A promoter can also optionally include enhancer or repressor elements. An inducible promoter is generally understood as a promoter that mediates transcription of an operably linked gene in response to a particular stimulus.

The term “enhancer” refers to a nucleic acid sequence which contains sequences capable of providing enhanced transcription and in some instances can function independent of their orientation relative to another control sequence. An enhancer can function cooperatively or additively with promoters and/or other enhancer elements.

The terms “heterologous gene” or “heterologous nucleic acid” or “heterologous sequence”, as used herein, refer to a sequence that originates from a source foreign to the particular host cell or, if from the same source, is modified from its original form. Thus, a heterologous nucleic acid in a host cell can include sequences that are endogenous to the particular host cell but where the sequences have been modified from their wild type forms. A heterologous sequence can also include a sequence that is endogenous to the particular host cell but is under the control of a promoter sequence that is not naturally associated with the sequence. The terms also include non-naturally occurring multiple copies of a naturally occurring DNA sequence.

“Operably-linked” or “functionally linked” refers to the association of nucleic acid sequences on a single nucleic acid fragment so that the function of one is affected by the other in an intended manner. For example, a regulatory DNA sequence (such as a promoter) is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell. In some embodiments, a sequence encoding a CBh promoter is operably linked to a sequence encoding a GPCR receptor, which may or may not be contiguous sequences, but are operably linked because the promoter is capable of driving expression of the GPCR receptor in a cell.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

A “transcribable nucleic acid molecule” as used herein refers to any nucleic acid molecule capable of being transcribed into a RNA molecule.

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

Methods are known for introducing constructs into a cell in such a manner that the transcribable nucleic acid molecule is transcribed into a functional mRNA molecule that is translated and therefore expressed as a protein product.

Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

As used herein, the term “genetically engineered” or “genetically modified” refers to the addition of extra genetic material in the form of DNA or RNA into the total genetic material in a cell. The terms, “genetically modified cells”, “modified cells”, and “redirected cells” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide.

“Wild-type” refers to a virus or organism found in nature without any known mutation.

Design, generation, and testing of the variant nucleotides, within transcriptional regulatory sequences (e.g., promoters) as well as encoded polypeptides, having the herein required percent identities and retaining a required promoter activity or activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 1 19-123; Ghadessy et al. (2001) Proc Natl Acad Sci USA 98(8) 4552-4557. Thus, one skilled in the art could generate a large number of nucleotide and/or polypeptide variants having, for example, at least 90-99% identity or 95-99% identity to the reference sequence described herein and screen such for desired phenotypes according to methods routine in the art.

In some embodiment, a CBh promoter variant sequences comprises one or more nucleotide insertions, deletions, substitutions, or modifications, relative to the specific CBh promoter sequences disclosed herein, such that increased or stabilized CBh promoter activity is achieved. In some embodiments, a CBh promoter sequence comprises 2 or more, 3 or more, 4 or more, 5 or more, 6 or more, 7 or more, 8 or more, 9 or more, 10 or more, 11 or more, 12 or more, 13 or more, 14 or more, 15 or more, 20 or more, or 25 or more, nucleotide insertions, deletions, substitutions, or modifications, relative to the specific CBh promoter sequences disclosed herein, such that increased or stabilized CBh promoter activity is achieved.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a vector or host cell include, for example, exogenous sequences or sequences which originate with or are present in the same species, but which are incorporated into recipient cells by genetic engineering methods. The term “exogenous” refers to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended.

For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

As used throughout the disclosure, the term “isolated” is meant to describe a sequence that is removed from its biological context but is otherwise unchanged in sequence.

As used throughout the disclosure, the term “derived” is meant to describe a sequence that has been modified from a naturally occurring sequence but retains sufficient sequence homology or identity to be recognized as preserving one or more structure-function relationships. In some embodiments, a sequence derived from a human sequence contains one or more modified or synthetic nucleic acids that do not occur in nature but may increase stability or reduce immunogenicity. In some embodiments, a sequence derived from a human sequence contains one or more silent mutations that improve manufacturability while retaining function. In some embodiments, a sequence derived from a human sequence is a recombinant sequence. In some embodiments, a sequence derived from a human sequence is a chimeric sequence.

### CBh Promoter

A CBh promoter sequence of the disclosure typically comprises: (i) a cytomegalovirus (CMV) enhancer sequence, (ii) a chicken beta actin (CBA) promoter sequence and (iii) a hybrid intron sequence comprising a CBA intron sequence and a Mirabilis mosaic virus (MMV) intron sequence. In some embodiments, a CBh promoter sequence comprises a sequence as set out in Grey et al. (Hum Gene Therapy 22(9): 1143-1153, 2011). In some embodiments of the present disclosure, the CBh promoter comprises or consists essentially of the nucleic acid sequence of SEQ ID NO: 1.

| (SEQ ID NO: 1) | |
|---|---|
| 1 | CGTTACATAA CTTACGGTAA ATGGCCCGCC TGGCTGACCG CCCAACGACC CCCGCCCATT |
| 61 | GACGTCAATA ATGACGTATG TTCCCATAGT AACGCCAATA GGGACTTTCC ATTGACGTCA |
| 121 | ATGGGTGGAG TATTTACGGT AAACTGCCCA CTTGGCAGTA CATCAAGTGT ATCATATGCC |
| 181 | AAGTACGCCC CCTATTGACG TCAATGACGG TAAATGGCCC GCCTGGCATT ATGCCCAGTA |
| 241 | CATGACCTTA TGGGACTTTC CTACTTGGCA GTACATCTAC GTATTAGTCA TCGCTATTAC |
| 301 | CATGGTCGAG GTGAGCCCCA CGTTCTGCTT CACTCTCCCC ATCTCCCCCC CCTCCCCACC |
| 361 | CCCAATTTTG TATTTATTTA TTTTTTAATT ATTTTGTGCA GCGATGGGGG CGGGGGGGGG |
| 421 | GGGGGGGCGC GCGCCAGGCG GGGCGGGGCG GGGCGAGGGG CGGGGCGGGG CGAGGCGGAG |
| 481 | AGGTGCGGCG GCAGCCAATC AGAGCGGCGC GCTCCGAAAG TTTCCTTTTA TGGCGAGGCG |
| 541 | GCGGCGGCGG CGGCCCTATA AAAAGCGAAG CGCGCGGCGG GCGGGAGTCG CTGCGACGCT |
| 601 | GCCTTCGCCC CGTGCCCCGC TCCGCCGCCG CCTCGCGCCG CCCGCCCCGG CTCTGACTGA |
| 661 | CCGCGTTACT CCCACAGGTG AGCGGGCGGG ACGGCCCTTC TCCTCCGGGC TGTAATTAGC |
| 721 | TGAGCAAGAG GTAAGGGTTT AAGGGATGGT TGGTTGGTGG GGTATTAATG TTTAATTACC |
| 781 | TGGAGCACCT GCCTGAAATC ACTTTTTTTC AG |

In some embodiments of the disclosure, a hybrid CBh promoter used according to the present disclosure comprises a nucleic acid sequence derived from a CBh promoter as set forth in SEQ ID NO: 1.

In some embodiments, the CMV enhancer sequence of a hybrid CBh promoter of the present disclosure comprises a sequence having at least 70%, at least 75%, at least 80%, at least 85%, at least 90%, at least 95%, at least 97%, at least 99% or any percentage identity in between to residues 1-305 of SEQ ID NO: 1, or any functional fragment thereof.

In some embodiments, the CBA promoter sequence of a hybrid CBh promoter of the present disclosure comprises a sequence having at least 70%, at least 75%, at least 80%, at least 85%, at least 90%, at least 95%, at least 97%, at least 99% or any percentage identity in between to residues 306-583 of SEQ ID NO: 1, or any functional fragment thereof.

In some embodiments, the intronic sequence of a hybrid CBh promoter of the present disclosure comprises a sequence having at least 70%, at least 75%, at least 80%, at least 85%, at least 90%, at least 95%, at least 97%, at least 99% or any percentage identity in between to residues 584-812 of SEQ ID NO: 1, or any functional fragment thereof.

In some embodiments, the sequence encoding a CBh promoter comprises or consists essentially of SEQ ID NO: 1, or any functional fragment thereof capable of directing expression of a heterologous sequence in the retina. A functional fragment may be essentially any length, including sequences comprising at least at least or no more than 100, at least or no more than 200, at least or no more than 300, at least or no more than 400, at least or no more than 500, at least or no more than 600, or at least or no more than 700 or more nucleic acid residues.

In some embodiments, the CBh promoter comprises or consists of a variant nucleic acid sequence having at least 70%, at least 75%, at least 80%, at least 85%, at least 90%, at least 95%, at least 97%, at least 99% or any percentage identity in between to a CBh promoter of SEQ ID NO: 1, or any functional fragment thereof effective for directing expression of a heterologous sequence in the retina.

### Affinity Tag

In some embodiments of the disclosure, the heterologous sequence under the control of the CBh promoter further comprises, in addition to a sequence encoding a GPCR, a sequence encoding an affinity tag. In some embodiments, the affinity tag comprises a SNAP polypeptide. In some embodiments, the SNAP polypeptide comprises the sequence of SEQ ID NO: 47 or SEQ ID NO: 48 below.

(SEQ ID NO: 47)
MDKDCEMKRTTLDSPLGKLELSGCEQGLHRIIFLGKGTSAADAVEVPAPA
AVLGGPEPLMQATAWLNAYFHQPEAIEEFPVPALHHPVFQQESFTRQVLW
KLLKVVKFGEVISYSHLAALAGNPAATAAVKTALSGNPVPILIPCHRVVQ
GDLDVGGYEGGLAVKEWLLAHEGHRLGKPGLG.
(SEQ ID NO: 48)
MDKDCEMKRTTLDSPLGKLELSGCEQGLHEIKLLGKGTSAADAVEVPAPA
AVLGGPEPLMQATAWLNAYFHQPEAIEEFPVPALHHPVFQQESFTRQVLW
KLLKVVKFGEVISYQQLAALAGNPAATAAVKTALSGNPVPILIPCHRVVS
SSGAVGGYEGGLAVKEWLLAHEGHRLGKPGLG.

In some embodiments, there is no methionine in the first position of the SNAP tag sequence because the start methionine is instead at the N-terminus of a signal peptide that is expressed in fusion with the SNAP tag, optionally with a linker sequence between the two.

In some embodiments, the CBh promoter sequence is operably linked to the sequence encoding the GPCR and to the sequence encoding the affinity tag. As such, the heterologous sequence encodes a fusion polypeptide comprising an affinity tag (e.g., SNAP) and a GPCR.

In some embodiments, the SNAP polypeptide is a sequence having at least 50%, at least 55%, at least 60%, at least 65%, at least 70%, at least 75%, at least 80%, at least 85%, at least 90%, at least 95%, at least 98%, at least 99%, or more amino acid sequence identity to the SNAP sequence set out herein. The SNAP polypeptides and variants used according to the present disclosure are generally those that retain binding to a molecule comprising benzylguanine.

In some embodiments, the nucleic acid vectors of the disclosure are used in conjunction with a photoisomerizable small molecule. In some embodiments, the heterologous sequence comprises a sequence encoding an affinity tag and the photoisomerizable small molecule is capable of binding to the affinity tag to generate an activated affinity tag. In some embodiments, the photoisomerizable small molecule is capable of binding to the affinity tag covalently. In some embodiments, the photoisomerizable small molecule is capable of binding to the affinity tag non-covalently. In some embodiments, the activated affinity tag is capable of binding to the GPCR to produce an activated GPCR. In some embodiments, a SNAP polypeptide of the disclosure binds to a benzylguanine molecule that is associated with a photoisomerizable small molecule. In some embodiments, the photoisomerizable small molecule comprises azobenzene.

In certain more specific embodiments of the present invention, a composition of the present invention, comprising a CBh promoter sequence and a heterologous sequence encoding a GPCR or encoding a fusion polypeptide such as a SNAP-GPCR fusion polypeptide, may be made and used in conjunction with photoisomerizable small molecules in accordance with the disclosures set forth in WO2019/060785 and/or WO2021/243086, the contents of which are incorporated herein by reference in their entireties.

### Metabotropic Glutamate Receptor

Metabotropic glutamate receptors (mGluRs) of the disclosure may be isolated or derived from any species. In some embodiments, the mGluR comprises one or more of mGluR1, mGluR2, mGluR3, mGluR4, mGluR5, mGluR6, mGluR7 and mGluR8, or a functional fragment or variant thereof.

In some embodiments, the sequence encoding a human mGluR1 comprises or consists of the nucleic acid sequence of the following sequence, or a functional fragment or variant thereof (UniProtKB Q13255-1; GenBank Accession No. NM_001278064.2 and SEQ ID NO: 2):

| | |
|---|---|
| 1 | aagctgttcc tgcagccgat atcaggatgt gccgaaatga aacggacaag gcaactgtta |
| 61 | acattataga ccccagagtt ttaacacagg tcctctgatg acaaggttgt gatttttctc |
| 121 | tgtcttttgt cagcagcatc tagctcaccg ctgccaacac gacttccact gtactcttga |
| 181 | tcaatttacc ttgatgcact accggtgaag aacggggact cgaattccct tacaaacgcc |
| 241 | tccagcttgt agaggcggtc gtggaggacc cagaggagga gacgaagggg aaggaggcgg |
| 301 | tggtggagga ggcaaaggcc ttggacgacc attgttggcg aggggcacca ctccgggaga |
| 361 | ggcggcgctg ggcgtcttgg gggtgcgcgc cgggagcctg cagcgggacc agcgtgggaa |
| 421 | cgcggctggc aggctgtgga cctcgtcctc accaccatgg tcgggctcct tttgtttttt |
| 481 | ttcccagcga tctttttgga ggtgtccctt ctccccagaa gccccggcag gaaagtgttg |
| 541 | ctggcaggag cgtcgtctca gcgctcggtg gccagaatgg acggagatgt catcattgga |
| 601 | gccctcttct cagtccatca ccagcctccg gccgagaaag tgcccgagag gaagtgtggg |
| 661 | gagatcaggg agcagtatgg catccagagg gtggaggcca tgttccacac gttggataag |
| 721 | atcaacgcgg acccggtcct cctgcccaac atcaccctgg gcagtgagat ccgggactcc |
| 781 | tgctggcact cttccgtggc tctggaacag agcattgagt tcattaggga ctctctgatt |
| 841 | tccattcgag atgagaagga tgggatcaac cggtgtctgc ctgacggcca gtccctcccc |
| 901 | ccaggcagga ctaagaagcc cattgcggga gtgatcggtc ccggctccag ctctgtagcc |
| 961 | attcaagtgc agaacctgct ccagctcttc gacatccccc agatcgctta ttcagccaca |
| 1021 | agcatcgacc tgagtgacaa aactttgtac aaatacttcc tgagggttgt cccttctgac |
| 1081 | actttgcagg caagggccat gcttgacata gtcaaacgtt acaattggac ctatgtctct |
| 1141 | gcagtccaca cggaagggaa ttatggggag agcggaatgg acgctttcaa agagctggct |
| 1201 | gcccaggaag gcctctgtat cgcccattct gacaaaatct acagcaacgc tggggagaag |
| 1261 | agctttgacc gactcttgcg caaactccga gagaggcttc ccaaggctag agtggtggtc |
| 1321 | tgcttctgtg aaggcatgac agtgcgagga ctcctgagcg ccatgcggcg ccttggcgtc |
| 1381 | gtgggcgagt tctcactcat tggaagtgat ggatgggcag acagagatga agtcattgaa |
| 1441 | ggttatgagg tggaagccaa cgggggaatc acgataaagc tgcagtctcc agaggtcagg |
| 1501 | tcatttgatg attatttcct gaaactgagg ctggacacta acacgaggaa tccctggttc |
| 1561 | cctgagttct ggcaacatcg gttccagtgc cgccttccag gacaccttct ggaaaatccc |
| 1621 | aactttaaac gaatctgcac aggcaatgaa agcttagaag aaaactatgt ccaggacagt |
| 1681 | aagatggggt ttgtcatcaa tgccatctat gccatggcac atgggctgca gaacatgcac |
| 1741 | catgccctct gccctggcca cgtgggcctc tgcgatgcca tgaagcccat cgacggcagc |
| 1801 | aagctgctgg acttcctcat caagtcctca ttcattggag tatctggaga ggaggtgtgg |
| 1861 | tttgatgaga aaggagacgc tcctggaagg tatgatatca tgaatctgca gtacactgaa |
| 1921 | gctaatcgct atgactatgt gcacgttgga acctggcatg aaggagtgct gaacattgat |
| 1981 | gattacaaaa tccagatgaa caagagtgga gtggtgcggt ctgtgtgcag tgagccttgc |
| 2041 | ttaaagggcc agattaaggt tatacggaaa ggagaagtga gctgctgctg gatttgcacg |
| 2101 | gcctgcaaag agaatgaata tgtgcaagat gagttcacct gcaaagcttg tgacttggga |
| 2161 | tggtggccca atgcagatct aacaggctgt gagcccattc ctgtgcgcta tcttgagtgg |
| 2221 | agcaacatcg aatccattat agccatcgcc ttttcatgcc tgggaatcct tgttaccttg |
| 2281 | tttgtcaccc taatctttgt actgtaccgg gacacaccag tggtcaaatc ctccagtcgg |
| 2341 | gagctctgct acatcatcct agctggcatc ttccttggtt atgtgtgccc attcactctc |
| 2401 | attgccaaac ctactaccac ctcctgctac ctccagcgcc tcttggttgg cctctcctct |
| 2461 | gcgatgtgct actctgcttt agtgactaaa accaatcgta ttgcacgcat cctggctggc |
| 2521 | agcaagaaga agatctgcac ccggaagccc aggttcatga gtgcctgggc tcaggtgatc |
| 2581 | attgcctcaa ttctgattag tgtgcaacta accctggtgg taaccctgat catcatggaa |
| 2641 | ccccctatgc ccattctgtc ctacccaagt atcaaggaag tctaccttat ctgcaatacc |
| 2701 | agcaacctgg gtgtggtggc ccctttgggc tacaatggac tcctcatcat gagctgtacc |
| 2761 | tactatgcct tcaagacccg caacgtgccc gccaacttca acgaggccaa atatatcgcg |
| 2821 | ttcaccatgt acaccacctg tatcatctgg ctagcttttg tgcccattta ctttgggagc |
| 2881 | aactacaaga tcatcacaac ttgctttgca gtgagtctca gtgtaacagt ggctctgggg |
| 2941 | tgcatgttca ctcccaagat gtacatcatt attgccaagc ctgagaggaa tgtccgcagt |
| 3001 | gccttcacca cctctgatgt tgtccgcatg catgttggcg atggcaagct gccctgccgc |
| 3061 | tccaacactt tcctcaacat cttccgaaga aagaaggcag gggcagggaa tgccaattct |
| 3121 | aatggcaagt ctgtgtcatg gtctgaacca ggtggaggac aggtgcccaa gggacagcat |
| 3181 | atgtggcacc gcctctctgt gcacgtgaag accaatgaga cggcctgcaa ccaaacagcc |
| 3241 | gtcatcaagc ccctcactaa aagttaccaa ggctctggca agagcctgac cttttcagat |
| 3301 | accagcacca agacccttta caacgtagag gaggaggagg atgcccagcc gattcgcttt |
| 3361 | agcccgcctg gtagcccttc catggtggtg cacaggcgcg tgccaagcgc ggcgaccact |
| 3421 | ccgcctctgc cgtcccacct gaccgcagag gagacccccc tcttcctggc cgaaccagcc |
| 3481 | ctccccaagg gcttgccccc tcctctccag cagcagcagc aaccccctcc acagcagaaa |
| 3541 | tcgctgatgg accagctcca gggagtggtc agcaacttca gtaccgcgat cccggatttt |
| 3601 | cacgcggtgc tggcaggccc cggtggtccc gggaacgggc tgcggtccct gtacccgccc |
| 3661 | ccgccacctc cgcagcacct gcagatgctg ccgctgcagc tgagcacctt tggggaggag |
| 3721 | ctggtctccc cgcccgcgga cgacgacgac gacagcgaga ggtttaagct cctccaggag |
| 3781 | tacgtgtatg agcacgagcg ggaagggaac acggaagaag acgaactgga agaggaggag |
| 3841 | gaggacctgc aggcggccag caaactgacc ccggatgatt cgcctgcgct gacgcctccg |
| 3901 | tcgcctttcc gcgactcggt ggcctcgggc agctcggtgc ccagctcccc cgtgtccgag |
| 3961 | tcggtgctct gcacccctcc caacgtatcc tacgcctctg tcattctgcg ggactacaag |
| 4021 | caaagctctt ccaccctgta agggggaagg gtccacatag aaaagcaaga caagccagag |
| 4081 | atctcccaca cctccagaga tgtgcaaaca gctgggagga aaagcctggg agtggggggc |
| 4141 | ctcgtcggga ggacaggaga ccgctgctgc tgctgccgct actgctgctg ctgccttaag |
| 4201 | taggaagaga gggaaggaca ccaagcaaaa aatgttccag gccaggattc ggattcttga |
| 4261 | attactcgaa gccttctctg ggaagaaagg gaattctgac aaagcacaat tccatatggt |
| 4321 | atgtaacttt tatcacaaat caaatagtga catcacaaac ataatgtcct cttttgcaca |
| 4381 | attgtgcata gatatatata tgcccacaca cactgggcca tgcttgccaa ggaacagccc |
| 4441 | acgtggacat gccagtcgga tcatgagttc acctgatggc attcggagtg agctggtgga |
| 4501 | gccagacaga gcaggtgcgg ggaagggaag ggcccaggcc agacccatcc caaacggatg |
| 4561 | atgggatgat gggacagcag ctccttgctc agaagccctt ctccccgctg ggctgacaga |
| 4621 | ctcctcatct tcaggagact caggaatgga gcggcacagg ggtctctctt catccactgc |
| 4681 | aacccatcca gtgccagctt tgagattgca cttgaagaaa ggtgcatgga ccccctgctg |
| 4741 | ctctgcagat tccctttatt taggaaaaca ggaataagag caaaattatc accaaaaagt |
| 4801 | gcttcatcag gcgtgctaca ggaggaagga gctagaaata gaacaatcca tcagcatgag |
| 4861 | actttgaaaa aaaaacacat gatcagcttc tcatgttcca tattcactta ttggcgattt |
| 4921 | ggggaaaagg ccggaacaag agattgttac gagagtggca gaaacccttt tgtagattga |
| 4981 | cttgtgtttg tgccaagcgg gctttccatt gaccttcagt taaagaacaa accatgtgac |
| 5041 | aaaattgtta ccttccactt actgtagcaa ataataccta caagttgaac ttctaagatg |
| 5101 | cgtatatgta caatttggtg ccattatttc tcctacgtat tagagaaaca aatccatctt |
| 5161 | tgaatctaat ggtgtactca tagcaactat tactggttta aatgacaaat aattctatcc |
| 5221 | tattgtcact gaagtccttg taactagcga gtgaatgtgt tcctgtgtcc ttgtatatgt |
| 5281 | gcgatcgtaa aatttgtgca atgtaatgtc aaattgactg gtcaatgtca acctagtagt |
| 5341 | caatctaact gcaattagaa attgtctttt gaatatacta tatatatttt ttatgttcca |
| 5401 | ataatgtttt gtacatcatt gtcatcaata tctacagaag ctctttgacg gtttgaatac |
| 5461 | tatggctcaa ggttttcata tgcagctcgg atggacattt ttcttctaag atggaactta |
| 5521 | tttttcagat attttctgat gtggagatat gttattaatg aagtggtttg aaaatttgtt |
| 5581 | atattaaaag tgcacaaaaa ctgagagtga aaataaaagg tacattttat aagcttgcac |
| 5641 | acattattaa cacataagat tgaacaaagc atttagatta ttccaggtta tatcattttt |
| 5701 | ttaaagattt tccacagcta cttgagtgtc taacatacag taacatctaa ctcagctaat |
| 5761 | aatttgtaaa atctttatca atcacatttt gccttctttt aatttttatg ttcatggact |
| 5821 | tttattcctg tgtcttggct gtcataactt tttatttctg ctatttgctg ttgtgtaata |
| 5881 | tccatggaca tgtaatccac ttactccatc tttacaatcc ctttttacca ccaataaaag |
| 5941 | gatttttctt gctgttttga tttcttctat tatttgtgga atgaattata ccccccttaa |
| 6001 | atatctttgt ttatgcctta tgttcagtca tattttaata tgcttccttc atattgaagc |
| 6061 | tgctgatttc tcagccaaaa atcatcttag aatctttaaa tatccattgc atcatttgtt |
| 6121 | cagaatttaa catccattcc aatgttggag gcttgtatta cttatatttc atcatattct |
| 6181 | attgccaagt ttagtcagtt ccacaccaag aatgaactgc atttccttta aaaattattt |
| 6241 | taaaacacct ttattgaaaa gatctcatga ctgagatgtg gactttggtt ccatgttttc |
| 6301 | attgtaagaa agcagagagc ggaaaatcaa tggctccagt gattaataga tgggttttta |
| 6361 | gtaattgaca aattcatgag ggaaagcata tgatctcttt attagtgaat catgcttatt |
| 6421 | ttttactctt aatgccacta atatacatcc ctaatatcac agggcttgtg cattcagatt |
| 6481 | tttaaaaaat taggatagat aaggaaacaa cttatattca agtgtaagat gatatcaggt |
| 6541 | tggtctaaga cttttggtga acacgttcat tcaactgtga tcactttatt actctgaatg |
| 6601 | cctactatta tcctgattat ggggtctcct gaataaatag agtattagtc cttatgtcat |
| 6661 | cattgttcaa aattggagat gtacacatac ataccctata ccaagagggc cgaaactctt |
| 6721 | caccttgatg tatgttctga tacaagttgt tcagcttctt gtaaatgtgt tttccttcgg |
| 6781 | cttgttactg ccttttgtca aataatcttg acaatgctgt ataataaata ttttctattt |
| 6841 | attaaa. |

In some embodiments, the human mGluR1 comprises or consists of the amino acid sequence of the following sequence, or a functional fragment or variant thereof (UniProtKB Q13255-1, isoform 1; and SEQ ID NO: 3):

| | |
|---|---|
| 1 | MVGLLLFFFP AIFLEVSLLP RSPGRKVLLA GASSQRSVAR MDGDVIIGAL FSVHHQPPAE |
| 61 | KVPERKCGEI REQYGIQRVE AMFHTLDKIN ADPVLLPNIT LGSEIRDSCW HSSVALEQSI |
| 121 | EFIRDSLISI RDEKDGINRC LPDGQSLPPG RTKKPIAGVI GPGSSSVAIQ VQNLLQLFDI |
| 181 | PQIAYSATSI DLSDKTLYKY FLRVVPSDTL QARAMLDIVK RYNWTYVSAV HTEGNYGESG |
| 241 | MDAFKELAAQ EGLCIAHSDK IYSNAGEKSF DRLLRKLRER LPKARVVVCF CEGMTVRGLL |
| 301 | SAMRRLGVVG EFSLIGSDGW ADRDEVIEGY EVEANGGITI KLQSPEVRSF DDYFLKLRLD |
| 361 | TNTRNPWFPE FWQHRFQCRL PGHLLENPNF KRICTGNESL EENYVQDSKM GFVINAIYAM |
| 421 | AHGLQNMHHA LCPGHVGLCD AMKPIDGSKL LDFLIKSSFI GVSGEEVWFD EKGDAPGRYD |
| 481 | IMNLQYTEAN RYDYVHVGTW HEGVLNIDDY KIQMNKSGVV RSVCSEPCLK GQIKVIRKGE |
| 541 | VSCCWICTAC KENEYVQDEF TCKACDLGWW PNADLTGCEP IPVRYLEWSN IESIIAIAFS |
| 601 | CLGILVTLFV TLIFVLYRDT PVVKSSSREL CYIILAGIFL GYVCPFTLIA KPTTTSCYLQ |
| 661 | RLLVGLSSAM CYSALVTKTN RIARILAGSK KKICTRKPRF MSAWAQVIIA SILISVQLTL |
| 721 | VVTLIIMEPP MPILSYPSIK EVYLICNTSN LGVVAPLGYN GLLIMSCTYY AFKTRNVPAN |
| 781 | FNEAKYIAFT MYTTCIIWLA FVPIYFGSNY KIITTCFAVS LSVTVALGCM FTPKMYIIIA |
| 841 | KPERNVRSAF TTSDVVRMHV GDGKLPCRSN TFLNIFRRKK AGAGNANSNG KSVSWSEPGG |
| 901 | GQVPKGQHMW HRLSVHVKTN ETACNQTAVI KPLTKSYQGS GKSLTFSDTS TKTLYNVEEE |
| 961 | EDAQPIRFSP PGSPSMVVHR RVPSAATTPP LPSHLTAEET PLFLAEPALP KGLPPPLQQQ |
| 1021 | QQPPPQQKSL MDQLQGVVSN FSTAIPDFHA VLAGPGGPGN GLRSLYPPPP PPQHLQMLPL |
| 1081 | QLSTFGEELV SPPADDDDDS ERFKLLQEYV YEHEREGNTE EDELEEEEED LQAASKLTPD |
| 1141 | DSPALTPPSP FRDSVASGSS VPSSPVSESV LCTPPNVSYA SVILRDYKQS SSTL. |

In some embodiments, the sequence encoding a human mGluR1 comprises or consists of the nucleic acid sequence of the following sequence, or a functional fragment or variant thereof (UniProtKB Q13255-2; GenBank Accession No. NM_001278065.2 and SEQ ID NO: 4):

| | |
|---|---|
| 1 | agagctgagg cgtctgcaag ccgagcgtgg ccacggtcct ctggccccgg gaccatagcg |
| 61 | ctgtctaccc cgactcaggt actcaggtat gtctcaagtc catgtcctcc aaacagactc |
| 121 | agcatctagc tcaccgctgc caacacgact tccactgtac tcttgatcaa tttaccttga |
| 181 | tgcactaccg gtgaagaacg gggactcgaa ttcccttaca aacgcctcca gcttgtagag |
| 241 | gcggtcgtgg aggacccaga ggaggagacg aaggggaagg aggcggtggt ggaggaggca |
| 301 | aaggccttgg acgaccattg ttggcgaggg gcaccactcc gggagaggcg gcgctgggcg |
| 361 | tcttgggggt gcgcgccggg agcctgcagc gggaccagcg tgggaacgcg gctggcaggc |
| 421 | tgtggacctc gtcctcacca ccatggtcgg gctccttttg ttttttttcc cagcgatctt |
| 481 | tttggaggtg tcccttctcc ccagaagccc cggcaggaaa gtgttgctgg caggagcgtc |
| 541 | gtctcagcgc tcggtggcca gaatggacgg agatgtcatc attggagccc tcttctcagt |
| 601 | ccatcaccag cctccggccg agaaagtgcc cgagaggaag tgtggggaga tcagggagca |
| 661 | gtatggcatc cagagggtgg aggccatgtt ccacacgttg gataagatca acgcggaccc |
| 721 | ggtcctcctg cccaacatca ccctgggcag tgagatccgg gactcctgct ggcactcttc |
| 781 | cgtggctctg gaacagagca ttgagttcat tagggactct ctgatttcca ttcgagatga |
| 841 | gaaggatggg atcaaccggt gtctgcctga cggccagtcc ctccccccag gcaggactaa |
| 901 | gaagcccatt gcgggagtga tcggtcccgg ctccagctct gtagccattc aagtgcagaa |
| 961 | cctgctccag ctcttcgaca tcccccagat cgcttattca gccacaagca tcgacctgag |
| 1021 | tgacaaaact ttgtacaaat acttcctgag ggttgtccct tctgacactt tgcaggcaag |
| 1081 | ggccatgctt gacatagtca aacgttacaa ttggacctat gtctctgcag tccacacgga |
| 1141 | agggaattat ggggagagcg gaatggacgc tttcaaagag ctggctgccc aggaaggcct |
| 1201 | ctgtatcgcc cattctgaca aaatctacag caacgctggg gagaagagct ttgaccgact |
| 1261 | cttgcgcaaa ctccgagaga ggcttcccaa ggctagagtg gtggtctgct tctgtgaagg |
| 1321 | catgacagtg cgaggactcc tgagcgccat gcggcgcctt ggcgtcgtgg gcgagttctc |
| 1381 | actcattgga agtgatggat gggcagacag agatgaagtc attgaaggtt atgaggtgga |
| 1441 | agccaacggg ggaatcacga taaagctgca gtctccagag gtcaggtcat ttgatgatta |
| 1501 | tttcctgaaa ctgaggctgg acactaacac gaggaatccc tggttccctg agttctggca |
| 1561 | acatcggttc cagtgccgcc ttccaggaca ccttctggaa aatcccaact ttaaacgaat |
| 1621 | ctgcacaggc aatgaaagct tagaagaaaa ctatgtccag gacagtaaga tggggtttgt |
| 1681 | catcaatgcc atctatgcca tggcacatgg gctgcagaac atgcaccatg ccctctgccc |
| 1741 | tggccacgtg ggcctctgcg atgccatgaa gcccatcgac ggcagcaagc tgctggactt |
| 1801 | cctcatcaag tcctcattca ttggagtatc tggagaggag gtgtggtttg atgagaaagg |
| 1861 | agacgctcct ggaaggtatg atatcatgaa tctgcagtac actgaagcta atcgctatga |
| 1921 | ctatgtgcac gttggaacct ggcatgaagg agtgctgaac attgatgatt acaaaatcca |
| 1981 | gatgaacaag agtggagtgg tgcggtctgt gtgcagtgag ccttgcttaa agggccagat |
| 2041 | taaggttata cggaaaggag aagtgagctg ctgctggatt tgcacggcct gcaaagagaa |
| 2101 | tgaatatgtg caagatgagt tcacctgcaa agcttgtgac ttgggatggt ggcccaatgc |
| 2161 | agatctaaca ggctgtgagc ccattcctgt gcgctatctt gagtggagca acatcgaatc |
| 2221 | cattatagcc atcgcctttt catgcctggg aatccttgtt accttgtttg tcaccctaat |
| 2281 | ctttgtactg taccgggaca caccagtggt caaatcctcc agtcgggagc tctgctacat |
| 2341 | catcctagct ggcatcttcc ttggttatgt gtgcccattc actctcattg ccaaacctac |
| 2401 | taccacctcc tgctacctcc agcgcctctt ggttggcctc tcctctgcga tgtgctactc |
| 2461 | tgctttagtg actaaaacca atcgtattgc acgcatcctg gctggcagca agaagaagat |
| 2521 | ctgcacccgg aagcccaggt tcatgagtgc ctgggctcag gtgatcattg cctcaattct |
| 2581 | gattagtgtg caactaaccc tggtggtaac cctgatcatc atggaacccc ctatgcccat |
| 2641 | tctgtcctac ccaagtatca aggaagtcta ccttatctgc aataccagca acctgggtgt |
| 2701 | ggtggcccct ttgggctaca atggactcct catcatgagc tgtacctact atgccttcaa |
| 2761 | gacccgcaac gtgcccgcca acttcaacga ggccaaatat atcgcgttca ccatgtacac |
| 2821 | cacctgtatc atctggctag cttttgtgcc catttacttt gggagcaact acaagatcat |
| 2881 | cacaacttgc tttgcagtga gtctcagtgt aacagtggct ctggggtgca tgttcactcc |
| 2941 | caagatgtac atcattattg ccaagcctga gaggaatgtc cgcagtgcct tcaccacctc |
| 3001 | tgatgttgtc cgcatgcatg ttggcgatgg caagctgccc tgccgctcca acactttcct |
| 3061 | caacatcttc cgaagaaaga aggcaggggc agggaatgcc aagaagaggc agccagaatt |
| 3121 | ctcgcccacc agccaatgtc cgtcggcaca tgtgcagctt tgaaaacccc cacactgcag |
| 3181 | tgaatgtttc taatggcaag tctgtgtcat ggtctgaacc aggtggagga caggtgccca |
| 3241 | agggacagca tatgtggcac cgcctctctg tgcacgtgaa gaccaatgag acggcctgca |
| 3301 | accaaacagc cgtcatcaag cccctcacta aaagttacca aggctctggc aagagcctga |
| 3361 | ccttttcaga taccagcacc aagacccttt acaacgtaga ggaggaggag gatgcccagc |
| 3421 | cgattcgctt tagcccgcct ggtagccctt ccatggtggt gcacaggcgc gtgccaagcg |
| 3481 | cggcgaccac tccgcctctg ccgtcccacc tgaccgcaga ggagaccccc ctcttcctgg |
| 3541 | ccgaaccagc cctccccaag ggcttgcccc ctcctctcca gcagcagcag caaccccctc |
| 3601 | cacagcagaa atcgctgatg gaccagctcc agggagtggt cagcaacttc agtaccgcga |
| 3661 | tcccggattt tcacgcggtg ctggcaggcc ccggtggtcc cgggaacggg ctgcggtccc |
| 3721 | tgtacccgcc cccgccacct ccgcagcacc tgcagatgct gccgctgcag ctgagcacct |
| 3781 | ttggggagga gctggtctcc ccgcccgcgg acgacgacga cgacagcgag aggtttaagc |
| 3841 | tcctccagga gtacgtgtat gagcacgagc gggaagggaa cacggaagaa gacgaactgg |
| 3901 | aagaggagga ggaggacctg caggcggcca gcaaactgac cccggatgat tcgcctgcgc |
| 3961 | tgacgcctcc gtcgcctttc cgcgactcgg tggcctcggg cagctcggtg cccagctccc |
| 4021 | ccgtgtccga gtcggtgctc tgcacccctc ccaacgtatc ctacgcctct gtcattctgc |
| 4081 | gggactacaa gcaaagctct tccaccctgt aagggggaag ggtccacata gaaaagcaag |
| 4141 | acaagccaga gatctcccac acctccagag atgtgcaaac agctgggagg aaaagcctgg |
| 4201 | gagtgggggg cctcgtcggg aggacaggag accgctgctg ctgctgccgc tactgctgct |
| 4261 | gctgccttaa gtaggaagag agggaaggac accaagcaaa aaatgttcca ggccaggatt |
| 4321 | cggattcttg aattactcga agccttctct gggaagaaag ggaattctga caaagcacaa |
| 4381 | ttccatatgg tatgtaactt ttatcacaaa tcaaatagtg acatcacaaa cataatgtcc |
| 4441 | tcttttgcac aattgtgcat agatatatat atgcccacac acactgggcc atgcttgcca |
| 4501 | aggaacagcc cacgtggaca tgccagtcgg atcatgagtt cacctgatgg cattcggagt |
| 4561 | gagctggtgg agccagacag agcaggtgcg gggaagggaa gggcccaggc cagacccatc |
| 4621 | ccaaacggat gatgggatga tgggacagca gctccttgct cagaagccct tctccccgct |
| 4681 | gggctgacag actcctcatc ttcaggagac tcaggaatgg agcggcacag gggtctctct |
| 4741 | tcatccactg caacccatcc agtgccagct ttgagattgc acttgaagaa aggtgcatgg |
| 4801 | accccctgct gctctgcaga ttccctttat ttaggaaaac aggaataaga gcaaaattat |
| 4861 | caccaaaaag tgcttcatca ggcgtgctac aggaggaagg agctagaaat agaacaatcc |
| 4921 | atcagcatga gactttgaaa aaaaaacaca tgatcagctt ctcatgttcc atattcactt |
| 4981 | attggcgatt tggggaaaag gccggaacaa gagattgtta cgagagtggc agaaaccctt |
| 5041 | ttgtagattg acttgtgttt gtgccaagcg ggctttccat tgaccttcag ttaaagaaca |
| 5101 | aaccatgtga caaaattgtt accttccact tactgtagca aataatacct acaagttgaa |
| 5161 | cttctaagat gcgtatatgt acaatttggt gccattattt ctcctacgta ttagagaaac |
| 5221 | aaatccatct ttgaatctaa tggtgtactc atagcaacta ttactggttt aaatgacaaa |
| 5281 | taattctatc ctattgtcac tgaagtcctt gtaactagcg agtgaatgtg ttcctgtgtc |
| 5341 | cttgtatatg tgcgatcgta aaatttgtgc aatgtaatgt caaattgact ggtcaatgtc |
| 5401 | aacctagtag tcaatctaac tgcaattaga aattgtcttt tgaatatact atatatattt |
| 5461 | tttatgttcc aataatgttt tgtacatcat tgtcatcaat atctacagaa gctctttgac |
| 5521 | ggtttgaata ctatggctca aggttttcat atgcagctcg gatggacatt tttcttctaa |
| 5581 | gatggaactt atttttcaga tattttctga tgtggagata tgttattaat gaagtggttt |
| 5641 | gaaaatttgt tatattaaaa gtgcacaaaa actgagagtg aaaataaaag gtacatttta |
| 5701 | taagcttgca cacattatta acacataaga ttgaacaaag catttagatt attccaggtt |
| 5761 | atatcatttt tttaaagatt ttccacagct acttgagtgt ctaacataca gtaacatcta |
| 5821 | actcagctaa taatttgtaa aatctttatc aatcacattt tgccttcttt taatttttat |
| 5881 | gttcatggac ttttattcct gtgtcttggc tgtcataact ttttatttct gctatttgct |
| 5941 | gttgtgtaat atccatggac atgtaatcca cttactccat ctttacaatc cctttttacc |
| 6001 | accaataaaa ggatttttct tgctgttttg atttcttcta ttatttgtgg aatgaattat |
| 6061 | acccccctta aatatctttg tttatgcctt atgttcagtc atattttaat atgcttcctt |
| 6121 | catattgaag ctgctgattt ctcagccaaa aatcatctta gaatctttaa atatccattg |
| 6181 | catcatttgt tcagaattta acatccattc caatgttgga ggcttgtatt acttatattt |
| 6241 | catcatattc tattgccaag tttagtcagt tccacaccaa gaatgaactg catttccttt |
| 6301 | aaaaattatt ttaaaacacc tttattgaaa agatctcatg actgagatgt ggactttggt |
| 6361 | tccatgtttt cattgtaaga aagcagagag cggaaaatca atggctccag tgattaatag |
| 6421 | atgggttttt agtaattgac aaattcatga gggaaagcat atgatctctt tattagtgaa |
| 6481 | tcatgcttat tttttactct taatgccact aatatacatc cctaatatca cagggcttgt |
| 6541 | gcattcagat ttttaaaaaa ttaggataga taaggaaaca acttatattc aagtgtaaga |
| 6601 | tgatatcagg ttggtctaag acttttggtg aacacgttca ttcaactgtg atcactttat |
| 6661 | tactctgaat gcctactatt atcctgatta tggggtctcc tgaataaata gagtattagt |
| 6721 | ccttatgtca tcattgttca aaattggaga tgtacacata cataccctat accaagaggg |
| 6781 | ccgaaactct tcaccttgat gtatgttctg atacaagttg ttcagcttct tgtaaatgtg |
| 6841 | ttttccttcg gcttgttact gccttttgtc aaataatctt gacaatgctg tataataaat |
| 6901 | attttctatt tattaaa. |

In some embodiments, the human mGluR1 comprises or consists of the amino acid sequence of the following sequence, or a functional fragment or variant thereof (UniProtKB Q13255-2, isoform 2; and SEQ ID NO: 5):

| | |
|---|---|
| 1 | MVGLLLFFFP AIFLEVSLLP RSPGRKVLLA GASSQRSVAR MDGDVIIGAL FSVHHQPPAE |
| 61 | KVPERKCGEI REQYGIQRVE AMFHTLDKIN ADPVLLPNIT LGSEIRDSCW HSSVALEQSI |
| 121 | EFIRDSLISI RDEKDGINRC LPDGQSLPPG RTKKPIAGVI GPGSSSVAIQ VQNLLQLFDI |
| 181 | PQIAYSATSI DLSDKTLYKY FLRVVPSDTL QARAMLDIVK RYNWTYVSAV HTEGNYGESG |
| 241 | MDAFKELAAQ EGLCIAHSDK IYSNAGEKSF DRLLRKLRER LPKARVVVCF CEGMTVRGLL |
| 301 | SAMRRLGVVG EFSLIGSDGW ADRDEVIEGY EVEANGGITI KLQSPEVRSF DDYFLKLRLD |
| 361 | TNTRNPWFPE FWQHRFQCRL PGHLLENPNF KRICTGNESL EENYVQDSKM GFVINAIYAM |
| 421 | AHGLQNMHHA LCPGHVGLCD AMKPIDGSKL LDFLIKSSFI GVSGEEVWFD EKGDAPGRYD |
| 481 | IMNLQYTEAN RYDYVHVGTW HEGVLNIDDY KIQMNKSGVV RSVCSEPCLK GQIKVIRKGE |
| 541 | VSCCWICTAC KENEYVQDEF TCKACDLGWW PNADLTGCEP IPVRYLEWSN IESIIAIAFS |
| 601 | CLGILVTLFV TLIFVLYRDT PVVKSSSREL CYIILAGIFL GYVCPFTLIA KPTTTSCYLQ |
| 661 | RLLVGLSSAM CYSALVTKTN RIARILAGSK KKICTRKPRF MSAWAQVIIA SILISVQLTL |
| 721 | VVTLIIMEPP MPILSYPSIK EVYLICNTSN LGVVAPLGYN GLLIMSCTYY AFKTRNVPAN |
| 781 | FNEAKYIAFT MYTTCIIWLA FVPIYFGSNY KIITTCFAVS LSVTVALGCM FTPKMYIIIA |
| 841 | KPERNVRSAF TTSDVVRMHV GDGKLPCRSN TFLNIFRRKK AGAGNAKKRQ PEFSPTSQCP |
| 901 | SAHVQL. |

In some embodiments, the sequence encoding a human mGluR1 comprises or consists of the nucleic acid sequence of the following sequence, or a functional fragment or variant thereof (UniProtKB Q13255-3; GenBank Accession No. NM_001278067.1 and SEQ ID NO: 6):

| | |
|---|---|
| 1 | catctagctc accgctgcca acacgacttc cactgtactc ttgatcaatt taccttgatg |
| 61 | cactaccggt gaagaacggg gactcgaatt cccttacaaa cgcctccagc ttgtagaggc |
| 121 | ggtcgtggag gacccagagg aggagacgaa ggggaaggag gcggtggtgg aggaggcaaa |
| 181 | ggccttggac gaccattgtt ggcgaggggc accactccgg gagaggcggc gctgggcgtc |
| 241 | ttgggggtgc gcgccgggag cctgcagcgg gaccagcgtg ggaacgcggc tggcaggctg |
| 301 | tggacctcgt cctcaccacc atggtcgggc tccttttgtt ttttttccca gcgatctttt |
| 361 | tggaggtgtc ccttctcccc agaagccccg gcaggaaagt gttgctggca ggagcgtcgt |
| 421 | ctcagcgctc ggtggccaga atggacggag atgtcatcat tggagccctc ttctcagtcc |
| 481 | atcaccagcc tccggccgag aaagtgcccg agaggaagtg tggggagatc agggagcagt |
| 541 | atggcatcca gagggtggag gccatgttcc acacgttgga taagatcaac gcggacccgg |
| 601 | tcctcctgcc caacatcacc ctgggcagtg agatccggga ctcctgctgg cactcttccg |
| 661 | tggctctgga acagagcatt gagttcatta gggactctct gatttccatt cgagatgaga |
| 721 | aggatgggat caaccggtgt ctgcctgacg gccagtccct ccccccaggc aggactaaga |
| 781 | agcccattgc gggagtgatc ggtcccggct ccagctctgt agccattcaa gtgcagaacc |
| 841 | tgctccagct cttcgacatc ccccagatcg cttattcagc cacaagcatc gacctgagtg |
| 901 | acaaaacttt gtacaaatac ttcctgaggg ttgtcccttc tgacactttg caggcaaggg |
| 961 | ccatgcttga catagtcaaa cgttacaatt ggacctatgt ctctgcagtc cacacggaag |
| 1021 | ggaattatgg ggagagcgga atggacgctt tcaaagagct ggctgcccag gaaggcctct |
| 1081 | gtatcgccca ttctgacaaa atctacagca acgctgggga gaagagcttt gaccgactct |
| 1141 | tgcgcaaact ccgagagagg cttcccaagg ctagagtggt ggtctgcttc tgtgaaggca |
| 1201 | tgacagtgcg aggactcctg agcgccatgc ggcgccttgg cgtcgtgggc gagttctcac |
| 1261 | tcattggaag tgatggatgg gcagacagag atgaagtcat tgaaggttat gaggtggaag |
| 1321 | ccaacggggg aatcacgata aagctgcagt ctccagaggt caggtcattt gatgattatt |
| 1381 | tcctgaaact gaggctggac actaacacga ggaatccctg gttccctgag ttctggcaac |
| 1441 | atcggttcca gtgccgcctt ccaggacacc ttctggaaaa tcccaacttt aaacgaatct |
| 1501 | gcacaggcaa tgaaagctta gaagaaaact atgtccagga cagtaagatg gggtttgtca |
| 1561 | tcaatgccat ctatgccatg gcacatgggc tgcagaacat gcaccatgcc ctctgccctg |
| 1621 | gccacgtggg cctctgcgat gccatgaagc ccatcgacgg cagcaagctg ctggacttcc |
| 1681 | tcatcaagtc ctcattcatt ggagtatctg gagaggaggt gtggtttgat gagaaaggag |
| 1741 | acgctcctgg aaggtatgat atcatgaatc tgcagtacac tgaagctaat cgctatgact |
| 1801 | atgtgcacgt tggaacctgg catgaaggag tgctgaacat tgatgattac aaaatccaga |
| 1861 | tgaacaagag tggagtggtg cggtctgtgt gcagtgagcc ttgcttaaag ggccagatta |
| 1921 | aggttatacg gaaaggagaa gtgagctgct gctggatttg cacggcctgc aaagagaatg |
| 1981 | aatatgtgca agatgagttc acctgcaaag cttgtgactt gggatggtgg cccaatgcag |
| 2041 | atctaacagg ctgtgagccc attcctgtgc gctatcttga gtggagcaac atcgaatcca |
| 2101 | ttatagccat cgccttttca tgcctgggaa tccttgttac cttgtttgtc accctaatct |
| 2161 | ttgtactgta ccgggacaca ccagtggtca aatcctccag tcgggagctc tgctacatca |
| 2221 | tcctagctgg catcttcctt ggttatgtgt gcccattcac tctcattgcc aaacctacta |
| 2281 | ccacctcctg ctacctccag cgcctcttgg ttggcctctc ctctgcgatg tgctactctg |
| 2341 | ctttagtgac taaaaccaat cgtattgcac gcatcctggc tggcagcaag aagaagatct |
| 2401 | gcacccggaa gcccaggttc atgagtgcct gggctcaggt gatcattgcc tcaattctga |
| 2461 | ttagtgtgca actaaccctg gtggtaaccc tgatcatcat ggaaccccct atgcccattc |
| 2521 | tgtcctaccc aagtatcaag gaagtctacc ttatctgcaa taccagcaac ctgggtgtgg |
| 2581 | tggccccttt gggctacaat ggactcctca tcatgagctg tacctactat gccttcaaga |
| 2641 | cccgcaacgt gcccgccaac ttcaacgagg ccaaatatat cgcgttcacc atgtacacca |
| 2701 | cctgtatcat ctggctagct tttgtgccca tttactttgg gagcaactac aagatcatca |
| 2761 | caacttgctt tgcagtgagt ctcagtgtaa cagtggctct ggggtgcatg ttcactccca |
| 2821 | agatgtacat cattattgcc aagcctgaga ggaatgtccg cagtgccttc accacctctg |
| 2881 | atgttgtccg catgcatgtt ggcgatggca agctgccctg ccgctccaac actttcctca |
| 2941 | acatcttccg aagaaagaag gcaggggcag ggaatgccaa gtggaggaca ggtgcccaag |
| 3001 | ggacagcata tgtggcaccg cctctctgtg cacgtgaaga ccaatgagac ggcctgcaac |
| 3061 | caaacagccg tcatcaagcc cctcactaaa agttaccaag gctctggcaa gagcctgacc |
| 3121 | ttttcagata ccagcaccaa gaccctttac aacgtagagg aggaggagga tgcccagccg |
| 3181 | attcgcttta gcccgcctgg tagcccttcc atggtggtgc acaggcgcgt gccaagcgcg |
| 3241 | gcgaccactc cgcctctgcc gtcccacctg accgcagagg agacccccct cttcctggcc |
| 3301 | gaaccagccc tccccaaggg cttgccccct cctctccagc agcagcagca accccctcca |
| 3361 | cagcagaaat cgctgatgga ccagctccag ggagtggtca gcaacttcag taccgcgatc |
| 3421 | ccggattttc acgcggtgct ggcaggcccc ggtggtcccg ggaacgggct gcggtccctg |
| 3481 | tacccgcccc cgccacctcc gcagcacctg cagatgctgc cgctgcagct gagcaccttt |
| 3541 | ggggaggagc tggtctcccc gcccgcggac gacgacgacg acagcgagag gtttaagctc |
| 3601 | ctccaggagt acgtgtatga gcacgagcgg gaagggaaca cggaagaaga cgaactggaa |
| 3661 | gaggaggagg aggacctgca ggcggccagc aaactgaccc cggatgattc gcctgcgctg |
| 3721 | acgcctccgt cgcctttccg cgactcggtg gcctcgggca gctcggtgcc cagctccccc |
| 3781 | gtgtccgagt cggtgctctg cacccctccc aacgtatcct acgcctctgt cattctgcgg |
| 3841 | gactacaagc aaagctcttc caccctgtaa gggggaaggg tccacataga aaagcaagac |
| 3901 | aagccagaga tctcccacac ctccagagat gtgcaaacag ctgggaggaa aagcctggga |
| 3961 | gtggggggcc tcgtcgggag gacaggagac cgctgctgct gctgccgcta ctgctgctgc |
| 4021 | tgccttaagt aggaagagag ggaaggacac caagcaaaaa atgttccagg ccaggattcg |
| 4081 | gattcttgaa ttactcgaag ccttctctgg gaagaaaggg aattctgaca aagcacaatt |
| 4141 | ccatatggta tgtaactttt atcacaaatc aaatagtgac atcacaaaca taatgtcctc |
| 4201 | ttttgcacaa ttgtgcatag atatatatat gcccacacac actgggccat gcttgccaag |
| 4261 | gaacagccca cgtggacatg ccagtcggat catgagttca cctgatggca ttcggagtga |
| 4321 | gctggtggag ccagacagag caggtgcggg gaagggaagg gcccaggcca gacccatccc |
| 4381 | aaacggatga tgggatgatg ggacagcagc tccttgctca gaagcccttc tccccgctgg |
| 4441 | gctgacagac tcctcatctt caggagactc aggaatggag cggcacaggg gtctctcttc |
| 4501 | atccactgca acccatccag tgccagcttt gagattgcac ttgaagaaag gtgcatggac |
| 4561 | cccctgctgc tctgcagatt ccctttattt aggaaaacag gaataagagc aaaattatca |
| 4621 | ccaaaaagtg cttcatcagg cgtgctacag gaggaaggag ctagaaatag aacaatccat |
| 4681 | cagcatgaga ctttgaaaaa aaaacacatg atcagcttct catgttccat attcacttat |
| 4741 | tggcgatttg gggaaaaggc cggaacaaga gattgttacg agagtggcag aaaccctttt |
| 4801 | gtagattgac ttgtgtttgt gccaagcggg ctttccattg accttcagtt aaagaacaaa |
| 4861 | ccatgtgaca aaattgttac cttccactta ctgtagcaaa taatacctac aagttgaact |
| 4921 | tctaagatgc gtatatgtac aatttggtgc cattatttct cctacgtatt agagaaacaa |
| 4981 | atccatcttt gaatctaatg gtgtactcat agcaactatt actggtttaa atgacaaata |
| 5041 | attctatcct attgtcactg aagtccttgt aactagcgag tgaatgtgtt cctgtgtcct |
| 5101 | tgtatatgtg cgatcgtaaa atttgtgcaa tgtaatgtca aattgactgg tcaatgtcaa |
| 5161 | cctagtagtc aatctaactg caattagaaa ttgtcttttg aatatactat atatattttt |
| 5221 | tatgttccaa taatgttttg tacatcattg tcatcaatat ctacagaagc tctttgacgg |
| 5281 | tttgaatact atggctcaag gttttcatat gcagctcgga tggacatttt tcttctaaga |
| 5341 | tggaacttat ttttcagata ttttctgatg tggagatatg ttattaatga agtggtttga |
| 5401 | aaatttgtta tattaaaagt gcacaaaaac tgagagtgaa aataaaaggt acattttata |
| 5461 | agcttgcaca cattattaac acataagatt gaacaaagca tttagattat tccaggttat |
| 5521 | atcatttttt taaagatttt ccacagctac ttgagtgtct aacatacagt aacatctaac |
| 5581 | tcagctaata atttgtaaaa tctttatcaa tcacattttg ccttctttta atttttatgt |
| 5641 | tcatggactt ttattcctgt gtcttggctg tcataacttt ttatttctgc tatttgctgt |
| 5701 | tgtgtaatat ccatggacat gtaatccact tactccatct ttacaatccc tttttaccac |
| 5761 | caataaaagg atttttcttg ctgttttgat ttcttctatt atttgtggaa tgaattatac |
| 5821 | cccccttaaa tatctttgtt tatgccttat gttcagtcat attttaatat gcttccttca |
| 5881 | tattgaagct gctgatttct cagccaaaaa tcatcttaga atctttaaat atccattgca |
| 5941 | tcatttgttc agaatttaac atccattcca atgttggagg cttgtattac ttatatttca |
| 6001 | tcatattcta ttgccaagtt tagtcagttc cacaccaaga atgaactgca tttcctttaa |
| 6061 | aaattatttt aaaacacctt tattgaaaag atctcatgac tgagatgtgg actttggttc |
| 6121 | catgttttca ttgtaagaaa gcagagagcg gaaaatcaat ggctccagtg attaatagat |
| 6181 | gggtttttag taattgacaa attcatgagg gaaagcatat gatctcttta ttagtgaatc |
| 6241 | atgcttattt tttactctta atgccactaa tatacatccc taatatcaca gggcttgtgc |
| 6301 | attcagattt ttaaaaaatt aggatagata aggaaacaac ttatattcaa gtgtaagatg |
| 6361 | atatcaggtt ggtctaagac ttttggtgaa cacgttcatt caactgtgat cactttatta |
| 6421 | ctctgaatgc ctactattat cctgattatg gggtctcctg aataaataga gtattagtcc |
| 6481 | ttatgtcatc attgttcaaa attggagatg tacacataca taccctatac caagagggcc |
| 6541 | gaaactcttc accttgatgt atgttctgat acaagttgtt cagcttcttg taaatgtgtt |
| 6601 | ttccttcggc ttgttactgc cttttgtcaa ataatcttga caatgctgta taataaatat |
| 6661 | tttctattta tt. |

In some embodiments, the human mGluR1 comprises or consists of the amino acid sequence of the following sequence, or a functional fragment or variant thereof (UniProtKB Q13255-3, isoform 3; and SEQ ID NO: 7):

| | |
|---|---|
| 1 | MVGLLLFFFP AIFLEVSLLP RSPGRKVLLA GASSQRSVAR MDGDVIIGAL FSVHHQPPAE |
| 61 | KVPERKCGEI REQYGIQRVE AMFHTLDKIN ADPVLLPNIT LGSEIRDSCW HSSVALEQSI |
| 121 | EFIRDSLISI RDEKDGINRC LPDGQSLPPG RTKKPIAGVI GPGSSSVAIQ VQNLLQLFDI |
| 181 | PQIAYSATSI DLSDKTLYKY FLRVVPSDTL QARAMLDIVK RYNWTYVSAV HTEGNYGESG |
| 241 | MDAFKELAAQ EGLCIAHSDK IYSNAGEKSF DRLLRKLRER LPKARVVVCF CEGMTVRGLL |
| 301 | SAMRRLGVVG EFSLIGSDGW ADRDEVIEGY EVEANGGITI KLQSPEVRSF DDYFLKLRLD |
| 361 | TNTRNPWFPE FWQHRFQCRL PGHLLENPNF KRICTGNESL EENYVQDSKM GFVINAIYAM |
| 421 | AHGLQNMHHA LCPGHVGLCD AMKPIDGSKL LDFLIKSSFI GVSGEEVWFD EKGDAPGRYD |
| 481 | IMNLQYTEAN RYDYVHVGTW HEGVLNIDDY KIQMNKSGVV RSVCSEPCLK GQIKVIRKGE |
| 541 | VSCCWICTAC KENEYVQDEF TCKACDLGWW PNADLTGCEP IPVRYLEWSN IESIIAIAFS |
| 601 | CLGILVTLFV TLIFVLYRDT PVVKSSSREL CYIILAGIFL GYVCPFTLIA KPTTTSCYLQ |
| 661 | RLLVGLSSAM CYSALVTKTN RIARILAGSK KKICTRKPRF MSAWAQVIIA SILISVQLTL |
| 721 | VVTLIIMEPP MPILSYPSIK EVYLICNTSN LGVVAPLGYN GLLIMSCTYY AFKTRNVPAN |
| 781 | FNEAKYIAFT MYTTCIIWLA FVPIYFGSNY KIITTCFAVS LSVTVALGCM FTPKMYIIIA |
| 841 | KPERNVRSAF TTSDVVRMHV GDGKLPCRSN TFLNIFRRKK AGAGNAKWRT GAQGTAYVAP |
| 901 | PLCAREDQ. |

In some embodiments, the mGluR comprises mGluR2. In some embodiments, the sequence encoding an mGluR comprises a sequence encoding a human mGluR2.

In some embodiments, the sequence encoding a human mGluR2 comprises or consists of the nucleic acid sequence of the following sequence, or a functional fragment or variant thereof (UniProtKB Q14416; GenBank Accession No. NM_000839.5 and SEQ ID NO: 8):

| | |
|---|---|
| 1 | gagcgcagag ccagcgagcc agcgagcgag cgagcgggag ccggagcctc gcgccccccg |
| 61 | ctccactccg attctctccg cgccagagcc agcgcgccag gagctgggtc ccttcgcatc |
| 121 | tctcttcttg tctgtccttt cctggtccct gtttcctcct ctctttgcct tcgctgcttc |
| 181 | taatctcatc ccctggagac ccaggtctgc gggacccatc catccccttt ggggccatgg |
| 241 | gatcgctgct tgcgctcctg gcactgctgc tgctgtgggg tgctgtggct gagggcccag |
| 301 | ccaagaaggt gctgaccctg gagggagact tggtgctggg tgggctgttc ccagtgcacc |
| 361 | agaagggcgg cccagcagag gactgtggtc ctgtcaatga gcaccgtggc atccagcgcc |
| 421 | tggaggccat gctttttgca ctggaccgca tcaaccgtga cccgcacctg ctgcctggcg |
| 481 | tgcgcctggg tgcacacatc ctcgacagtt gctccaagga cacacatgcg ctggagcagg |
| 541 | cactggactt tgtgcgtgcc tcactcagcc gtggtgctga tggctcacgc cacatctgcc |
| 601 | ccgacggctc ttatgcgacc catggtgatg ctcccactgc catcactggt gttattggcg |
| 661 | gttcctacag tgatgtctcc atccaggtgg ccaacctctt gaggctattt cagatcccac |
| 721 | agattagcta cgcctctacc agtgccaagc tgagtgacaa gtcccgctat gactactttg |
| 781 | cccgcacagt gcctcctgac ttcttccaag ccaaggccat ggctgagatt ctccgcttct |
| 841 | tcaactggac ctatgtgtcc actgtggcgt ctgagggcga ctatggcgag acaggcattg |
| 901 | aggcctttga gctagaggct cgtgcccgca acatctgtgt ggccacctcg gagaaagtgg |
| 961 | gccgtgccat gagccgcgcg gcctttgagg gtgtggtgcg agccctgctg cagaagccca |
| 1021 | gtgcccgcgt ggctgtcctg ttcacccgtt ctgaggatgc ccgggagctg cttgctgcca |
| 1081 | gccagcgcct caatgccagc ttcacctggg tggccagtga tggttggggg gccctggaga |
| 1141 | gtgtggtggc aggcagtgag ggggctgctg agggtgctat caccatcgag ctggcctcct |
| 1201 | accccatcag tgactttgcc tcctacttcc agagcctgga cccttggaac aacagccgga |
| 1261 | acccctggtt ccgtgaattc tgggagcaga ggttccgctg cagcttccgg cagcgagact |
| 1321 | gcgcagccca ctctctccgg gctgtgccct ttgagcagga gtccaagatc atgtttgtgg |
| 1381 | tcaatgcagt gtacgccatg gcccatgcgc tccacaacat gcaccgtgcc ctctgcccca |
| 1441 | acaccacccg gctctgtgac gcgatgcggc cagttaacgg gcgccgcctc tacaaggact |
| 1501 | ttgtgctcaa cgtcaagttt gatgccccct ttcgcccagc tgacacccac aatgaggtcc |
| 1561 | gctttgaccg ctttggtgat ggtattggcc gctacaacat cttcacctat ctgcgtgcag |
| 1621 | gcagtgggcg ctatcgctac cagaaggtgg gctactgggc agaaggcttg actctggaca |
| 1681 | ccagcctcat cccatgggcc tcaccctcag ccggccccct gcccgcctct cgctgcagtg |
| 1741 | agccctgcct ccagaatgag gtgaagagtg tgcagccggg cgaagtctgc tgctggctct |
| 1801 | gcattccgtg ccagccctat gagtaccgat tggacgaatt cacttgcgct gattgtggcc |
| 1861 | tgggctactg gcccaatgcc agcctgactg gctgcttcga actgccccag gagtacatcc |
| 1921 | gctggggcga tgcctgggct gtgggacctg tcaccatcgc ctgcctcggt gccctggcca |
| 1981 | ccctctttgt gctgggtgtc tttgtgcggc acaatgccac accagtggtc aaggcctcag |
| 2041 | gtcgggagct ctgctacatc ctgctgggtg gtgtcttcct ctgctactgc atgaccttca |
| 2101 | tcttcattgc caagccatcc acggcagtgt gtaccttacg gcgtcttggt ttgggcactg |
| 2161 | ccttctctgt ctgctactca gccctgctca ccaagaccaa ccgcattgca cgcatcttcg |
| 2221 | gtggggcccg ggagggtgcc cagcggccac gcttcatcag tcctgcctca caggtggcca |
| 2281 | tctgcctggc acttatctcg ggccagctgc tcatcgtggt cgcctggctg gtggtggagg |
| 2341 | caccgggcac aggcaaggag acagcccccg aacggcggga ggtggtgaca ctgcgctgca |
| 2401 | accaccgcga tgcaagtatg ttgggctcgc tggcctacaa tgtgctcctc atcgcgctct |
| 2461 | gcacgcttta tgccttcaag actcgcaagt gccccgaaaa cttcaacgag gccaagttca |
| 2521 | ttggcttcac catgtacacc acctgcatca tctggctggc attcctgccc atcttctatg |
| 2581 | tcacctccag tgactaccgg gtacagacca ccaccatgtg cgtgtcagtc agcctcagcg |
| 2641 | gctccgtggt gcttggctgc ctctttgcgc ccaagctgca catcatcctc ttccagccgc |
| 2701 | agaagaacgt ggttagccac cgggcaccca ccagccgctt tggcagtgct gctgccaggg |
| 2761 | ccagctccag ccttggccaa gggtctggct cccagtttgt ccccactgtt tgcaatggcc |
| 2821 | gtgaggtggt ggactcgaca acgtcatcgc tttgaagacc ccatactccc gccctgacac |
| 2881 | agctgctcct gggaacctag tgcagaccca cgtccagggc caggaggaag ttggctggag |
| 2941 | cactgcaata atttattacc caccctatgt ctgcccccaa agtcacttac ccacctcctt |
| 3001 | accccagctc ttcagactca gaagtcagga gccttggcca ggagcctctg cagtggccac |
| 3061 | taactgccct tgtagctgtg tttcctcctg gccaggccca gggctcagag aggagcaagc |
| 3121 | cagggttcac tctgccctgg acccgggtgg ctgaggacgg caggccccag tcctaaccag |
| 3181 | caaaggtgct tccagcccag cccctccccc caactagggc cttttttatt ttttatataa |
| 3241 | gttactctgg gatggggagg gtggttattg tgggggctgc ccctccccct gcacagtagt |
| 3301 | ttgtcctgtg gtttattttg tattacctgt aaataaagtg gctttatttt aaaaaa. |

In some embodiments, the human mGluR2 comprises or consists of the amino acid sequence of the following sequence, or a functional fragment or variant thereof (UniProtKB Q14416; GenBank Accession No. NP_000830.2 and SEQ ID NO: 9):

| | |
|---|---|
| 1 | mgsllallal lllwgavaeg pakkvltleg dlvlgglfpv hqkggpaedc gpvnehrgiq |
| 61 | rleamlfald rinrdphllp gvrlgahild scskdthale qaldfvrasl srgadgsrhi |
| 121 | cpdgsyathg daptaitgvi ggsysdvsiq vanllrlfqi pqisyastsa klsdksrydy |
| 181 | fartvppdff qakamaeilr ffnwtyvstv asegdygetg ieafeleara rnicvatsek |
| 241 | vgramsraaf egvvrallqk psarvavlft rsedarella asqrlnasft wvasdgwgal |
| 301 | esvvagsega aegaitiela sypisdfasy fqsldpwnns rnpwfrefwe qrfrcsfrqr |
| 361 | dcaahslrav pfeqeskimf vvnavyamah alhnmhralc pnttrlcdam rpvngrrlyk |
| 421 | dfvlnvkfda pfrpadthne vrfdrfgdgi gryniftylr agsgryryqk vgywaegltl |
| 481 | dtslipwasp sagplpasrc sepclqnevk svqpgevccw lcipcqpyey rldeftcadc |
| 541 | glgywpnasl tgcfelpqey irwgdawavg pvtiaclgal atlfvlgvfv rhnatpvvka |
| 601 | sgrelcyill ggvflcycmt fifiakpsta vctlrrlglg tafsvcysal ltktnriari |
| 661 | fggaregaqr prfispasqv aiclalisgq lllvvawlvv eapgtgketa perrevvtlr |
| 721 | cnhrdasmlg slaynvllia lctlyafktr kcpenfneak figftmyttc iiwlafipif |
| 781 | yvtssdyrvq tttmcvsvsl sgsvvlgclf apklhiilfq pqknvvshra ptsrfgsaaa |
| 841 | rassslgqgs gsqfvptvcn grevvdstts sl. |

In some embodiments, the signal peptide of mGluR2 is replaced with a signal peptide from another glutamate receptor, such as mGluR5. In some embodiments, the signal peptide from mGluR2 is replaced with the signal peptide from another GPCR, such as mGluR5. For example, in a specific embodiment, a vector of the present disclosure encodes a fusion polypeptide comprising, from N-terminus to C-terminus, a signal peptide derived from mGluR5 (replacing the mGluR2 signal peptide sequence), a linker sequence, a SNAP tag sequence and an mGluR2 sequence. In a more specific embodiment, the fusion polypeptide the following amino acid sequence:

In even more particular embodiments, a heterologous polypeptide sequence encoded within a vector of the disclosure which is driven by a CBh promoter comprises a mGluR5 signal peptide sequence shown below in bold and/or a linker sequence shown below as underlined text and/or a SNAP tag sequence shown below in italics and/or a mGluR2 sequence shown below in normal text, or a functional fragment or variant of any of the foregoing. In a more specific embodiment, the heterologous polypeptide sequence encoded within a vector of the disclosure comprises the sequence set out below as SEQ ID NO: 49.

(SEQ ID NO: 49)
MVLLLILSVLLLKEDVRGSAQSTRTRGSDKDCEMKRTTLDSPLGKLELS
GCEQGLHEIKLLGKGTSAADAVEVPAPAAVLGGPEPLMQATAWLNAYFH
QPEAIEEFPVPALHHPVFQQESFTRQVLWKLLKWKFGEVISYQQLAALA
GNPAATAAVKTALSGNPVPILIPCHRWSSSGAVGGYEGGLAVKEWLLAH
EGHRLGKPGLGTRKKVLTLEGDLVLGGLFPVHQKGGPAEDCGPVNEHRG
IQRLEAMLFALDRINRDPHLLPGVRLGAHILDSCSKDTHALEQALDFVR
ASLSRGADGSRHICPDGSYATHGDAPTAITGVIGGSYSDVSIQVANLLR
LFQIPQISYASTSAKLSDKSRYDYFARTVPPDFFQAKAMAEILRFFNWT
YVSTVASEGDYGETGIEAFELEARARNICVATSEKVGRAMSRAAFEGVV
RALLQKPSARVAVLFTRSEDARELLAASQRLNASFTWVASDGWGALESV
VAGSEGAAEGAITIELASYPISDFASYFQSLDPWNNSRNPWFREFWEQR
FRCSFRQRDCAAHSLRAVPFEQESKIMFVVNAVYAMAHALHNMHRALCP
NTTRLCDAMRPVNGRRLYKDFVLNVKFDAPFRPADTHNEVRFDRFGDGI
GRYNIFTYLRAGSGRYRYQKVGYWAEGLTLDTSLIPWASPSAGPLPASR
CSEPCLQNEVKSVQPGEVCCWLCIPCQPYEYRLDEFTCADCGLGYWPNA
SLTGCFELPQEYIRWGDAWAVGPVTIACLGALATLFVLGVFVRHNATPV
VKASGRELCYILLGGVFLCYCMTFIFIAKPSTAVCTLRRLGLGTAFSVC
YSALLTKTNRIARIFGGAREGAQRPRFISPASQVAICLALISGQLLIVV
AWLVVEAPGTGKETAPERREVVTLRCNHRDASMLGSLAYNVLLIALCTL
YAFKTRKCPENFNEAKFIGFTMYTTCIIWLAFLPIFYVTSSDYRVQTTT
MCVSVSLSGSVVLGCLFAPKLHIILFQPQKNVVSHRAPTSRFGSAAARA
SSSLGQGSGSQFVPTVCNGREVVDSTTSSL*.

In some embodiments, the mGluR comprises mGluR3. In some embodiments, the sequence encoding an mGluR comprises a sequence encoding a human mGluR3.

In some embodiments, the sequence encoding a human mGluR3 comprises or consists of the nucleic acid sequence of the following sequence, or a functional fragment or variant thereof (UniProtKB Q14832-1; GenBank Accession No. NM_000840.2 and SEQ ID NO: 10):

| | |
|---|---|
| 1 | gtgcagttga gtcgcgagta cggctgagct gcgtaccggc ctccctggct ctcacactcc |
| 61 | ctctctgctc ccgctctcct aatctcctct ggcatgcggt cagccccctg cccagggacc |
| 121 | acaggagagt tcttgtaagg actgttagtc cctgcttacc tgaaagccaa gcgctctagc |
| 181 | agagctttaa agttggagcc gccaccctcc ctaccgcccc atgccccttc accccactcc |
| 241 | gaaattcacc gacctttgca tgcactgcct aaggatttca gagtgaggca aagcagtcgg |
| 301 | caaatctacc ctggcttttc gtataaaaat cctctcgtct aggtaccctg gctcactgaa |
| 361 | gactctgcag atataccctt ataagaggga gggtggggga gggaaaagaa cgagagaggg |
| 421 | aggaaagaat gaaaaggaga ggatgccagg aggtccgtgc ttctgccaag agtcccaatt |
| 481 | agatgcgacg gcttcagcct ggtcaaggtg aaggaaagtt gcttccgcgc ctaggaagtg |
| 541 | ggtttgcctg ataagagaag gaggagggga ctcggctggg aagagctccc ctcccctccg |
| 601 | cggaagacca ctgggtcccc tctttcccca acctcctccc tctcttctac tccacccctc |
| 661 | cgttttccca ctccccactg actcggatgc ctggatgttc tgccaccggg cagtggtcca |
| 721 | gcgtgcagcc gggagggggc aggggcaggg ggcactgtga caggaagctg cgcgcacaag |
| 781 | ttggccattt cgagggcaaa ataagttctc ccttggattt ggaaaggaca aagccagtaa |
| 841 | gctacctctt ttgtgtcgga tgaggaggac caaccatgag ccagagcccg ggtgcaggct |
| 901 | caccgccgcc gctgccaccg cggtcagctc cagttcctgc caggagttgt cggtgcgagg |
| 961 | aattttgtga caggctctgt tagtctgttc ctcccttatt tgaaggacag gccaaagatc |
| 1021 | cagtttggaa atgagagagg actagcatga cacattggct ccaccattga tatctcccag |
| 1081 | aggtacagaa acaggattca tgaagatgtt gacaagactg caagttctta ccttagcttt |
| 1141 | gttttcaaag ggatttttac tctctttagg ggaccataac tttctaagga gagagattaa |
| 1201 | aatagaaggt gaccttgttt tagggggcct gtttcctatt aacgaaaaag gcactggaac |
| 1261 | tgaagaatgt gggcgaatca atgaagaccg agggattcaa cgcctggaag ccatgttgtt |
| 1321 | tgctattgat gaaatcaaca aagatgatta cttgctacca ggagtgaagt tgggtgttca |
| 1381 | cattttggat acatgttcaa gggataccta tgcattggag caatcactgg agtttgtcag |
| 1441 | ggcatctttg acaaaagtgg atgaagctga gtatatgtgt cctgatggat cctatgccat |
| 1501 | tcaagaaaac atcccacttc tcattgcagg ggtcattggt ggctcttata gcagtgtttc |
| 1561 | catacaggtg gcaaacctgc tgcggctctt ccagatccct cagatcagct acgcatccac |
| 1621 | cagcgccaaa ctcagtgata agtcgcgcta tgattacttt gccaggaccg tgccccccga |
| 1681 | cttctaccag gccaaagcca tggctgagat cttgcgcttc ttcaactgga cctacgtgtc |
| 1741 | cacagtagcc tccgagggtg attacgggga gacagggatc gaggccttcg agcaggaagc |
| 1801 | ccgcctgcgc aacatctgca tcgctacggc ggagaaggtg ggccgctcca acatccgcaa |
| 1861 | gtcctacgac agcgtgatcc gagaactgtt gcagaagccc aacgcgcgcg tcgtggtcct |
| 1921 | cttcatgcgc agcgacgact cgcgggagct cattgcagcc gccagccgcg ccaatgcctc |
| 1981 | cttcacctgg gtggccagcg acggctgggg cgcgcaggag agcatcatca agggcagcga |
| 2041 | gcatgtggcc tacggcgcca tcaccctgga gctggcctcc cagcctgtcc gccagttcga |
| 2101 | ccgctacttc cagagcctca acccctacaa caaccaccgc aacccctggt tccgggactt |
| 2161 | ctgggagcaa aagtttcagt gcagcctcca gaacaaacgc aaccacaggc gcgtctgcga |
| 2221 | caagcacctg gccatcgaca gcagcaacta cgagcaagag tccaagatca tgtttgtggt |
| 2281 | gaacgcggtg tatgccatgg cccacgcttt gcacaaaatg cagcgcaccc tctgtcccaa |
| 2341 | cactaccaag ctttgtgatg ctatgaagat cctggatggg aagaagttgt acaaggatta |
| 2401 | cttgctgaaa atcaacttca cggctccatt caacccaaat aaagatgcag atagcatagt |
| 2461 | caagtttgac acttttggag atggaatggg gcgatacaac gtgttcaatt tccaaaatgt |
| 2521 | aggtggaaag tattcctact tgaaagttgg tcactgggca gaaaccttat cgctagatgt |
| 2581 | caactctatc cactggtccc ggaactcagt ccccacttcc cagtgcagcg acccctgtgc |
| 2641 | ccccaatgaa atgaagaata tgcaaccagg ggatgtctgc tgctggattt gcatcccctg |
| 2701 | tgaaccctac gaatacctgg ctgatgagtt tacctgtatg gattgtgggt ctggacagtg |
| 2761 | gcccactgca gacctaactg gatgctatga ccttcctgag gactacatca ggtgggaaga |
| 2821 | cgcctgggcc attggcccag tcaccattgc ctgtctgggt tttatgtgta catgcatggt |
| 2881 | tgtaactgtt tttatcaagc acaacaacac acccttggtc aaagcatcgg gccgagaact |
| 2941 | ctgctacatc ttattgtttg gggttggcct gtcatactgc atgacattct tcttcattgc |
| 3001 | caagccatca ccagtcatct gtgcattgcg ccgactcggg ctggggagtt ccttcgctat |
| 3061 | ctgttactca gccctgctga ccaagacaaa ctgcattgcc cgcatcttcg atggggtcaa |
| 3121 | gaatggcgct cagaggccaa aattcatcag ccccagttct caggttttca tctgcctggg |
| 3181 | tctgatcctg gtgcaaattg tgatggtgtc tgtgtggctc atcctggagg ccccaggcac |
| 3241 | caggaggtat acccttgcag agaagcggga aacagtcatc ctaaaatgca atgtcaaaga |
| 3301 | ttccagcatg ttgatctctc ttacctacga tgtgatcctg gtgatcttat gcactgtgta |
| 3361 | cgccttcaaa acgcggaagt gcccagaaaa tttcaacgaa gctaagttca taggttttac |
| 3421 | catgtacacc acgtgcatca tctggttggc cttcctccct atattttatg tgacatcaag |
| 3481 | tgactacaga gtgcagacga caaccatgtg catctctgtc agcctgagtg gctttgtggt |
| 3541 | cttgggctgt ttgtttgcac ccaaggttca catcatcctg tttcaacccc agaagaatgt |
| 3601 | tgtcacacac agactgcacc tcaacaggtt cagtgtcagt ggaactggga ccacatactc |
| 3661 | tcagtcctct gcaagcacgt atgtgccaac ggtgtgcaat gggcgggaag tcctcgactc |
| 3721 | caccacctca tctctgtgat tgtgaattgc agttcagttc ttgtgttttt agactgttag |
| 3781 | acaaaagtgc tcacgtgcag ctccagaata tggaaacaga gcaaaagaac aaccctagta |
| 3841 | ccttttttta gaaacagtac gataaattat ttttgaggac tgtatatagt gatgtgctag |
| 3901 | aactttctag gctgagtcta gtgcccctat tattaacaat tcccccagaa catggaaata |
| 3961 | accattgttt acagagctga gcattggtga cagggtctga catggtcagt ctactaaaaa |
| 4021 | acaaaaaaaa aaaacaaaaa aaaaaaaaca aaagaaaaaa ataaaaatac ggtggcaata |
| 4081 | ttatgtaacc ttttttccta tgaagttttt tgtaggtcct tgttgtaact aatttaggat |
| 4141 | gagtttctat gttgtatatt aaagttacat tatgtgtaac agattgattt tctcagcaca |
| 4201 | aaataaaaag catctgtatt aatgtaaaga tactgagaat aaaaccttca aggttttcca |

In some embodiments, the human mGluR3 comprises or consists of the amino acid sequence of the following sequence, or a functional fragment or variant thereof (UniProtKB Q14832-1; GenBank Accession No. NP_000831.2 and SEQ ID NO: 11):

| | |
|---|---|
| 1 | mkmltrlqvl tlalfskgf1 Islgdhnfir reikiegdlv igglfpinek gtgteecgri |
| 61 | nedrgiqrle amlfaidein kddyllpgvk igvhildtcs rdtyaleqsl efvrasltkv |
| 121 | deaeymepdg syaiqenipl liagviggsy ssvsiqvanl irlfqipqis yastsaklsd |
| 181 | ksrydyfart vppdfyqaka maeilrffnw tyvstvaseg dygetgieaf eqearlrnic |
| 241 | iataekvgrs nirksydsvi rellqkpnar vvvlfmrsdd sreliaaasr anasftwvas |
| 301 | dgwgaqesii kgsehvayga itlelasqpv rqfdryfqsl npynnhrnpw frdfweqkfq |
| 361 | cslqnkrnhr rvcdkhlaid ssnyeqeski mfvvnavyam ahalhkmqrt icpnttklcd |
| 421 | amkildgkkl ykdyllkinf tapfnpnkda dsivkfdtfg dgmgrynvfn fqnvggkysy |
| 481 | Ikvghwaetl sldvnsihws rnsvptsqcs dpcapnemkn mqpgdvccwi cipcepyeyl |
| 541 | adeftcmdcg sgqwptadlt gcydlpedyi rwedawaigp vtiaclgfmc tcmvvtvfik |
| 601 | hnntplvkas grelcyillf gvglsycmtf ffiakpspvi calrrlglgs sfaicysall |
| 661 | tktnciarif dgvkngaqrp kfispssqvf iciglilvqi vmvsvwlile apgtrrytla |
| 721 | ekretvilkc nvkdssmlis itydvilvil ctvyafktrk cpenfneakf igftmyttci |
| 781 | iwlafipify vtssdyrvqt ttmcisvsls gfvvlgclfa pkvhiilfqp qknvvthrlh |
| 841 | Inrfsvsgtg ttysqssast yvptvcngre vldsttssl. |

In some embodiments, the sequence encoding a human mGluR3 comprises or consists of the nucleic acid sequence of the following sequence, or a functional fragment or variant thereof (UniProtKB Q14832-2; GenBank Accession No. NM_001363522.2 and SEQ ID NO: 12):

| | |
|---|---|
| 1 | gcagtgtgca gttgagtcgc gagtacggct gagctgcgta ccggcctccc tggctctcac |
| 61 | actccctctc tgctcccgct ctcctaatct cctctggcat gcggtcagcc ccctgcccag |
| 121 | ggaccacagg agagttcttg taaggactgt tagtccctgc ttacctgaaa gccaagcgct |
| 181 | ctagcagagc tttaaagttg gagccgccac cctccctacc gccccatgcc ccttcacccc |
| 241 | actccgaaat tcaccgacct ttgcatgcac tgcctaagga tttcagagtg aggcaaagca |
| 301 | gtcggcaaat ctaccctggc ttttcgtata aaaatcctct cgtctaggta ccctggctca |
| 361 | ctgaagactc tgcagatata cccttataag agggagggtg ggggagggaa aagaacgaga |
| 421 | gagggaggaa agaatgaaaa ggagaggatg ccaggaggtc cgtgcttctg ccaagagtcc |
| 481 | caattagatg cgacggcttc agcctggtca aggtgaagga aagttgcttc cgcgcctagg |
| 541 | aagtgggttt gcctgataag agaaggagga ggggactcgg ctgggaagag ctcccctccc |
| 601 | ctccgcggaa gaccactggg tcccctcttt ccccaacctc ctccctctct tctactccac |
| 661 | ccctccgttt tcccactccc cactgactcg gatgcctgga tgttctgcca ccgggcagtg |
| 721 | gtccagcgtg cagccgggag ggggcagggg cagggggcac tgtgacagga agctgcgcgc |
| 781 | acaagttggc catttcgagg gcaaaataag ttctcccttg gatttggaaa ggacaaagcc |
| 841 | agtaagctac ctcttttgtg tcggatgagg aggaccaacc atgagccaga gcccgggtgc |
| 901 | aggctcaccg ccgccgctgc caccgcggtc agctccagtt cctgccagga gttgtcggtg |
| 961 | cgaggaattt tgtgacaggc tctgttagtc tgttcctccc ttatttgaag gacaggccaa |
| 1021 | agatccagtt tggaaatgag agaggactag catgacacat tggctccacc attgatatct |
| 1081 | cccagaggta cagaaacagg attcatgaag atgttgacaa gactgcaagt tcttacctta |
| 1141 | gctttgtttt caaagggatt tttactctct ttaggggacc ataactttct aaggagagag |
| 1201 | attaaaatag aaggtgacct tgttttaggg ggcctgtttc ctattaacga aaaaggcact |
| 1261 | ggaactgaag aatgtgggcg aatcaatgaa gaccgaggga ttcaacgcct ggaagccatg |
| 1321 | ttgtttgcta ttgatgaaat caacaaagat gattacttgc taccaggagt gaagttgggt |
| 1381 | gttcacattt tggatacatg ttcaagggat acctatgcat tggagcaatc actggagttt |
| 1441 | gtcagggcat ctttgacaaa agtggatgaa gctgagtata tgtgtcctga tggatcctat |
| 1501 | gccattcaag aaaacatccc acttctcatt gcaggggtca ttggtggctc ttatagcagt |
| 1561 | gtttccatac aggtggcaaa cctgctgcgg ctcttccaga tccctcagat cagctacgca |
| 1621 | tccaccagcg ccaaactcag tgataagtcg cgctatgatt actttgccag gaccgtgccc |
| 1681 | cccgacttct accaggccaa agccatggct gagatcttgc gcttcttcaa ctggacctac |
| 1741 | gtgtccacag tagcctccga gggtgattac ggggagacag ggatcgaggc cttcgagcag |
| 1801 | gaagcccgcc tgcgcaacat ctgcatcgct acggcggaga aggtgggccg ctccaacatc |
| 1861 | cgcaagtcct acgacagcgt gatccgagaa ctgttgcaga agcccaacgc gcgcgtcgtg |
| 1921 | gtcctcttca tgcgcagcga cgactcgcgg gagctcattg cagccgccag ccgcgccaat |
| 1981 | gcctccttca cctgggtggc cagcgacggc tggggcgcgc aggagagcat catcaagggc |
| 2041 | agcgagcatg tggcctacgg cgccatcacc ctggagctgg cctcccagcc tgtccgccag |
| 2101 | ttcgaccgct acttccagag cctcaacccc tacaacaacc accgcaaccc ctggttccgg |
| 2161 | gacttctggg agcaaaagtt tcagtgcagc ctccagaaca aacgcaacca caggcgcgtc |
| 2221 | tgcgacaagc acctggccat cgacagcagc aactacgagc aagagtccaa gatcatgttt |
| 2281 | gtggtgaacg cggtgtatgc catggcccac gctttgcaca aaatgcagcg caccctctgt |
| 2341 | cccaacacta ccaagctttg tgatgctatg aagatcctgg atgggaagaa gttgtacaag |
| 2401 | gattacttgc tgaaaatcaa cttcacgggt gcagacgaca accatgtgca tctctgtcag |
| 2461 | cctgagtggc tttgtggtct tgggctgttt gtttgcaccc aaggttcaca tcatcctgtt |
| 2521 | tcaaccccag aagaatgttg tcacacacag actgcacctc aacaggttca gtgtcagtgg |
| 2581 | aactgggacc acatactctc agtcctctgc aagcacgtat gtgccaacgg tgtgcaatgg |
| 2641 | gcgggaagtc ctcgactcca ccacctcatc tctgtgattg tgaattgcag ttcagttctt |
| 2701 | gtgtttttag actgttagac aaaagtgctc acgtgcagct ccagaatatg gaaacagagc |
| 2761 | aaaagaacaa ccctagtacc tttttttaga aacagtacga taaattattt ttgaggactg |
| 2821 | tatatagtga tgtgctagaa ctttctaggc tgagtctagt gcccctatta ttaacaattc |
| 2881 | ccccagaaca tggaaataac cattgtttac agagctgagc attggtgaca gggtctgaca |
| 2941 | tggtcagtct actaaaaaac aaaaaaaaaa aacaaaaaaa aaaaaacaaa agaaaaaaat |
| 3001 | aaaaatacgg tggcaatatt atgtaacctt ttttcctatg aagttttttg taggtccttg |
| 3061 | ttgtaactaa tttaggatga gtttctatgt tgtatattaa agttacatta tgtgtaacag |
| 3121 | attgattttc tcagcacaaa ataaaaagca tctgtattaa tgtaaagata ctgagaataa |
| 3181 | aaccttcaag gttttccagc a. |

In some embodiments, the human mGluR3 comprises or consists of the amino acid sequence of the following sequence, or a functional fragment or variant thereof (UniProtKB Q14832-2; GenBank Accession No. NP_001350451.1 and SEQ ID NO: 13):

| | |
|---|---|
| 1 | mkmltrlqvl tlalfskgfl lslgdhnflr rcikiegdlv lgglfpinek gtgteecgri |
| 61 | nedrgiqrle amlfaidein kddyllpgvk lgvhildtcs rdtyaleqsl cfvrasltkv |
| 121 | dcaeymcpdg syaiqenipl liagviggsy ssvsiqvanl lrlfqipqis yastsaklsd |
| 181 | ksrydyfart vppdfyqaka macilrffnw tyvstvaseg dygetgieaf eqearlrnic |
| 241 | iataekvgrs nirksydsvi rellqkpnar vvvlfmrsdd sreliaaasr anasftwvas |
| 301 | dgwgaqesii kgschvayga itlelasqpv rqfdryfqsl npynnhrnpw frdfweqkfq |
| 361 | cslqnkrnhr rvcdkhlaid ssnyeqeski mfvvnavyam ahalhkmqrt lcpnttklcd |
| 421 | amkildgkkl ykdyllkinf tgaddnhvhl cqpewlcglg lfvctqgshh pvstpecech |
| 481 | tqtapqqvqc qwnwdhilsv lckhvcangv qwagsprlhh lisvivncss vlvfldc. |

In some embodiments, the mGluR comprises mGluR4. In some embodiments, the sequence encoding an mGluR comprises a sequence encoding a human mGluR4.

In some embodiments, the sequence encoding a human mGluR4 comprises or consists of the nucleic acid sequence of the following sequence, or a functional fragment or variant thereof (UniProtKB Q14833-1; GenBank Accession No. NM_000841.4 and SEQ ID NO: 14):

| | |
|---|---|
| 1 | gctgtacttt tctgggtgtg tgttagggag gctatgttcc tgaccctccc cctctggggt |
| 61 | gagaaggggt ccccgccatg tcctcggggt tggtaggagg agaggattgg agctgttttc |
| 121 | tccttgatgc caagatacgc caagctagga gcattctgcc ctttccacag tcatccaccg |
| 181 | agaacaggcc tgcaggacgg gacaaggatc agagccttcc tgcaaccccg gccactgcct |
| 241 | gctgtctgtg ggcctggact gtgcgggcaa ctgtgcttgg cccgagtgac aaggaggtgg |
| 301 | gagagggtag cagcatgggc tacgcggttg gctgccctca gtccccctgc tgctgaagct |
| 361 | gccctgccca tgcccaccca ggccgtgggg ccaggggcct gccagggcta ggagtgggcc |
| 421 | tgccgttcat gggtctctag ggatttccga gatgcctggg aagagaggct tgggctggtg |
| 481 | gtgggcccgg ctgccccttt gcctgctcct cagcctttac ggcccctgga tgccttcctc |
| 541 | cctgggaaag cccaaaggcc accctcacat gaattccatc cgcatagatg gggacatcac |
| 601 | actgggaggc ctgttcccgg tgcatggccg gggctcagag ggcaagccct gtggagaact |
| 661 | taagaaggaa aagggcatcc accggctgga ggccatgctg ttcgccctgg atcgcatcaa |
| 721 | caacgacccg gacctgctgc ctaacatcac gctgggcgcc cgcattctgg acacctgctc |
| 781 | cagggacacc catgccctcg agcagtcgct gacctttgtg caggcgctca tcgagaagga |
| 841 | tggcacagag gtccgctgtg gcagtggcgg cccacccatc atcaccaagc ctgaacgtgt |
| 901 | ggtgggtgtc atcggtgctt cagggagctc ggtctccatc atggtggcca acatccttcg |
| 961 | cctcttcaag ataccccaga tcagctacgc ctccacagcg ccagacctga gtgacaacag |
| 1021 | ccgctacgac ttcttctccc gcgtggtgcc ctcggacacg taccaggccc aggccatggt |
| 1081 | ggacatcgtc cgtgccctca agtggaacta tgtgtccaca gtggcctcgg agggcagcta |
| 1141 | tggtgagagc ggtgtggagg ccttcatcca gaagtcccgt gaggacgggg gcgtgtgcat |
| 1201 | cgcccagtcg gtgaagatac cacgggagcc caaggcaggc gagttcgaca agatcatccg |
| 1261 | ccgcctcctg gagacttcga acgccagggc agtcatcatc tttgccaacg aggatgacat |
| 1321 | caggcgtgtg ctggaggcag cacgaagggc caaccagaca ggccatttct tctggatggg |
| 1381 | ctctgacagc tggggctcca agattgcacc tgtgctgcac ctggaggagg tggctgaggg |
| 1441 | tgctgtcacg atcctcccca agaggatgtc cgtacgaggc ttcgaccgct acttctccag |
| 1501 | ccgcacgctg gacaacaacc ggcgcaacat ctggtttgcc gagttctggg aggacaactt |
| 1561 | ccactgcaag ctgagccgcc acgccctcaa gaagggcagc cacgtcaaga agtgcaccaa |
| 1621 | ccgtgagcga attgggcagg attcagctta tgagcaggag gggaaggtgc agtttgtgat |
| 1681 | cgatgccgtg tacgccatgg gccacgcgct gcacgccatg caccgtgacc tgtgtcccgg |
| 1741 | ccgcgtgggg ctctgcccgc gcatggaccc tgtagatggc acccagctgc ttaagtacat |
| 1801 | ccgaaacgtc aacttctcag gcatcgcagg gaaccctgtg accttcaatg agaatggaga |
| 1861 | tgcgcctggg cgctatgaca tctaccaata ccagctgcgc aacgattctg ccgagtacaa |
| 1921 | ggtcattggc tcctggactg accacctgca ccttagaata gagcggatgc actggccggg |
| 1981 | gagcgggcag cagctgcccc gctccatctg cagcctgccc tgccaaccgg gtgagcggaa |
| 2041 | gaagacagtg aagggcatgc cttgctgctg gcactgcgag ccttgcacag ggtaccagta |
| 2101 | ccaggtggac cgctacacct gtaagacgtg tccctatgac atgcggccca cagagaaccg |
| 2161 | cacgggctgc cggcccatcc ccatcatcaa gcttgagtgg ggctcgccct gggccgtgct |
| 2221 | gcccctcttc ctggccgtgg tgggcatcgc tgccacgttg ttcgtggtga tcacctttgt |
| 2281 | gcgctacaac gacacgccca tcgtcaaggc ctcgggccgt gaactgagct acgtgctgct |
| 2341 | ggcaggcatc ttcctgtgct atgccaccac cttcctcatg atcgctgagc ccgaccttgg |
| 2401 | cacctgctcg ctgcgccgaa tcttcctggg actagggatg agcatcagct atgcagccct |
| 2461 | gctcaccaag accaaccgca tctaccgcat cttcgagcag ggcaagcgct cggtcagtgc |
| 2521 | cccacgcttc atcagccccg cctcacagct ggccatcacc ttcagcctca tctcgctgca |
| 2581 | gctgctgggc atctgtgtgt ggtttgtggt ggacccctcc cactcggtgg tggacttcca |
| 2641 | ggaccagcgg acactcgacc cccgcttcgc caggggtgtg ctcaagtgtg acatctcgga |
| 2701 | cctgtcgctc atctgcctgc tgggctacag catgctgctc atggtcacgt gcaccgtgta |
| 2761 | tgccatcaag acacgcggcg tgcccgagac cttcaatgag gccaagccca ttggcttcac |
| 2821 | catgtacacc acttgcatcg tctggctggc cttcatcccc atcttctttg gcacctcgca |
| 2881 | gtcggccgac aagctgtaca tccagacgac gacgctgacg gtctcggtga gtctgagcgc |
| 2941 | ctcggtgtcc ctgggaatgc tctacatgcc caaagtctac atcatcctct tccacccgga |
| 3001 | gcagaacgtg cccaagcgca agcgcagcct caaagccgtc gttacggcgg ccaccatgtc |
| 3061 | caacaagttc acgcagaagg gcaacttccg gcccaacgga gaggccaagt ctgagctctg |
| 3121 | cgagaacctt gaggccccag cgctggccac caaacagact tacgtcactt acaccaacca |
| 3181 | tgcaatctag cgagtccatg gagctgagca gcaggaggag gagccgtgac cctgtggaag |
| 3241 | gtgcgtcggg ccagggccac acccaagggc ccagctgtct tgcctgcccg tgggcaccca |
| 3301 | cggacgtggc ttggtgctga ggatagcaga gcccccagcc atcactgctg gcagcctggg |
| 3361 | caaaccgggt gagcaacagg aggacgaggg gccggggcgg tgccaggcta ccacaagaac |
| 3421 | ctgcgtcttg gaccattgcc cctcccggcc ccaaaccaca ggggctcagg tcgtgtgggc |
| 3481 | cccagtgcta gatctctccc tcccttcgtc tctgtctgtg ctgttggcga cccctctgtc |
| 3541 | tgtctccagc cctgtctttc tgttctctta tctctttgtt tcaccttttc cctctctggc |
| 3601 | gtccccggct gcttgtactc ttggcctttt ctgtgtctcc tttctggctc ttgcctccgc |
| 3661 | ctctctctct catcctcttt gtcctcagct cctcctgctt tcttgggtcc caccagtgtc |
| 3721 | acttttctgc cgttttcttt cctgttctcc tctgcttcat tctcgtccag ccattgctcc |
| 3781 | cctctccctg ccacccttcc ccagttcacc aaaccttaca tgttgcaaaa gagaaaaaag |
| 3841 | gaaaaaaaat caaaacacaa aaaagccaaa acgaaaacaa atctcgagtg tgttgccaag |
| 3901 | tgctgcgtcc tcctggtggc ctctgtgtgt gtccctgtgg cccgcagcct gcccgcctgc |
| 3961 | cccgcccatc tgccgtgtgt cttgcccgcc tgccccgccc gtctgccgtc tgtcttgccc |
| 4021 | gcctgcccgc ctgcccctcc tgccgaccac acggagttca gtgcctgggt gtttggtgat |
| 4081 | ggttattgac gacaatgtgt agcgcatgat tgtttttata ccaagaacat ttctaataaa |
| 4141 | aataaacaca tggttttgca cccgggctcc acatccactg agggtcctgc catgggacca |
| 4201 | caggctcagc ctgcagctgg agggcttaga cctagaggga agcgggaact gggctctgga |
| 4261 | gacccagggc ttgggggctg tggagactgc tccctaggct gggatctagt gtggtgtggt |
| 4321 | gaggccttgg gcatggaggg gccagattcc caggtaaggg gcagggacat tgcaggaaat |
| 4381 | tccaggaatc agcacctagt agtcccctaa ttagggggta tgctctgtcc cctgccctgc |
| 4441 | agccctggga gggtaacatt tctgccttgc ctgtcctctg tctcacaccc ctcacacctg |
| 4501 | ggactgccct tccacccctg cccccataac ctgtgcctct ctccttccag ccaggaagtc |
| 4561 | ctcttcttga gaagttagct tcccgggctg ccagcactca tagccgtccc ctcctgcttg |
| 4621 | tgttggctcc aggctcgggt gctaagaaga tgtgtgtctg tcctggagat cagtgtgttg |
| 4681 | ttatgtgtcc acgtgggccc acaagtgcac ggcacaggca tggccgtgtg gctgtgttgg |
| 4741 | ctgtgttggc tgtgtgtctg tgtgcacgtc cagcgcctcc atgcgcatgc gtgcctgtct |
| 4801 | tgtttgcgtg tctgatcatc tgtttgggcc ccggtggctc atgcagatgc ctgtctcagg |
| 4861 | cccatggcga gtgttcacct cagctggctt ccctggcagg ttgggaggtg ggaaacagga |
| 4921 | gcgcttaggg gctgggctct ggctggggta aattatagag ccagaaacac aatgaggcca |
| 4981 | taggcagcag ctggagcctg ggctgcctgt gccgtcccct cctgccctgc ccctgggtcc |
| 5041 | tgcaccccct cccacctcca ggctagctga cagcgctatg gagcacagtg gaagggactg |
| 5101 | gaggaaccct aggcaggggg ccacgcaggg acagagtatg agagtgtgtg tataactgag |
| 5161 | gctgggacat tgaatcatgc caggtatgtc ttctccatca gcccactctt actcctggcc |
| 5221 | tgggcatctc acacatctgt gcataggaaa tctcttcttc cctggggtct gtgtgcagca |
| 5281 | cctagtagat gctcaataaa tgtttgtgtg agggaaggag acaggaaagg aagtgtctcg |
| 5341 | ctgatcatct tgcggaatgg ttcctaagac ctctgcccag gaaagattcc acccagtgct |
| 5401 | ccagcccggt caggcagaac taggttgcca gatcaagggt atctcccaaa agcttccagg |
| 5461 | gcagttgggg gtgggggggt ggggggtagg gatggggaat gcagaagcgg gtgcagccag |
| 5521 | ctctccccca gggtgactct ggcagcaccc ccatcctggg caccctgcct gctctgtggc |
| 5581 | tcacgcccct cctgaagtga ctgatgctct gaggcccaag gctaggtcca gggcagggcc |
| 5641 | tgcaggggtt tcatgctcag tccaggactt gcctaggtcc ccctacatct gtggggcccc |
| 5701 | catctaggtt ctaacaggag aatcacctct ccaaggggga tgctgcccct cggctcccct |
| 5761 | tggctctcag gaggggccct cagggactac cagtcccctg ccagtgggaa gaaatagccc |
| 5821 | tgccctcagg gagcttccag tgtgatgggg gagatacagc agactgtgtc ccaaagtaaa |
| 5881 | atgactgtta gaatgaggtg ggtggaggag ggaagccttg ggtgggtgtg actttgggca |
| 5941 | tctgagcctg gggtgcagag gtgggctctg tgggcctgag gtggacagga gggaaccagg |
| 6001 | ccctagcaag acttttgcca gctagacctg ctgcagcagt tgggagggtg ggtgctgctg |
| 6061 | gagtcctggg tccatcacct agaaggctca ggccagtgca gccagggctg gggcccacag |
| 6121 | ctggcctggg tgggacctgc cctgatgccc atggcaggag ggacgcctgg cccttcacaa |
| 6181 | ttggcttggc tgctcacctt tgctctcatc ctcaattatt aatgactgga gaaagctgct |
| 6241 | aagtatcttc agaatgttag atttcaacaa gatggggggt tcagggtccc tggcaccctg |
| 6301 | gatagggagc cagcggcccc tagagacctt tgctgtgtgc agggggtatg tgctcacccc |
| 6361 | cgtggcctca gcctcctcaa tgtctgaatg aaggattggg ctagcagaca tcccacccca |
| 6421 | cagcacactt tctaaccagc aggggaactt ctagacaata gagacgctgg gctccctcca |
| 6481 | gaacactgga cctgaacttc tggggggagg gctgggcacg ggcatatttt aaaagctccc |
| 6541 | cagcagatgg gccgtgcagt caagtgggcc aagagtggca ccagactttg gggcttgtga |
| 6601 | agtcaggagg gagcaacagt gcccactcga gcttgcctgg ggctcaagcc caaggctggg |
| 6661 | ctgctgccag cctgagcaga cacccaggag cttccaggcc agctggatgc acagggcacc |
| 6721 | tttgtggaac tcctaggacc ctggggagac ccacctcagg agcagagtct caggtccctt |
| 6781 | ccggctctga ggggctgttc tgagctctaa tgtcttatgg tctgcccctc ccatccttac |
| 6841 | ttctcaggcc ctggaggcag aggcatagag ccaggcagga cagaggtctc agtgggccac |
| 6901 | atgccagctg cccccacact gcctcagcct ccaggcctcc aaggggtcct ggggagcccc |
| 6961 | tgagaagatg ctgagcctgc ataaggctgg gcgcccctct ttctgacacc ctcactggct |
| 7021 | ccacggctcc cccttcccat cccaggtttc catctgccca ctgaacaggg aggggaaact |
| 7081 | gaggcactcc cctggcactg agggctcctt ctgtcatcct gcctgccctg gatggtcctg |
| 7141 | gctgcccctc agggcttggc cctggcactg tgagcctcac agggctcaga cccccacccc |
| 7201 | caacccagca ctaaatggca ctcggcacca gaatctcact tcagttggca aaagcagcaa |
| 7261 | ttagcatgta atgaggcttc ttgctttatt tttaggtaac ctccaaggcc ctgcctgtgt |
| 7321 | aattcagccc gccattgctc ggtggataat taaagcatgt caccataa. |

In some embodiments, the human mGluR4 comprises or consists of the amino acid sequence of the following sequence, or a functional fragment or variant thereof (UniProtKB Q14833-1 and SEQ ID NO: 15):

| | |
|---|---|
| 1 | MPGKRGLGWW WARLPLCLLL SLYGPWMPSS LGKPKGHPHM NSIRIDGDIT LGGLFPVHGR |
| 61 | GSEGKPCGEL KKEKGIHRLE AMLFALDRIN NDPDLLPNIT LGARILDTCS RDTHALEQSL |
| 121 | TFVQALIEKD GTEVRCGSGG PPIITKPERV VGVIGASGSS VSIMVANILR LFKIPQISYA |
| 181 | STAPDLSDNS RYDFFSRVVP SDTYQAQAMV DIVRALKWNY VSTVASEGSY GESGVEAFIQ |
| 241 | KSREDGGVCI AQSVKIPREP KAGEFDKIIR RLLETSNARA VIIFANEDDI RRVLEAARRA |
| 301 | NQTGHFFWMG SDSWGSKIAP VLHLEEVAEG AVTILPKRMS VRGFDRYFSS RTLDNNRRNI |
| 361 | WFAEFWEDNF HCKLSRHALK KGSHVKKCTN RERIGQDSAY EQEGKVQFVI DAVYAMGHAL |
| 421 | HAMHRDLCPG RVGLCPRMDP VDGTQLLKYI RNVNFSGIAG NPVTFNENGD APGRYDIYQY |
| 481 | QLRNDSAEYK VIGSWTDHLH LRIERMHWPG SGQQLPRSIC SLPCQPGERK KTVKGMPCCW |
| 541 | HCEPCTGYQY QVDRYTCKTC PYDMRPTENR TGCRPIPIIK LEWGSPWAVL PLFLAVVGIA |
| 601 | ATLFVVITFV RYNDTPIVKA SGRELSYVLL AGIFLCYATT FLMIAEPDLG TCSLRRIFLG |
| 661 | LGMSISYAAL LTKTNRIYRI FEQGKRSVSA PRFISPASQL AITFSLISLQ LLGICVWFVV |
| 721 | DPSHSVVDFQ DQRTLDPRFA RGVLKCDISD LSLICLLGYS MLLMVTCTVY AIKTRGVPET |
| 781 | FNEAKPIGFT MYTTCIVWLA FIPIFFGTSQ SADKLYIQTT TLTVSVSLSA SVSLGMLYMP |
| 841 | KVYIILFHPE QNVPKRKRSL KAVVTAATMS NKFTQKGNFR PNGEAKSELC ENLEAPALAT |
| 901 | KQTYVTYTNH AI. |

In some embodiments, the human mGluR4 comprises or consists of the amino acid sequence of the following sequence, or a functional fragment or variant thereof (UniProtKB Q14833-2 and SEQ ID NO: 17):

| | |
|---|---|
| 1 | MVQTLPKLFP HDGAKRKKRT LRTSGPCFGG GGQIPQISYA STAPDLSDNS RYDFFSRVVP |
| 61 | SDTYQAQAMV DIVRALKWNY VSTVASEGSY GESGVEAFIQ KSREDGGVCI AQSVKIPREP |
| 121 | KAGEFDKIIR RLLETSNARA VIIFANEDDI RRVLEAARRA NQTGHFFWMG SDSWGSKIAP |
| 181 | VLHLEEVAEG AVTILPKRMS VRGFDRYFSS RTLDNNRRNI WFAEFWEDNF HCKLSRHALK |
| 241 | KGSHVKKCTN RERIGQDSAY EQEGKVQFVI DAVYAMGHAL HAMHRDLCPG RVGLCPRMDP |
| 301 | VDGTQLLKYI RNVNFSGIAG NPVTFNENGD APGRYDIYQY QLRNDSAEYK VIGSWTDHLH |
| 361 | LRIERMHWPG SGQQLPRSIC SLPCQPGERK KTVKGMPCCW HCEPCTGYQY QVDRYTCKTC |
| 421 | PYDMRPTENR TGCRPIPIIK LEWGSPWAVL PLFLAVVGIA ATLFVVITFV RYNDTPIVKA |
| 481 | SGRELSYVLL AGIFLCYATT FLMIAEPDLG TCSLRRIFLG LGMSISYAAL LTKTNRIYRI |
| 541 | FEQGKRSVSA PRFISPASQL AITFSLISLQ LLGICVWFVV DPSHSVVDFQ DQRTLDPRFA |
| 601 | RGVLKCDISD LSLICLLGYS MLLMVTCTVY AIKTRGVPET FNEAKPIGFT MYTTCIVWLA |
| 661 | FIPIFFGTSQ SADKLYIQTT TLTVSVSLSA SVSLGMLYMP KVYIILFHPE QNVPKRKRSL |
| 721 | KAVVTAATMS NKFTQKGNFR PNGEAKSELC ENLEAPALAT KQTYVTYTNH AI. |

In some embodiments, the sequence encoding a human mGluR4 comprises or consists of the nucleic acid sequence of the following sequence, or a functional fragment or variant thereof (UniProtKB Q14833-3; GenBank Accession No. NM_001256812.2 and SEQ ID NO: 18):

| | |
|---|---|
| 1 | gatgccctgg ggactcacgg gctgcaggtg ctaccagccc agcaccccgg cacagcgggg |
| 61 | aggctgaggc agacacacgc tcctggagag acatgtcatg taagataccc cagatcagct |
| 121 | acgcctccac agcgccagac ctgagtgaca acagccgcta cgacttcttc tcccgcgtgg |
| 181 | tgccctcgga cacgtaccag gcccaggcca tggtggacat cgtccgtgcc ctcaagtgga |
| 241 | actatgtgtc cacagtggcc tcggagggca gctatggtga gagcggtgtg gaggccttca |
| 301 | tccagaagtc ccgtgaggac gggggcgtgt gcatcgccca gtcggtgaag ataccacggg |
| 361 | agcccaaggc aggcgagttc gacaagatca tccgccgcct cctggagact tcgaacgcca |
| 421 | gggcagtcat catctttgcc aacgaggatg acatcaggcg tgtgctggag gcagcacgaa |
| 481 | gggccaacca gacaggccat ttcttctgga tgggctctga cagctggggc tccaagattg |
| 541 | cacctgtgct gcacctggag gaggtggctg agggtgctgt cacgatcctc cccaagagga |
| 601 | tgtccgtacg aggcttcgac cgctacttct ccagccgcac gctggacaac aaccggcgca |
| 661 | acatctggtt tgccgagttc tgggaggaca acttccactg caagctgagc cgccacgccc |
| 721 | tcaagaaggg cagccacgtc aagaagtgca ccaaccgtga gcgaattggg caggattcag |
| 781 | cttatgagca ggaggggaag gtgcagtttg tgatcgatgc cgtgtacgcc atgggccacg |
| 841 | cgctgcacgc catgcaccgt gacctgtgtc ccggccgcgt ggggctctgc ccgcgcatgg |
| 901 | accctgtaga tggcacccag ctgcttaagt acatccgaaa cgtcaacttc tcaggcatcg |
| 961 | cagggaaccc tgtgaccttc aatgagaatg gagatgcgcc tgggcgctat gacatctacc |
| 1021 | aataccagct gcgcaacgat tctgccgagt acaaggtcat tggctcctgg actgaccacc |
| 1081 | tgcaccttag aatagagcgg atgcactggc cggggagcgg gcagcagctg ccccgctcca |
| 1141 | tctgcagcct gccctgccaa ccgggtgagc ggaagaagac agtgaagggc atgccttgct |
| 1201 | gctggcactg cgagccttgc acagggtacc agtaccaggt ggaccgctac acctgtaaga |
| 1261 | cgtgtcccta tgacatgcgg cccacagaga accgcacggg ctgccggccc atccccatca |
| 1321 | tcaagcttga gtggggctcg ccctgggccg tgctgcccct cttcctggcc gtggtgggca |
| 1381 | tcgctgccac gttgttcgtg gtgatcacct ttgtgcgcta caacgacacg cccatcgtca |
| 1441 | aggcctcggg ccgtgaactg agctacgtgc tgctggcagg catcttcctg tgctatgcca |
| 1501 | ccaccttcct catgatcgct gagcccgacc ttggcacctg ctcgctgcgc cgaatcttcc |
| 1561 | tgggactagg gatgagcatc agctatgcag ccctgctcac caagaccaac cgcatctacc |
| 1621 | gcatcttcga gcagggcaag cgctcggtca gtgccccacg cttcatcagc cccgcctcac |
| 1681 | agctggccat caccttcagc ctcatctcgc tgcagctgct gggcatctgt gtgtggtttg |
| 1741 | tggtggaccc ctcccactcg gtggtggact tccaggacca gcggacactc gacccccgct |
| 1801 | tcgccagggg tgtgctcaag tgtgacatct cggacctgtc gctcatctgc ctgctgggct |
| 1861 | acagcatgct gctcatggtc acgtgcaccg tgtatgccat caagacacgc ggcgtgcccg |
| 1921 | agaccttcaa tgaggccaag cccattggct tcaccatgta caccacttgc atcgtctggc |
| 1981 | tggccttcat ccccatcttc tttggcacct cgcagtcggc cgacaagctg tacatccaga |
| 2041 | cgacgacgct gacggtctcg gtgagtctga gcgcctcggt gtccctggga atgctctaca |
| 2101 | tgcccaaagt ctacatcatc ctcttccacc cggagcagaa cgtgcccaag cgcaagcgca |
| 2161 | gcctcaaagc cgtcgttacg gcggccacca tgtccaacaa gttcacgcag aagggcaact |
| 2221 | tccggcccaa cggagaggcc aagtctgagc tctgcgagaa ccttgaggcc ccagcgctgg |
| 2281 | ccaccaaaca gacttacgtc acttacacca accatgcaat ctagcgagtc catggagctg |
| 2341 | agcagcagga ggaggagccg tgaccctgtg gaaggtgcgt cgggccaggg ccacacccaa |
| 2401 | gggcccagct gtcttgcctg cccgtgggca cccacggacg tggcttggtg ctgaggatag |
| 2461 | cagagccccc agccatcact gctggcagcc tgggcaaacc gggtgagcaa caggaggacg |
| 2521 | aggggccggg gcggtgccag gctaccacaa gaacctgcgt cttggaccat tgcccctccc |
| 2581 | ggccccaaac cacaggggct caggtcgtgt gggccccagt gctagatctc tccctccctt |
| 2641 | cgtctctgtc tgtgctgttg gcgacccctc tgtctgtctc cagccctgtc tttctgttct |
| 2701 | cttatctctt tgtttcacct tttccctctc tggcgtcccc ggctgcttgt actcttggcc |
| 2761 | ttttctgtgt ctcctttctg gctcttgcct ccgcctctct ctctcatcct ctttgtcctc |
| 2821 | agctcctcct gctttcttgg gtcccaccag tgtcactttt ctgccgtttt ctttcctgtt |
| 2881 | ctcctctgct tcattctcgt ccagccattg ctcccctctc cctgccaccc ttccccagtt |
| 2941 | caccaaacct tacatgttgc aaaagagaaa aaaggaaaaa aaatcaaaac acaaaaaagc |
| 3001 | caaaacgaaa acaaatctcg agtgtgttgc caagtgctgc gtcctcctgg tggcctctgt |
| 3061 | gtgtgtccct gtggcccgca gcctgcccgc ctgccccgcc catctgccgt gtgtcttgcc |
| 3121 | cgcctgcccc gcccgtctgc cgtctgtctt gcccgcctgc ccgcctgccc ctcctgccga |
| 3181 | ccacacggag ttcagtgcct gggtgtttgg tgatggttat tgacgacaat gtgtagcgca |
| 3241 | tgattgtttt tataccaaga acatttctaa taaaaataaa cacatggttt tgcacccggg |
| 3301 | ctccacatcc actgagggtc ctgccatggg accacaggct cagcctgcag ctggagggct |
| 3361 | tagacctaga gggaagcggg aactgggctc tggagaccca gggcttgggg gctgtggaga |
| 3421 | ctgctcccta ggctgggatc tagtgtggtg tggtgaggcc ttgggcatgg aggggccaga |
| 3481 | ttcccaggta aggggcaggg acattgcagg aaattccagg aatcagcacc tagtagtccc |
| 3541 | ctaattaggg ggtatgctct gtcccctgcc ctgcagccct gggagggtaa catttctgcc |
| 3601 | ttgcctgtcc tctgtctcac acccctcaca cctgggactg cccttccacc cctgccccca |
| 3661 | taacctgtgc ctctctcctt ccagccagga agtcctcttc ttgagaagtt agcttcccgg |
| 3721 | gctgccagca ctcatagccg tcccctcctg cttgtgttgg ctccaggctc gggtgctaag |
| 3781 | aagatgtgtg tctgtcctgg agatcagtgt gttgttatgt gtccacgtgg gcccacaagt |
| 3841 | gcacggcaca ggcatggccg tgtggctgtg ttggctgtgt tggctgtgtg tctgtgtgca |
| 3901 | cgtccagcgc ctccatgcgc atgcgtgcct gtcttgtttg cgtgtctgat catctgtttg |
| 3961 | ggccccggtg gctcatgcag atgcctgtct caggcccatg gcgagtgttc acctcagctg |
| 4021 | gcttccctgg caggttggga ggtgggaaac aggagcgctt aggggctggg ctctggctgg |
| 4081 | ggtaaattat agagccagaa acacaatgag gccataggca gcagctggag cctgggctgc |
| 4141 | ctgtgccgtc ccctcctgcc ctgcccctgg gtcctgcacc ccctcccacc tccaggctag |
| 4201 | ctgacagcgc tatggagcac agtggaaggg actggaggaa ccctaggcag ggggccacgc |
| 4261 | agggacagag tatgagagtg tgtgtataac tgaggctggg acattgaatc atgccaggta |
| 4321 | tgtcttctcc atcagcccac tcttactcct ggcctgggca tctcacacat ctgtgcatag |
| 4381 | gaaatctctt cttccctggg gtctgtgtgc agcacctagt agatgctcaa taaatgtttg |
| 4441 | tgtgagggaa ggagacagga aaggaagtgt ctcgctgatc atcttgcgga atggttccta |
| 4501 | agacctctgc ccaggaaaga ttccacccag tgctccagcc cggtcaggca gaactaggtt |
| 4561 | gccagatcaa gggtatctcc caaaagcttc cagggcagtt gggggtgggg gggtgggggg |
| 4621 | tagggatggg gaatgcagaa gcgggtgcag ccagctctcc cccagggtga ctctggcagc |
| 4681 | acccccatcc tgggcaccct gcctgctctg tggctcacgc ccctcctgaa gtgactgatg |
| 4741 | ctctgaggcc caaggctagg tccagggcag ggcctgcagg ggtttcatgc tcagtccagg |
| 4801 | acttgcctag gtccccctac atctgtgggg cccccatcta ggttctaaca ggagaatcac |
| 4861 | ctctccaagg gggatgctgc ccctcggctc cccttggctc tcaggagggg ccctcaggga |
| 4921 | ctaccagtcc cctgccagtg ggaagaaata gccctgccct cagggagctt ccagtgtgat |
| 4981 | gggggagata cagcagactg tgtcccaaag taaaatgact gttagaatga ggtgggtgga |
| 5041 | ggagggaagc cttgggtggg tgtgactttg ggcatctgag cctggggtgc agaggtgggc |
| 5101 | tctgtgggcc tgaggtggac aggagggaac caggccctag caagactttt gccagctaga |
| 5161 | cctgctgcag cagttgggag ggtgggtgct gctggagtcc tgggtccatc acctagaagg |
| 5221 | ctcaggccag tgcagccagg gctggggccc acagctggcc tgggtgggac ctgccctgat |
| 5281 | gcccatggca ggagggacgc ctggcccttc acaattggct tggctgctca cctttgctct |
| 5341 | catcctcaat tattaatgac tggagaaagc tgctaagtat cttcagaatg ttagatttca |
| 5401 | acaagatggg gggttcaggg tccctggcac cctggatagg gagccagcgg cccctagaga |
| 5461 | cctttgctgt gtgcaggggg tatgtgctca cccccgtggc ctcagcctcc tcaatgtctg |
| 5521 | aatgaaggat tgggctagca gacatcccac cccacagcac actttctaac cagcagggga |
| 5581 | acttctagac aatagagacg ctgggctccc tccagaacac tggacctgaa cttctggggg |
| 5641 | gagggctggg cacgggcata ttttaaaagc tccccagcag atgggccgtg cagtcaagtg |
| 5701 | ggccaagagt ggcaccagac tttggggctt gtgaagtcag gagggagcaa cagtgcccac |
| 5761 | tcgagcttgc ctggggctca agcccaaggc tgggctgctg ccagcctgag cagacaccca |
| 5821 | ggagcttcca ggccagctgg atgcacaggg cacctttgtg gaactcctag gaccctgggg |
| 5881 | agacccacct caggagcaga gtctcaggtc ccttccggct ctgaggggct gttctgagct |
| 5941 | ctaatgtctt atggtctgcc cctcccatcc ttacttctca ggccctggag gcagaggcat |
| 6001 | agagccaggc aggacagagg tctcagtggg ccacatgcca gctgccccca cactgcctca |
| 6061 | gcctccaggc ctccaagggg tcctggggag cccctgagaa gatgctgagc ctgcataagg |
| 6121 | ctgggcgccc ctctttctga caccctcact ggctccacgg ctcccccttc ccatcccagg |
| 6181 | tttccatctg cccactgaac agggagggga aactgaggca ctcccctggc actgagggct |
| 6241 | ccttctgtca tcctgcctgc cctggatggt cctggctgcc cctcagggct tggccctggc |
| 6301 | actgtgagcc tcacagggct cagaccccca cccccaaccc agcactaaat ggcactcggc |
| 6361 | accagaatct cacttcagtt ggcaaaagca gcaattagca tgtaatgagg cttcttgctt |
| 6421 | tatttttagg taacctccaa ggccctgcct gtgtaattca gcccgccatt gctcggtgga |
| 6481 | taattaaagc atgtcaccat aaaaaaaaaa aaaaaaaa. |

In some embodiments, the human mGluR4 comprises or consists of the amino acid sequence of the following sequence, or a functional fragment or variant thereof (UniProtKB Q14833-3 and SEO ID NO: 19):

| | |
|---|---|
| 1 | MSCKIPQISY ASTAPDLSDN SRYDFFSRVV PSDTYQAQAM VDIVRALKWN YVSTVASEGS |
| 61 | YGESGVEAFI QKSREDGGVC IAQSVKIPRE PKAGEFDKII RRLLETSNAR AVIIFANEDD |
| 121 | IRRVLEAARR ANQTGHFFWM GSDSWGSKIA PVLHLEEVAE GAVTILPKRM SVRGFDRYFS |
| 181 | SRTLDNNRRN IWFAEFWEDN FHCKLSRHAL KKGSHVKKCT NRERIGQDSA YEQEGKVQFV |
| 241 | IDAVYAMGHA LHAMHRDLCP GRVGLCPRMD PVDGTQLLKY IRNVNFSGIA GNPVTFNENG |
| 301 | DAPGRYDIYQ YQLRNDSAEY KVIGSWTDHL HLRIERMHWP GSGQQLPRSI CSLPCQPGER |
| 361 | KKTVKGMPCC WHCEPCTGYQ YQVDRYTCKT CPYDMRPTEN RTGCRPIPII KLEWGSPWAV |
| 421 | LPLFLAVVGI AATLFVVITF VRYNDTPIVK ASGRELSYVL LAGIFLCYAT TFLMIAEPDL |
| 481 | GTCSLRRIFL GLGMSISYAA LLTKTNRIYR IFEQGKRSVS APRFISPASQ LAITFSLISL |
| 541 | QLLGICVWFV VDPSHSVVDF QDQRTLDPRF ARGVLKCDIS DLSLICLLGY SMLLMVTCTV |
| 601 | YAIKTRGVPE TFNEAKPIGF TMYTTCIVWL AFIPIFFGTS QSADKLYIQT TTLTVSVSLS |
| 661 | ASVSLGMLYM PKVYIILFHP EQNVPKRKRS LKAVVTAATM SNKFTQKGNF RPNGEAKSEL |
| 721 | CENLEAPALA TKQTYVTYTN HAI. |

In some embodiments, the sequence encoding a human mGluR4 comprises or consists of the nucleic acid sequence of the following sequence, or a functional fragment or variant thereof (UniProtKB Q14833-4; GenBank Accession No. NM_001256813.3 and SEQ ID NO: 20):

| | |
|---|---|
| 1 | agagatgctg gagctgtgga cctgggcggt cctggtggag gagtgcctga cacatcccag |
| 61 | tctttcaccg tggtgcctgt ggacagatga ggagactgag gctcacaaag gaggtgactt |
| 121 | gcccaaggtc acacagcgag gcagcttctc acctatggga gttggacttt gagccctcct |
| 181 | tggaaaaggg ggcatggctg tgccccttgg ggctccttgc tgggcctctg ccctgcctgc |
| 241 | ctgggctcct cccggcctcc cccacagatc actgttgaca aggttactga gtcagcatgt |
| 301 | aaaacctgca aaaatacccc agatcagcta cgcctccaca gcgccagacc tgagtgacaa |
| 361 | cagccgctac gacttcttct cccgcgtggt gccctcggac acgtaccagg cccaggccat |
| 421 | ggtggacatc gtccgtgccc tcaagtggaa ctatgtgtcc acagtggcct cggagggcag |
| 481 | ctatggtgag agcggtgtgg aggccttcat ccagaagtcc cgtgaggacg ggggcgtgtg |
| 541 | catcgcccag tcggtgaaga taccacggga gcccaaggca ggcgagttcg acaagatcat |
| 601 | ccgccgcctc ctggagactt cgaacgccag ggcagtcatc atctttgcca acgaggatga |
| 661 | catcaggcgt gtgctggagg cagcacgaag ggccaaccag acaggccatt tcttctggat |
| 721 | gggctctgac agctggggct ccaagattgc acctgtgctg cacctggagg aggtggctga |
| 781 | gggtgctgtc acgatcctcc ccaagaggat gtccgtacga ggcttcgacc gctacttctc |
| 841 | cagccgcacg ctggacaaca accggcgcaa catctggttt gccgagttct gggaggacaa |
| 901 | cttccactgc aagctgagcc gccacgccct caagaagggc agccacgtca agaagtgcac |
| 961 | caaccgtgag cgaattgggc aggattcagc ttatgagcag gaggggaagg tgcagtttgt |
| 1021 | gatcgatgcc gtgtacgcca tgggccacgc gctgcacgcc atgcaccgtg acctgtgtcc |
| 1081 | cggccgcgtg gggctctgcc cgcgcatgga ccctgtagat ggcacccagc tgcttaagta |
| 1141 | catccgaaac gtcaacttct caggcatcgc agggaaccct gtgaccttca atgagaatgg |
| 1201 | agatgcgcct gggcgctatg acatctacca ataccagctg cgcaacgatt ctgccgagta |
| 1261 | caaggtcatt ggctcctgga ctgaccacct gcaccttaga atagagcgga tgcactggcc |
| 1321 | ggggagcggg cagcagctgc cccgctccat ctgcagcctg ccctgccaac cgggtgagcg |
| 1381 | gaagaagaca gtgaagggca tgccttgctg ctggcactgc gagccttgca cagggtacca |
| 1441 | gtaccaggtg gaccgctaca cctgtaagac gtgtccctat gacatgcggc ccacagagaa |
| 1501 | ccgcacgggc tgccggccca tccccatcat caagcttgag tggggctcgc cctgggccgt |
| 1561 | gctgcccctc ttcctggccg tggtgggcat cgctgccacg ttgttcgtgg tgatcacctt |
| 1621 | tgtgcgctac aacgacacgc ccatcgtcaa ggcctcgggc cgtgaactga gctacgtgct |
| 1681 | gctggcaggc atcttcctgt gctatgccac caccttcctc atgatcgctg agcccgacct |
| 1741 | tggcacctgc tcgctgcgcc gaatcttcct gggactaggg atgagcatca gctatgcagc |
| 1801 | cctgctcacc aagaccaacc gcatctaccg catcttcgag cagggcaagc gctcggtcag |
| 1861 | tgccccacgc ttcatcagcc ccgcctcaca gctggccatc accttcagcc tcatctcgct |
| 1921 | gcagctgctg ggcatctgtg tgtggtttgt ggtggacccc tcccactcgg tggtggactt |
| 1981 | ccaggaccag cggacactcg acccccgctt cgccaggggt gtgctcaagt gtgacatctc |
| 2041 | ggacctgtcg ctcatctgcc tgctgggcta cagcatgctg ctcatggtca cgtgcaccgt |
| 2101 | gtatgccatc aagacacgcg gcgtgcccga gaccttcaat gaggccaagc ccattggctt |
| 2161 | caccatgtac accacttgca tcgtctggct ggccttcatc cccatcttct ttggcacctc |
| 2221 | gcagtcggcc gacaagctgt acatccagac gacgacgctg acggtctcgg tgagtctgag |
| 2281 | cgcctcggtg tccctgggaa tgctctacat gcccaaagtc tacatcatcc tcttccaccc |
| 2341 | ggagcagaac gtgcccaagc gcaagcgcag cctcaaagcc gtcgttacgg cggccaccat |
| 2401 | gtccaacaag ttcacgcaga agggcaactt ccggcccaac ggagaggcca agtctgagct |
| 2461 | ctgcgagaac cttgaggccc cagcgctggc caccaaacag acttacgtca cttacaccaa |
| 2521 | ccatgcaatc tagcgagtcc atggagctga gcagcaggag gaggagccgt gaccctgtgg |
| 2581 | aaggtgcgtc gggccagggc cacacccaag ggcccagctg tcttgcctgc ccgtgggcac |
| 2641 | ccacggacgt ggcttggtgc tgaggatagc agagccccca gccatcactg ctggcagcct |
| 2701 | gggcaaaccg ggtgagcaac aggaggacga ggggccgggg cggtgccagg ctaccacaag |
| 2761 | aacctgcgtc ttggaccatt gcccctcccg gccccaaacc acaggggctc aggtcgtgtg |
| 2821 | ggccccagtg ctagatctct ccctcccttc gtctctgtct gtgctgttgg cgacccctct |
| 2881 | gtctgtctcc agccctgtct ttctgttctc ttatctcttt gtttcacctt ttccctctct |
| 2941 | ggcgtccccg gctgcttgta ctcttggcct tttctgtgtc tcctttctgg ctcttgcctc |
| 3001 | cgcctctctc tctcatcctc tttgtcctca gctcctcctg ctttcttggg tcccaccagt |
| 3061 | gtcacttttc tgccgttttc tttcctgttc tcctctgctt cattctcgtc cagccattgc |
| 3121 | tcccctctcc ctgccaccct tccccagttc accaaacctt acatgttgca aaagagaaaa |
| 3181 | aaggaaaaaa aatcaaaaca caaaaaagcc aaaacgaaaa caaatctcga gtgtgttgcc |
| 3241 | aagtgctgcg tcctcctggt ggcctctgtg tgtgtccctg tggcccgcag cctgcccgcc |
| 3301 | tgccccgccc atctgccgtg tgtcttgccc gcctgccccg cccgtctgcc gtctgtcttg |
| 3361 | cccgcctgcc cgcctgcccc tcctgccgac cacacggagt tcagtgcctg ggtgtttggt |
| 3421 | gatggttatt gacgacaatg tgtagcgcat gattgttttt ataccaagaa catttctaat |
| 3481 | aaaaataaac acatggtttt gcacccgggc tccacatcca ctgagggtcc tgccatggga |
| 3541 | ccacaggctc agcctgcagc tggagggctt agacctagag ggaagcggga actgggctct |
| 3601 | ggagacccag ggcttggggg ctgtggagac tgctccctag gctgggatct agtgtggtgt |
| 3661 | ggtgaggcct tgggcatgga ggggccagat tcccaggtaa ggggcaggga cattgcagga |
| 3721 | aattccagga atcagcacct agtagtcccc taattagggg gtatgctctg tcccctgccc |
| 3781 | tgcagccctg ggagggtaac atttctgcct tgcctgtcct ctgtctcaca cccctcacac |
| 3841 | ctgggactgc ccttccaccc ctgcccccat aacctgtgcc tctctccttc cagccaggaa |
| 3901 | gtcctcttct tgagaagtta gcttcccggg ctgccagcac tcatagccgt cccctcctgc |
| 3961 | ttgtgttggc tccaggctcg ggtgctaaga agatgtgtgt ctgtcctgga gatcagtgtg |
| 4021 | ttgttatgtg tccacgtggg cccacaagtg cacggcacag gcatggccgt gtggctgtgt |
| 4081 | tggctgtgtt ggctgtgtgt ctgtgtgcac gtccagcgcc tccatgcgca tgcgtgcctg |
| 4141 | tcttgtttgc gtgtctgatc atctgtttgg gccccggtgg ctcatgcaga tgcctgtctc |
| 4201 | aggcccatgg cgagtgttca cctcagctgg cttccctggc aggttgggag gtgggaaaca |
| 4261 | ggagcgctta ggggctgggc tctggctggg gtaaattata gagccagaaa cacaatgagg |
| 4321 | ccataggcag cagctggagc ctgggctgcc tgtgccgtcc cctcctgccc tgcccctggg |
| 4381 | tcctgcaccc cctcccacct ccaggctagc tgacagcgct atggagcaca gtggaaggga |
| 4441 | ctggaggaac cctaggcagg gggccacgca gggacagagt atgagagtgt gtgtataact |
| 4501 | gaggctggga cattgaatca tgccaggtat gtcttctcca tcagcccact cttactcctg |
| 4561 | gcctgggcat ctcacacatc tgtgcatagg aaatctcttc ttccctgggg tctgtgtgca |
| 4621 | gcacctagta gatgctcaat aaatgtttgt gtgagggaag gagacaggaa aggaagtgtc |
| 4681 | tcgctgatca tcttgcggaa tggttcctaa gacctctgcc caggaaagat tccacccagt |
| 4741 | gctccagccc ggtcaggcag aactaggttg ccagatcaag ggtatctccc aaaagcttcc |
| 4801 | agggcagttg ggggtggggg ggtggggggt agggatgggg aatgcagaag cgggtgcagc |
| 4861 | cagctctccc ccagggtgac tctggcagca cccccatcct gggcaccctg cctgctctgt |
| 4921 | ggctcacgcc cctcctgaag tgactgatgc tctgaggccc aaggctaggt ccagggcagg |
| 4981 | gcctgcaggg gtttcatgct cagtccagga cttgcctagg tccccctaca tctgtggggc |
| 5041 | ccccatctag gttctaacag gagaatcacc tctccaaggg ggatgctgcc cctcggctcc |
| 5101 | ccttggctct caggaggggc cctcagggac taccagtccc ctgccagtgg gaagaaatag |
| 5161 | ccctgccctc agggagcttc cagtgtgatg ggggagatac agcagactgt gtcccaaagt |
| 5221 | aaaatgactg ttagaatgag gtgggtggag gagggaagcc ttgggtgggt gtgactttgg |
| 5281 | gcatctgagc ctggggtgca gaggtgggct ctgtgggcct gaggtggaca ggagggaacc |
| 5341 | aggccctagc aagacttttg ccagctagac ctgctgcagc agttgggagg gtgggtgctg |
| 5401 | ctggagtcct gggtccatca cctagaaggc tcaggccagt gcagccaggg ctggggccca |
| 5461 | cagctggcct gggtgggacc tgccctgatg cccatggcag gagggacgcc tggcccttca |
| 5521 | caattggctt ggctgctcac ctttgctctc atcctcaatt attaatgact ggagaaagct |
| 5581 | gctaagtatc ttcagaatgt tagatttcaa caagatgggg ggttcagggt ccctggcacc |
| 5641 | ctggataggg agccagcggc ccctagagac ctttgctgtg tgcagggggt atgtgctcac |
| 5701 | ccccgtggcc tcagcctcct caatgtctga atgaaggatt gggctagcag acatcccacc |
| 5761 | ccacagcaca ctttctaacc agcaggggaa cttctagaca atagagacgc tgggctccct |
| 5821 | ccagaacact ggacctgaac ttctgggggg agggctgggc acgggcatat tttaaaagct |
| 5881 | ccccagcaga tgggccgtgc agtcaagtgg gccaagagtg gcaccagact ttggggcttg |
| 5941 | tgaagtcagg agggagcaac agtgcccact cgagcttgcc tggggctcaa gcccaaggct |
| 6001 | gggctgctgc cagcctgagc agacacccag gagcttccag gccagctgga tgcacagggc |
| 6061 | acctttgtgg aactcctagg accctgggga gacccacctc aggagcagag tctcaggtcc |
| 6121 | cttccggctc tgaggggctg ttctgagctc taatgtctta tggtctgccc ctcccatcct |
| 6181 | tacttctcag gccctggagg cagaggcata gagccaggca ggacagaggt ctcagtgggc |
| 6241 | cacatgccag ctgcccccac actgcctcag cctccaggcc tccaaggggt cctggggagc |
| 6301 | ccctgagaag atgctgagcc tgcataaggc tgggcgcccc tctttctgac accctcactg |
| 6361 | gctccacggc tcccccttcc catcccaggt ttccatctgc ccactgaaca gggaggggaa |
| 6421 | actgaggcac tcccctggca ctgagggctc cttctgtcat cctgcctgcc ctggatggtc |
| 6481 | ctggctgccc ctcagggctt ggccctggca ctgtgagcct cacagggctc agacccccac |
| 6541 | ccccaaccca gcactaaatg gcactcggca ccagaatctc acttcagttg gcaaaagcag |
| 6601 | caattagcat gtaatgaggc ttcttgcttt atttttaggt aacctccaag gccctgcctg |
| 6661 | tgtaattcag cccgccattg ctcggtggat aattaaagca tgtcaccata a. |

In some embodiments, the human mGluR4 comprises or consists of the amino acid sequence of the following sequence, or a functional fragment or variant thereof (UniProtKB Q14833-4 and SEQ ID NO: 21):

| | |
|---|---|
| 1 | MAVPLGAPCW ASALPAWAPP GLPHRSLLTR LLSQHVKPAK IPQISYASTA PDLSDNSRYD |
| 61 | FFSRVVPSDT YQAQAMVDIV RALKWNYVST VASEGSYGES GVEAFIQKSR EDGGVCIAQS |
| 121 | VKIPREPKAG EFDKIIRRLL ETSNARAVII FANEDDIRRV LEAARRANQT GHFFWMGSDS |
| 181 | WGSKIAPVLH LEEVAEGAVT ILPKRMSVRG FDRYFSSRTL DNNRRNIWFA EFWEDNFHCK |
| 241 | LSRHALKKGS HVKKCTNRER IGQDSAYEQE GKVQFVIDAV YAMGHALHAM HRDLCPGRVG |
| 301 | LCPRMDPVDG TQLLKYIRNV NFSGIAGNPV TFNENGDAPG RYDIYQYQLR NDSAEYKVIG |
| 361 | SWTDHLHLRI ERMHWPGSGQ QLPRSICSLP CQPGERKKTV KGMPCCWHCE PCTGYQYQVD |
| 421 | RYTCKTCPYD MRPTENRTGC RPIPIIKLEW GSPWAVLPLF LAVVGIAATL FVVITFVRYN |
| 481 | DTPIVKASGR ELSYVLLAGI FLCYATTFLM IAEPDLGTCS LRRIFLGLGM SISYAALLTK |
| 541 | TNRIYRIFEQ GKRSVSAPRF ISPASQLAIT FSLISLQLLG ICVWFVVDPS HSVVDFQDQR |
| 601 | TLDPRFARGV LKCDISDLSL ICLLGYSMLL MVTCTVYAIK TRGVPETFNE AKPIGFTMYT |
| 661 | TCIVWLAFIP IFFGTSQSAD KLYIQTTTLT VSVSLSASVS LGMLYMPKVY IILFHPEQNV |
| 721 | PKRKRSLKAV VTAATMSNKF TQKGNFRPNG EAKSELCENL EAPALATKQT YVTYTNHAI. |

In some embodiments, the sequence encoding a human mGluR4 comprises or consists of the nucleic acid sequence of the following sequence, or a functional fragment or variant thereof (UniProtKB Q14833-5; GenBank Accession No. NM_001256809.3 and SEQ ID NO: 22):

| | |
|---|---|
| 1 | aatcccagcg ctttgctgga ggatcgcttg agcccaggaa ttcaagacca gcctgggcaa |
| 61 | catggcgaga ccctgtctgt cccaaaacaa aaaaacagat tttaattggt ttgggatgcg |
| 121 | gcctggacac agggaaattt taaagctctc taggtaactc caatgtgcgt cgaggttgcg |
| 181 | aaccgccggc ctgtcacaca gagggagctc aggagatgct gagagtggga ggatgccggc |
| 241 | ttgggagcct ggagttgcag cttcctgcgg ctggcgcgcc cctccttgct cccctctacg |
| 301 | cctctgcatc gcaccccacc cctgtacccc accttcctcc caccaaggaa acctcacctg |
| 361 | ccgcctcccg cccaggtcct ttggattttg ccggtgtgtg tgggtgcgca caagaggccc |
| 421 | ctccctgcca ggagagcagg tgagcctggc cgcgcacgaa tccgaggggg cggccgccca |
| 481 | gctgggaagc agccctgaaa tagacccccg ccgcccccgc tgcctccttc cggaatctgc |
| 541 | tcagataccc cagatcagct acgcctccac agcgccagac ctgagtgaca acagccgcta |
| 601 | cgacttcttc tcccgcgtgg tgccctcgga cacgtaccag gcccaggcca tggtggacat |
| 661 | cgtccgtgcc ctcaagtgga actatgtgtc cacagtggcc tcggagggca gctatggtga |
| 721 | gagcggtgtg gaggccttca tccagaagtc ccgtgaggac gggggcgtgt gcatcgccca |
| 781 | gtcggtgaag ataccacggg agcccaaggc aggcgagttc gacaagatca tccgccgcct |
| 841 | cctggagact tcgaacgcca gggcagtcat catctttgcc aacgaggatg acatcaggcg |
| 901 | tgtgctggag gcagcacgaa gggccaacca gacaggccat ttcttctgga tgggctctga |
| 961 | cagctggggc tccaagattg cacctgtgct gcacctggag gaggtggctg agggtgctgt |
| 1021 | cacgatcctc cccaagagga tgtccgtacg agaccgtgag cgaattgggc aggattcagc |
| 1081 | ttatgagcag gaggggaagg tgcagtttgt gatcgatgcc gtgtacgcca tgggccacgc |
| 1141 | gctgcacgcc atgcaccgtg acctgtgtcc cggccgcgtg gggctctgcc cgcgcatgga |
| 1201 | ccctgtagat ggcacccagc tgcttaagta catccgaaac gtcaacttct caggcatcgc |
| 1261 | agggaaccct gtgaccttca atgagaatgg agatgcgcct gggcgctatg acatctacca |
| 1321 | ataccagctg cgcaacgatt ctgccgagta caaggtcatt ggctcctgga ctgaccacct |
| 1381 | gcaccttaga atagagcgga tgcactggcc ggggagcggg cagcagctgc cccgctccat |
| 1441 | ctgcagcctg ccctgccaac cgggtgagcg gaagaagaca gtgaagggca tgccttgctg |
| 1501 | ctggcactgc gagccttgca cagggtacca gtaccaggtg gaccgctaca cctgtaagac |
| 1561 | gtgtccctat gacatgcggc ccacagagaa ccgcacgggc tgccggccca tccccatcat |
| 1621 | caagcttgag tggggctcgc cctgggccgt gctgcccctc ttcctggccg tggtgggcat |
| 1681 | cgctgccacg ttgttcgtgg tgatcacctt tgtgcgctac aacgacacgc ccatcgtcaa |
| 1741 | ggcctcgggc cgtgaactga gctacgtgct gctggcaggc atcttcctgt gctatgccac |
| 1801 | caccttcctc atgatcgctg agcccgacct tggcacctgc tcgctgcgcc gaatcttcct |
| 1861 | gggactaggg atgagcatca gctatgcagc cctgctcacc aagaccaacc gcatctaccg |
| 1921 | catcttcgag cagggcaagc gctcggtcag tgccccacgc ttcatcagcc ccgcctcaca |
| 1981 | gctggccatc accttcagcc tcatctcgct gcagctgctg ggcatctgtg tgtggtttgt |
| 2041 | ggtggacccc tcccactcgg tggtggactt ccaggaccag cggacactcg acccccgctt |
| 2101 | cgccaggggt gtgctcaagt gtgacatctc ggacctgtcg ctcatctgcc tgctgggcta |
| 2161 | cagcatgctg ctcatggtca cgtgcaccgt gtatgccatc aagacacgcg gcgtgcccga |
| 2221 | gaccttcaat gaggccaagc ccattggctt caccatgtac accacttgca tcgtctggct |
| 2281 | ggccttcatc cccatcttct ttggcacctc gcagtcggcc gacaagctgt acatccagac |
| 2341 | gacgacgctg acggtctcgg tgagtctgag cgcctcggtg tccctgggaa tgctctacat |
| 2401 | gcccaaagtc tacatcatcc tcttccaccc ggagcagaac gtgcccaagc gcaagcgcag |
| 2461 | cctcaaagcc gtcgttacgg cggccaccat gtccaacaag ttcacgcaga agggcaactt |
| 2521 | ccggcccaac ggagaggcca agtctgagct ctgcgagaac cttgaggccc cagcgctggc |
| 2581 | caccaaacag acttacgtca cttacaccaa ccatgcaatc tagcgagtcc atggagctga |
| 2641 | gcagcaggag gaggagccgt gaccctgtgg aaggtgcgtc gggccagggc cacacccaag |
| 2701 | ggcccagctg tcttgcctgc ccgtgggcac ccacggacgt ggcttggtgc tgaggatagc |
| 2761 | agagccccca gccatcactg ctggcagcct gggcaaaccg ggtgagcaac aggaggacga |
| 2821 | ggggccgggg cggtgccagg ctaccacaag aacctgcgtc ttggaccatt gcccctcccg |
| 2881 | gccccaaacc acaggggctc aggtcgtgtg ggccccagtg ctagatctct ccctcccttc |
| 2941 | gtctctgtct gtgctgttgg cgacccctct gtctgtctcc agccctgtct ttctgttctc |
| 3001 | ttatctcttt gtttcacctt ttccctctct ggcgtccccg gctgcttgta ctcttggcct |
| 3061 | tttctgtgtc tcctttctgg ctcttgcctc cgcctctctc tctcatcctc tttgtcctca |
| 3121 | gctcctcctg ctttcttggg tcccaccagt gtcacttttc tgccgttttc tttcctgttc |
| 3181 | tcctctgctt cattctcgtc cagccattgc tcccctctcc ctgccaccct tccccagttc |
| 3241 | accaaacctt acatgttgca aaagagaaaa aaggaaaaaa aatcaaaaca caaaaaagcc |
| 3301 | aaaacgaaaa caaatctcga gtgtgttgcc aagtgctgcg tcctcctggt ggcctctgtg |
| 3361 | tgtgtccctg tggcccgcag cctgcccgcc tgccccgccc atctgccgtg tgtcttgccc |
| 3421 | gcctgccccg cccgtctgcc gtctgtcttg cccgcctgcc cgcctgcccc tcctgccgac |
| 3481 | cacacggagt tcagtgcctg ggtgtttggt gatggttatt gacgacaatg tgtagcgcat |
| 3541 | gattgttttt ataccaagaa catttctaat aaaaataaac acatggtttt gcacccgggc |
| 3601 | tccacatcca ctgagggtcc tgccatggga ccacaggctc agcctgcagc tggagggctt |
| 3661 | agacctagag ggaagcggga actgggctct ggagacccag ggcttggggg ctgtggagac |
| 3721 | tgctccctag gctgggatct agtgtggtgt ggtgaggcct tgggcatgga ggggccagat |
| 3781 | tcccaggtaa ggggcaggga cattgcagga aattccagga atcagcacct agtagtcccc |
| 3841 | taattagggg gtatgctctg tcccctgccc tgcagccctg ggagggtaac atttctgcct |
| 3901 | tgcctgtcct ctgtctcaca cccctcacac ctgggactgc ccttccaccc ctgcccccat |
| 3961 | aacctgtgcc tctctccttc cagccaggaa gtcctcttct tgagaagtta gcttcccggg |
| 4021 | ctgccagcac tcatagccgt cccctcctgc ttgtgttggc tccaggctcg ggtgctaaga |
| 4081 | agatgtgtgt ctgtcctgga gatcagtgtg ttgttatgtg tccacgtggg cccacaagtg |
| 4141 | cacggcacag gcatggccgt gtggctgtgt tggctgtgtt ggctgtgtgt ctgtgtgcac |
| 4201 | gtccagcgcc tccatgcgca tgcgtgcctg tcttgtttgc gtgtctgatc atctgtttgg |
| 4261 | gccccggtgg ctcatgcaga tgcctgtctc aggcccatgg cgagtgttca cctcagctgg |
| 4321 | cttccctggc aggttgggag gtgggaaaca ggagcgctta ggggctgggc tctggctggg |
| 4381 | gtaaattata gagccagaaa cacaatgagg ccataggcag cagctggagc ctgggctgcc |
| 4441 | tgtgccgtcc cctcctgccc tgcccctggg tcctgcaccc cctcccacct ccaggctagc |
| 4501 | tgacagcgct atggagcaca gtggaaggga ctggaggaac cctaggcagg gggccacgca |
| 4561 | gggacagagt atgagagtgt gtgtataact gaggctggga cattgaatca tgccaggtat |
| 4621 | gtcttctcca tcagcccact cttactcctg gcctgggcat ctcacacatc tgtgcatagg |
| 4681 | aaatctcttc ttccctgggg tctgtgtgca gcacctagta gatgctcaat aaatgtttgt |
| 4741 | gtgagggaag gagacaggaa aggaagtgtc tcgctgatca tcttgcggaa tggttcctaa |
| 4801 | gacctctgcc caggaaagat tccacccagt gctccagccc ggtcaggcag aactaggttg |
| 4861 | ccagatcaag ggtatctccc aaaagcttcc agggcagttg ggggtggggg ggtggggggt |
| 4921 | agggatgggg aatgcagaag cgggtgcagc cagctctccc ccagggtgac tctggcagca |
| 4981 | cccccatcct gggcaccctg cctgctctgt ggctcacgcc cctcctgaag tgactgatgc |
| 5041 | tctgaggccc aaggctaggt ccagggcagg gcctgcaggg gtttcatgct cagtccagga |
| 5101 | cttgcctagg tccccctaca tctgtggggc ccccatctag gttctaacag gagaatcacc |
| 5161 | tctccaaggg ggatgctgcc cctcggctcc ccttggctct caggaggggc cctcagggac |
| 5221 | taccagtccc ctgccagtgg gaagaaatag ccctgccctc agggagcttc cagtgtgatg |
| 5281 | ggggagatac agcagactgt gtcccaaagt aaaatgactg ttagaatgag gtgggtggag |
| 5341 | gagggaagcc ttgggtgggt gtgactttgg gcatctgagc ctggggtgca gaggtgggct |
| 5401 | ctgtgggcct gaggtggaca ggagggaacc aggccctagc aagacttttg ccagctagac |
| 5461 | ctgctgcagc agttgggagg gtgggtgctg ctggagtcct gggtccatca cctagaaggc |
| 5521 | tcaggccagt gcagccaggg ctggggccca cagctggcct gggtgggacc tgccctgatg |
| 5581 | cccatggcag gagggacgcc tggcccttca caattggctt ggctgctcac ctttgctctc |
| 5641 | atcctcaatt attaatgact ggagaaagct gctaagtatc ttcagaatgt tagatttcaa |
| 5701 | caagatgggg ggttcagggt ccctggcacc ctggataggg agccagcggc ccctagagac |
| 5761 | ctttgctgtg tgcagggggt atgtgctcac ccccgtggcc tcagcctcct caatgtctga |
| 5821 | atgaaggatt gggctagcag acatcccacc ccacagcaca ctttctaacc agcaggggaa |
| 5881 | cttctagaca atagagacgc tgggctccct ccagaacact ggacctgaac ttctgggggg |
| 5941 | agggctgggc acgggcatat tttaaaagct ccccagcaga tgggccgtgc agtcaagtgg |
| 6001 | gccaagagtg gcaccagact ttggggcttg tgaagtcagg agggagcaac agtgcccact |
| 6061 | cgagcttgcc tggggctcaa gcccaaggct gggctgctgc cagcctgagc agacacccag |
| 6121 | gagcttccag gccagctgga tgcacagggc acctttgtgg aactcctagg accctgggga |
| 6181 | gacccacctc aggagcagag tctcaggtcc cttccggctc tgaggggctg ttctgagctc |
| 6241 | taatgtctta tggtctgccc ctcccatcct tacttctcag gccctggagg cagaggcata |
| 6301 | gagccaggca ggacagaggt ctcagtgggc cacatgccag ctgcccccac actgcctcag |
| 6361 | cctccaggcc tccaaggggt cctggggagc ccctgagaag atgctgagcc tgcataaggc |
| 6421 | tgggcgcccc tctttctgac accctcactg gctccacggc tcccccttcc catcccaggt |
| 6481 | ttccatctgc ccactgaaca gggaggggaa actgaggcac tcccctggca ctgagggctc |
| 6541 | cttctgtcat cctgcctgcc ctggatggtc ctggctgccc ctcagggctt ggccctggca |
| 6601 | ctgtgagcct cacagggctc agacccccac ccccaaccca gcactaaatg gcactcggca |
| 6661 | ccagaatctc acttcagttg gcaaaagcag caattagcat gtaatgaggc ttcttgcttt |
| 6721 | atttttaggt aacctccaag gccctgcctg tgtaattcag cccgccattg ctcggtggat |
| 6781 | aattaaagca tgtcaccata a. |

In some embodiments, the human mGluR4 comprises or consists of the amino acid sequence of the following sequence, or a functional fragment or variant thereof (UniProtKB Q14833-5 and SEQ ID NO: 23):

| | |
|---|---|
| 1 | MPAWEPGVAA SCGWRAPPCS PLRLCIAPHP CTPPSSHQGN LTCRLPPRSF GFCRCVWVRT |
| 61 | RGPSLPGEOV SLAAHESEGA AAQLGSSPEI DPRRPRCLLP ESAQIPQISY ASTAPDLSDN |
| 121 | SRYDFFSRVV PSDTYQAQAM VDIVRALKWN YVSTVASEGS YGESGVEAFI QKSREDGGVC |
| 181 | IAQSVKIPRE PKAGEFDKII RRLLETSNAR AVIIFANEDD IRRVLEAARR ANQTGHFFWM |
| 241 | GSDSWGSKIA PVLHLEEVAE GAVTILPKRM SVRDRERIGQ DSAYEQEGKV QFVIDAVYAM |
| 301 | GHALHAMHRD LCPGRVGLCP RMDPVDGTQL LKYIRNVNFS GIAGNPVTFN ENGDAPGRYD |
| 361 | IYQYQLRNDS AEYKVIGSWT DHLHLRIERM HWPGSGQQLP RSICSLPCQP GERKKTVKGM |
| 421 | PCCWHCEPCT GYQYQVDRYT CKTCPYDMRP TENRTGCRPI PIIKLEWGSP WAVLPLFLAV |
| 481 | VGIAATLFVV ITFVRYNDTP IVKASGRELS YVLLAGIFLC YATTFLMIAE PDLGTCSLRR |
| 541 | IFLGLGMSIS YAALLTKTNR IYRIFEQGKR SVSAPRFISP ASQLAITFSL ISLQLLGICV |
| 601 | WFVVDPSHSV VDFQDQRTLD PRFARGVLKC DISDLSLICL LGYSMLLMVT CTVYAIKTRG |
| 661 | VPETFNEAKP IGFTMYTTCI VWLAFIPIFF GTSQSADKLY IQTTTLTVSV SLSASVSLGM |
| 721 | LYMPKVYIIL FHPEQNVPKR KRSLKAVVTA ATMSNKFTQK GNFRPNGEAK SELCENLEAP |
| 781 | ALATKQTYVT YTNHAI. |

In some embodiments, the mGluR comprises mGLuR5. In some embodiments, the sequence encoding an mGluR comprises a sequence encoding a human mGluR5.

In some embodiments, the sequence encoding a human mGluR5 comprises or consists of the nucleic acid sequence of the following sequence, or a functional fragment or variant thereof (UniProtKB P41594-1; GenBank Accession No. NM_001143831.3 and SEQ ID NO: 24):

| | |
|---|---|
| 1 | gcgtctgccc gagcgcggca cgtgctcccg gcgctggcgc gagagagcga gcgccaccgc |
| 61 | cgcgggcccc gcagccgttc tgcctgctgt caccgctgcc tccatcgccg acactagcgc |
| 121 | tccagctgca gccaaggccg ctacgagagc gcaggaagcc ctcgaggagc ggctgcctgg |
| 181 | gggcgcaagg ctcagggcgc gcacctggtt tagaagatca tgaccacatg gatcatctaa |
| 241 | ctaaatggta catggggaca aaatggtcct ttagaaaata catctgaatt gctggctaat |
| 301 | ttcttgattt gcgactcaac gtaggacatc gcttgttcgt agctatcaga accctcctga |
| 361 | attttcccca ccatgctatc tttattggct tgaactcctt tcctaaaatg gtccttctgt |
| 421 | tgatcctgtc agtcttactt ttgaaagaag atgtccgtgg gagtgcacag tccagtgaga |
| 481 | ggagggtggt ggctcacatg ccgggtgaca tcattattgg agctctcttt tctgttcatc |
| 541 | accagcctac tgtggacaaa gttcatgaga ggaagtgtgg ggcggtccgt gaacagtatg |
| 601 | gcattcagag agtggaggcc atgctgcata ccctggaaag gatcaattca gaccccacac |
| 661 | tcttgcccaa catcacactg ggctgtgaga taagggactc ctgctggcat tcggctgtgg |
| 721 | ccctagagca gagcattgag ttcataagag attccctcat ttcttcagaa gaggaagaag |
| 781 | gcttggtacg ctgtgtggat ggctcctcct cttccttccg ctccaagaag cccatagtag |
| 841 | gggtcattgg gcctggctcc agttctgtag ccattcaggt ccagaatttg ctccagcttt |
| 901 | tcaacatacc tcagattgct tactcagcaa ccagcatgga tctgagtgac aagactctgt |
| 961 | tcaaatattt catgagggtt gtgccttcag atgctcagca ggcaagggcc atggtggaca |
| 1021 | tagtgaagag gtacaactgg acctatgtat cagccgtgca cacagaaggc aactatggag |
| 1081 | aaagtgggat ggaagccttc aaagatatgt cagcgaagga agggatttgc atcgcccact |
| 1141 | cttacaaaat ctacagtaat gcaggggagc agagctttga taagctgctg aagaagctca |
| 1201 | caagtcactt gcccaaggcc cgggtggtgg cctgcttctg tgagggcatg acggtgagag |
| 1261 | gtctgctgat ggccatgagg cgcctgggtc tagcgggaga atttctgctt ctgggcagtg |
| 1321 | atggctgggc tgacaggtat gatgtgacag atggatatca gcgagaagct gttggtggca |
| 1381 | tcacaatcaa gctccaatct cccgatgtca agtggtttga tgattattat ctgaagctcc |
| 1441 | ggccagaaac aaaccaccga aacccttggt ttcaagaatt ttggcagcat cgttttcagt |
| 1501 | gccgactgga agggtttcca caggagaaca gcaaatacaa caagacttgc aatagttctc |
| 1561 | tgactctgaa aacacatcat gttcaggatt ccaaaatggg atttgtgatc aacgccatct |
| 1621 | attcgatggc ctatgggctc cacaacatgc agatgtccct ctgcccaggc tatgcaggac |
| 1681 | tctgtgatgc catgaagcca attgatggac ggaaactttt ggagtccctg atgaaaacca |
| 1741 | attttactgg ggtttctgga gatacgatcc tattcgatga gaatggagac tctccaggaa |
| 1801 | ggtatgaaat aatgaatttc aaggaaatgg gaaaagatta ctttgattat atcaacgttg |
| 1861 | gaagttggga caatggagaa ttaaaaatgg atgatgatga agtatggtcc aagaaaagca |
| 1921 | acatcatcag atctgtgtgc agtgaaccat gtgagaaagg ccagatcaag gtgatccgaa |
| 1981 | agggagaagt cagctgttgt tggacctgta caccttgtaa ggagaatgag tatgtctttg |
| 2041 | atgagtacac atgcaaggca tgccaactgg ggtcttggcc cactgatgat ctcacaggtt |
| 2101 | gtgacttgat cccagtacag tatcttcgat ggggtgaccc tgaacccatt gcagctgtgg |
| 2161 | tgtttgcctg ccttggcctc ctggccaccc tgtttgttac tgtagtcttc atcatttacc |
| 2221 | gtgatacacc agtagtcaag tcctcaagca gggaactctg ctacattatc cttgctggca |
| 2281 | tctgcctggg ctacttatgt accttctgcc tcattgcgaa gcccaaacag atttactgct |
| 2341 | accttcagag aattggcatt ggtctctccc cagccatgag ctactcagcc cttgtaacaa |
| 2401 | agaccaaccg tattgcaagg atcctggctg gcagcaagaa gaagatctgt accaaaaagc |
| 2461 | ccagattcat gagtgcctgt gcccagctag tgattgcttt cattctcata tgcatccagt |
| 2521 | tgggcatcat cgttgccctc tttataatgg agcctcctga cataatgcat gactacccaa |
| 2581 | gcattcgaga agtctacctg atctgtaaca ccaccaacct aggagttgtc actccacttg |
| 2641 | gatacaatgg attgttgatt ttgagctgca ccttctatgc gttcaagacc agaaatgttc |
| 2701 | cagctaactt caacgaggcc aagtatatcg ccttcacaat gtacacgacc tgcattatat |
| 2761 | ggctagcttt tgtgccaatc tactttggca gcaactacaa aatcatcacc atgtgtttct |
| 2821 | cggtcagcct cagtgccaca gtggccctag gctgcatgtt tgtgccgaag gtgtacatca |
| 2881 | tcctggccaa accagagaga aacgtgcgca gcgccttcac cacatctacc gtggtgcgca |
| 2941 | tgcatgtagg ggatggcaag tcatcctccg cagccagcag atccagcagc ctagtcaacc |
| 3001 | tgtggaagag aaggggctcc tctggggaaa ccttaaggta caaagacagg agactggccc |
| 3061 | agcacaagtc ggaaatagag tgtttcaccc ccaaagggag tatggggaat ggtgggagag |
| 3121 | caacaatgag cagttccaat ggaaaatccg tcacgtgggc ccagaatgag aagagcagcc |
| 3181 | gggggcagca cctgtggcag cgcctgtcca tccacatcaa caagaaagaa aaccccaacc |
| 3241 | aaacggccgt catcaagccc ttccccaaga gcacggagag ccgtggcctg ggcgctggcg |
| 3301 | ctggcgcagg cgggagcgct gggggcgtgg gggccacggg cggtgcgggc tgcgcaggcg |
| 3361 | ccggcccagg cgggcccgag tccccagacg ccggccccaa ggcgctgtat gatgtggccg |
| 3421 | aggctgagga gcacttcccg gcgcccgcgc ggccgcgctc accgtcgccc atcagcacgc |
| 3481 | tgagccaccg cgcgggctcg gccagccgca cggacgacga tgtgccgtcg ctgcactcgg |
| 3541 | agcctgtggc gcgcagcagc tcctcgcagg gctccctcat ggagcagatc agcagtgtgg |
| 3601 | tcacccgctt cacggccaac atcagcgagc tcaactccat gatgctgtcc accgcggccc |
| 3661 | ccagccccgg cgtcggcgcc ccgctctgct cgtcctacct gatccccaaa gagatccagt |
| 3721 | tgcccacgac catgacgacc tttgccgaaa tccagcctct gccggccatc gaagtcacgg |
| 3781 | gaggcgcgca gcccgcggca ggggcgcagg cggctgggga cgcggcccgg gagagccccg |
| 3841 | cggccggtcc cgaggctgcg gccgccaagc cagacctgga ggagctggtg gctctcaccc |
| 3901 | cgccgtcccc cttcagagac tcggtggact cggggagcac aacccccaac tcgccagtgt |
| 3961 | ccgagtcggc cctctgtatc ccgtcgtctc ccaaatatga cactcttatc ataagagatt |
| 4021 | acactcagag ctcctcgtcg ttgtgaatgt ccctggaaag cacgccggcc tgcgcgtgcg |
| 4081 | gagcggagcc ccccgtgttc acacacacac aatggcaagc atagtcgcct ggttacggcc |
| 4141 | cagggggaag atgccaaggg caccccttaa tggaaacacg agatcagtag tgctatctca |
| 4201 | tgacaaccga cgaagaaacc gacgacaaat cttttggcag attttcttct agtggcctta |
| 4261 | gaaaacatgg gcttttaaga aacacggctg atatctttga gggctgacaa ggcgtctctt |
| 4321 | caaacagttc cataccaagt gctttgctct agggaagcag tgcgtgtgaa acagcgtaac |
| 4381 | ggagggtgaa gagcatagtt aataagcaac tgtaaaaagt tttatttgtt tactttaatt |
| 4441 | cttttcccag aagagtcttt gattcaccaa acatgaatgt acattttcta acaaactcaa |
| 4501 | aatctgggac caaaacatca acttttttct ttcttttttc tttctttttg ttttttcttt |
| 4561 | cctgtaaaga ccttgaaaag cagtaacttg ggtccagtat ttacggaggc gttgtgaatg |
| 4621 | tgtcccatgc ataacacact actggatagt gagtgctgcg ctaatgtact acgtagggct |
| 4681 | tctaccagag attttcctct ccaattgggt tgtgaaatac tcttccaaaa gcctgcatcg |
| 4741 | gggattccac ctacttattt cagattcacc tccattaacc aagaaaacca gtggaagatt |
| 4801 | tcttgactat ttcaccatgt tgccaatcaa tactggagta gcaaaaaaaa tattttctgg |
| 4861 | aatactgttt tgtaattccc tcactggggt gcattgtagc tggaaattct ctttataaaa |
| 4921 | atcattcttg agctccagcc tggctatctc tttcaagaaa catggccact ctttaggaat |
| 4981 | gctgttgcgt ttgcattgcc aactaaaata ttaaaatatg cattggggct tcttcattcc |
| 5041 | tttattttga gaacctgatg cacaaagagc tcctttgttc ttttcgagtc ccaccactgg |
| 5101 | aagagtggtc catagacccc atgaagacat tgtcatgatt tgagagactg ttgttgaaag |
| 5161 | gattaacaca atcttaatac actgaaaatt ttaactgtgt caagtcagct tagtggagat |
| 5221 | ttagctatgc cagtgagcag tgattttaac tattcttggc tgcttaaaca gggcagctat |
| 5281 | gaactatgac aaatgtagat ttttcaaagc aatacaaaat actaaaaaag aggaacctta |
| 5341 | atgaatatta accacacagt ctttcttagc cattccaaaa agaggcaaag caattcttat |
| 5401 | tttctttttt aaaataatga ttaatatgat tttgtgcact tcatactgtc actttttaaa |
| 5461 | actacagaaa agagatttag agtataacag aaacaagtgt gctttgatag tctcaaatag |
| 5521 | gtagaattca tagttcaaga cctgaatcca ctgtcatctc tttcttcctc ccattgcagc |
| 5581 | tatcctcagg taccaaatgt tttgattttt aaataaggat agtaataaat ggaggaggtg |
| 5641 | tcctataaat ttaaagttca gttgacccag ccttatactt aagatagcct tatgaaaaat |
| 5701 | atgtgctgtg aggcagaagt atattttggc agagagaata ataaataaaa ctttttcttt |
| 5761 | tagctcaata tccttacttt ggtaagtatt tttttttatt tcacatctac ttaacagaaa |
| 5821 | ataaactgag aaatagaagt cagtccattg gcataattta tcattcttca ctttaaaaaa |
| 5881 | ttctaataaa tattctgctt gagttttctt ttctgctatt tgttcttact tgcaacttta |
| 5941 | agtcaaacct cccaatacaa aacattaaaa gctaacatta atgtactaaa gtattaattt |
| 6001 | aaaagaaatc gaacctccca tgctagattt gaaaataaca tcatcacagc accctgatcc |
| 6061 | caaatattac accgaggctt ttaaaatgta agtgaaatct agctaagttt catggtttca |
| 6121 | ttaaaagcaa atgtctgcct ctatctgaaa aacaaatgga aatcttttga ggtgttaata |
| 6181 | ccctttggat cctcatcaaa aggatggcat tcacctgagg attcctatct tgacttctta |
| 6241 | ggtattaaaa acctttcttg atatgctcta cattttaaaa tttgttttat aaaatcctta |
| 6301 | tgttgatttt cattttattc tcaagtacaa tacgtttcac tctagaccag ttgaagaaca |
| 6361 | tgtttaaact ttgttcatgg tcaaattcat tttctatttt tttagtaaca tatctcttaa |
| 6421 | aaagcacact accttataaa aaacttcatc agaaattaaa tttaatgcaa gtaaattgcc |
| 6481 | atctgatact tccacatgct atcataatca actgtaataa taaaaatgat ttatccaatt |
| 6541 | agaaaaggac aagatatatt tttctctgta tttctataac ttttgccact ccattgaata |
| 6601 | cattgtatgt tggacataag attattagta atgcattctt gagatctttt attttggaat |
| 6661 | gatgctaact ctgtctcttt gccaattcta ataccaggtt ccaagtaata actctacagt |
| 6721 | acaaagagaa ctgaatattc attctagggc tataggatat gaacttcaca attcatttgg |
| 6781 | gtacattctc attgaatttc cttcaaaaca atctgttcct ggtgcccagt gataattcag |
| 6841 | tcgggaccag catgactaaa aggaagggga tatgctaagg ctcagcaaag tgaccctaaa |
| 6901 | tgagagatat gtcccaggat ggaaagaaga agacgtggtt taaccaagtt atactgacta |
| 6961 | atctaagcag tccactcatc cttccatttt gggaaaggag tgggggcagc ctaagaagaa |
| 7021 | catatctgga ttgggaagaa ccgtctttct gggctaggga tggggaacag aaagggagta |
| 7081 | tggaaagaaa aattataaga gatttgactg aagcaaggaa aaaaagcaaa tccccaaacg |
| 7141 | tgctaatcct tgaaagtaac tatctttccc aaactactgc tgttaccagc aagtgatcag |
| 7201 | gaagactagg agctatttct gactgtaaat gaattgtata atagctctgc tgcagttctg |
| 7261 | tgacttccaa gccaggaatt aaatgctctt tttaagaata acaaaaaaca aaagcatttc |
| 7321 | ctatgctagt ctcccagtaa aatgtacatg ttttggagac ttcaaaggta ttatgtgagt |
| 7381 | tcacatttag caacagctta ttaataaccc tcaagctgtc agaatctcta tagttaccat |
| 7441 | ttacaatttt atactgtgaa aaaatacaga tcagtgaaag cataaagaca agtcagaatt |
| 7501 | cactttgaag agggtctgag gcctgggaga gtctctactg tctattgaag aatgaggcat |
| 7561 | gtataaaata gttggttgaa tttcactgat cttcccaatg tgaacaaata tactatgtat |
| 7621 | attgtgtgta tttctagaaa tcaatggcag ctgctgatgg tgttgtaatt agaaatctat |
| 7681 | atagattata gatgttttag aaagatggtg ccaatcctaa aagatttgtg tgggctaaaa |
| 7741 | gtgcttgtac ttactttttt ctgcacttat aactgatttg gttttaaaat tgtgtgcgtg |
| 7801 | tatctgttct ttctctgttg tggcagcttg tactattaaa ataatagaga atgttaaatt |
| 7861 | attttgatgt gaactgcaaa tgattttttt tcataaagtt taacattttt atcagcattg |
| 7921 | ttttgctttg tacttgtata aatatgtttt attttagcac ttcaaaatat acttgcctgt |
| 7981 | ttctcagttg tctaaa. |

In some embodiments, the human mGluR5 comprises or consists of the amino acid sequence of the following sequence, or a functional fragment or variant thereof (UniProtKB P41594-1 and SEQ ID NO: 25):

| | |
|---|---|
| 1 | MVLLLILSVL LLKEDVRGSA QSSERRVVAH MPGDIIIGAL FSVHHQPTVD KVHERKCGAV |
| 61 | REQYGIQRVE AMLHTLERIN SDPTLLPNIT LGCEIRDSCW HSAVALEQSI EFIRDSLISS |
| 121 | EEEEGLVRCV DGSSSSFRSK KPIVGVIGPG SSSVAIQVQN LLQLFNIPQI AYSATSMDLS |
| 181 | DKTLFKYFMR VVPSDAQQAR AMVDIVKRYN WTYVSAVHTE GNYGESGMEA FKDMSAKEGI |
| 241 | CIAHSYKIYS NAGEQSFDKL LKKLTSHLPK ARVVACFCEG MTVRGLLMAM RRLGLAGEFL |
| 301 | LLGSDGWADR YDVTDGYQRE AVGGITIKLQ SPDVKWFDDY YLKLRPETNH RNPWFQEFWQ |
| 361 | HRFQCRLEGF PQENSKYNKT CNSSLTLKTH HVQDSKMGFV INAIYSMAYG LHNMQMSLCP |
| 421 | GYAGLCDAMK PIDGRKLLES LMKTNFTGVS GDTILFDENG DSPGRYEIMN FKEMGKDYFD |
| 481 | YINVGSWDNG ELKMDDDEVW SKKSNIIRSV CSEPCEKGQI KVIRKGEVSC CWTCTPCKEN |
| 541 | EYVFDEYTCK ACQLGSWPTD DLTGCDLIPV QYLRWGDPEP IAAVVFACLG LLATLFVTVV |
| 601 | FIIYRDTPVV KSSSRELCYI ILAGICLGYL CTFCLIAKPK QIYCYLQRIG IGLSPAMSYS |
| 661 | ALVTKTNRIA RILAGSKKKI CTKKPRFMSA CAQLVIAFIL ICIQLGIIVA LFIMEPPDIM |
| 721 | HDYPSIREVY LICNTTNLGV VTPLGYNGLL ILSCTFYAFK TRNVPANFNE AKYIAFTMYT |
| 781 | TCIIWLAFVP IYFGSNYKII TMCFSVSLSA TVALGCMFVP KVYIILAKPE RNVRSAFTTS |
| 841 | TVVRMHVGDG KSSSAASRSS SLVNLWKRRG SSGETLRYKD RRLAQHKSEI ECFTPKGSMG |
| 901 | NGGRATMSSS NGKSVTWAQN EKSSRGQHLW QRLSIHINKK ENPNQTAVIK PFPKSTESRG |
| 961 | LGAGAGAGGS AGGVGATGGA GCAGAGPGGP ESPDAGPKAL YDVAEAEEHF PAPARPRSPS |
| 1021 | PISTLSHRAG SASRTDDDVP SLHSEPVARS SSSQGSLMEQ ISSVVTRFTA NISELNSMML |
| 1081 | STAAPSPGVG APLCSSYLIP KEIQLPTTMT TFAEIQPLPA IEVTGGAQPA AGAQAAGDAA |
| 1141 | RESPAAGPEA AAAKPDLEEL VALTPPSPFR DSVDSGSTTP NSPVSESALC IPSSPKYDTL |
| 1201 | IIRDYTOSSS SL. |

In some embodiments, the signal peptide corresponding to residues from about 1-22 of SEQ ID NO: 25 above is used in place of the signal peptide of another glutamate receptor, such as mGluR2.

In some embodiments, the sequence encoding a human mGluR5 comprises or consists of the nucleic acid sequence of the following sequence, or a functional fragment or variant thereof (UniProtKB P41594-2; GenBank Accession No. NM_001384268.1 and SEQ ID NO: 26):

| | |
|---|---|
| 1 | gcgtctgccc gagcgcggca cgtgctcccg gcgctggcgc gagagagcga gcgccaccgc |
| 61 | cgcgggcccc gcagccgttc tgcctgctgt caccgctgcc tccatcgccg acactagcgc |
| 121 | tccagctgca gccaaggccg ctacgagagc gcaggaagcc ctcgaggagc ggctgcctgg |
| 181 | gggcgcaagg ctcagggcgc gcacctggtt tagaagatca tgaccacatg gatcatctaa |
| 241 | ctaaatggta catggggaca aaatggtcct ttagaaaata catctgaatt gctggctaat |
| 301 | ttcttgattt gcgactcaac gtaggacatc gcttgttcgt agctatcaga accctcctga |
| 361 | attttcccca ccatgctatc tttattggct tgaactcctt tcctaaaatg gtccttctgt |
| 421 | tgatcctgtc agtcttactt ttgaaagaag atgtccgtgg gagtgcacag tccagtgaga |
| 481 | ggagggtggt ggctcacatg ccgggtgaca tcattattgg agctctcttt tctgttcatc |
| 541 | accagcctac tgtggacaaa gttcatgaga ggaagtgtgg ggcggtccgt gaacagtatg |
| 601 | gcattcagag agtggaggcc atgctgcata ccctggaaag gatcaattca gaccccacac |
| 661 | tcttgcccaa catcacactg ggctgtgaga taagggactc ctgctggcat tcggctgtgg |
| 721 | ccctagagca gagcattgag ttcataagag attccctcat ttcttcagaa gaggaagaag |
| 781 | gcttggtacg ctgtgtggat ggctcctcct cttccttccg ctccaagaag cccatagtag |
| 841 | gggtcattgg gcctggctcc agttctgtag ccattcaggt ccagaatttg ctccagcttt |
| 901 | tcaacatacc tcagattgct tactcagcaa ccagcatgga tctgagtgac aagactctgt |
| 961 | tcaaatattt catgagggtt gtgccttcag atgctcagca ggcaagggcc atggtggaca |
| 1021 | tagtgaagag gtacaactgg acctatgtat cagccgtgca cacagaaggc aactatggag |
| 1081 | aaagtgggat ggaagccttc aaagatatgt cagcgaagga agggatttgc atcgcccact |
| 1141 | cttacaaaat ctacagtaat gcaggggagc agagctttga taagctgctg aagaagctca |
| 1201 | caagtcactt gcccaaggcc cgggtggtgg cctgcttctg tgagggcatg acggtgagag |
| 1261 | gtctgctgat ggccatgagg cgcctgggtc tagcgggaga atttctgctt ctgggcagtg |
| 1321 | atggctgggc tgacaggtat gatgtgacag atggatatca gcgagaagct gttggtggca |
| 1381 | tcacaatcaa gctccaatct cccgatgtca agtggtttga tgattattat ctgaagctcc |
| 1441 | ggccagaaac aaaccaccga aacccttggt ttcaagaatt ttggcagcat cgttttcagt |
| 1501 | gccgactgga agggtttcca caggagaaca gcaaatacaa caagacttgc aatagttctc |
| 1561 | tgactctgaa aacacatcat gttcaggatt ccaaaatggg atttgtgatc aacgccatct |
| 1621 | attcgatggc ctatgggctc cacaacatgc agatgtccct ctgcccaggc tatgcaggac |
| 1681 | tctgtgatgc catgaagcca attgatggac ggaaactttt ggagtccctg atgaaaacca |
| 1741 | attttactgg ggtttctgga gatacgatcc tattcgatga gaatggagac tctccaggaa |
| 1801 | ggtatgaaat aatgaatttc aaggaaatgg gaaaagatta ctttgattat atcaacgttg |
| 1861 | gaagttggga caatggagaa ttaaaaatgg atgatgatga agtatggtcc aagaaaagca |
| 1921 | acatcatcag atctgtgtgc agtgaaccat gtgagaaagg ccagatcaag gtgatccgaa |
| 1981 | agggagaagt cagctgttgt tggacctgta caccttgtaa ggagaatgag tatgtctttg |
| 2041 | atgagtacac atgcaaggca tgccaactgg ggtcttggcc cactgatgat ctcacaggtt |
| 2101 | gtgacttgat cccagtacag tatcttcgat ggggtgaccc tgaacccatt gcagctgtgg |
| 2161 | tgtttgcctg ccttggcctc ctggccaccc tgtttgttac tgtagtcttc atcatttacc |
| 2221 | gtgatacacc agtagtcaag tcctcaagca gggaactctg ctacattatc cttgctggca |
| 2281 | tctgcctggg ctacttatgt accttctgcc tcattgcgaa gcccaaacag atttactgct |
| 2341 | accttcagag aattggcatt ggtctctccc cagccatgag ctactcagcc cttgtaacaa |
| 2401 | agaccaaccg tattgcaagg atcctggctg gcagcaagaa gaagatctgt accaaaaagc |
| 2461 | ccagattcat gagtgcctgt gcccagctag tgattgcttt cattctcata tgcatccagt |
| 2521 | tgggcatcat cgttgccctc tttataatgg agcctcctga cataatgcat gactacccaa |
| 2581 | gcattcgaga agtctacctg atctgtaaca ccaccaacct aggagttgtc actccacttg |
| 2641 | gatacaatgg attgttgatt ttgagctgca ccttctatgc gttcaagacc agaaatgttc |
| 2701 | cagctaactt caacgaggcc aagtatatcg ccttcacaat gtacacgacc tgcattatat |
| 2761 | ggctagcttt tgtgccaatc tactttggca gcaactacaa aatcatcacc atgtgtttct |
| 2821 | cggtcagcct cagtgccaca gtggccctag gctgcatgtt tgtgccgaag gtgtacatca |
| 2881 | tcctggccaa accagagaga aacgtgcgca gcgccttcac cacatctacc gtggtgcgca |
| 2941 | tgcatgtagg ggatggcaag tcatcctccg cagccagcag atccagcagc ctagtcaacc |
| 3001 | tgtggaagag aaggggctcc tctggggaaa ccttaagttc caatggaaaa tccgtcacgt |
| 3061 | gggcccagaa tgagaagagc agccgggggc agcacctgtg gcagcgcctg tccatccaca |
| 3121 | tcaacaagaa agaaaacccc aaccaaacgg ccgtcatcaa gcccttcccc aagagcacgg |
| 3181 | agagccgtgg cctgggcgct ggcgctggcg caggcgggag cgctgggggc gtgggggcca |
| 3241 | cgggcggtgc gggctgcgca ggcgccggcc caggcgggcc cgagtcccca gacgccggcc |
| 3301 | ccaaggcgct gtatgatgtg gccgaggctg aggagcactt cccggcgccc gcgcggccgc |
| 3361 | gctcaccgtc gcccatcagc acgctgagcc accgcgcggg ctcggccagc cgcacggacg |
| 3421 | acgatgtgcc gtcgctgcac tcggagcctg tggcgcgcag cagctcctcg cagggctccc |
| 3481 | tcatggagca gatcagcagt gtggtcaccc gcttcacggc caacatcagc gagctcaact |
| 3541 | ccatgatgct gtccaccgcg gcccccagcc ccggcgtcgg cgccccgctc tgctcgtcct |
| 3601 | acctgatccc caaagagatc cagttgccca cgaccatgac gacctttgcc gaaatccagc |
| 3661 | ctctgccggc catcgaagtc acgggaggcg cgcagcccgc ggcaggggcg caggcggctg |
| 3721 | gggacgcggc ccgggagagc cccgcggccg gtcccgaggc tgcggccgcc aagccagacc |
| 3781 | tggaggagct ggtggctctc accccgccgt cccccttcag agactcggtg gactcgggga |
| 3841 | gcacaacccc caactcgcca gtgtccgagt cggccctctg tatcccgtcg tctcccaaat |
| 3901 | atgacactct tatcataaga gattacactc agagctcctc gtcgttgtga atgtccctgg |
| 3961 | aaagcacgcc ggcctgcgcg tgcggagcgg agccccccgt gttcacacac acacaatggc |
| 4021 | aagcatagtc gcctggttac ggcccagggg gaagatgcca agggcacccc ttaatggaaa |
| 4081 | cacgagatca gtagtgctat ctcatgacaa ccgacgaaga aaccgacgac aaatcttttg |
| 4141 | gcagattttc ttctagtggc cttagaaaac atgggctttt aagaaacacg gctgatatct |
| 4201 | ttgagggctg acaaggcgtc tcttcaaaca gttccatacc aagtgctttg ctctagggaa |
| 4261 | gcagtgcgtg tgaaacagcg taacggaggg tgaagagcat agttaataag caactgtaaa |
| 4321 | aagttttatt tgtttacttt aattcttttc ccagaagagt ctttgattca ccaaacatga |
| 4381 | atgtacattt tctaacaaac tcaaaatctg ggaccaaaac atcaactttt ttctttcttt |
| 4441 | tttctttctt tttgtttttt ctttcctgta aagaccttga aaagcagtaa cttgggtcca |
| 4501 | gtatttacgg aggcgttgtg aatgtgtccc atgcataaca cactactgga tagtgagtgc |
| 4561 | tgcgctaatg tactacgtag ggcttctacc agagattttc ctctccaatt gggttgtgaa |
| 4621 | atactcttcc aaaagcctgc atcggggatt ccacctactt atttcagatt cacctccatt |
| 4681 | aaccaagaaa accagtggaa gatttcttga ctatttcacc atgttgccaa tcaatactgg |
| 4741 | agtagcaaaa aaaatatttt ctggaatact gttttgtaat tccctcactg gggtgcattg |
| 4801 | tagctggaaa ttctctttat aaaaatcatt cttgagctcc agcctggcta tctctttcaa |
| 4861 | gaaacatggc cactctttag gaatgctgtt gcgtttgcat tgccaactaa aatattaaaa |
| 4921 | tatgcattgg ggcttcttca ttcctttatt ttgagaacct gatgcacaaa gagctccttt |
| 4981 | gttcttttcg agtcccacca ctggaagagt ggtccataga ccccatgaag acattgtcat |
| 5041 | gatttgagag actgttgttg aaaggattaa cacaatctta atacactgaa aattttaact |
| 5101 | gtgtcaagtc agcttagtgg agatttagct atgccagtga gcagtgattt taactattct |
| 5161 | tggctgctta aacagggcag ctatgaacta tgacaaatgt agatttttca aagcaataca |
| 5221 | aaatactaaa aaagaggaac cttaatgaat attaaccaca cagtctttct tagccattcc |
| 5281 | aaaaagaggc aaagcaattc ttattttctt ttttaaaata atgattaata tgattttgtg |
| 5341 | cacttcatac tgtcactttt taaaactaca gaaaagagat ttagagtata acagaaacaa |
| 5401 | gtgtgctttg atagtctcaa ataggtagaa ttcatagttc aagacctgaa tccactgtca |
| 5461 | tctctttctt cctcccattg cagctatcct caggtaccaa atgttttgat ttttaaataa |
| 5521 | ggatagtaat aaatggagga ggtgtcctat aaatttaaag ttcagttgac ccagccttat |
| 5581 | acttaagata gccttatgaa aaatatgtgc tgtgaggcag aagtatattt tggcagagag |
| 5641 | aataataaat aaaacttttt cttttagctc aatatcctta ctttggtaag tatttttttt |
| 5701 | tatttcacat ctacttaaca gaaaataaac tgagaaatag aagtcagtcc attggcataa |
| 5761 | tttatcattc ttcactttaa aaaattctaa taaatattct gcttgagttt tcttttctgc |
| 5821 | tatttgttct tacttgcaac tttaagtcaa acctcccaat acaaaacatt aaaagctaac |
| 5881 | attaatgtac taaagtatta atttaaaaga aatcgaacct cccatgctag atttgaaaat |
| 5941 | aacatcatca cagcaccctg atcccaaata ttacaccgag gcttttaaaa tgtaagtgaa |
| 6001 | atctagctaa gtttcatggt ttcattaaaa gcaaatgtct gcctctatct gaaaaacaaa |
| 6061 | tggaaatctt ttgaggtgtt aatacccttt ggatcctcat caaaaggatg gcattcacct |
| 6121 | gaggattcct atcttgactt cttaggtatt aaaaaccttt cttgatatgc tctacatttt |
| 6181 | aaaatttgtt ttataaaatc cttatgttga ttttcatttt attctcaagt acaatacgtt |
| 6241 | tcactctaga ccagttgaag aacatgttta aactttgttc atggtcaaat tcattttcta |
| 6301 | tttttttagt aacatatctc ttaaaaagca cactacctta taaaaaactt catcagaaat |
| 6361 | taaatttaat gcaagtaaat tgccatctga tacttccaca tgctatcata atcaactgta |
| 6421 | ataataaaaa tgatttatcc aattagaaaa ggacaagata tatttttctc tgtatttcta |
| 6481 | taacttttgc cactccattg aatacattgt atgttggaca taagattatt agtaatgcat |
| 6541 | tcttgagatc ttttattttg gaatgatgct aactctgtct ctttgccaat tctaatacca |
| 6601 | ggttccaagt aataactcta cagtacaaag agaactgaat attcattcta gggctatagg |
| 6661 | atatgaactt cacaattcat ttgggtacat tctcattgaa tttccttcaa aacaatctgt |
| 6721 | tcctggtgcc cagtgataat tcagtcggga ccagcatgac taaaaggaag gggatatgct |
| 6781 | aaggctcagc aaagtgaccc taaatgagag atatgtccca ggatggaaag aagaagacgt |
| 6841 | ggtttaacca agttatactg actaatctaa gcagtccact catccttcca ttttgggaaa |
| 6901 | ggagtggggg cagcctaaga agaacatatc tggattggga agaaccgtct ttctgggcta |
| 6961 | gggatgggga acagaaaggg agtatggaaa gaaaaattat aagagatttg actgaagcaa |
| 7021 | ggaaaaaaag caaatcccca aacgtgctaa tccttgaaag taactatctt tcccaaacta |
| 7081 | ctgctgttac cagcaagtga tcaggaagac taggagctat ttctgactgt aaatgaattg |
| 7141 | tataatagct ctgctgcagt tctgtgactt ccaagccagg aattaaatgc tctttttaag |
| 7201 | aataacaaaa aacaaaagca tttcctatgc tagtctccca gtaaaatgta catgttttgg |
| 7261 | agacttcaaa ggtattatgt gagttcacat ttagcaacag cttattaata accctcaagc |
| 7321 | tgtcagaatc tctatagtta ccatttacaa ttttatactg tgaaaaaata cagatcagtg |
| 7381 | aaagcataaa gacaagtcag aattcacttt gaagagggtc tgaggcctgg gagagtctct |
| 7441 | actgtctatt gaagaatgag gcatgtataa aatagttggt tgaatttcac tgatcttccc |
| 7501 | aatgtgaaca aatatactat gtatattgtg tgtatttcta gaaatcaatg gcagctgctg |
| 7561 | atggtgttgt aattagaaat ctatatagat tatagatgtt ttagaaagat ggtgccaatc |
| 7621 | ctaaaagatt tgtgtgggct aaaagtgctt gtacttactt ttttctgcac ttataactga |
| 7681 | tttggtttta aaattgtgtg cgtgtatctg ttctttctct gttgtggcag cttgtactat |
| 7741 | taaaataata gagaatgtta aattattttg atgtgaactg caaatgattt tttttcataa |
| 7801 | agtttaacat ttttatcagc attgttttgc tttgtacttg tataaatatg ttttatttta |
| 7861 | gcacttcaaa atatacttgc ctgtttctca gttgtctaaa. |

In some embodiments, the human mGluR5 comprises or consists of the amino acid sequence of the following sequence, or a functional fragment or variant thereof (UniProtKB P41594-2 and SEQ ID NO: 27):

| | |
|---|---|
| 1 | MVLLLILSVL LLKEDVRGSA QSSERRVVAH MPGDIIIGAL FSVHHQPTVD KVHERKCGAV |
| 61 | REQYGIQRVE AMLHTLERIN SDPTLLPNIT LGCEIRDSCW HSAVALEQSI EFIRDSLISS |
| 121 | EEEEGLVRCV DGSSSSFRSK KPIVGVIGPG SSSVAIQVQN LLQLFNIPQI AYSATSMDLS |
| 181 | DKTLFKYFMR VVPSDAQQAR AMVDIVKRYN WTYVSAVHTE GNYGESGMEA FKDMSAKEGI |
| 241 | CIAHSYKIYS NAGEQSFDKL LKKLTSHLPK ARVVACFCEG MTVRGLLMAM RRLGLAGEFL |
| 301 | LLGSDGWADR YDVTDGYQRE AVGGITIKLQ SPDVKWFDDY YLKLRPETNH RNPWFQEFWQ |
| 361 | HRFQCRLEGF PQENSKYNKT CNSSLTLKTH HVQDSKMGFV INAIYSMAYG LHNMQMSLCP |
| 421 | GYAGLCDAMK PIDGRKLLES LMKTNFTGVS GDTILFDENG DSPGRYEIMN FKEMGKDYFD |
| 481 | YINVGSWDNG ELKMDDDEVW SKKSNIIRSV CSEPCEKGQI KVIRKGEVSC CWTCTPCKEN |
| 541 | EYVFDEYTCK ACQLGSWPTD DLTGCDLIPV QYLRWGDPEP IAAVVFACLG LLATLFVTVV |
| 601 | FIIYRDTPVV KSSSRELCYI ILAGICLGYL CTFCLIAKPK QIYCYLQRIG IGLSPAMSYS |
| 661 | ALVTKTNRIA RILAGSKKKI CTKKPRFMSA CAQLVIAFIL ICIQLGIIVA LFIMEPPDIM |
| 721 | HDYPSIREVY LICNTTNLGV VTPLGYNGLL ILSCTFYAFK TRNVPANFNE AKYIAFTMYT |
| 781 | TCIIWLAFVP IYFGSNYKII TMCFSVSLSA TVALGCMFVP KVYIILAKPE RNVRSAFTTS |
| 841 | TVVRMHVGDG KSSSAASRSS SLVNLWKRRG SSGETLSSNG KSVTWAQNEK SSRGQHLWQR |
| 901 | LSIHINKKEN PNQTAVIKPF PKSTESRGLG AGAGAGGSAG GVGATGGAGC AGAGPGGPES |
| 961 | PDAGPKALYD VAEAEEHFPA PARPRSPSPI STLSHRAGSA SRTDDDVPSL HSEPVARSSS |
| 1021 | SQGSLMEQIS SVVTRFTANI SELNSMMLST AAPSPGVGAP LCSSYLIPKE IQLPTTMTTF |
| 1081 | AEIQPLPAIE VTGGAQPAAG AQAAGDAARE SPAAGPEAAA AKPDLEELVA LTPPSPFRDS |
| 1141 | VDSGSTTPNS PVSESALCIP SSPKYDTLII RDYTQSSSSL. |

In some embodiments, the human mGluR5 comprises or consists of the amino acid sequence of the following sequence, or a functional fragment or variant thereof (UniProtKB P41594-3 and SEQ ID NO: 29):

| | |
|---|---|
| 1 | MVLLLILSVL LLKEDVRGSA QSSERRVVAH MPGDIIIGAL FSVHHQPTVD KVHERKCGAV |
| 61 | REQYGIQRVE AMLHTLERIN SDPTLLPNIT LGCEIRDSCW HSAVALEQSI EFIRDSLISS |
| 121 | EEEEGLVRCV DGSSSSFRSK KPIVGVIGPG SSSVAIQVQN LLQLFNIPQI AYSATSMDLS |
| 181 | DKTLFKYFMR VVPSDAQQAR AMVDIVKRYN WTYVSAVHTE GNYGESGMEA FKDMSAKEGI |
| 241 | CIAHSYKIYS NAGEQSFDKL LKKLTSHLPK ARVVACFCEG MTVRGLLMAM RRLGLAGEFL |
| 301 | LLGSDGWADR YDVTDGYQRE AVGGITIKLQ SPDVKWFDDY YLKLRPETNH RNPWFQEFWQ |
| 361 | HRFQCRLEGF PQENSKYNKT CNSSLTLKTH HVQDSKMGFV INAIYSMAYG LHNMQMSLCP |
| 421 | GYAGLCDAMK PIDGRKLLES LMKTNFTGVS GDTILFDENG DSPGRYEIMN FKEMGKDYFD |
| 481 | YINVGSWDNG ELKMDDDEVW SKKSNIIRSV CSEPCEKGQI KVIRKGEVSC CWTCTPCKEN |
| 541 | EYVFDEYTCK ACQLGSWPTD DLTGCDLIPV QYLRWGDPEP IAAVVFACLG LLATLFVTVV |
| 601 | FIIYRDTPVV KSSSRELCYI ILAGICLGYL CTFCLIAKPK QIYCYLQRIG IGLSPAMSYS |
| 661 | ALVTKTNRIA RILAGSKKKI CTKKPRFMSA CAQLVIAFIL ICIQLGIIVA LFIMEPPDIM |
| 721 | HDYPSIREVY LICNTTNLGV VTPLGYNGLL ILSCTFYAFK TRNVPANFNE AKYIAFTMYT |
| 781 | TCIIWLAFVP IYFGSNYKII TMCFSVSLSA TVALGCMFVP KVYIILAKPE RNVRSAFTTS |
| 841 | TVVRMHVGDG KSSSAASRSS SLVNLWKRRG SSGETLRYKD RRLAQHKSEI ECFTPPSPFR |
| 901 | DSVDSGSTTP NSPVSESALC IPSSPKYDTL IIRDYTQSSS SL. |

In some embodiments, the mGluR comprises mGluR6. In some embodiments, the sequence encoding an mGluR comprises a sequence encoding a human mGluR6.

In some embodiments, the sequence encoding a human mGluR6 comprises or consists of the nucleic acid sequence of the following sequence, or a functional fragment or variant thereof (UniProtKB O15303-1; GenBank Accession No. NM_000843.3 and SEQ ID NO: 30):

| | |
|---|---|
| 1 | cggaggcccg ggcaggccgg ctgagctaac tccccagagc cgaagtggaa ggcgcgcccc |
| 61 | gagcgccttc tccccaggac cccggtgtcc ctccccgcgc cccgagcccg cgctctcctt |
| 121 | cccccgccct cagagcgctc cccgcccctc tgtctccccg cagcccgcta gacgagccga |
| 181 | tggcgcggcc ccggagagcc cgggagccgc tgctcgtggc gctgctgccg ctggcgtggc |
| 241 | tggcgcaggc gggcctggcg cgcgcggcgg gctctgtgcg cctggcgggc ggcctgacgc |
| 301 | tgggcggcct gttcccggtg cacgcgcggg gcgcggcggg ccgggcgtgc gggcagctga |
| 361 | agaaggagca gggcgtgcac cggctggagg ccatgctgta cgcgctggac cgcgtcaacg |
| 421 | ccgaccccga gctgctgccc ggcgtgcgcc tgggcgcgcg gctgctggac acctgctcgc |
| 481 | gggacaccta cgcgctggag caggcgctga gcttcgtgca ggcgctgatc cgcggccgcg |
| 541 | gcgacggcga cgaggtgggc gtgcgctgcc cgggaggcgt ccctccgctg cgccccgcgc |
| 601 | cccccgagcg cgtcgtggcc gtcgtgggcg cctcggccag ctccgtctcc atcatggtcg |
| 661 | ccaacgtgct gcgcctgttt gcgatacccc agatcagcta tgcctccaca gccccggagc |
| 721 | tcagcgactc cacacgctat gacttcttct cccgggtggt gccacccgac tcctaccagg |
| 781 | cgcaggccat ggtggacatc gtgagggcac tgggatggaa ctatgtgtcc acgctggcct |
| 841 | ccgagggcaa ctatggcgaa agtggggttg aggccttcgt tcagatctcc cgagaggctg |
| 901 | ggggggtctg tattgcccag tctatcaaga ttcccaggga accaaagcca ggagagttca |
| 961 | gcaaggtgat caggagactc atggagacgc ccaacgcccg gggcatcatc atctttgcca |
| 1021 | atgaggatga catcaggcgg gtcctggagg cagctcgcca ggccaacctg accggccact |
| 1081 | tcctgtgggt cggctcagac agctggggag ccaagacctc acccatcttg agcctggagg |
| 1141 | acgtggccgt tggggccatc accatcctgc ccaaaagggc ctccatcgac ggatttgacc |
| 1201 | agtacttcat gactcgatcc ctggagaaca accgcaggaa catctggttc gccgagttct |
| 1261 | gggaagagaa ttttaactgc aaactgacca gctcaggtac ccagtcagac gattccaccc |
| 1321 | gcaaatgcac aggcgaggaa cgcatcggcc gggactccac ctacgagcag gagggcaagg |
| 1381 | tgcagtttgt gattgatgcg gtgtacgcca ttgcccacgc cctccacagc atgcaccagg |
| 1441 | cgctctgccc tgggcacaca ggcctgtgcc cggcgatgga acccactgat gggcggatgc |
| 1501 | ttctgcagta cattcgagct gtccgcttca atggcagcgc aggaacccct gtgatgttca |
| 1561 | acgagaacgg agatgcgccc gggcggtacg acatcttcca gtaccaggcg accaatggca |
| 1621 | gtgccagcag tggcgggtac caggcagtgg gccagtgggc agagaccctc agactggatg |
| 1681 | tggaggccct gcagtggtct ggcgaccccc acgaggtgcc ctcgtctctg tgcagcctgc |
| 1741 | cctgcgggcc gggggagcgg aagaagatgg tgaagggcgt cccctgctgt tggcactgcg |
| 1801 | aggcctgtga cgggtaccgc ttccaggtgg acgagttcac atgcgaggcc tgtcctgggg |
| 1861 | acatgaggcc cacgcccaac cacacgggct gccgccccac acctgtggtg cgcctgagct |
| 1921 | ggtcctcccc ctgggcagcc ccgccgctcc tcctggccgt gctgggcatc gtggccacta |
| 1981 | ccacggtggt ggccaccttc gtgcggtaca acaacacgcc catcgtccgg gcctcgggcc |
| 2041 | gagagctcag ctacgtcctc ctcaccggca tcttcctcat ctacgccatc accttcctca |
| 2101 | tggtggctga gcctggggcc gcggtctgtg ccgcccgcag gctcttcctg ggcctgggca |
| 2161 | cgaccctcag ctactctgcc ctgctcacca agaccaaccg tatctaccgc atctttgagc |
| 2221 | agggcaagcg ctcggtcaca ccccctccct tcatcagccc cacctcacag ctggtcatca |
| 2281 | ccttcagcct cacctccctg caggtggtgg ggatgatagc atggctgggg gcccggcccc |
| 2341 | cacacagcgt gattgactat gaggaacagc ggacggtgga ccccgagcag gccagagggg |
| 2401 | tgctcaagtg cgacatgtcg gatctgtctc tcatcggctg cctgggctac agcctcctgc |
| 2461 | tcatggtcac gtgcacagtg tacgccatca aggcccgtgg cgtgcccgag accttcaacg |
| 2521 | aggccaagcc catcggcttc accatgtaca ccacctgcat catctggctg gcattcgtgc |
| 2581 | ccatcttctt tggcactgcc cagtcagctg aaaagatcta catccagaca accacgctaa |
| 2641 | ccgtgtcctt gagcctgagt gcctcggtgt ccctcggcat gctctacgta cccaaaacct |
| 2701 | acgtcatcct cttccatcca gagcagaatg tgcagaagcg aaagcggagc ctcaaggcca |
| 2761 | cctccacggt ggcagcccca cccaagggcg aggatgcaga ggcccacaag tagcagggca |
| 2821 | ggtgggaacg ggactgcttg ctgcctctcc tttcttcctc ttgcctcgag gtggaagctg |
| 2881 | tatagagccc gggtccacgg tgaacagtca gtggcaggga gtttgccaag accatgctcc |
| 2941 | gcgtcggtgg ggctggcctt gagaaggaac tggacccagc tctaccccga ttccagcatg |
| 3001 | tgagcttcat gcttcctcac cacagaccag actcgcttcc catggtggga aacagccacc |
| 3061 | gagaaggttc tagctctaga aagggactaa acttattctc tcatccgaag tccaaagagg |
| 3121 | atgatgaagc cctgggcttt gcctggtttg cgggagattt cctcccctca gtcaaccccc |
| 3181 | ataacctggg gattgggcag tgtggaagaa cgtgtagacc ccagaatgaa acatggggtt |
| 3241 | ggagtggagg aggagctgtc tcagcaagag gagacctggg gctgtgcatc tggatggagg |
| 3301 | cactcaggcc tgggtaggat tcctctggca cggagggaga gaccctgggt gagacccctg |
| 3361 | tgagcatggg aagggcctgc agtgggcgcg ggagtgagct gaggaactgg ggtgcgcccc |
| 3421 | catgagattc ccaatgccat gggctttccc ccatcccccc gggattgggc aaggtcagac |
| 3481 | ttagagtaca gctgttttcc tcccctctgt gtactccctt aaatcacccc aaccttggcc |
| 3541 | aggcatggtg gctcacacct gtaatcccag cactttggga ggccgaggca ggtggatcac |
| 3601 | ctgaggtccg gagttcgaga ccagcctggc caatgtggtg aaaccctgtc tctactaaaa |
| 3661 | atacaaaaat tagccaggtg tgatggtggg tgcctgtaat cccagttact tgggaggctg |
| 3721 | aggcaggaga atcgcttgaa cctgggaggt ggaggttgca gtgagctgtg attgtgccac |
| 3781 | tgtactccag cctgggtgac agagcgagac tctgtctcaa aaaaacaaaa caaaaaaaca |
| 3841 | ccaaaaaaac ccccaaacct gaagaaattc agatacacgt gtgtaatgtt agtgatgtga |
| 3901 | gaacaaggag caggggtgca tttgtgttgt gttcgggttg gggatgggtt taggagctcc |
| 3961 | aggttgggag cagtgacaga gagtcatggc cgtggtgagg gtgaatccca agtggatggc |
| 4021 | tcaggacggg tatggaaacc cttcattcct cataggtact gggaagtcca tttgcaagct |
| 4081 | gagcgccagg cctggggagg aagaggcttg ggctgcagat gcacgcacat ttgtttttca |
| 4141 | ctgatagttt ttacaaaaag cttggtttaa gttatggagt tttatgtccc tgggagtaga |
| 4201 | atttacattt gttaaattga ccactgttta agatcagtat acattctcta gtctgtgatg |
| 4261 | tctggagcta gttttgaggg tgaaccacac tttatccaac atacaaactt tcccatgcag |
| 4321 | cttctctggt gcgcagttgg ttttgaccgt gggactaggt gcttctgcag gttttaagta |
| 4381 | attaacttaa aagcttctcc tctgagaaac atttctgttg cgctactgac tctccttctc |
| 4441 | cacatttgtt gtgttcctag ggcttctcta tagtgcacat taggacgttt catttgttgc |
| 4501 | tgaatgcttt ccagaattat ttattccata gggtttctct cctgtgcagc tctctcatgg |
| 4561 | gtaatggggc gtgttttctt gccaaaggcg gttccaccct cgtgattgta tagggctctt |
| 4621 | ctcctgtatg aactctgaga tcagtgagct ctgatctcca agggaaagtt ttcctgcatt |
| 4681 | tgctgttttc tcatgtctct cccagtgtga attctttggt gtgcagtgcg ctctggcttc |
| 4741 | tagctgaaaa cttttccaca gttttacatt catgtggttt tctccactgt gaactctgtg |
| 4801 | attcagaatc agaagcagtt cttagtagag gcatttctac actgattgca ctgaggattt |
| 4861 | ctccccagtg tgaagtttct ggcatagagt cctggcttcc cgcagacgac tttcacactc |
| 4921 | tgccatgttc atgcctgtgg gcctctctgg caggaactct gatgcaccgc gaggcccatg |
| 4981 | tactcctgtg gctttctcac attcggtcta cttgcagggt atctccacag catgcaccat |
| 5041 | tctgggtaca gggggacatc ctctgttact gaagatgttg tcatatttag taccttcaca |
| 5101 | aggtttctct ccttccagaa ttttctgatg tacacaaata actgacttcc acaagagggc |
| 5161 | ttttccacac tcggtgtgtg catacagttt ctgcctgtga tcatttcttt atgttattat |
| 5221 | tttatttttt cgagataggg tcttgctcaa tttcttaggc tggagtgcag tggcacgatc |
| 5281 | atagctcact gaagtttcga cctgggctca agcaatcctc ccgcttcagc ctcctgagta |
| 5341 | gctggtgcgc acgaccatac ccagctaatg ttttattttt tgtagagacg aggtctcact |
| 5401 | atgttgccca ggctggtctc gaacttctga gctcgagcga tcctcctgcc tccacctccc |
| 5461 | aaagtgttcg gattacaaac gtgagccatc gcacctagcc tctttgatca tttctgtggt |
| 5521 | gttcagtgga ggttgacagc tccctaaaga ttttcctgtt tttttgcatg catgggtttg |
| 5581 | aattctttga ggtccaattt atttggaccc ctgaataaag ttttgtgggt tttcttctat |
| 5641 | gtgtggaatt tataaggcat tcttccagtg tggtttctct tatgtcgagt gagagctgac |
| 5701 | ctgcaccgaa ggttttgtcc catttgttgc ccttgaatta tttgtatgaa ttatatgttc |
| 5761 | cagtgaaaat ggagttctgg gttggaggct tattccatgt ttacacaatt aaaattgcag |
| 5821 | tgttcctctc tgggatgaga gctctaaagc agagtaagat tacgttctga tgtaagcttt |
| 5881 | aaccacctat ttataaggtc tcacctgtgg tccactgtgt tgagacttct acagaagagc |
| 5941 | ttctgtatag taaccatttt cttaggctgt ctcacttgtg tgaatcttct gacacattta |
| 6001 | ttatagcttt gtcccatttc ttatcctttt tgctctttag aaatttccct ttaatttatt |
| 6061 | acattcattg cttactgtaa agagtccagg taactgactt tattcagtta cttcctgttc |
| 6121 | aataaattta acttttccca aaa. |

In some embodiments, the human mGluR6 comprises or consists of the amino acid sequence of the following sequence, or a functional fragment or variant thereof (UniProtKB O15303-1; GenBank Accession No. NP_000834.2 and SEQ ID NO: 31):

| | |
|---|---|
| 1 | MARPRRAREP LLVALLPLAW LAQAGLARAA GSVRLAGGLT LGGLFPVHAR GAAGRACGQL |
| 61 | KKEQGVHRLE AMLYALDRVN ADPELLPGVR LGARLLDTCS RDTYALEQAL SFVQALIRGR |
| 121 | GDGDEVGVRC PGGVPPLRPA PPERVVAVVG ASASSVSIMV ANVLRLFAIP QISYASTAPE |
| 181 | LSDSTRYDFF SRVVPPDSYQ AQAMVDIVRA LGWNYVSTLA SEGNYGESGV EAFVQISREA |
| 241 | GGVCIAQSIK IPREPKPGEF SKVIRRLMET PNARGIIIFA NEDDIRRVLE AARQANLTGH |
| 301 | FLWVGSDSWG AKTSPILSLE DVAVGAITIL PKRASIDGFD QYFMTRSLEN NRRNIWFAEF |
| 361 | WEENFNCKLT SSGTQSDDST RKCTGEERIG RDSTYEQEGK VQFVIDAVYA IAHALHSMHQ |
| 421 | ALCPGHTGLC PAMEPTDGRM LLQYIRAVRF NGSAGTPVMF NENGDAPGRY DIFQYQATNG |
| 481 | SASSGGYQAV GQWAETLRLD VEALQWSGDP HEVPSSLCSL PCGPGERKKM VKGVPCCWHC |
| 541 | EACDGYRFQV DEFTCEACPG DMRPTPNHTG CRPTPVVRLS WSSPWAAPPL LLAVLGIVAT |
| 601 | TTVVATFVRY NNTPIVRASG RELSYVLLTG IFLIYAITFL MVAEPGAAVC AARRLFLGLG |
| 661 | TTLSYSALLT KTNRIYRIFE QGKRSVTPPP FISPTSQLVI TFSLTSLQVV GMIAWLGARP |
| 721 | PHSVIDYEEQ RTVDPEQARG VLKCDMSDLS LIGCLGYSLL LMVTCTVYAI KARGVPETFN |
| 781 | EAKPIGFTMY TTCIIWLAFV PIFFGTAQSA EKIYIQTTTL TVSLSLSASV SLGMLYVPKT |
| 841 | YVILFHPEQN VQKRKRSLKA TSTVAAPPKG EDAEAHK. |

In some embodiments, the mGluR comprises mGluR7. In some embodiments, the sequence encoding an mGluR comprises a sequence encoding a human mGluR7.

In some embodiments, the sequence encoding a human mGluR7 comprises or consists of the nucleic acid sequence of the following sequence, or a functional fragment or variant thereof (UniProtKB Q14831-1; GenBank Accession No. NM_000844.4 and SEQ ID NO: 32):

| | |
|---|---|
| 1 | agtgctgggc tgttggagag agcgagcagc aagccggtga gcgcgagcgc ggcgcgccgg |
| 61 | ccggctaacc cgagagcgcg aggcgcccca ggctggcagg cgccgcggga cccctcaccc |
| 121 | tctctggtcg cccctccccg gattccccca ccctccgtgc ctgcaggagc ccctgggctt |
| 181 | tcccggagga gctcgccctg aagggcccgg acctcggcga gcccaccacc gttccctcca |
| 241 | gcgccgccgc cgccaccgca gcagccggag cagcatggtc cagctgagga agctgctccg |
| 301 | cgtcctgact ttgatgaagt tcccctgctg cgtgctggag gtgctcctgt gcgcgctggc |
| 361 | ggcggcggcg cgcggccagg agatgtacgc cccgcactca atccggatcg agggggacgt |
| 421 | caccctcggg gggctgttcc ccgtgcacgc caagggtccc agcggagtgc cctgcggcga |
| 481 | catcaagagg gaaaacggga tccacaggct ggaagcgatg ctctacgccc tggaccagat |
| 541 | caacagtgat cccaacctac tgcccaacgt gacgctgggc gcgcggatcc tggacacttg |
| 601 | ttccagggac acttacgcgc tcgaacagtc gcttactttc gtccaggcgc tcatccagaa |
| 661 | ggacacctcc gacgtgcgct gcaccaacgg cgaaccgccg gttttcgtca agccggagaa |
| 721 | agtagttgga gtgattgggg cttcggggag ttcggtctcc atcatggtag ccaacatcct |
| 781 | gaggctcttc cagatccccc agattagtta tgcatcaacg gcacccgagc taagtgatga |
| 841 | ccggcgctat gacttcttct ctcgcgtggt gccacccgat tccttccaag cccaggccat |
| 901 | ggtagacatt gtaaaggccc taggctggaa ttatgtgtct accctcgcat cggaaggaag |
| 961 | ttatggagag aaaggtgtgg agtccttcac gcagatttcc aaagaggcag gtggactctg |
| 1021 | cattgcccag tccgtgagaa tcccccagga acgcaaagac aggaccattg actttgatag |
| 1081 | aattatcaaa cagctcctgg acacccccaa ctccagggcc gtcgtgattt ttgccaacga |
| 1141 | tgaggatata aagcagatcc ttgcagcagc caaaagagct gaccaagttg gccattttct |
| 1201 | ttgggtggga tcagacagct ggggatccaa aataaaccca ctgcaccagc atgaagatat |
| 1261 | cgcagaaggg gccatcacca ttcagcccaa gcgagccacg gtggaagggt ttgatgccta |
| 1321 | ctttacgtcc cgtacacttg aaaacaacag aagaaatgta tggtttgccg aatactggga |
| 1381 | ggaaaacttc aactgcaagt tgacgattag tgggtcaaaa aaagaagaca cagatcgcaa |
| 1441 | atgcacagga caggagagaa ttggaaaaga ttccaactat gagcaggagg gtaaagtcca |
| 1501 | gttcgtgatt gacgcagtct atgctatggc tcacgccctt caccacatga acaaggatct |
| 1561 | ctgtgctgac taccggggtg tctgcccaga gatggagcaa gctggaggca agaagttgct |
| 1621 | gaagtatata cgcaatgtta atttcaatgg tagtgctggc actccagtga tgtttaacaa |
| 1681 | gaacggggat gcacctgggc gttatgacat ctttcagtac cagaccacaa acaccagcaa |
| 1741 | cccgggttac cgtctgatcg ggcagtggac agacgaactt cagctcaata tagaagacat |
| 1801 | gcagtggggt aaaggagtcc gagagatacc cgcctcagtg tgcacactac catgtaagcc |
| 1861 | aggacagaga aagaagacac agaaaggaac tccttgctgt tggacctgtg agccttgcga |
| 1921 | tggttaccag taccagtttg atgagatgac atgccagcat tgcccctatg accagaggcc |
| 1981 | caatgaaaat cgaaccggat gccaggatat tcccatcatc aaactggagt ggcactcccc |
| 2041 | ctgggctgtg attcctgtct tcctggcaat gttggggatc attgccacca tctttgtcat |
| 2101 | ggccactttc atccgctaca atgacacgcc cattgtccgg gcatctgggc gggaactcag |
| 2161 | ctatgttctt ttgacgggca tctttctttg ctacatcatc actttcctga tgattgccaa |
| 2221 | accagatgtg gcagtgtgtt ctttccggcg agttttcttg ggcttgggta tgtgcatcag |
| 2281 | ttatgcagcc ctcttgacga aaacaaatcg gatttatcgc atatttgagc agggcaagaa |
| 2341 | atcagtaaca gctcccagac tcataagccc aacatcacaa ctggcaatca cttccagttt |
| 2401 | aatatcagtt cagcttctag gggtgttcat ttggtttggt gttgatccac ccaacatcat |
| 2461 | catagactat gatgaacaca agacaatgaa ccctgagcaa gccagagggg ttctcaagtg |
| 2521 | tgacattaca gatctccaaa tcatttgctc cttgggatat agcattcttc tcatggtcac |
| 2581 | atgtactgtg tatgccatca agactcgggg tgtacccgag aattttaacg aagccaagcc |
| 2641 | cattggattc actatgtaca cgacatgtat agtatggctt gccttcattc caattttttt |
| 2701 | tggcaccgct caatcagcgg aaaagctcta catacaaact accacgctta caatctccat |
| 2761 | gaacctaagt gcatcagtgg cgctggggat gctatacatg ccgaaagtgt acatcatcat |
| 2821 | tttccaccct gaactcaatg tccagaaacg gaagcgaagc ttcaaggcgg tagtcacagc |
| 2881 | agccaccatg tcatcgaggc tgtcacacaa acccagtgac agacccaacg gtgaggcaaa |
| 2941 | gaccgagctc tgtgaaaacg tagacccaaa cagccctgct gcaaaaaaga agtatgtcag |
| 3001 | ttataataac ctggttatct aacctgttcc attccatgga accatggagg aggaagaccc |
| 3061 | tcagttattt tgtcacccaa cctggcatag gactctttgg tcctacccgc ttcccatcac |
| 3121 | cggaggagct tccccggccg ggagaccagt gttagaggat ccaagcgacc taaacagctg |
| 3181 | ctttatgaaa tatccttact ttatctgggc ttaataagtc actgacatca gcactgccaa |
| 3241 | ctcggctgca attgtggacc ttccctacca aagggagtgt tgaaactcaa gtcccgccct |
| 3301 | ggctctttag aatggaccac tgagagccac aggaccgttt tggggctgac ctgtcttatt |
| 3361 | acgtatgtac ttctaggttg caaggttttg aaattttctg tacagtttgt gaggaccttt |
| 3421 | gcactttgcc atctgatgtc gtacctcggt tcactgtttg ttttcgaatg ccttgttttc |
| 3481 | atagagccct attctctcag acggtggaat atttggaaaa attttaaaac aattaaaatt |
| 3541 | ttaaagcaat cttggcagac taaaacaagt acatctgtac atgactgtat aattacgatt |
| 3601 | atagtaccac tgcacatcat gttttttttt ttaagacaaa aaagatgttt aaagaccaaa |
| 3661 | aactgtgctg agaaagtatg ccccacctat ctttggtata tgataggtta cataaaagga |
| 3721 | aggtattggc tgaactgaat agaggtcttg atctttggaa tgcatgccag taatgtattt |
| 3781 | tacagtacat gtttattatg ttcaatattt gtatttgtgt tctcttttgt tatttttaat |
| 3841 | tagggtatat gaatattttg caataatttt aataattatt aagctgtttg aaggaaagaa |
| 3901 | tatggatttt tcatgtcttg aggttttgtt catgccccct ttgactgatc agtgtgataa |
| 3961 | ggactttagg aaaaaaagca tgtatgtttt ttactgtttg taataagtac tttcgttaat |
| 4021 | cttgctgctt atgtgccaat ttagtggaaa aaaacaaccc ttgctgaaaa attccctctt |
| 4081 | tccattctct ttcaattctg tgatattgtc caagaatgta tcaataaaat actttggtta |
| 4141 | actttttta. |

In some embodiments, the human mGluR7 comprises or consists of the amino acid sequence of the following sequence, or a functional fragment or variant thereof (UniProtKB Q14831-1 and SEO ID NO: 33):

| | |
|---|---|
| 1 | MVQLRKLLRV LTLMKFPCCV LEVLLCALAA AARGQEMYAP HSIRIEGDVT LGGLFPVHAK |
| 61 | GPSGVPCGDI KRENGIHRLE AMLYALDQIN SDPNLLPNVT LGARILDTCS RDTYALEQSL |
| 121 | TFVQALIQKD TSDVRCTNGE PPVFVKPEKV VGVIGASGSS VSIMVANILR LFQIPQISYA |
| 181 | STAPELSDDR RYDFFSRVVP PDSFQAQAMV DIVKALGWNY VSTLASEGSY GEKGVESFTQ |
| 241 | ISKEAGGLCI AQSVRIPQER KDRTIDFDRI IKQLLDTPNS RAVVIFANDE DIKQILAAAK |
| 301 | RADQVGHFLW VGSDSWGSKI NPLHQHEDIA EGAITIQPKR ATVEGFDAYF TSRTLENNRR |
| 361 | NVWFAEYWEE NFNCKLTISG SKKEDTDRKC TGQERIGKDS NYEQEGKVQF VIDAVYAMAH |
| 421 | ALHHMNKDLC ADYRGVCPEM EQAGGKKLLK YIRNVNFNGS AGTPVMFNKN GDAPGRYDIF |
| 481 | QYQTTNTSNP GYRLIGQWTD ELQLNIEDMQ WGKGVREIPA SVCTLPCKPG QRKKTQKGTP |
| 541 | CCWTCEPCDG YQYQFDEMTC QHCPYDQRPN ENRTGCQDIP IIKLEWHSPW AVIPVFLAML |
| 601 | GIIATIFVMA TFIRYNDTPI VRASGRELSY VLLTGIFLCY IITFLMIAKP DVAVCSFRRV |
| 661 | FLGLGMCISY AALLTKTNRI YRIFEQGKKS VTAPRLISPT SQLAITSSLI SVQLLGVFIW |
| 721 | FGVDPPNIII DYDEHKTMNP EQARGVLKCD ITDLQIICSL GYSILLMVTC TVYAIKTRGV |
| 781 | PENFNEAKPI GFTMYTTCIV WLAFIPIFFG TAQSAEKLYI QTTTLTISMN LSASVALGML |
| 841 | YMPKVYIIIF HPELNVQKRK RSFKAVVTAA TMSSRLSHKP SDRPNGEAKT ELCENVDPNS |
| 901 | PAAKKKYVSY NNLVI . |

In some embodiments, the sequence encoding a human mGluR7 comprises or consists of the nucleic acid sequence of the following sequence, or a functional fragment or variant thereof (UniProtKB Q14831-2; GenBank Accession No. NM_181874.3 and SEQ ID NO: 34):

| | |
|---|---|
| 1 | agtgctgggc tgttggagag agcgagcagc aagccggtga gcgcgagcgc ggcgcgccgg |
| 61 | ccggctaacc cgagagcgcg aggcgcccca ggctggcagg cgccgcggga cccctcaccc |
| 121 | tctctggtcg cccctccccg gattccccca ccctccgtgc ctgcaggagc ccctgggctt |
| 181 | tcccggagga gctcgccctg aagggcccgg acctcggcga gcccaccacc gttccctcca |
| 241 | gcgccgccgc cgccaccgca gcagccggag cagcatggtc cagctgagga agctgctccg |
| 301 | cgtcctgact ttgatgaagt tcccctgctg cgtgctggag gtgctcctgt gcgcgctggc |
| 361 | ggcggcggcg cgcggccagg agatgtacgc cccgcactca atccggatcg agggggacgt |
| 421 | caccctcggg gggctgttcc ccgtgcacgc caagggtccc agcggagtgc cctgcggcga |
| 481 | catcaagagg gaaaacggga tccacaggct ggaagcgatg ctctacgccc tggaccagat |
| 541 | caacagtgat cccaacctac tgcccaacgt gacgctgggc gcgcggatcc tggacacttg |
| 601 | ttccagggac acttacgcgc tcgaacagtc gcttactttc gtccaggcgc tcatccagaa |
| 661 | ggacacctcc gacgtgcgct gcaccaacgg cgaaccgccg gttttcgtca agccggagaa |
| 721 | agtagttgga gtgattgggg cttcggggag ttcggtctcc atcatggtag ccaacatcct |
| 781 | gaggctcttc cagatccccc agattagtta tgcatcaacg gcacccgagc taagtgatga |
| 841 | ccggcgctat gacttcttct ctcgcgtggt gccacccgat tccttccaag cccaggccat |
| 901 | ggtagacatt gtaaaggccc taggctggaa ttatgtgtct accctcgcat cggaaggaag |
| 961 | ttatggagag aaaggtgtgg agtccttcac gcagatttcc aaagaggcag gtggactctg |
| 1021 | cattgcccag tccgtgagaa tcccccagga acgcaaagac aggaccattg actttgatag |
| 1081 | aattatcaaa cagctcctgg acacccccaa ctccagggcc gtcgtgattt ttgccaacga |
| 1141 | tgaggatata aagcagatcc ttgcagcagc caaaagagct gaccaagttg gccattttct |
| 1201 | ttgggtggga tcagacagct ggggatccaa aataaaccca ctgcaccagc atgaagatat |
| 1261 | cgcagaaggg gccatcacca ttcagcccaa gcgagccacg gtggaagggt ttgatgccta |
| 1321 | ctttacgtcc cgtacacttg aaaacaacag aagaaatgta tggtttgccg aatactggga |
| 1381 | ggaaaacttc aactgcaagt tgacgattag tgggtcaaaa aaagaagaca cagatcgcaa |
| 1441 | atgcacagga caggagagaa ttggaaaaga ttccaactat gagcaggagg gtaaagtcca |
| 1501 | gttcgtgatt gacgcagtct atgctatggc tcacgccctt caccacatga acaaggatct |
| 1561 | ctgtgctgac taccggggtg tctgcccaga gatggagcaa gctggaggca agaagttgct |
| 1621 | gaagtatata cgcaatgtta atttcaatgg tagtgctggc actccagtga tgtttaacaa |
| 1681 | gaacggggat gcacctgggc gttatgacat ctttcagtac cagaccacaa acaccagcaa |
| 1741 | cccgggttac cgtctgatcg ggcagtggac agacgaactt cagctcaata tagaagacat |
| 1801 | gcagtggggt aaaggagtcc gagagatacc cgcctcagtg tgcacactac catgtaagcc |
| 1861 | aggacagaga aagaagacac agaaaggaac tccttgctgt tggacctgtg agccttgcga |
| 1921 | tggttaccag taccagtttg atgagatgac atgccagcat tgcccctatg accagaggcc |
| 1981 | caatgaaaat cgaaccggat gccaggatat tcccatcatc aaactggagt ggcactcccc |
| 2041 | ctgggctgtg attcctgtct tcctggcaat gttggggatc attgccacca tctttgtcat |
| 2101 | ggccactttc atccgctaca atgacacgcc cattgtccgg gcatctgggc gggaactcag |
| 2161 | ctatgttctt ttgacgggca tctttctttg ctacatcatc actttcctga tgattgccaa |
| 2221 | accagatgtg gcagtgtgtt ctttccggcg agttttcttg ggcttgggta tgtgcatcag |
| 2281 | ttatgcagcc ctcttgacga aaacaaatcg gatttatcgc atatttgagc agggcaagaa |
| 2341 | atcagtaaca gctcccagac tcataagccc aacatcacaa ctggcaatca cttccagttt |
| 2401 | aatatcagtt cagcttctag gggtgttcat ttggtttggt gttgatccac ccaacatcat |
| 2461 | catagactat gatgaacaca agacaatgaa ccctgagcaa gccagagggg ttctcaagtg |
| 2521 | tgacattaca gatctccaaa tcatttgctc cttgggatat agcattcttc tcatggtcac |
| 2581 | atgtactgtg tatgccatca agactcgggg tgtacccgag aattttaacg aagccaagcc |
| 2641 | cattggattc actatgtaca cgacatgtat agtatggctt gccttcattc caattttttt |
| 2701 | tggcaccgct caatcagcgg aaaagctcta catacaaact accacgctta caatctccat |
| 2761 | gaacctaagt gcatcagtgg cgctggggat gctatacatg ccgaaagtgt acatcatcat |
| 2821 | tttccaccct gaactcaatg tccagaaacg gaagcgaagc ttcaaggcgg tagtcacagc |
| 2881 | agccaccatg tcatcgaggc tgtcacacaa acccagtgac agacccaacg gtgaggcaaa |
| 2941 | gaccgagctc tgtgaaaacg tagacccaaa caactgtata ccaccagtaa gaaagagtgt |
| 3001 | acaaaagtct gttacttggt acactatccc accaacagta tagcttttga ctgctttccc |
| 3061 | aaaggccctg ctgcaaaaaa gaagtatgtc agttataata acctggttat ctaacctgtt |
| 3121 | ccattccatg gaaccatgga ggaggaagac cctcagttat tttgtcaccc aacctggcat |
| 3181 | aggactcttt ggtcctaccc gcttcccatc accggaggag cttccccggc cgggagacca |
| 3241 | gtgttagagg atccaagcga cctaaacagc tgctttatga aatatcctta ctttatctgg |
| 3301 | gcttaataag tcactgacat cagcactgcc aactcggctg caattgtgga ccttccctac |
| 3361 | caaagggagt gttgaaactc aagtcccgcc ctggctcttt agaatggacc actgagagcc |
| 3421 | acaggaccgt tttggggctg acctgtctta ttacgtatgt acttctaggt tgcaaggttt |
| 3481 | tgaaattttc tgtacagttt gtgaggacct ttgcactttg ccatctgatg tcgtacctcg |
| 3541 | gttcactgtt tgttttcgaa tgccttgttt tcatagagcc ctattctctc agacggtgga |
| 3601 | atatttggaa aaattttaaa acaattaaaa ttttaaagca atcttggcag actaaaacaa |
| 3661 | gtacatctgt acatgactgt ataattacga ttatagtacc actgcacatc atgttttttt |
| 3721 | ttttaagaca aaaaagatgt ttaaagacca aaaactgtgc tgagaaagta tgccccacct |
| 3781 | atctttggta tatgataggt tacataaaag gaaggtattg gctgaactga atagaggtct |
| 3841 | tgatctttgg aatgcatgcc agtaatgtat tttacagtac atgtttatta tgttcaatat |
| 3901 | ttgtatttgt gttctctttt gttattttta attagggtat atgaatattt tgcaataatt |
| 3961 | ttaataatta ttaagctgtt tgaaggaaag aatatggatt tttcatgtct tgaggttttg |
| 4021 | ttcatgcccc ctttgactga tcagtgtgat aaggacttta ggaaaaaaag catgtatgtt |
| 4081 | ttttactgtt tgtaataagt actttcgtta atcttgctgc ttatgtgcca atttagtgga |
| 4141 | aaaaaacaac ccttgctgaa aaattccctc tttccattct ctttcaattc tgtgatattg |
| 4201 | tccaagaatg tatcaataaa atactttggt taactttttt a |

In some embodiments, the human mGluR7 comprises or consists of the amino acid sequence of the following sequence, or a functional fragment or variant thereof (UniProtKB Q14831-2 and SEQ ID NO: 35):

| | |
|---|---|
| 1 | MVQLRKLLRV LTLMKFPCCV LEVLLCALAA AARGQEMYAP HSIRIEGDVT LGGLFPVHAK |
| 61 | GPSGVPCGDI KRENGIHRLE AMLYALDQIN SDPNLLPNVT LGARILDTCS RDTYALEQSL |
| 121 | TFVQALIQKD TSDVRCTNGE PPVFVKPEKV VGVIGASGSS VSIMVANILR LFQIPQISYA |
| 181 | STAPELSDDR RYDFFSRVVP PDSFQAQAMV DIVKALGWNY VSTLASEGSY GEKGVESFTQ |
| 241 | ISKEAGGLCI AQSVRIPQER KDRTIDFDRI IKQLLDTPNS RAVVIFANDE DIKQILAAAK |
| 301 | RADQVGHFLW VGSDSWGSKI NPLHQHEDIA EGAITIQPKR ATVEGFDAYF TSRTLENNRR |
| 361 | NVWFAEYWEE NFNCKLTISG SKKEDTDRKC TGQERIGKDS NYEQEGKVQF VIDAVYAMAH |
| 421 | ALHHMNKDLC ADYRGVCPEM EQAGGKKLLK YIRNVNFNGS AGTPVMFNKN GDAPGRYDIF |
| 481 | QYQTTNTSNP GYRLIGQWTD ELQLNIEDMQ WGKGVREIPA SVCTLPCKPG QRKKTQKGTP |
| 541 | CCWTCEPCDG YQYQFDEMTC QHCPYDQRPN ENRTGCQDIP IIKLEWHSPW AVIPVFLAML |
| 601 | GIIATIFVMA TFIRYNDTPI VRASGRELSY VLLTGIFLCY IITFLMIAKP DVAVCSFRRV |
| 661 | FLGLGMCISY AALLTKTNRI YRIFEQGKKS VTAPRLISPT SQLAITSSLI SVQLLGVFIW |
| 721 | FGVDPPNIII DYDEHKTMNP EQARGVLKCD ITDLQIICSL GYSILLMVTC TVYAIKTRGV |
| 781 | PENFNEAKPI GFTMYTTCIV WLAFIPIFFG TAQSAEKLYI QTTTLTISMN LSASVALGML |
| 841 | YMPKVYIIIF HPELNVQKRK RSFKAVVTAA TMSSRLSHKP SDRPNGEAKT ELCENVDPNN |
| 901 | CIPPVRKSVQ KSVTWYTIPP TV. |

In some embodiments, the human mGluR7 comprises or consists of the amino acid sequence of the following sequence, or a functional fragment or variant thereof (UniProtKB Q14831-3; SEQ ID NO: 37):

| | |
|---|---|
| 1 | MVQLRKLLRV LTLMKFPCCV LEVLLCALAA AARGQEMYAP HSIRIEGDVT LGGLFPVHAK |
| 61 | GPSGVPCGDI KRENGIHRLE AMLYALDQIN SDPNLLPNVT LGARILDTCS RDTYALEQSL |
| 121 | TFVQALIQKD TSDVRCTNGE PPVFVKPEKV VGVIGASGSS VSIMVANILR LFQIPQISYA |
| 181 | STAPELSDDR RYDFFSRVVP PDSFQAQAMV DIVKALGWNY VSTLASEGSY GEKGVESFTQ |
| 241 | ISKEAGGLCI AQSVRIPQER KDRTIDFDRI IKQLLDTPNS RAVVIFANDE DIKQILAAAK |
| 301 | RADQVGHFLW VGSDSWGSKI NPLHQHEDIA EGAITIQPKR ATVEGFDAYF TSRTLENNRR |
| 361 | NVWFAEYWEE NFNCKLTISG SKKEDTDRKC TGQERIGKDS NYEQEGKVQF VIDAVYAMAH |
| 421 | ALHHMNKDLC ADYRGVCPEM EQAGGKKLLK YIRNVNFNGS AGTPVMFNKN GDAPGRYDIF |
| 481 | QYQTTNTSNP GYRLIGQWTD ELQLNIEDMQ WGKGVREIPA SVCTLPCKPG QRKKTQKGTP |
| 541 | CCWTCEPCDG YQYQFDEMTC QHCPYDQRPN ENRTGCQDIP IIKLEWHSPW AVIPVFLAML |
| 601 | GIIATIFVMA TFIRYNDTPI VRASGRELSY VLLTGIFLCY IITFLMIAKP DVAVCSFRRV |
| 661 | FLGLGMCISY AALLTKTNRI YRIFEQGKKS VTAPRLISPT SQLAITSSLI SVQLLGVFIW |
| 721 | FGVDPPNIII DYDEHKTMNP EQARGVLKCD ITDLQIICSL GYSILLMVTC TVYAIKTRGV |
| 781 | PENFNEAKPI GFTMYTTCIV WLAFIPIFFG TAQSAEKLYI QTTTLTISMN LSASVALGML |
| 841 | YMPKVYIIIF HPELNVQKRK RSFKAVVTAA TMSSRLSHKP SDRPNGEAKT ELCENVDPNN |
| 901 | FFFWLYSGTW. |

In some embodiments, the human mGluR7 comprises or consists of the amino acid sequence of the following sequence, or a functional fragment or variant thereof (UniProtKB Q14831-4 and SEQ ID NO: 39):

| | |
|---|---|
| 1 | MVQLRKLLRV LTLMKFPCCV LEVLLCALAA AARGQEMYAP HSIRIEGDVT LGGLFPVHAK |
| 61 | GPSGVPCGDI KRENGIHRLE AMLYALDQIN SDPNLLPNVT LGARILDTCS RDTYALEQSL |
| 121 | TFVQALIQKD TSDVRCTNGE PPVFVKPEKV VGVIGASGSS VSIMVANILR LFQIPQISYA |
| 181 | STAPELSDDR RYDFFSRVVP PDSFQAQAMV DIVKALGWNY VSTLASEGSY GEKGVESFTQ |
| 241 | ISKEAGGLCI AQSVRIPQER KDRTIDFDRI IKQLLDTPNS RAVVIFANDE DIKQILAAAK |
| 301 | RADQVGHFLW VGSDSWGSKI NPLHQHEDIA EGAITIQPKR ATVEGFDAYF TSRTLENNRR |
| 361 | NVWFAEYWEE NFNCKLTISG SKKEDTDRKC TGQERIGKDS NYEQEGKVQF VIDAVYAMAH |
| 421 | ALHHMNKDLC ADYRGVCPEM EQAGGKKLLK YIRNVNFNGS AGTPVMFNKN GDAPGRYDIF |
| 481 | QYQTTNTSNP GYRLIGQWTD ELQLNIEDMQ WGKGVREIPA SVCTLPCKPG QRKKTQKGTP |
| 541 | CCWTCEPCDG YQYQFDEMTC QHCPYDQRPN ENRTGCQDIP IIKLEWHSPW AVIPVFLAML |
| 601 | GIIATIFVMA TFIRYNDTPI VRASGRELSY VLLTGIFLCY IITFLMIAKP DVAVCSFRRV |
| 661 | FLGLGMCISY AALLTKTNRI YRIFEQGKKS VTAPRLISPT SQLAITSSLI SVQLLGVFIW |
| 721 | FGVDPPNIII DYDEHKTMNP EQARGVLKCD ITDLQIICSL GYSILLMVTC TVYAIKTRGV |
| 781 | PENFNEAKPI GFTMYTTCIV WLAFIPIFFG TAQSAEKLYI QTTTLTISMN LSASVALGML |
| 841 | YMPKVYIIIF HPELNVQKRK RSFKAVVTAA TMSSRLSHKP SDRPNGEAKT ELCENVDPNI |
| 901 | TSEDLSLHKE D. |

In some embodiments, the human mGluR7 comprises or consists of the amino acid sequence of the following sequence, or a functional fragment or variant thereof (UniProtKB Q14831-5; and SEQ ID NO: 41):

| | |
|---|---|
| 1 | MVQLRKLLRV LTLMKFPCCV LEVLLCALAA AARGQEMYAP HSIRIEGDVT LGGLFPVHAK |
| 61 | GPSGVPCGDI KRENGIHRLE AMLYALDQIN SDPNLLPNVT LGARILDTCS RDTYALEQSL |
| 121 | TFVQALIQKD TSDVRCTNGE PPVFVKPEKV VGVIGASGSS VSIMVANILR LFQIPQISYA |
| 181 | STAPELSDDR RYDFFSRVVP PDSFQAQAMV DIVKALGWNY VSTLASEGSY GEKGVESFTQ |
| 241 | ISKEAGGLCI AQSVRIPQER KDRTIDFDRI IKQLLDTPNS RAVVIFANDE DIKQILAAAK |
| 301 | RADQVGHFLW VGSDSWGSKI NPLHQHEDIA EGAITIQPKR ATVEGFDAYF TSRTLENNRR |
| 361 | NVWFAEYWEE NFNCKLTISG SKKEDTDRKC TGQERIGKDS NYEQEGKVQF VIDAVYAMAH |
| 421 | ALHHMNKDLC ADYRGVCPEM EQAGGKKLLK YIRNVNFNGS AGTPVMFNKN GDAPGRYDIF |
| 481 | QYQTTNTSNP GYRLIGQWTD ELQLNIEDMQ WGKGVREIPA SVCTLPCKPG QRKKTQKGTP |
| 541 | CCWTCEPCDG YQYQFDEMTC QHCPYDQRPN ENRTGCQDIP IIKLEWHSPW AVIPVFLAML |
| 601 | GIIATIFVMA TFIRYNDTPI VRASGRELSY VLLTGIFLCY IITFLMIAKP DVAVCSFRRV |
| 661 | FLGLGMCISY AALLTKTNRI YRIFEQGKKS VTAPRLISPT SQLAITSSLI SVQLLGVFIW |
| 721 | FGVDPPNIII DYDEHKTMNP EQARGVLKCD ITDLQIICSL GYSILLMVTC TVYAIKTRGV |
| 781 | PENFNEAKPI GFTMYTTCIV WLAFIPIFFG TAQSAEKLYI QTTTLTISMN LSASVALGML |
| 841 | YMPKVYIIIF HPELNVQKRK RSFKAVVTAA TMSSRLSHKP SDRPNGEAKT ELCENVDPNS |
| 901 | EKCNCY. |

In some embodiments, the mGluR comprises mGluR8. In some embodiments, the sequence encoding an mGluR comprises a sequence encoding a human mGluR8.

In some embodiments, the sequence encoding a human mGluR8 comprises or consists of the nucleic acid sequence of the following sequence, or a functional fragment or variant thereof (UniProtKB O00222-1; GenBank Accession No. NM_001371084.1 and SEQ ID NO: 42):

| | |
|---|---|
| 1 | gcgctcggcg ctctgactgg gcgtgcggcg gcaggatttt aaagcgctct gcctcggatc |
| 61 | gtctgccctg ggtgacctcc cggacctgcc ctggtggaat ccggacttgc cccgccgaga |
| 121 | tgacgaggaa taattctgct acaaggctga tttcaaggac atgaattgtt gacctcatcc |
| 181 | caacatcaga acctcagatg ttctaatttt tgcaccattc caggcaagtt gatcttataa |
| 241 | ggaaataaaa ttgaacctta ggggtctgat ggaaattcac tgtgacattc aaatcaagaa |
| 301 | aacttgctaa tgcccacaga gccttttccc catgggccct gatggtagcc tccagaaggt |
| 361 | gcagcctcag gtggtgccct ttcttctgtg gcaagaataa actttgggtc ttggattgca |
| 421 | ataccacctg tggagaaaat ggtatgcgag ggaaagcgat cagcctcttg cccttgtttc |
| 481 | ttcctcttga ccgccaagtt ctactggatc ctcacaatga tgcaaagaac tcacagccag |
| 541 | gagtatgccc attccatacg ggtggatggg gacattattt tggggggtct cttccctgtc |
| 601 | cacgcaaagg gagagagagg ggtgccttgt ggggagctga agaaggaaaa ggggattcac |
| 661 | agactggagg ccatgcttta tgcaattgac cagattaaca aggaccctga tctcctttcc |
| 721 | aacatcactc tgggtgtccg catcctcgac acgtgctcta gggacaccta tgctttggag |
| 781 | cagtctctaa cattcgtgca ggcattaata gagaaagatg cttcggatgt gaagtgtgct |
| 841 | aatggagatc cacccatttt caccaagccc gacaagattt ctggcgtcat aggtgctgca |
| 901 | gcaagctccg tgtccatcat ggttgctaac attttaagac tttttaagat acctcaaatc |
| 961 | agctatgcat ccacagcccc agagctaagt gataacacca ggtatgactt tttctctcga |
| 1021 | gtggttccgc ctgactccta ccaagcccaa gccatggtgg acatcgtgac agcactggga |
| 1081 | tggaattatg tttcgacact ggcttctgag gggaactatg gtgagagcgg tgtggaggcc |
| 1141 | ttcacccaga tctcgaggga gattggtggt gtttgcattg ctcagtcaca gaaaatccca |
| 1201 | cgtgaaccaa gacctggaga atttgaaaaa attatcaaac gcctgctaga aacacctaat |
| 1261 | gctcgagcag tgattatgtt tgccaatgag gatgacatca ggaggatatt ggaagcagca |
| 1321 | aaaaaactaa accaaagtgg gcattttctc tggattggct cagatagttg gggatccaaa |
| 1381 | atagcacctg tctatcagca agaggagatt gcagaagggg ctgtgacaat tttgcccaaa |
| 1441 | cgagcatcaa ttgatggatt tgatcgatac tttagaagcc gaactcttgc caataatcga |
| 1501 | agaaatgtgt ggtttgcaga attctgggag gagaattttg gctgcaagtt aggatcacat |
| 1561 | gggaaaagga acagtcatat aaagaaatgc acagggctgg agcgaattgc tcgggattca |
| 1621 | tcttatgaac aggaaggaaa ggtccaattt gtaattgatg ctgtatattc catggcttac |
| 1681 | gccctgcaca atatgcacaa agatctctgc cctggataca ttggcctttg tccacgaatg |
| 1741 | agtaccattg atgggaaaga gctacttggt tatattcggg ctgtaaattt taatggcagt |
| 1801 | gctggcactc ctgtcacttt taatgaaaac ggagatgctc ctggacgtta tgatatcttc |
| 1861 | cagtatcaaa taaccaacaa aagcacagag tacaaagtca tcggccactg gaccaatcag |
| 1921 | cttcatctaa aagtggaaga catgcagtgg gctcatagag aacatactca cccggcgtct |
| 1981 | gtctgcagcc tgccgtgtaa gccaggggag aggaagaaaa cggtgaaagg ggtcccttgc |
| 2041 | tgctggcact gtgaacgctg tgaaggttac aactaccagg tggatgagct gtcctgtgaa |
| 2101 | ctttgccctc tggatcagag acccaacatg aaccgcacag gctgccagct tatccccatc |
| 2161 | atcaaattgg agtggcattc tccctgggct gtggtgcctg tgtttgttgc aatattggga |
| 2221 | atcatcgcca ccacctttgt gatcgtgacc tttgtccgct ataatgacac acctatcgtg |
| 2281 | agggcttcag gacgcgaact tagttacgtg ctcctaacgg ggatttttct ctgttattca |
| 2341 | atcacgtttt taatgattgc agcaccagat acaatcatat gctccttccg acgggtcttc |
| 2401 | ctaggacttg gcatgtgttt cagctatgca gcccttctga ccaaaacaaa ccgtatccac |
| 2461 | cgaatatttg agcaggggaa gaaatctgtc acagcgccca agttcattag tccagcatct |
| 2521 | cagctggtga tcaccttcag cctcatctcc gtccagctcc ttggagtgtt tgtctggttt |
| 2581 | gttgtggatc ccccccacat catcattgac tatggagagc agcggacact agatccagag |
| 2641 | aaggccaggg gagtgctcaa gtgtgacatt tctgatctct cactcatttg ttcacttgga |
| 2701 | tacagtatcc tcttgatggt cacttgtact gtttatgcca ttaaaacgag aggtgtccca |
| 2761 | gagactttca atgaagccaa acctattgga tttaccatgt ataccacctg catcatttgg |
| 2821 | ttagctttca tccccatctt ttttggtaca gcccagtcag cagaaaagat gtacatccag |
| 2881 | acaacaacac ttactgtctc catgagttta agtgcttcag tatctctggg catgctctat |
| 2941 | atgcccaagg tttatattat aatttttcat ccagaacaga atgttcaaaa acgcaagagg |
| 3001 | agcttcaagg ctgtggtgac agctgccacc atgcaaagca aactgatcca aaaaggaaat |
| 3061 | gacagaccaa atggcgaggt gaaaagtgaa ctctgtgaga gtcttgaaac caacacttcc |
| 3121 | tctaccaaga caacatatat cagttacagc aatcattcaa tctgaaacag ggaaatggca |
| 3181 | caatctgaag agatgtggta tatgatctta aatgatgaac atgagaccgc aaaaattcac |
| 3241 | tcctggagat ctccgtagac tacaatcaat caaatcaata gtcagtcttg taaggaacaa |
| 3301 | aaattagcca tgagccaaaa gtatcaataa acggggagtg aagaaacccg ttttatacaa |
| 3361 | taaaaccaat gagtgtcaag ctaaagtatt gcttattcat gagcagttaa aacaaatcac |
| 3421 | aaaaggaaaa ctaatgttag ctcgtgaaaa aaaatgctgt tgaaataaat aatgtctgat |
| 3481 | gttattcttg tatttttctg tgattgtgag aactcccgtt cctgtcccac attgtttaac |
| 3541 | ttgtataaga caatgagtct gtttcttgta atggctgacc agattgaagc cctgggttgt |
| 3601 | gctaaaaata aatgcaatga ttgatgcatg caatttttta tacaaataat ttatttctaa |
| 3661 | taataaagga atgttttgca aatgtt. |

In some embodiments, the human mGluR8 comprises or consists of the amino acid sequence of the following sequence, or a functional fragment or variant thereof (UniProtKB O00222-1 and SEQ ID NO: 43):

| | |
|---|---|
| 1 | MVCEGKRSAS CPCFFLLTAK FYWILTMMQR THSQEYAHSI RVDGDIILGG LFPVHAKGER |
| 61 | GVPCGELKKE KGIHRLEAML YAIDQINKDP DLLSNITLGV RILDTCSRDT YALEQSLTFV |
| 121 | QALIEKDASD VKCANGDPPI FTKPDKISGV IGAAASSVSI MVANILRLFK IPQISYASTA |
| 181 | PELSDNTRYD FFSRVVPPDS YQAQAMVDIV TALGWNYVST LASEGNYGES GVEAFTQISR |
| 241 | EIGGVCIAQS QKIPREPRPG EFEKIIKRLL ETPNARAVIM FANEDDIRRI LEAAKKLNQS |
| 301 | GHFLWIGSDS WGSKIAPVYQ QEEIAEGAVT ILPKRASIDG FDRYFRSRTL ANNRRNVWFA |
| 361 | EFWEENFGCK LGSHGKRNSH IKKCTGLERI ARDSSYEQEG KVQFVIDAVY SMAYALHNMH |
| 421 | KDLCPGYIGL CPRMSTIDGK ELLGYIRAVN FNGSAGTPVT FNENGDAPGR YDIFQYQITN |
| 481 | KSTEYKVIGH WTNQLHLKVE DMQWAHREHT HPASVCSLPC KPGERKKTVK GVPCCWHCER |
| 541 | CEGYNYQVDE LSCELCPLDQ RPNMNRTGCQ LIPIIKLEWH SPWAVVPVFV AILGIIATTF |
| 601 | VIVTFVRYND TPIVRASGRE LSYVLLTGIF LCYSITFLMI AAPDTIICSF RRVFLGLGMC |
| 661 | FSYAALLTKT NRIHRIFEQG KKSVTAPKFI SPASQLVITF SLISVQLLGV FVWFVVDPPH |
| 721 | IIIDYGEQRT LDPEKARGVL KCDISDLSLI CSLGYSILLM VTCTVYAIKT RGVPETFNEA |
| 781 | KPIGFTMYTT CIIWLAFIPI FFGTAQSAEK MYIQTTTLTV SMSLSASVSL GMLYMPKVYI |
| 841 | IIFHPEQNVQ KRKRSFKAVV TAATMQSKLI QKGNDRPNGE VKSELCESLE TNTSSTKTTY |
| 901 | ISYSNHSI . |

In some embodiments, the sequence encoding a human mGluR8 comprises or consists of the nucleic acid sequence of the following sequence, or a functional fragment or variant thereof (UniProtKB O00222-2; GenBank Accession No. NM_001371085.1 and SEQ ID NO: 44):

| | |
|---|---|
| 1 | agctcagccc cctcagccca gagatcagcc acaagtgcgg ccgctgtgct cgcctcacgc |
| 61 | ggcggcggcg gcggcggcgg cggcgctgac atggagctgc gggcccccgg cgggcttcct |
| 121 | caccgcgccc tctgcgggga gcagggaata attctgctac aaggctgatt tcaaggacat |
| 181 | gaattgttga cctcatccca acatcagaac ctcagatgtt ctaatttttg caccattcca |
| 241 | ggcaagttga tcttataagg aaataaaatt gaaccttagg ggtctgatgg aaattcactg |
| 301 | tgacattcaa atcaagaaaa cttgctaatg cccacagagc cttttcccca tgggccctga |
| 361 | tggtagcctc cagaaggtgc agcctcaggt ggtgcccttt cttctgtggc aagaataaac |
| 421 | tttgggtctt ggattgcaat accacctgtg gagaaaatgg tatgcgaggg aaagcgatca |
| 481 | gcctcttgcc cttgtttctt cctcttgacc gccaagttct actggatcct cacaatgatg |
| 541 | caaagaactc acagccagga gtatgcccat tccatacggg tggatgggga cattattttg |
| 601 | gggggtctct tccctgtcca cgcaaaggga gagagagggg tgccttgtgg ggagctgaag |
| 661 | aaggaaaagg ggattcacag actggaggcc atgctttatg caattgacca gattaacaag |
| 721 | gaccctgatc tcctttccaa catcactctg ggtgtccgca tcctcgacac gtgctctagg |
| 781 | gacacctatg ctttggagca gtctctaaca ttcgtgcagg cattaataga gaaagatgct |
| 841 | tcggatgtga agtgtgctaa tggagatcca cccattttca ccaagcccga caagatttct |
| 901 | ggcgtcatag gtgctgcagc aagctccgtg tccatcatgg ttgctaacat tttaagactt |
| 961 | tttaagatac ctcaaatcag ctatgcatcc acagccccag agctaagtga taacaccagg |
| 1021 | tatgactttt tctctcgagt ggttccgcct gactcctacc aagcccaagc catggtggac |
| 1081 | atcgtgacag cactgggatg gaattatgtt tcgacactgg cttctgaggg gaactatggt |
| 1141 | gagagcggtg tggaggcctt cacccagatc tcgagggaga ttggtggtgt ttgcattgct |
| 1201 | cagtcacaga aaatcccacg tgaaccaaga cctggagaat ttgaaaaaat tatcaaacgc |
| 1261 | ctgctagaaa cacctaatgc tcgagcagtg attatgtttg ccaatgagga tgacatcagg |
| 1321 | aggatattgg aagcagcaaa aaaactaaac caaagtgggc attttctctg gattggctca |
| 1381 | gatagttggg gatccaaaat agcacctgtc tatcagcaag aggagattgc agaaggggct |
| 1441 | gtgacaattt tgcccaaacg agcatcaatt gatggatttg atcgatactt tagaagccga |
| 1501 | actcttgcca ataatcgaag aaatgtgtgg tttgcagaat tctgggagga gaattttggc |
| 1561 | tgcaagttag gatcacatgg gaaaaggaac agtcatataa agaaatgcac agggctggag |
| 1621 | cgaattgctc gggattcatc ttatgaacag gaaggaaagg tccaatttgt aattgatgct |
| 1681 | gtatattcca tggcttacgc cctgcacaat atgcacaaag atctctgccc tggatacatt |
| 1741 | ggcctttgtc cacgaatgag taccattgat gggaaagagc tacttggtta tattcgggct |
| 1801 | gtaaatttta atggcagtgc tggcactcct gtcactttta atgaaaacgg agatgctcct |
| 1861 | ggacgttatg atatcttcca gtatcaaata accaacaaaa gcacagagta caaagtcatc |
| 1921 | ggccactgga ccaatcagct tcatctaaaa gtggaagaca tgcagtgggc tcatagagaa |
| 1981 | catactcacc cggcgtctgt ctgcagcctg ccgtgtaagc caggggagag gaagaaaacg |
| 2041 | gtgaaagggg tcccttgctg ctggcactgt gaacgctgtg aaggttacaa ctaccaggtg |
| 2101 | gatgagctgt cctgtgaact ttgccctctg gatcagagac ccaacatgaa ccgcacaggc |
| 2161 | tgccagctta tccccatcat caaattggag tggcattctc cctgggctgt ggtgcctgtg |
| 2221 | tttgttgcaa tattgggaat catcgccacc acctttgtga tcgtgacctt tgtccgctat |
| 2281 | aatgacacac ctatcgtgag ggcttcagga cgcgaactta gttacgtgct cctaacgggg |
| 2341 | atttttctct gttattcaat cacgttttta atgattgcag caccagatac aatcatatgc |
| 2401 | tccttccgac gggtcttcct aggacttggc atgtgtttca gctatgcagc ccttctgacc |
| 2461 | aaaacaaacc gtatccaccg aatatttgag caggggaaga aatctgtcac agcgcccaag |
| 2521 | ttcattagtc cagcatctca gctggtgatc accttcagcc tcatctccgt ccagctcctt |
| 2581 | ggagtgtttg tctggtttgt tgtggatccc ccccacatca tcattgacta tggagagcag |
| 2641 | cggacactag atccagagaa ggccagggga gtgctcaagt gtgacatttc tgatctctca |
| 2701 | ctcatttgtt cacttggata cagtatcctc ttgatggtca cttgtactgt ttatgccatt |
| 2761 | aaaacgagag gtgtcccaga gactttcaat gaagccaaac ctattggatt taccatgtat |
| 2821 | accacctgca tcatttggtt agctttcatc cccatctttt ttggtacagc ccagtcagca |
| 2881 | gaaaagatgt acatccagac aacaacactt actgtctcca tgagtttaag tgcttcagta |
| 2941 | tctctgggca tgctctatat gcccaaggtt tatattataa tttttcatcc agaacagaat |
| 3001 | gttcaaaaac gcaagaggag cttcaaggct gtggtgacag ctgccaccat gcaaagcaaa |
| 3061 | ctgatccaaa aaggaaatga cagaccaaat ggcgaggtga aaagtgaact ctgtgagagt |
| 3121 | cttgaaacca acagtaagtc atctgtagag tttccgatgg tcaagagcgg gagcacttcc |
| 3181 | taatagatct tcctctacca agacaacata tatcagttac agcaatcatt caatctgaaa |
| 3241 | cagggaaatg gcacaatctg aagagatgtg gtatatgatc ttaaatgatg aacatgagac |
| 3301 | cgcaaaaatt cactcctgga gatctccgta gactacaatc aatcaaatca atagtcagtc |
| 3361 | ttgtaaggaa caaaaattag ccatgagcca aaagtatcaa taaacgggga gtgaagaaac |
| 3421 | ccgttttata caataaaacc aatgagtgtc aagctaaagt attgcttatt catgagcagt |
| 3481 | taaaacaaat cacaaaagga aaactaatgt tagctcgtga aaaaaaatgc tgttgaaata |
| 3541 | aataatgtct gatgttattc ttgtattttt ctgtgattgt gagaactccc gttcctgtcc |
| 3601 | cacattgttt aacttgtata agacaatgag tctgtttctt gtaatggctg accagattga |
| 3661 | agccctgggt tgtgctaaaa ataaatgcaa tgattgatgc atgcaatttt ttatacaaat |
| 3721 | aatttatttc taataataaa ggaatgtttt gcaaatgtt. |

In some embodiments, the human mGluR8 comprises or consists of the amino acid sequence of the following sequence, or a functional fragment or variant thereof (UniProtKB O00222-2 and SEQ ID NO: 45):

| | |
|---|---|
| 1 | MVCEGKRSAS CPCFFLLTAK FYWILTMMQR THSQEYAHSI RVDGDIILGG LFPVHAKGER |
| 61 | GVPCGELKKE KGIHRLEAML YAIDQINKDP DLLSNITLGV RILDTCSRDT YALEQSLTFV |
| 121 | QALIEKDASD VKCANGDPPI FTKPDKISGV IGAAASSVSI MVANILRLFK IPQISYASTA |
| 181 | PELSDNTRYD FFSRVVPPDS YQAQAMVDIV TALGWNYVST LASEGNYGES GVEAFTQISR |
| 241 | EIGGVCIAQS QKIPREPRPG EFEKIIKRLL ETPNARAVIM FANEDDIRRI LEAAKKLNQS |
| 301 | GHFLWIGSDS WGSKIAPVYQ QEEIAEGAVT ILPKRASIDG FDRYFRSRTL ANNRRNVWFA |
| 361 | EFWEENFGCK LGSHGKRNSH IKKCTGLERI ARDSSYEQEG KVQFVIDAVY SMAYALHNMH |
| 421 | KDLCPGYIGL CPRMSTIDGK ELLGYIRAVN FNGSAGTPVT FNENGDAPGR YDIFQYQITN |
| 481 | KSTEYKVIGH WTNQLHLKVE DMQWAHREHT HPASVCSLPC KPGERKKTVK GVPCCWHCER |
| 541 | CEGYNYQVDE LSCELCPLDQ RPNMNRTGCQ LIPIIKLEWH SPWAVVPVFV AILGIIATTF |
| 601 | VIVTFVRYND TPIVRASGRE LSYVLLTGIF LCYSITFLMI AAPDTIICSF RRVFLGLGMC |
| 661 | FSYAALLTKT NRIHRIFEQG KKSVTAPKFI SPASQLVITF SLISVQLLGV FVWFVVDPPH |
| 721 | IIIDYGEQRT LDPEKARGVL KCDISDLSLI CSLGYSILLM VTCTVYAIKT RGVPETFNEA |
| 781 | KPIGFTMYTT CIIWLAFIPI FFGTAQSAEK MYIQTTTLTV SMSLSASVSL GMLYMPKVYI |
| 841 | IIFHPEONVQ KRKRSFKAVV TAATMQSKLI QKGNDRPNGE VKSELCESLE TNSKSSVEFP |
| 901 | MVKSGSTS. |

In some embodiments, the human mGluR8 comprises or consists of the amino acid sequence of the following sequence, or a functional fragment or variant thereof (UniProtKB O00222-3 and SEQ ID NO: 46):

| | |
|---|---|
| 1 | MVCEGKRSAS CPCFFLLTAK FYWILTMMQR THSQEYAHSI RVDGDIILGG LFPVHAKGER |
| 61 | GVPCGELKKE KGIHRLEAML YAIDQINKDP DLLSNITLGV RILDTCSRDT YALEQSLTFV |
| 121 | QALIEKDASD VKCANGDPPI FTKPDKISGV IGAAASSVSI MVANILRLFK IPQISYASTA |
| 181 | PELSDNTRYD FFSRVVPPDS YQAQAMVDIV TALGWNYVST LASEGNYGES GVEAFTQISR |
| 241 | EIGGVCIAQS QKIPREPRPG EFEKIIKRLL ETPNARAVIM FANEDDIRRI LEAAKKLNQS |
| 301 | GHFLWIGSDS WGSKIAPVYQ QEEIAEGAVT ILPKRASIDG FDRYFRSRTL ANNRRNVWFA |
| 361 | EFWEENFGCK LGSHGKRNSH IKKCTGLERI ARDSSYEQEG KVQFVIDAVY SMAYALHNMH |
| 421 | KDLCPGYIGL CPRMSTIDGK ELLGYIRAVN FNGCRRGIQM SLPWPTLFTP SFSSSWAVLA |
| 481 | LLSLLMKTEM LLDVMISSSI K. |

In some embodiments, the sequence encoding the mGluR comprises one or more of: (a) a nucleic acid sequence isolated or derived from a human mGluR sequence; (b) a nucleic acid sequence having at least 70% identity to a human mGluR sequence; (c) a nucleic acid sequence having at least 70% identity to the sequence of (a); and (d) a codon-optimized sequence derived from the sequence of any one of (a)-(c).

In some embodiments of the compositions of the disclosure, the mGluR comprises one or more of: (a) an amino acid sequence isolated or derived from a human mGluR sequence; (b) an amino acid sequence having at least 70% identity to a human mGluR sequence; (c) an amino acid sequence having at least 70% identity to the amino acid sequence of (a); (d) an amino acid sequence having one or more variations conserved between a human mGluR sequence and at least one non-human mammal; and (e) an amino acid sequence having one or more silent mutations when compared to the sequence of any one of (a)-(c).

In some embodiments, the mGluR comprises one or more of mGluR1, mGluR2, mGluR3, mGluR4, mGluR5, mGluR6, mGluR7, and mGluR8. In some embodiments, the mGluR comprises mGluR2. In some embodiments, the sequence encoding an mGluR comprises a sequence encoding a human mGluR2.

In some embodiments, the mGluR comprises or consists of a nucleic acid sequence having at least 70%, at least 75%, at least 80%, at least 85%, at least 90%, at least 95%, at least 97%, at least 99% or any percentage identity in between to a human mGluR. In some embodiments, the human mGluR comprises or consists of the sequence of one or more of SEQ ID NO: 2, 4, 6, 8, 10, 12, 14, 18, 20, 22, 24, 26, 30, 32, 34, 42, and 44.

In some embodiments, the mGluR comprises or consists of an amino acid sequence having at least 70%, at least 75%, at least 80%, at least 85%, at least 90%, at least 95%, at least 97%, at least 99% or any percentage identity in between to a human mGluR. In some embodiments, the human mGluR comprises or consists of the sequence of one or more of SEQ ID NO: 3, 5, 7, 9, 11, 13, 15, 17, 19, 21, 23, 25, 27, 29, 31, 33, 35, 37, 39, 41, 43, 45, and 47.

### Regulatory Elements

In some embodiments, the nucleic acid vector may further comprise one or more of a sequence comprising an enhancer, a sequence comprising an intron or any portion thereof, a sequence comprising an exon or any portion thereof, a sequence comprising a Kozak sequence, a sequence comprising a post-transcriptional response element (PRE), a sequence comprising an inverted terminal repeat (ITR) sequence, a sequence comprising a long terminal repeat (LTR) sequence, and a poly-A sequence.

In some embodiments, the nucleic acid vector further comprises a linking element that links one or more elements that are present in a vector of the disclosure and/or a polypeptide encoded by a vector of the disclosure. A linking element of the disclosure may link the sequence encoding the promoter to the sequence encoding the mGluR. Alternatively, or in addition, a linking element of the disclosure may link, reversible or irreversibly the composition to one or more of a surface, a tag, a label (detectable or sequence barcode), a ligand, an epitope, a capture probe, a selectable marker, or a delivery vehicle of the disclosure.

In some embodiments, the nucleic acid vector further comprises a cleaving element. In some embodiments, the cleaving element comprises as self-cleaving element. A cleaving element of the disclosure may be positioned between the sequence encoding the promoter to the sequence encoding the mGluR. Alternatively, or in addition, a cleaving element of the disclosure may be positioned further 5′ or 3′ to the sequence comprising the promoter and the mGluR. In some embodiments, the cleaving element may link, reversible or irreversibly, two or more sequences of the composition. In some embodiments, the cleaving element may link, reversible or irreversibly the composition to one or more of a surface, a tag, a label (detectable or sequence barcode), a ligand, an epitope, a capture probe, a selectable marker, or a delivery vehicle of the disclosure. In some embodiments, the cleaving element may link, reversible or irreversibly, two or more sequences of the composition. In some embodiments, the cleaving element may de-link or un-link one or more of a surface, a tag, a label (detectable or sequence barcode), a ligand, an epitope, a capture probe, a selectable marker, or a delivery vehicle of the disclosure by cleavage of the element. In some embodiments, the cleaving element may de-link or un-link two or more sequences of the composition. In some embodiments, the cleavable element comprises a nucleic acid sequence and the nucleic acid sequence may encode a multicistronic element. In some embodiments, the cleavable element comprises a self-cleaving element. In some embodiments, the cleavable element comprises a sequence encoding a self-cleaving peptide.

In some embodiments, the nucleic acid vector further comprises a multicistronic element. In some embodiments, the multicistronic element comprises an IRES sequence.

### Expression and Delivery Vectors

The disclosure also provides expression and delivery vectors comprising the nucleic acid vectors described herein. Expression vectors include, but are not limited to, any vector suitable for in vitro or ex vivo delivery of a composition of the disclosure to a cell of the disclosure, by any means. In some embodiments, an expression vector comprises a plasmid. In some embodiments, the plasmid is electroporated into a cell of the disclosure. Expression vectors of the disclosure may also comprise delivery vectors of the disclosure when used to introduce a composition in vitro or ex vivo.

Delivery vectors include, but are not limited to, any vector suitable for in vivo delivery of a composition of the disclosure to a cell of the disclosure when in vivo or in situ (in the context of an intact eye). Delivery vectors of the disclosure include, but are not limited, to viral vectors and non-viral vectors. Exemplary viral vectors include, but are not limited to, adeno-associated vectors of any serotype. Exemplary non-viral vectors include, but are not limited to, lipid vectors, polymer vectors and particle vectors. Lipid vectors include, but are not limited to, liposomes, lipid nanoparticles, micelles, lipid polymersomes, and exosomes. Polymer vectors include, but are not limited to, polymersomes, lipid nanoparticles, and nanoparticles. Particle vectors include, but are not limited to, nanoparticles of all geometries and compositions.

In some embodiments, a delivery vector of the disclosure comprises a composition of the disclosure, such as nucleic acid vector comprising a CBh promoter operably linked to a sequence encoding a GPCR, such as an mGluR. In some embodiments of the delivery vectors of the disclosure, the vector is a viral vector. In some embodiments, the viral vector is an adeno-associated vector (AAV). In some embodiments, the AAV is a recombinant AAV (rAAV). In some embodiments, the rAAV comprises a sequence isolated or derived from an AAV of a first serotype and a sequence isolated or derived from an AAV of a second serotype. In some embodiments, the rAAV comprises a capsid sequence isolated or derived from an AAV of a first serotype and a capsid insert sequence isolated or derived from an AAV of a second serotype. In some embodiments, the heterologous capsid insert sequence is neither isolated nor derived from an AAV of any known serotype. In some embodiments, the heterologous capsid insert sequence comprises a random sequence.

Exemplary AAV serotypes include, but are not limited to, AAV1, AAV2, AAV3, AAV4, AAV5, AAV6, AAV7, AAV8, AAV9, and any combination thereof. In some embodiments, an AAV vector of the disclosure comprises a sequence isolated or derived from one or more of AAV2, AAV4, AAV5 and AAV8. In some embodiments, an AAV vector of the disclosure comprises a wild type sequence from one or more of AAV1, AAV2, AAV3, AAV4, AAV5, AAV6, AAV7, AAV8 and AAV9. In some embodiments, an AAV vector of the disclosure comprises a capsid sequence isolated or derived from one or more of AAV1, AAV2, AAV3, AAV4, AAV5, AAV6, AAV7, AAV8 and AAV9. In some embodiments, an AAV vector of the disclosure comprises a capsid sequence isolated or derived from AAV2 and AAV4. In some embodiments, an AAV vector of the disclosure comprises a capsid sequence isolated or derived from AAV2 and AAV5. In some embodiments, an AAV vector of the disclosure comprises a capsid sequence isolated or derived from AAV2 and AAV8. In some embodiments, an AAV vector of the disclosure comprises a recombinant or chimeric capsid sequence comprising two or more sequences isolated or derived from AAV1, AAV2, AAV3, AAV4, AAV5, AAV6, AAV7, AAV8 and AAV9.

In certain specific embodiments of the present disclosure, modified adeno-associated vectors (AAV) are used as described in WO2018/022905 and/or WO2021243085A2, the contents of which are incorporated herein by reference in their entireties.

In certain specific embodiments of the present disclosure, modified adeno-associated vectors (AAV) are used as described in any of WO 2012/145601, WO 2018/022905, WO 2019/006182 and/or WO2021243085A2, the contents of which are incorporated herein by reference in their entireties. As certain non-limiting examples, in some cases, a modified AAV comprises a variant AAV capsid protein comprising an insertion of a peptide in the GH loop of the capsid protein, e.g., where the insertion site is within amino acids 570-611 (e.g., between amino acids 587 and 588) of an AAV2 capsid protein, or a corresponding site in another AAV serotype. In some cases, the peptide inserted into the GH loop of the capsid protein comprises the amino acid sequence LGETTRP (SEQ ID NO: 50). As another example, in some cases, a peptide inserted into the GH loop of an AAV capsid protein comprises an amino acid sequence selected from the group consisting of LATTSQNKPA (SEQ ID NO: 51), LAVDGAQRSA (SEQ ID NO: 52), LAKSDQSKPA (SEQ ID NO: 53) and LAANQPSKPA (SEQ ID NO: 54) as described in WO2018/022905.

As another example, in some cases, a peptide inserted into the GH loop of an AAV capsid protein comprises an amino acid sequence selected from the group consisting of LAHQDTTKNS (SEQ ID NO: 55), LAHQDSTKNA (SEQ ID NO: 56), LAHQDATKNA (SEQ ID NO: 57), LALSEATRPA (SEQ ID NO: 58), LAKDETKNSA (SEQ ID NO: 59), LQRGNRQTTTADVNTQ (SEQ ID NO: 60), LQRGNRQATTEDVNTQ (SEQ ID NO: 61), SRTNTPSGTTTQPTLQFSQ (SEQ ID NO: 62) and SKTDTPSGTTTQSRLQFSQ (SEQ ID NO: 63), as described in WO2021243085A2.

In some embodiments, delivery vectors, including AAV vectors, target a retinal cell type. In some embodiments, delivery vectors, including AAV vectors, have a tropism for a retinal cell type. In some embodiments, the retinal cell type is a neuron. In some embodiments, the retinal cell type is a retinal ganglion cell. In some embodiments, the retinal cell type is a horizontal cell. In some embodiments, the retinal cell type is an amacrine cell. In some embodiments, the retinal cell type is a bipolar cell. In some embodiments, the retinal cell type is a photoreceptor cell. In some embodiments, the retinal cell type is not a photoreceptor. Photoreceptor cells include rod cells and cone cells.

In some embodiments, the term “targeting” is meant to describe a specific and/or selective binding to the retinal cell type resulting in higher expression of the composition of the disclosure in that retinal cell type than in any other retinal cell type or non-retinal cell type.

In some embodiments, the cell is a retinal neuron or a progenitor cell thereof. In some embodiments, the progenitor cell is a neural fold cell, an early retinal progenitor cell (RPC), a late RPC, an embryonic stem cell (ESC), an induced pluripotent stem cell (iPSC), or a retinal pigmented epithelial (RPE) cell. In some embodiments, ESCs of the disclosure are neither isolated nor derived from a human embryo or human tissue.

In some embodiments, a composition of the disclosure may be delivered to a differentiated cell and/or a progenitor cell capable of becoming the differentiated cell type.

Therapeutic Indications

Compositions, vectors, cells and pharmaceutical compositions of the disclosure may be administered as a monotherapy. Alternatively, compositions, vectors, cells and pharmaceutical compositions of the disclosure may be administered as combination therapies.

Compositions, vectors, cells and pharmaceutical compositions of the disclosure may be used for the manufacture of a medicament to treat or may be used in a method for the treatment of a disease or disorder. In some embodiments, the disease or disorder is an ocular disease or disorder. In some embodiments, the disease or disorder is a retinal disease or disorder.

Retinal diseases or disorders may be congenital, degenerative or traumatic. Compositions, vectors, cells and pharmaceutical compositions of the disclosure may be used to restore cellular function or activity to any retinal neuron of an intact or diseased retina. In some embodiments, compositions, vectors, cells and pharmaceutical compositions of the disclosure may be used to restore vision to a subject by inducing a new function or activity to any retinal neuron of an intact or diseased retina to compensate for a missing or lost function or activity in any retinal neuron.

In some embodiments, methods are provided for the enhancement and/or restoration of vision in a subject comprising administering a vector of the present disclosure to a subject in need thereof in order to drive the expression of a fusion polypeptide comprising an affinity tag (e.g., a SNAP tag sequence) and a GPCR (e.g., mGluR2) in the retinal cells of the subject. In related embodiments, in addition to the administration of a vector of the disclosure, a photoswitch conjugate is also administered to the subject before, concurrent with or after administration of the vector. Exemplary photoswitch conjugates for use in such methods can be found described, for example, in WO2019/060785 and WO2021/243086, the contents of which are incorporated herein by reference in their entireties. For example, in some specific embodiments, a vector administered to a subject comprises a CBh promoter operably linked to a SNAP-mGluR2 fusion polypeptide as described herein. In addition, a photoswitch conjugate administered to the subject is a BGAG conjugate. In more particular embodiments, the BGAG conjugate comprises benzylguanine, azobenzene and at least one glutamate ligand. In even more particular embodiments, the BGAG conjugate is a branched BGAG molecule, such as 4X-BGAG (as described in WO2021/243086) or 9X-BGAG (as described in U.S. Provisional Application No. 63/283,022, the content of which is incorporated herein by reference in its entirety).

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the disclosure have been illustrated and described, various other changes and modifications can be made without departing from the spirit and scope of the disclosure. The scope of the appended claims includes all such changes and modifications that are within the scope of this disclosure.

### EXAMPLES

### Example 1: Expression Analysis of CBh Promoters in Non-Human Primates (NHP)

Cynomolgus macaques and African Green monkeys between 3-10 years of age were used. Bilateral intravitreal injections were performed using a 30 g needle to deliver 5.0E+11 viral genomes per eye in a 50 μL volume.

Onset and progression of GFP expression is monitored weekly by confocal scanning laser ophthalmoscopy (cSLO) imaging using the autofluorescence function of the Heidelberg Spectralis HRA/OCT system.

Six to eight weeks after intravitreal injection the primates were euthanized and both eyes (whole globes) were carefully harvested. After enucleation, excess orbital tissue was carefully trimmed and removed. A small (5 mm) slit was made ˜2 mm from the limbus and the whole eye was placed in a vial containing 4% paraformaldehyde (PFA) and incubated at 4° C. overnight. After overnight fixation, the PFA was decanted and replaced with phosphate buffered saline (PBS). The whole eye was dissected to remove the anterior structures (cornea, lens, and ciliary body) and then 4 cuts were made to the posterior eye to enable the tissue to lie nearly flat. A fluorescent dissection microscope was used to visualize GFP expression in the entire retina, by direct fluorescence upon filtered UV excitation. The retinal tissue was then dissected into central and peripheral pieces, separated from the underlying tissues, additionally rinsed in PBS, embedded in agarose, sectioned, mounted on microscope slides, and examined by laser-scanning confocal microscopy. After sectioning 4′,6-diamidino-2-phenylindole (DAPI) was used to label cell nuclei. GFP expression is detected by direct fluorescence. Images are acquired at different magnifications to evaluate transduction in the different cell layers.

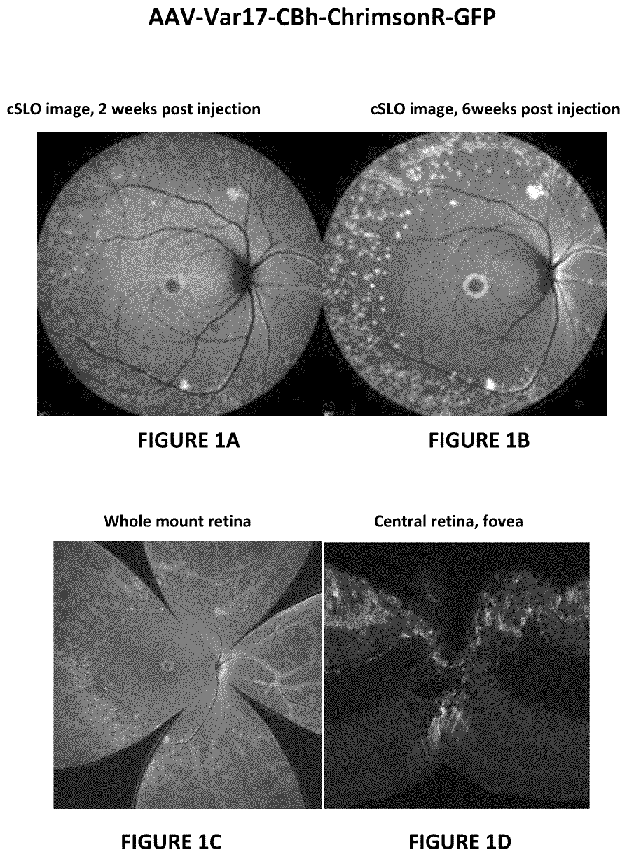

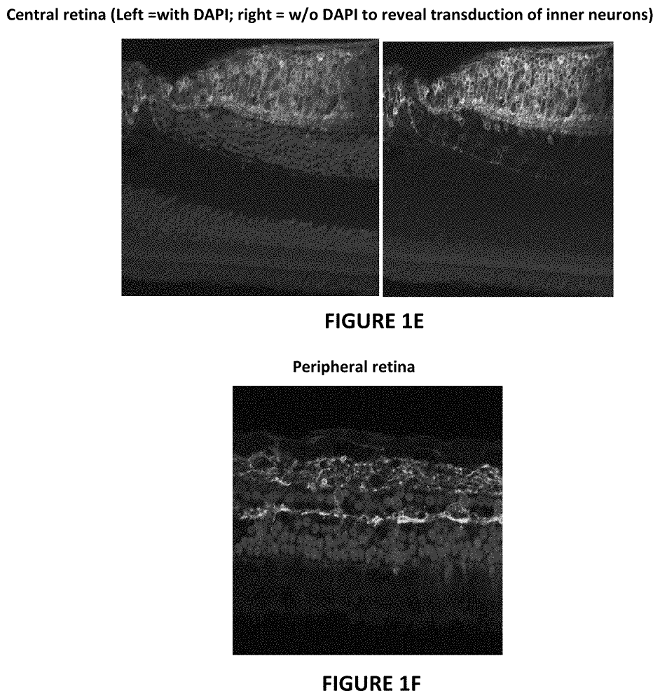

For the construct AAV-Var17-CBh-ChrimsonR-GFP, containing a CBh promoter of SEQ ID NO: 1, the following results were obtained. FIGS. 1A and 1B provides cSLO images taken the Heidelberg Spectralis HRA/OCT 2 weeks (A) and 6 weeks (B) following intravitreal injection of 5.0E+11 vg of rAAV. FIG. 1C shows the extent of GFP expression in central and peripheral retina surface, by direct fluorescence imaging. FIGS. 1D-1F provides confocal images obtained from 100 um retinal section showing robust transduction of RGCs, inner neurons, Muller cells and foveal cones (1D-1E); 100 um retinal section showing transduction of RGCs, inner neurons and photoreceptors, in the peripheral retina (1F).

### Example 2

### The CBh Promoter Drives Robust Expression of a GPCR Protein in the Retinal Cells of Non-Human Primates

A. Vectors:

The following vectors were used in rd1 mice and non-human primates:
AAV-Var17-CBh-ChrimsonR-GFP
AAV-Var17-CBh-SNAP-human mGluR2
AAV-Var17-Syn1-ChrimsonR-GFP
AAV-Var17-Syn1-SNAP-human mGluR2

B. Expression Analyses in NHP Retinas:

Cynomolgus macaques and African Green monkeys between 3-10 years of age were used. Bilateral intravitreal injections of the vectors described above were performed using a 30 g needle to deliver 3.0E+11 to 5.0E+11 viral genomes per eye in a 50 μL volume. Onset and progression of GFP expression was monitored weekly by confocal scanning laser ophthalmoscopy (cSLO) imaging using the autofluorescence function of the Heidelberg Spectralis HRA/OCT system.

Six to eight weeks after intravitreal injection the primates were euthanized and both eyes (whole globes) were carefully harvested. After enucleation, excess orbital tissue was carefully trimmed and removed. A small (5 mm) slit was made ˜2 mm from the limbus and the whole eye was placed in a vial containing 4% paraformaldehyde (PFA) and incubated at 4° C. overnight. After overnight fixation, the PFA was decanted and replaced with phosphate buffered saline (PBS). The whole eye was dissected to remove the anterior structures (cornea, lens, and ciliary body) and then 4 cuts were made to the posterior eye to enable the tissue to lie nearly flat.

A fluorescent dissection microscope was used to visualize GFP expression in the entire retina, by direct fluorescence upon filtered UV excitation. The retinal tissue was then dissected into central and peripheral pieces, separated from the underlying tissues, additionally rinsed in PBS, embedded in agarose, sectioned, mounted on microscope slides, and examined by laser-scanning confocal microscopy. After sectioning, 4′,6-diamidino-2-phenylindole (DAPI) was used to label cell nuclei. GFP expression was detected by direct fluorescence. Images were acquired at different magnifications to evaluate transduction in the different cell layers.

SNAP immunostaining was carried out to visualize SNAP-mGluR2 expression on agarose section. Sections were blocked and permeabilized with Triton-X overnight, incubated with the primary anti-SNAP antibody, rinsed in PBS, incubated with an Alexa-488 conjugated secondary antibody, rinsed, then counterstained with DAPI. Sections were mounted on microscope slides and examined by laser-scanning confocal microscopy.

C. Expression Analyses in Rd1 Mouse Retinas:

Five to seven weeks old rd1 mice were used, which represent a mouse model of blindness due to retinal photoreceptor degeneration. Bilateral intravitreal injections were performed using a 30 g needle to deliver 1.5E+10 viral genomes per eye in a 1.5 μL volume. Six to eight weeks after intravitreal injection of the vectors described above the mice were euthanized and both eyes (whole globes) were carefully harvested. After enucleation, excess orbital tissue was carefully trimmed and removed. A small (5 mm) slit was made ˜2 mm from the limbus and the whole eye was placed in a vial containing 4% paraformaldehyde (PFA) and incubated at 4° C. overnight. After overnight fixation, the PFA was decanted and replaced with phosphate buffered saline (PBS).

The whole eye was dissected to remove the anterior structures (cornea, lens, and ciliary body). For some eyes, the retina was gently detached from posterior eyecup and then 4 cuts were made to enable the tissue to lie nearly flat (“flat-mount”). For some eyes, the posterior eyecup was placed in a 30% sucrose solution overnight to cryoprotect the tissue before embedding in Optimal Cutting Temperature (OCT) medium, freezing and cryosectioning (16 μm cryosections).

A fluorescent dissection microscope was used to visualize GFP expression by direct fluorescence upon filtered UV excitation on retinal flat-mount and cryosections, or SNAP immunostaining was done on retinal flat-mount to visualize SNAP-mGluR2 expression. Retinas were blocked and permeabilized with Triton-X overnight, incubated with the primary anti-SNAP antibody, rinsed in PBS, incubated with an Alexa-488 conjugated secondary antibody, rinsed, then counterstained with DAPI. Sections were mounted on microscope slides and examined by laser-scanning confocal microscopy.

D. Discussion

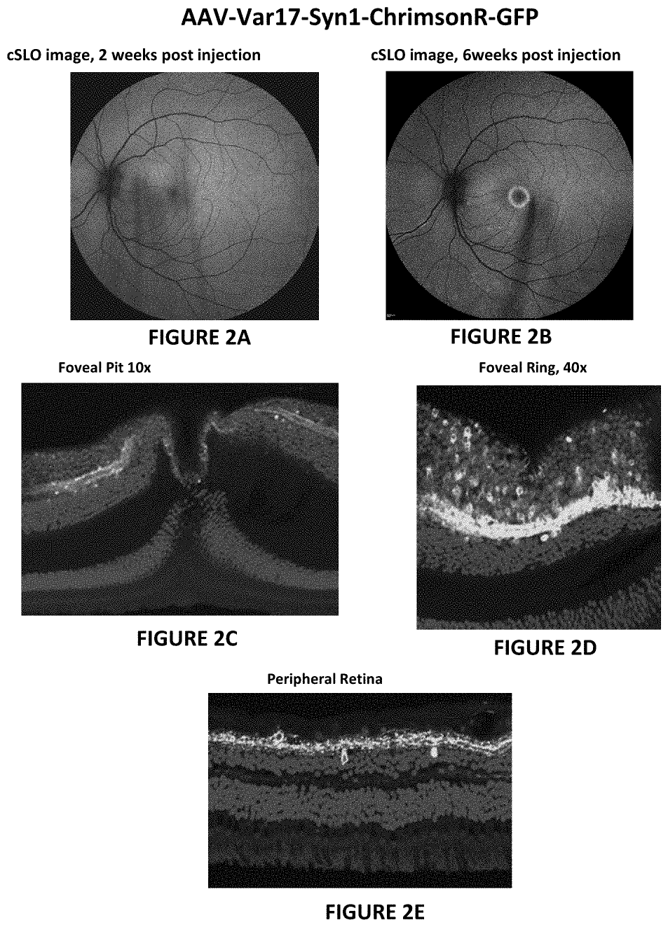

The results of these studies are shown in FIGS. 2-8 and summarized below.

When the vector AAV-Var17-CBh-ChrimsonR-GFP was used in non-human primates, the results shown in FIGS. 1A-1D were obtained. FIGS. 1A-1D provide confocal images obtained from 100 um retinal sections showing robust transduction of RGCs, inner neurons, Muller cells and foveal cones (A-C); and obtained from 100 um retinal sections showing transduction of RGCs, inner neurons and photoreceptors, in the peripheral retina (D).

When the vector AAV-Var17-Syn1-ChrimsonR-GFP was used in non-human primates, the results of FIGS. 2A-2E were obtained. FIGS. 2A and 2B provide cSLO images taken the Heidelberg Spectralis HRA/OCT 2 weeks (A) and 6 weeks (B) following intravitreal injection of 5.0E+11 vg of AAV-Var17-Syn1-ChrimsonR-GFP in non-human primates in non-human primates. FIGS. 2C-2D provide confocal images (10×, 40×) obtained from 100 um retinal sections showing limited and specific transduction of RGCs. FIG. 2E shows a 100 um retinal section with limited and specific transduction of RGCs in the peripheral retina.

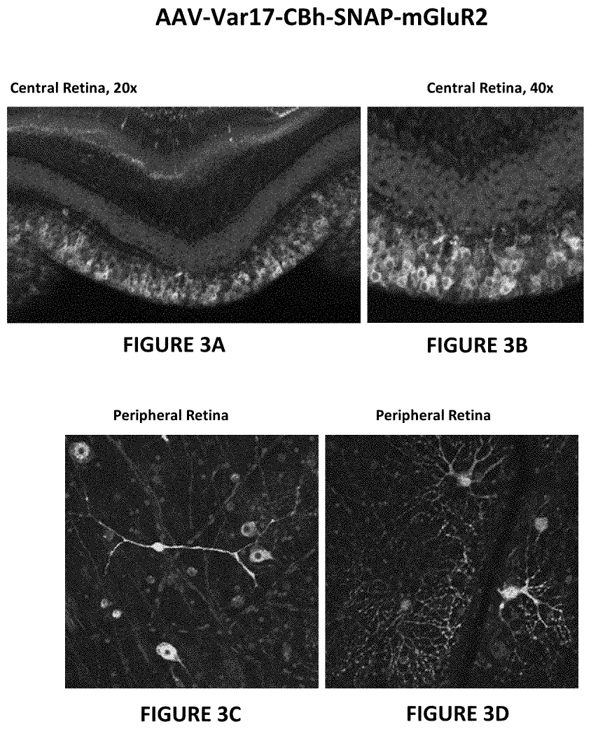

When the vector AAV-Var17-CBh-SNAP-mGluR2 was used in non-human primates, the results shown in FIGS. 3A-3C were obtained. FIGS. 3A and B provide confocal images (20× and 40×) obtained from 100 um retinal sections in the central retina showing robust transduction of RGCs. A few SNAP-positive photoreceptors were observed. FIGS. 3C and 3D provide confocal images obtained from flat-mounts of the peripheral retina showing expression in RGCs (indicated by visible dendritic trees).

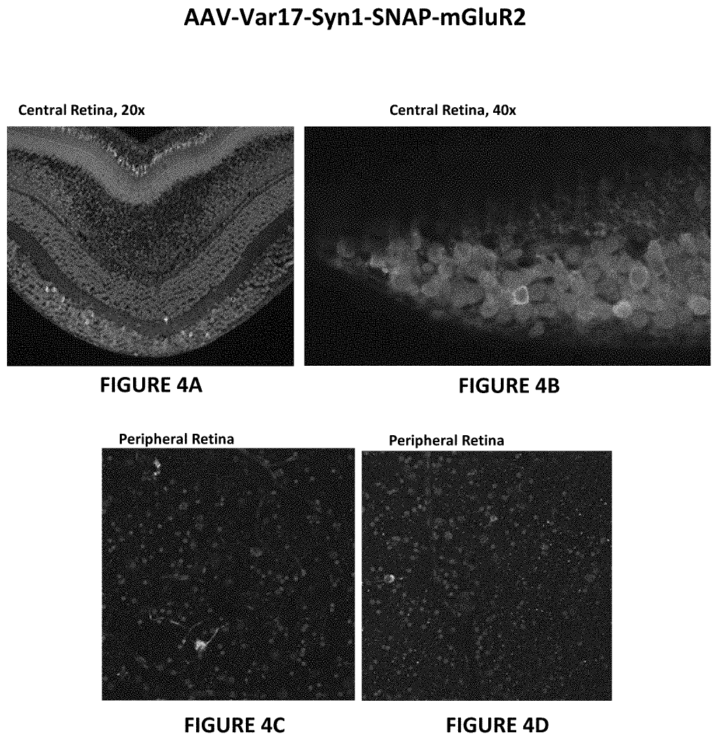

When the vector AAV-Var17-Syn1-SNAP-mGluR2 was used in non-human primates, the results shown in FIGS. 4A-4C were obtained. FIGS. 4A and B provide confocal images (20× and 40×) obtained from 100 um retinal sections in the central retina showing limited transduction of RGCs when transgene expression was driven by the neuron specific Syn1 promoter. Non-specific signal was seen in photoreceptors outer segments. FIGS. 4C and 4D provide confocal images obtained from flat-mounts of the peripheral retina showing only rare cells were found to be SNAP positive.

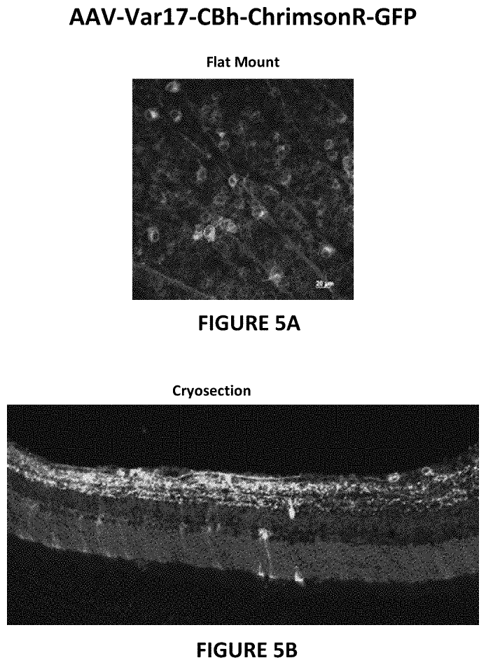

When the vector AAV-Var17-CBh-ChrimsonR-GFP was used in rd1 mice, the results shown in FIGS. 5A-5B were obtained. FIG. 5A shows 40×image of retinal flat-mounts showing ChrimsonR-GFP expression in RGCs. FIG. 5B provides confocal images (20×) obtained from 16 um cryosections showing ChrimsonR-GFP expression in RGCs and some Muller cells.

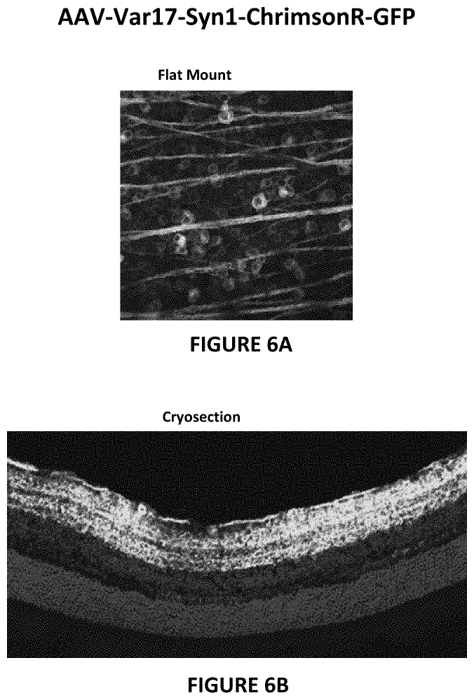

When the vector AAV-Var17-Syn1-ChrimsonR-GFP was used in rd1 mice, the results shown in FIGS. 6A-6B were obtained. FIG. 6A is a 40×image of retinal flat-mount showing strong ChrimsonR-GFP expression in RGCs and their axons. FIG. 6B is a 20×image obtained from a 16 um cryosection showing robust and specific expression of ChrimsonR-GFP in RGCs.

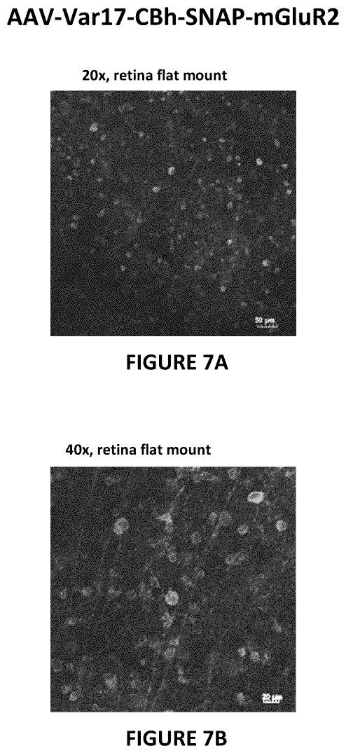

When the vector AAV-Var17-CBh-SNAP-mGluR2 was used in rd1 mice, the results shown in FIGS. 7A-7B were obtained. FIGS. 7A and 7B are 20× and 40×images of retinal flat-mount showing weak SNAP-positive expression in a limited number of RGCs.

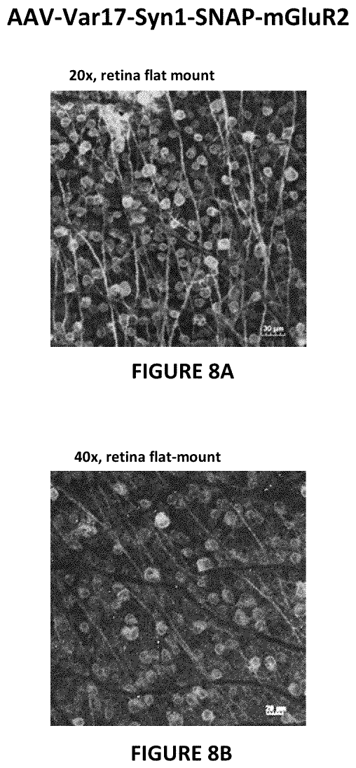

When the vector AAV-Var17-Syn1-SNAP-mGluR2 was used in rd1 mice, the results shown in FIGS. 8A-8B were obtained. FIGS. 8A and 8B show 20× and 40×images of retinal flat-mounts showing a significant higher numbers of SNAP-positive RGCs than with the CBh promoter.

Based on the observation that the synapsin promoter drove much higher levels of expression of the SNAP-mGluR2 transgene in rd1 mice than did the CBh promoter, a similar pattern was expected in the more clinically relevant model system of non-human primates. However, this did not hold true. Instead, unexpectedly, in non-human primates, the CBh promoter drove much more robust expression of a SNAP-mGluR2 transgene in the particular cell types of therapeutic interest in both the central and peripheral retina. These findings make the CBh promoter a highly desirable and advantageous regulatory sequence for driving expression of a heterologous sequence encoding a GPCR (e.g., a SNAP-mGluR2 fusion protein) for addressing ocular disorders and developing vision restoration strategies in humans.

1. A nucleic acid vector comprising a CBh promoter sequence operably linked to a heterologous sequence encoding a G-protein coupled receptor (GPCR), wherein the CBh promoter comprises: (i) a cytomegalovirus (CMV) enhancer sequence and (ii) a chicken beta actin (CBA) promoter sequence.

2. (canceled)

3. The nucleic acid vector of claim 1, wherein the CBh promoter further comprises an intron sequence selected the group consisting of (i) CBA intron sequence and (ii) a Mirabilis mosaic virus (MMV) intron sequence.

4. The nucleic acid vector of claim 1, wherein the CBh promoter comprises the sequence of SEQ ID NO: 1 or a functional fragment or variant thereof having at least 90% identity thereto, where the functional fragment or variant is capable of directing expression of the heterologous sequence in the retina.

5. The nucleic acid vector of claim 1, wherein the CBh promoter comprises the sequence of SEQ ID NO: 1.

6. The nucleic acid vector of claim 1, wherein the heterologous sequence further comprises a sequence encoding an affinity tag.

7. The nucleic acid vector of claim 6, wherein the affinity tag comprises a SNAP polypeptide.

8. The nucleic acid vector of claim 7, wherein the SNAP polypeptide comprises the sequence of SEQ ID NO: 47 or SEQ ID NO: 48 or a functional fragment or variant thereof having at least 90% identity thereto.

9. The nucleic acid vector of claim 1, wherein the heterologous sequence encodes a fusion protein comprising the affinity tag and the GPCR.

10. The nucleic acid vector of claim 9, wherein the fusion protein comprises, from amino (N) to carboxy (C) ends, the SNAP sequence and the GPCR sequence.

11. The nucleic acid vector of claim 10, wherein the GPCR is an inhibitory G-protein (Gᵢ)-coupled GPCR.

12. The nucleic acid vector of claim 11, wherein the GPCR is a stimulatory G-protein (Gₛ)-coupled GPCR.

13. The nucleic acid vector of claim 12, wherein the GPCR comprises a metabotropic glutamate receptor (mGluR).

14. The nucleic acid vector of claim 13, wherein the mGluR comprises one or more of mGluR1, mGluR2, mGluR3, mGluR4, mGluR5, mGluR6, mGluR7, and mGluR8, or a functional fragment or variant thereof.

15. The nucleic acid vector of claim 13, wherein the mGluR comprises mGluR2, or a functional fragment or variant thereof.

16. The nucleic acid vector of claim 13, wherein the mGluR comprises mGluR2 and wherein the mGluR2 comprises the amino acid sequence of SEQ ID NO: 9.

17. A delivery vector comprising a nucleic acid vector of claim 1.

18. The delivery vector of claim 17, wherein the vector is a viral vector.

19. The delivery vector of claim 17, wherein the vector is an adeno-associated vector (AAV).

20. The delivery vector of claim 17, wherein the delivery vector targets a retinal cell type.

21. A cell comprising a nucleic acid vector of claim 1 or a delivery vector of claim 17.

22. A pharmaceutical composition comprising a pharmaceutically acceptable carrier and: (i) a nucleic acid vector of claim 1, (ii) a delivery vector of claim 17, or (iii) a cell of claim 21.

23. A method of treating an ocular disease or disorder, comprising administering to a subject in need thereof, a therapeutically effective amount of a pharmaceutical composition of claim 22.

24. The method of claim 23, wherein the disease or disorder is a retinal disease or disorder.

25. The method of claim 23, wherein the retinal disease or disorder is associated with a decrease or an inhibition of a function of one or more retinal neurons.

26. The method of claim 23, wherein the subject has experienced or is at risk of experiencing loss of visual acuity.

27. The method of claim 23, wherein the administering comprises an intraocular route, an intravitreal route or a subretinal route.

28. The method of claim 23, wherein the administering comprises an injection, infusion, engraftment or implantation.
